(19) United States
(12) Patent Application Publication
(10) Pub. No.: US 2026/0248923 A1
(43) Pub. Date: Aug. 27, 2026

(54) BI-SPECIFIC ADAPTERS AND THEIR USE WITH UNIVERSAL CAR-T CELLS IN THE TREATMENT OF TUMORS AND THE INHIBITION OF CANCER-ASSOCIATED FIBROBLASTS

(71) Applicant: Purdue Research Foundation, West Lafayette, IN (US)

(72) Inventors: Philip Low, West Lafayette, IN (US); Haiyan A. Chu, Seattle, WA (US); Bo Huang, Somerset, NJ (US); Sudarsan Kasireddy, West Lafayette, IN (US); Yashapal Singh, Austin, TX (US); Suilan Zheng, West Lafayette, IN (US); Madduri Srinivasarao, West Lafayette, IN (US); Ramesh Mukkamala, West Lafayette, IN (US)

(21) Appl. No.: 19/164,176

(22) PCT Filed: Mar. 11, 2024
(86) PCT No.: PCT/US2024/019321

§ 371 Date: Sep. 10, 2025

Related U.S. Application Data
(60) Provisional application No. 63/451,448, filed on Mar. 10, 2023.
(60) Provisional application No. 63/451,453, filed on Mar. 10, 2023.
(60) Provisional application No. 63/451,457, filed on Mar. 10, 2023.
(60) Provisional application No. 63/451,462, filed on Mar. 10, 2023.
(60) Provisional application No. 63/595,456, filed on Nov. 2, 2023.
(60) Provisional application No. 63/600,612, filed on Nov. 17, 2023.
(60) Provisional application No. 63/600,616, filed on Nov. 17, 2023.

(51) Int. Cl. A61K 47/54; A61K 35/17; A61K 38/00; A61K 40/11; A61K 40/31; A61K 40/40; A61K 40/42; A61P 35/00; C07K 16/44; C12N 5/0783; C12N 9/64

(52) U.S. Cl. CPC A61K 47/545; A61K 35/17; A61K 40/11; A61K 40/31; A61K 40/4276; A61K 40/429; A61P 35/00; C07K 16/44; C12N 5/0636; C12N 9/6408; A61K 38/00; A61K 2239/13; A61K 2239/49; C07K 2317/622; C12N 2510/00

(57) ABSTRACT

Chimeric antigen receptor (CAR)-T cells, bispecific adapters that link the CAR-T cells to tumor cells and/or cancer-associated fibroblasts (CAFs), and methods of treating cancer using same.

### PRIORITY

This patent application is related to and claims the priority benefit of U.S. Provisional Patent Application No. 63/451,448 filed Mar. 10, 2023, U.S. Provisional Patent Application No. 63/595,456 filed Nov. 2, 2023, U.S. Provisional Patent Application No. 63/451,453 filed Mar. 10, 2023, U.S. Provisional Patent Application No. 63/600,612 filed Nov. 17, 2023, U.S. Provisional Patent Application No. 63/451,457 filed Mar. 10, 2023. U.S. Provisional Patent Application No. 63/600,616 filed Nov. 17, 2023, and U.S. Provisional Patent Application No. 63/451,462 filed Mar. 10, 2023. The contents of each of the foregoing applications are hereby incorporated by reference in their entireties into this disclosure.

### TECHNICAL FIELD

The present disclosure relates to chimeric antigen receptor (CAR) T-cells and bi-specific adapters that can link the CAR-T cells to tumors expressing prostate-specific membrane antigen (PSMA) and/or folate receptors (FR) or cancer-associated fibroblasts (CAFs) expressing fibroblast activation protein (FAP), and combinations of bispecific adapters that link the CAR-T cells to PSMA- or FR-expressing tumor cells and, optionally, FAP-expressing CAFs, and methods of treating cancer using same.

### BACKGROUND

Chimeric antigen receptor (CAR)-T cell therapy has proven effective in hematological cancer but has limited efficacy in solid tumors due, at least in part, to limited CAR-T cell infiltration. This CAR-T cell infiltration is possibly due to cancer-associated fibroblasts (CAFs), which are found in most solid tumors and have been proven to remodel the extracellular matrix, secrete immunosuppressive cytokines, promote tumor cell proliferation by growth factor secretion, promote tumor invasion, inhibit the immune response, and form a physical barrier to prevent T-cell infiltration.

Fibroblast activation protein (FAP) is expressed on the surfaces of CAFs and has been proven to be correlated with poor patient prognosis in multiple solid tumors. In addition, virtually every human solid cancer over-expresses FAP.

Traditionally, a T-cell expressing a single CAR has been used to target a cell-surface receptor on a tumor cell. When the CAR on the T-cell binds the cell-surface receptor on the tumor cell, the T-cell can kill the tumor cell to which it is bound. While effective, this approach can be quite costly, given that CAR-T cells must be produced for different cancers that express different cell-surface receptors.

Despite the clear need for the prevention and treatment of cancer, it remains a significant cause of death and suffering worldwide because no effective therapeutic options presently exist that can cure the condition. Further, where drugs or other therapies are available, such treatments typically employ highly potent drugs that risk systemic toxicity in the underlying subject as they are poorly selective for the cancer cells of interest.

What is needed are bispecific adapters that can improve the immune response effected by CAR-T cells bound to tumor cells and inhibit the effect of CAFs. This and other objects and advantages, as well as inventive features, will be apparent from the detailed description provided herein.

### SUMMARY

Provided is a bispecific adapter for use with anti-fluorescein chimeric antigen receptor (CAR)-T cells in the treatment of fibroblast activation protein (FAP)-expressing (e.g., FAPα-expressing) and/or prostate-specific membrane antigen (PSMA) cancer and, optionally, for use in combination with cancer-associated fibroblasts (CAFs) expressing fibroblast activation protein (FAP). In certain embodiments, the bispecific adapter comprises the following structure:

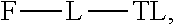

wherein F comprises a fluorescein, L comprises a linker, and TL comprises a targeting ligand. The fluorescein can comprise fluorescein, fluorescein isothiocyanate (FITC), or N-hydroxysuccinimide (NHS)-fluorescein. The targeting ligand can comprise a radical of a FAP ligand comprising a structure of:

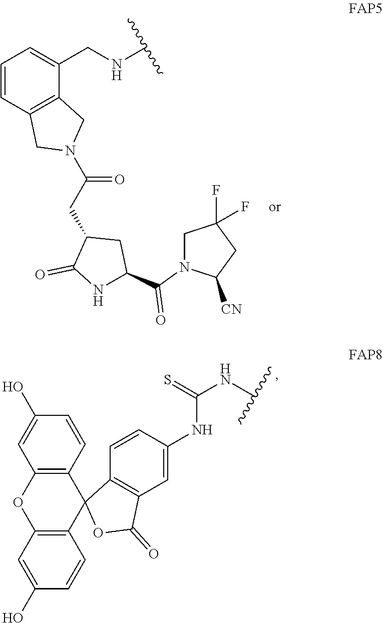

wherein

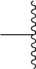

is the point of attachment to the linker.

Also provided is a bispecific adapter for use with anti-fluorescein (e.g., fluorescein, FITC, or NHS-fluorescein) chimeric antigen receptor (CAR)-T cells in the treatment of fibroblast activation protein (FAP)-expressing (e.g., FAPa-expressing) cancer and/or tumors expressing prostate-specific membrane antigen (PSMA) tumors.

In certain embodiments, the adapter can comprise fluorescein-linker-FAP ligand (e.g., fluorescein=fluorescein, FITC, or NHS-fluorescein), wherein the FAP ligand is:

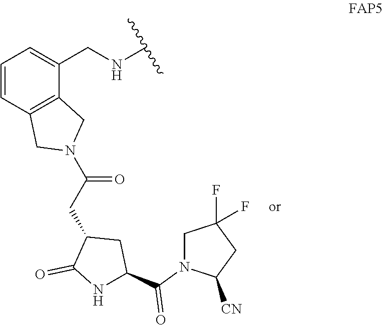 [SMILES: *NCc1cccc2c1CN(C(=O)C[C@@H]1C[C@@H](C(=O)N3CC(F)(F)C[C@H]3C#N)NC1=O)C2]

and the linker comprises (or consists essentially of or consists of) polyethylene glycol (PEG), and wherein the adapter can be a pharmaceutically acceptable salt or hydrate thereof. The linker can comprise (or consist essentially of or consist of) PEG₄ to PEG₁₆. The linker can comprise (or consist essentially of or consist of) PEG₆. The linker can comprise (or consist essentially of or consist of) PEG₁₆. The FAP ligand can have a structure represented by the formula I-B:

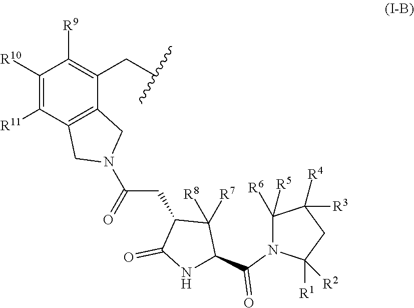

T is substituted or unsubstituted methylene (—CH₂—), substituted or unsubstituted amino (—NH—), —O—, or —S—;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F and 5-tetrazolyl;
R³ and R⁴ are each independently selected from the group consisting of —H, —OH, F, Cl, Br, I, —C₁₋₆alkyl, —O—C₁₋₆alkyl, and —S—C₁₋₆alkyl;
R⁵, R⁶, R⁷, and R⁸ are each independently selected from the group consisting of H, alkyl and halo; and
R⁹, R¹⁰, and R¹¹ are each independently selected from the group consisting of H, —C₁₋₆alkyl, —O—C₁₋₆alkyl, —S—C₁₋₆ alkyl, F, Cl, Br and I.

The FAP ligand can have a structure represented by the formula I-C:

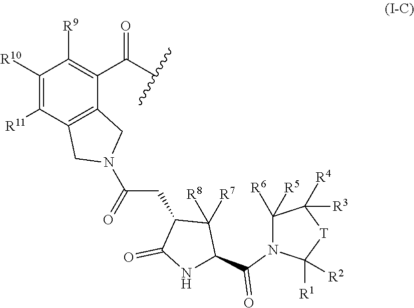

T is substituted or unsubstituted methylene (—CH₂—), substituted or unsubstituted amino (—NH—), —O—, or —S—;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, and 5-tetrazolyl;
R³ and R⁴ are each independently selected from the group consisting of —H, —OH, F. Cl. Br, I, —C₁₋₆alkyl, —O—C₁₋₆alkyl, and —S—C₁₋₆alkyl;
R⁵, R⁶, R⁷, and R⁸ are each independently selected from the group consisting of H, alkyl and halo; and
R⁹, R¹⁰, and R¹¹ are each independently selected from the group consisting of H, —C₁₋₆alkyl, —O—C₁₋₆alkyl, —S—C₁₋₆ alkyl, F, Cl, Br and I. The bispecific adapter can have the one of the structures shown in FIG. 25.

Provided is another bispecific adapter for use with anti-fluorescein (e.g., fluorescein, FITC, or NHS-fluorescein) CAR-T cells in the treatment of FAP-expressing (e.g., FAPα-expressing) cancer, which adapter comprises fluorescein-linker-FAP ligand (e.g., fluorescein=fluorescein, FITC, or NHS-fluorescein), wherein the FAP ligand is or comprises:

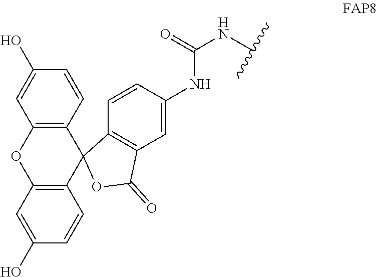 [SMILES: *NC(=O)Nc1ccc2c(c1)C(=O)OC21c2ccc(O)cc2Oc2cc(O)ccc21]

and the linker comprises (or consists essentially of or consists of) PEG, and wherein the adapter can be a pharmaceutically acceptable salt or hydrate thereof. The linker can comprise (or consist essentially of or consist of) PEG₃ to PEG₁₅. The linker can comprise (or consist essentially of or consist of) PEG₁₅. The linker can comprise (or consist essentially of or consist of) PEG₁₆. The FAP8 ligand can have the structure:

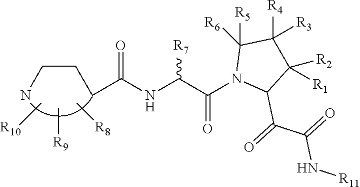

wherein:

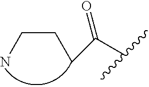 [SMILES: *C(=O)CCCN]

represents a functionalized 5- to 10-membered N-containing aromatic or non-aromatic mono- or bi-cyclic heterocycle, which optionally further comprises 1-3 heteroatoms selected from O, N. and S;
R₁ and R₂ are independently selected from the group consisting of —H, -D, —OH, —F, —Cl, —Br, —I, —C₁₋₆ alkyl, —O—C₁₋₆ alkyl, and —S—C₁₋₆ alkyl;
R₃ and R₄ are independently selected from the group consisting of —H, —OH, —F, —Cl, —Br, —I, —C₁₋₆ alkyl, —O—C₁₋₆ alkyl, and —S—C₁₋₆ alkyl;
R₅ and R₆ are independently selected from group consisting of —H, —OH, —F, —Cl, —Br, —I, —C₁₋₆ alkyl, —O—C₁₋₆ alkyl, and —S—C₁₋₆ alkyl;
R₇ is selected from the group consisting of —H, -D, OH, CH₂═, —CH₃, CH₃CH₂—, (CH₃)₂CH—, (CH₃)₃C—, —CH₂Ph, and substituted —CH₂Ph;
R₈-R₁₀ are independently selected from group consisting of —H, —OH, —F, —Cl, —Br.
—I, —NO₂, —SO₃H, —SO₂NH₂, —NH₂, —N₃, —NH═NH, —C₁₋₆ alkyl, —O—C₁₋₆ alkyl, and —S—C₁₋₆ alkyl; and
R₁₁ is selected from the group consisting of —H, -D, C₁-C₁₀ alkyl, C₃-C₁₀ cycloalkyl,

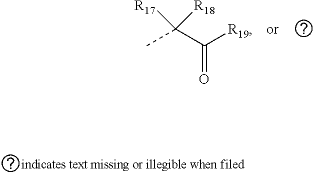

substituted or unsubstituted aryl, substituted or unsubstituted C₇-C₂₀ alkyl aryl, wherein the aryl is:

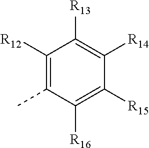

wherein:
R₁₂ and R₁₆ are independently selected from the group consisting of —H, -D, halogen, C₁-C₃ alkyl, C₁-C₃ alkoxy, —CF₃, and —C(═O)—OR₂₃, wherein R₂₃ is selected from the group consisting of H, D, halogen, C₁-C₄ alkyl, and C₁-C₃ alkoxy;
R₁₃, R₁₄ and R₁₅ are independently selected from the group consisting of —H, -D, halogen, —OMe, C₁-C₃ alkyl, C₁-C₃ alkoxy, —CF₃, and —C(═O)—OR₂₃, wherein R₂₃ is selected from the group consisting of —H, -D, halogen, C₁-C₄ alkyl, and C₁-C₃ alkoxy;
R₁₇, R₁₈, R₂₀, and R₂₁ are independently selected from —H and —CH₃; and
R₁₉ and R₂₂ are independently selected from the group consisting of phenyl, dimethoxyphenyl, and aryl.

A bispecific adapter can comprise the following structure:

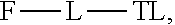

or a pharmaceutically acceptable salt or hydrate thereof, wherein:
F comprises a fluorescein, FITC, or NHS-fluorescein,
L comprises a linker, and
TL comprises a targeting ligand comprising a radical of a FAP ligand or a radical of a PSMA ligand.

The targeting ligand can comprise a radical of a FAP ligand comprising a structure of:

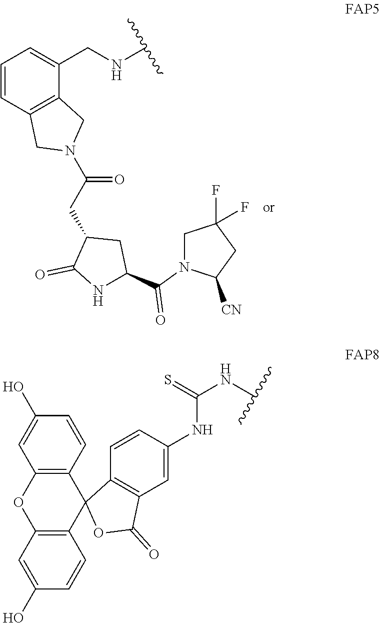

wherein

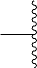

is the point of attachment to the linker.

The targeting ligand can comprise a radical of a FAP5 ligand comprising a structure represented by formula I-B:

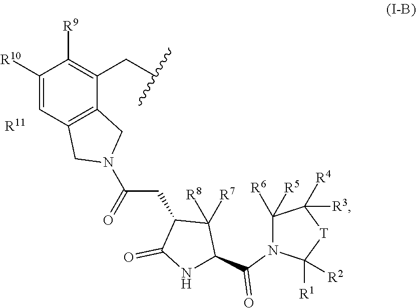

wherein:

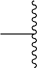

is the point of attachment to the linker;
T is substituted or unsubstituted methylene (—CH₂—), substituted or unsubstituted amino (—NH—), —O—, or —S—;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F and 5-tetrazolyl;
R³ and R⁴ are each independently selected from the group consisting of —H, —OH, F, Cl, Br, I, —C₁₋₆alkyl, —O—C₁₋₆alkyl, and —S—C₁₋₆alkyl;
R⁵, R⁶, R⁷, and R⁸ are each independently selected from group consisting of H, alkyl and halo; and
R⁹, R¹⁰, and R¹¹ are each independently selected from the group consisting of H, —C₁₋₆ alkyl, —O—C₁₋₆alkyl, —S—C₁₋₆ alkyl, F, Cl, Br and I.

The targeting ligand of the bispecific adapter can comprise a radical of a FAP5 ligand comprising a structure represented by the formula I-C:

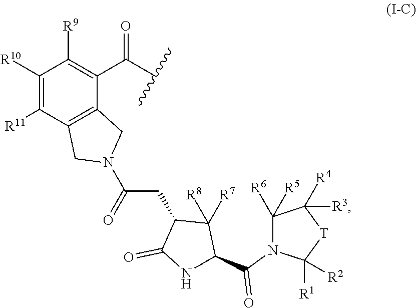

wherein:

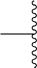

is the point of attachment to the linker;
T is substituted or unsubstituted methylene (—CH₂—), substituted or unsubstituted amino (—NH—), —O—, or —S—;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, and 5-tetrazolyl;
R³ and R⁴ are each independently selected from the group consisting of —H, —OH, F. Cl, Br, I, —C₁₋₆alkyl, —O—C₁₋₆alkyl, and —S—C₁₋₆alkyl;
R⁵, R⁶, R⁷, and R⁸ are each independently selected from group consisting of H, alkyl and halo; and
R⁹, R¹⁰, and R¹¹ are each independently selected from group consisting of H, —C₁₋₆alkyl, —O—C₁₋₆alkyl, —S—C₁₋₆ alkyl, F. Cl, Br and I.

The targeting ligand can comprise a radical of a FAP8 ligand comprising a structure:

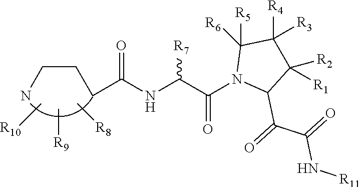

wherein:

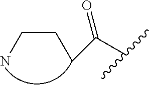 [SMILES: *C(=O)CCCN]

represents a functionalized 5- to 10-membered N-containing aromatic or non-aromatic mono- or bi-cyclic heterocycle, which optionally further comprises 1-3 heteroatoms selected from O, N, and S;
R₁ and R₂ are independently selected from the group consisting of —H, -D, —OH, —F, —Cl, —Br, —I, —C₁₋₆ alkyl, —O—C₁₋₆ alkyl, and —S—C₁₋₆ alkyl;
R₃ and R₄ are independently selected from the group consisting of —H, —OH, —F, —Cl, —Br, —I, —C₁₋₆ alkyl, —O—C₁₋₆ alkyl, and —S—C₁₋₆ alkyl;
R₅ and R₆ are independently selected from group consisting of —H, —OH, —F, —Cl, —Br, —I, —C₁₋₆ alkyl, —O—C₁₋₆ alkyl, and —S—C₁₋₆ alkyl;
R₇ is selected from the group consisting of —H, -D, OH, CH₂═, —CH₃, CH₃CH₂—, (CH₃)₂CH—(CH₃)₃C—, —CH₂Ph, and substituted —CH₂Ph;
R₈-R₁₀ are independently selected from group consisting of —H, —OH, —F, —Cl, —Br, —I, —NO₂, —SO₃H, —SO₂NH₂, —NH₂, —N₃, —NH═NH, —C₁₋₆ alkyl, —O—C₁₋₆ alkyl, and —S—C₁₋₆ alkyl; and
R₁₁ is selected from the group consisting of —H, -D, C₁-C₁₀ alkyl, C₃-C₁₀ cycloalkyl, adamantyl,

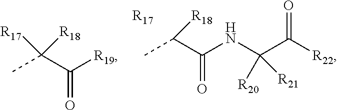

substituted or unsubstituted aryl, substituted or unsubstituted C₇-C₂₀ alkyl aryl, wherein the aryl is:

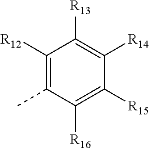

wherein:
R₁₂ and R₁₆ are independently selected from the group consisting of —H, -D, halogen, C₁-C₃ alkyl, C₁-C₃ alkoxy, —CF₃, and —C(═O)—OR₂₃, wherein R₂₃ is selected from the group consisting of H, D, halogen, C₁-C₄ alkyl, and C₁-C₃ alkoxy;
R₁₃, R₁₄ and R₁₅ are independently selected from the group consisting of —H, -D, halogen, —OMe, C₁-C₃ alkyl. C₁-C₃ alkoxy, —C₃, and —C(═O)—OR₂₃, wherein R₂₃ is selected from the group consisting of —H, -D, halogen, C₁-C₄ alkyl, and C₁-C₃ alkoxy;
R₁₇, R₁₈, R₂₀, and R₂₁ are independently selected from —H and —CH₃; and
R₁₉ and R₂₂ are independently selected from the group consisting of phenyl, dimethoxyphenyl, and aryl.

In certain embodiments, the targeting ligand comprises a radical of a PSMA ligand and is PSMAL1 or DUPA.

The linker can comprise or consists essentially of polyethylene glycol (PEG) or a PEG derivative such as, optionally: PEG₃ to PEG₁₆ and, optionally, PEG₄ to PEG₁₅ or PEG₃ to PEG₁₂; PEG₁₂, PEG₁₅, PEG₁₆, or PEG₁₈; PEG₄ to PEG₁₆; PEG₁₆; PEG₃ to PEG₁₅; PEG₁₅; PEG₃ to PEG₁₂; PEG₆; PEG₃ to PEG₈; or PEG₆.

The bispecific adaptor can be for use with an anti-fluorescein CAR-T cell in the treatment of cancer.

The bispecific adaptor can be for use with an anti-fluorescein CAR-T cell in the treatment of FAP-expressing cancer, wherein optionally the linker comprises or consists essentially of PEG₃ to PEG₁₅ and, optionally, PEG₁₅.

The bispecific adaptor can be for use with an anti-fluorescein CAR-T cell in the treatment of PSMA-expressing cancer, wherein optionally the linker comprises or consists essentially of PEG₃ to PEG₁₂ and, optionally, PEG₆ or PEG₃ to PEG₈ and, optionally, PEG₆.

In certain embodiments, a bispecific adapter for use with an anti-fluorescein CAR-T cell in the treatment of a FAP-expressing cancer is provided, which adapter has or comprises one of the following structures:

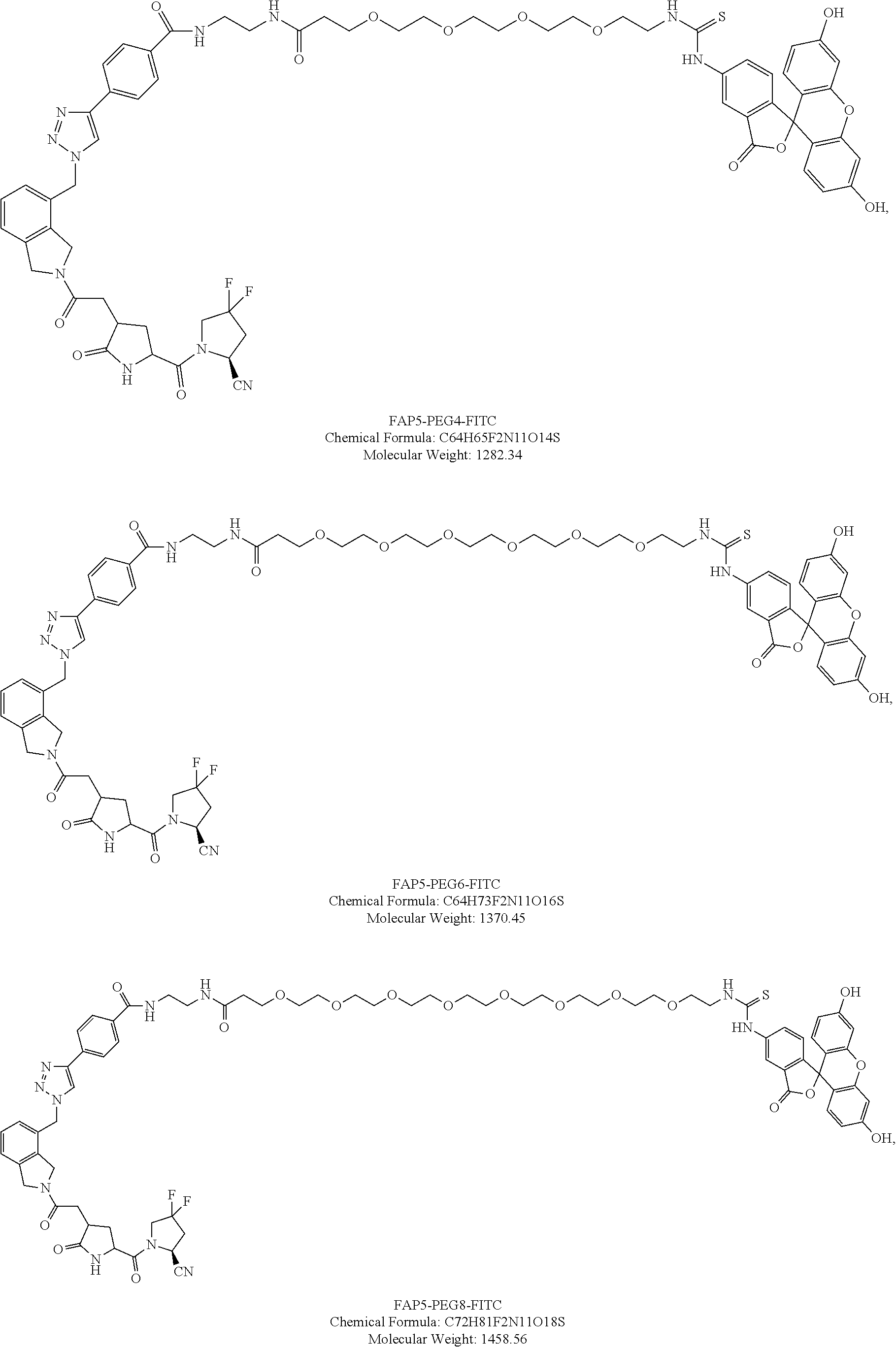

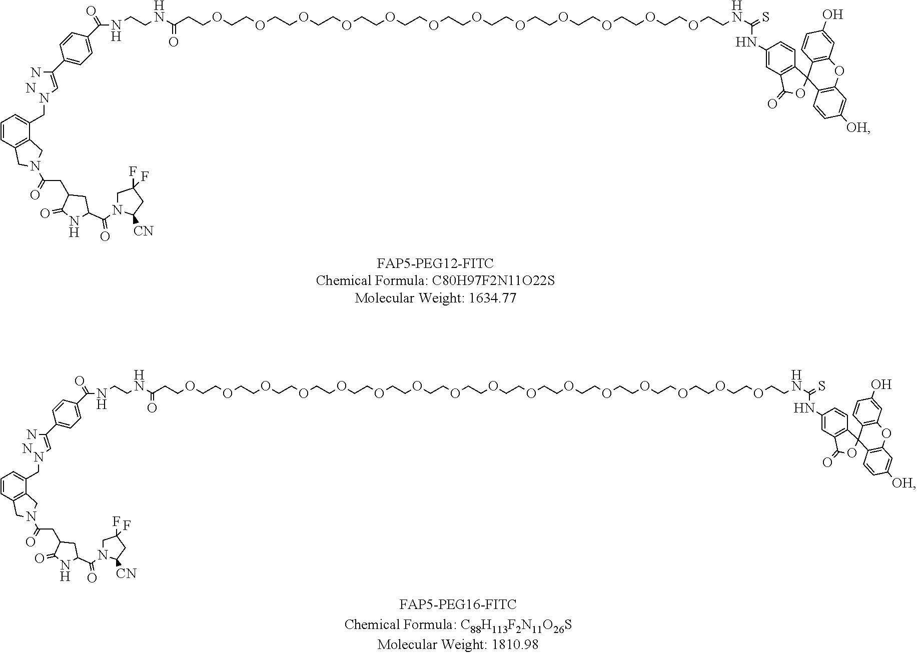 [SMILES: N#C[C@@H]1CC(F)(F)CN1C(=O)C1CC(CC(=O)N2Cc3cccc(Cn4cc(-c5ccc(C(=O)NCCNC(=O)CCOCCOCCOCCOCCOCCOCCOCCOCCOCCOCCOCCOCCNC(=S)Nc6ccc7c(c6)C(=O)OC76c7ccc(O)cc7Oc7cc(O)ccc76)cc5)nn4)c3C2)C(=O)N1 ; N#C[C@@H]1CC(F)(F)CN1C(=O)C1CC(CC(=O)N2Cc3cccc(Cn4cc(-c5ccc(C(=O)NCCNC(=O)CCOCCOCCOCCOCCOCCOCCOCCOCCOCCOCCOCCOCCOCCOCCOCCOCCNC(=S)Nc6ccc7c(c6)C(=O)OC76c7ccc(O)cc7Oc7cc(O)ccc76)cc5)nn4)c3C2)C(=O)N1]

or comprising a pharmaceutically acceptable salt of hydrate of any of the foregoing structures.

In certain embodiments, a bispecific adapter for use with an anti-fluorescein CAR-T cell in the treatment of a FAP-expressing cancer is provided, which adapter has or comprises one of the following structures:

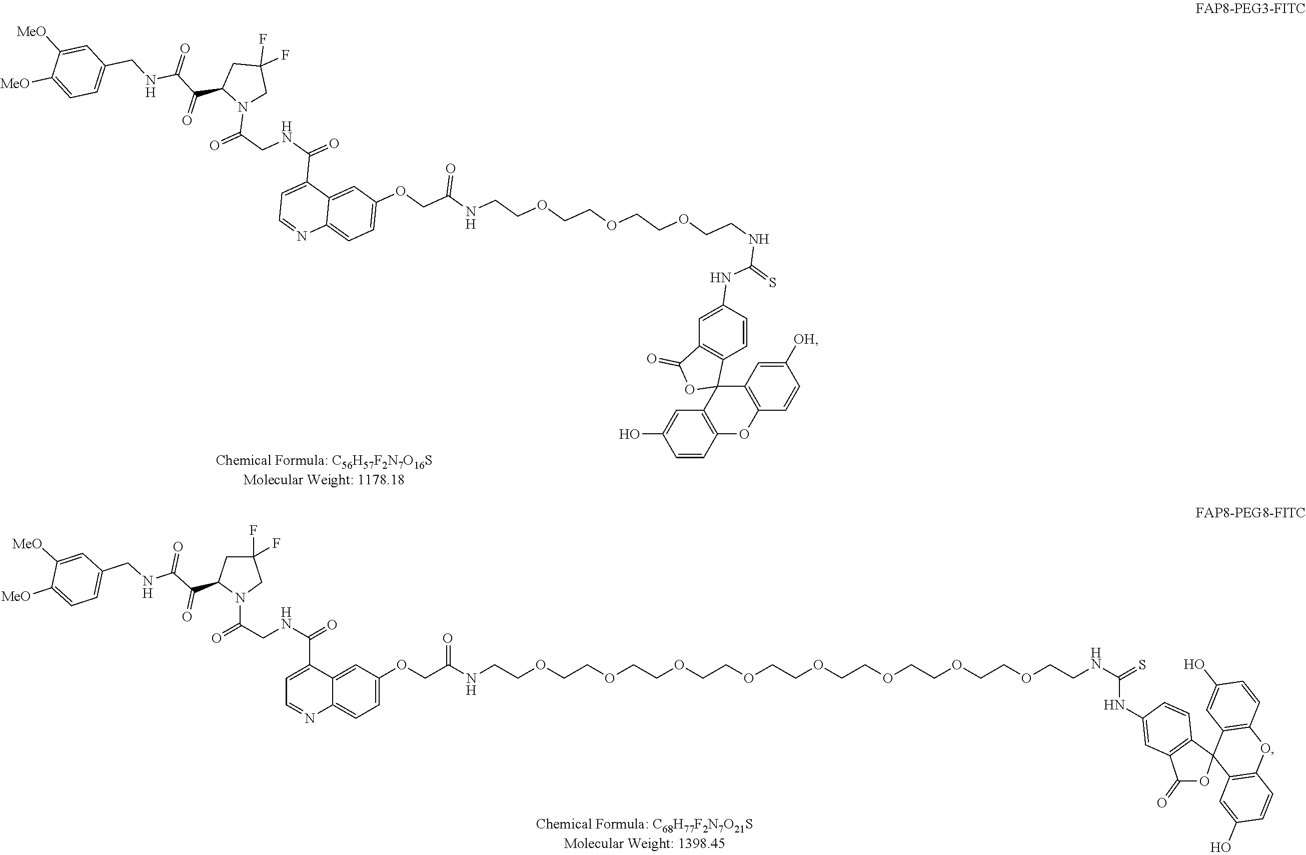 [SMILES: COc1ccc(CNC(=O)C(=O)[C@H]2CC(F)(F)CN2C(=O)CNC(=O)c2ccnc3ccc(OCC(=O)NCCOCCOCCOCCNC(=S)Nc4ccc5c(c4)C(=O)OC54c5cc(O)ccc5Oc5ccc(O)cc54)cc23)cc1OC ; COc1ccc(CNC(=O)C(=O)[C@H]2CC(F)(F)CN2C(=O)CNC(=O)c2ccnc3ccc(OCC(=O)NCCOCCOCCOCCOCCOCCOCCOCCOCCNC(=S)Nc4ccc5c(c4)C(=O)OC54c5cc(O)ccc5Oc5ccc(O)cc54)cc23)cc1OC]

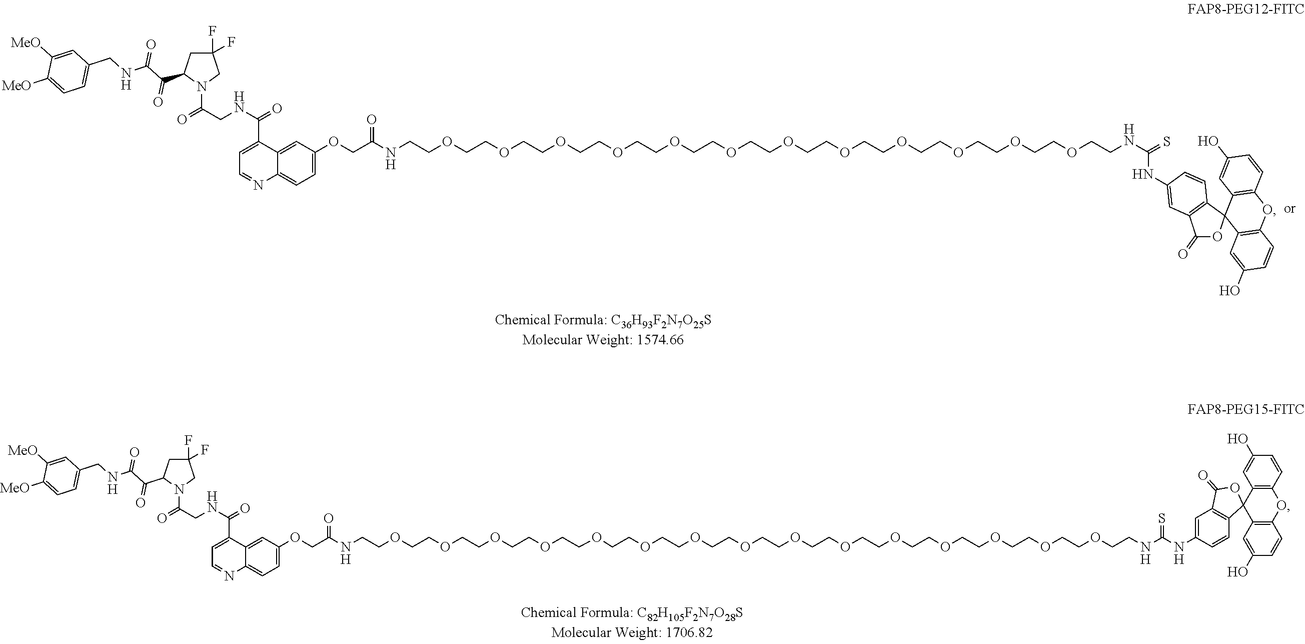 [SMILES: COc1ccc(CNC(=O)C(=O)[C@H]2CC(F)(F)CN2C(=O)CNC(=O)c2ccnc3ccc(OCC(=O)NCCOCCOCCOCCOCCOCCOCCOCCOCCOCCOCCOCCOCCNC(=S)Nc4ccc5c(c4)C(=O)OC54c5cc(O)ccc5Oc5ccc(O)cc54)cc23)cc1OC ; "or" ; COc1ccc(CNC(=O)C(=O)C2CC(F)(F)CN2C(=O)CNC(=O)c2ccnc3ccc(OCC(=O)NCCOCCOCCOCCOCCOCCOCCOCCOCCOCCOCCOCCOCCOCCOCCOCCNC(=S)Nc4ccc5c(c4)C(=O)OC54c5cc(O)ccc5Oc5ccc(O)cc54)cc23)cc1OC]

or comprising a pharmaceutically acceptable salt or hydrate of any of the foregoing structures.

In certain embodiments, a bispecific adapter for use with an anti-fluorescein CAR-T cell in the treatment of a PSMA cancer is provided, which adapter has or comprises one of the following structures:

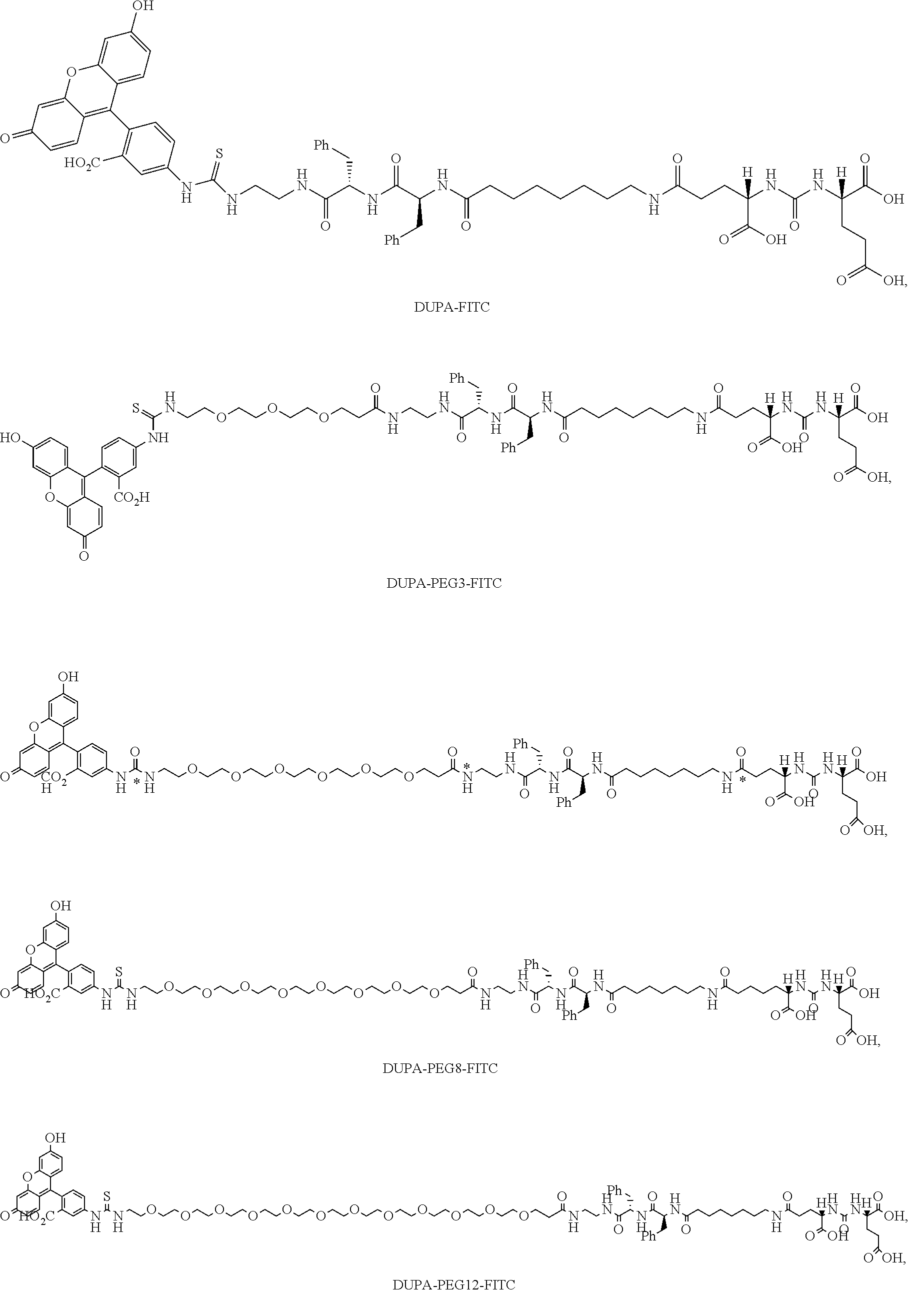

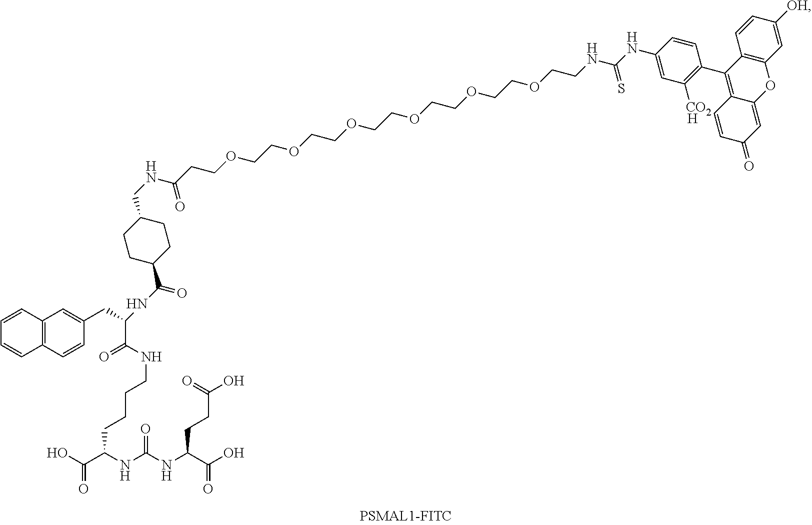

or comprises a pharmaceutically acceptable salt or hydrate of any of the foregoing.

In certain embodiments, a bispecific adapter for use with an anti-fluorescein CAR-T cell in the treatment of PSMA-expressing cancer is provided, which adapter has the structure:

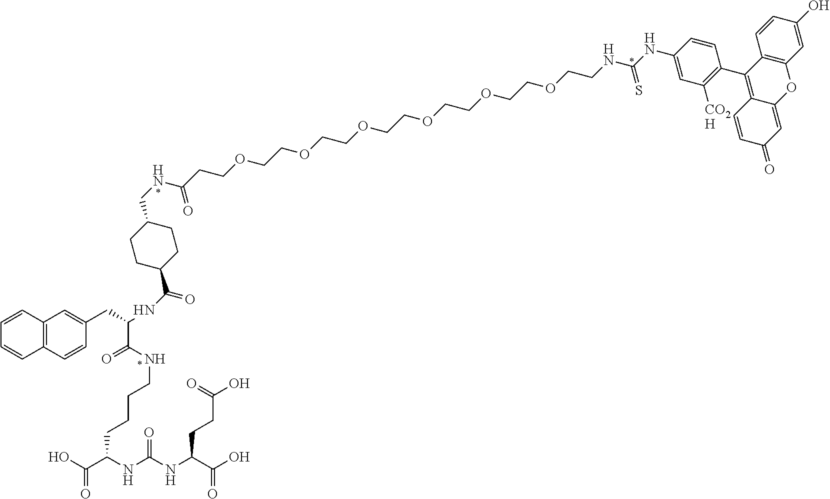 [SMILES: O=C(O)CC[C@H](NC(=O)N[C@@H](CCCCNC(=O)[C@H](Cc1ccc2ccccc2c1)NC(=O)[C@H]1CC[C@H](CNC(=O)CCOCCOCCOCCOCCOCCOCCNC(=S)Nc2ccc(-c3c4ccc(=O)cc-4oc4cc(O)ccc34)c(C(=O)O)c2)CC1)C(=O)O)C(=O)O]

or is a pharmaceutically acceptable salt or hydrate thereof.

In certain embodiments, a bispecific adapter for use with an anti-fluorescein CAR-T cell in the treatment of PSMA-expressing cancer, which adapter has the structure:

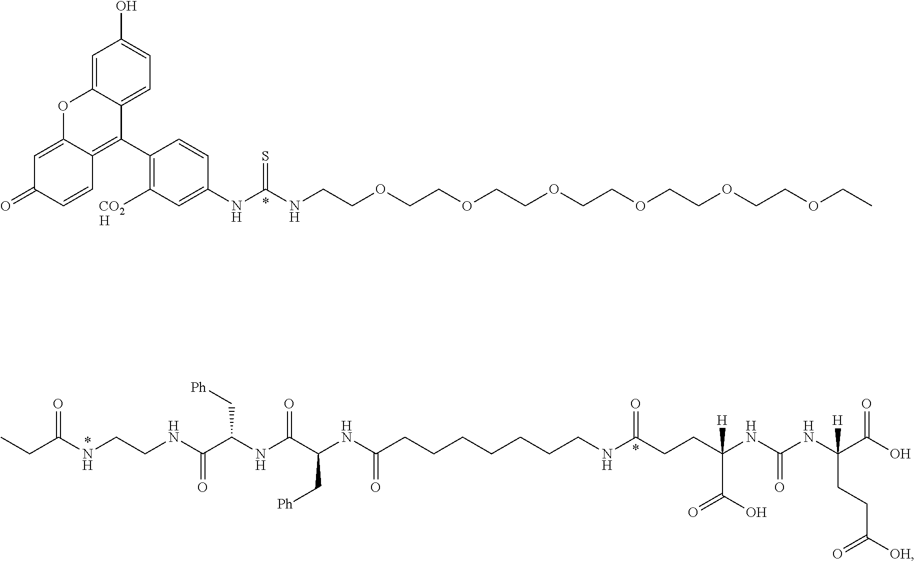

or is a pharmaceutically acceptable salt or hydrate thereof.

Also provided is a pharmaceutical composition for the treatment of FAP-expressing (e.g., FAPα-expressing) cancer comprising an above-described bispecific adapter and a pharmaceutically acceptable carrier or excipient.

In certain embodiments, the pharmaceutical composition for the treatment of cancer comprises any of the bispecific adapters described herein and a pharmaceutically acceptable carrier or excipient.

A combination of bispecific adaptors for use with anti-fluorescein CAR-T cells in the treatment of cancer is also provided. In certain embodiments, the combination comprises:
(i) a first bispecific adaptor comprising any bispecific adapter described herein or a pharmaceutically acceptable salt or hydrate thereof, wherein the targeting ligand of the first bispecific adapter comprises a radical of a FAP ligand having a formula of:

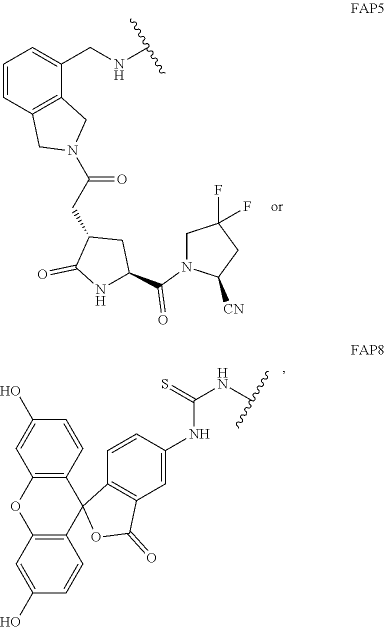

wherein

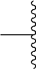

is the point of attachment to the linker; and
(ii) a second bispecific adapter comprising the following structure:

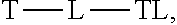

or a pharmaceutically acceptable salt or hydrate thereof, wherein:
F comprises a fluorescein, FITC, or NHS-fluorescein,
L comprises a linker, and
TL comprises a targeting ligand comprising a radical of a FR ligand or a PSMA ligand.

The radical of the FAP ligand of the first bispecific adaptor of the combination can have a structure represented by the formula I-B:

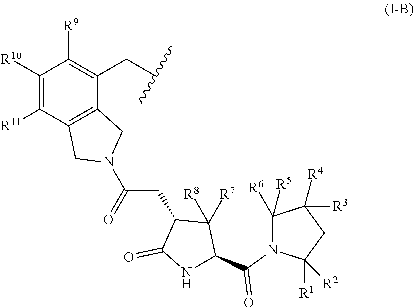

wherein:
T is substituted or unsubstituted methylene (—CH₂—), substituted or unsubstituted amino (—NH—),
   —O—, or —S—;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F and 5-tetrazolyl;
R³ and R⁴ are each independently selected from the group consisting of —H, —OH, F, Cl, Br, I. —C₁₋₆alkyl, —O—C₁₋₆alkyl, and —S—C₁₋₆alkyl;
R⁵, R⁶, R⁷, and R⁸ are each independently selected from group consisting of H, alkyl and halo; and
R⁹, R¹⁰, and R¹¹ are each independently selected from group consisting of H, —C₁₋₆alkyl, —O—C₁₋₆alkyl, —S—C₁₋₆ alkyl, F, Cl, Br and I; or
a structure represented by the formula I-C:

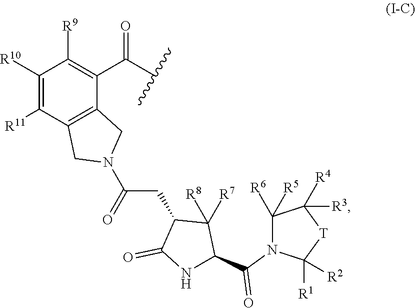

wherein:

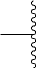

is the point of attachment to the linker;
T is substituted or unsubstituted methylene (—CH₂—), substituted or unsubstituted amino (—NH—), —O—, or —S—;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, and 5-tetrazolyl;
R³ and R⁴ are each independently selected from the group consisting of —H, —OH, F, Cl, Br, I, —C₁₋₆alkyl, —O—C₁₋₆alkyl, and —S—C₁₋₆alkyl;
R⁵, R⁶, R⁷, and R⁸ are each independently selected from group consisting of H, alkyl and halo; and
R⁹, R¹⁰, and R¹¹ are each independently selected from group consisting of H, —C₁₋₆ alkyl, —O—C₁₋₆alkyl, —S—C₁₋₆ alkyl, F, Cl, Br and I; or
a structure represented comprising the following formula:

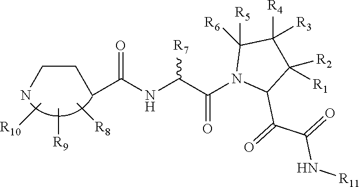

wherein:

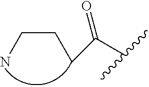 [SMILES: *C(=O)CCCN]

represents a functionalized 5- to 10-membered N-containing aromatic or non-aromatic mono- or bi-cyclic heterocycle, which optionally further comprises 1-3 heteroatoms selected from O, N, and S;
R₁ and R₂ are independently selected from the group consisting of —H, -D, —OH, —F, —Cl, —Br, —I, —C₁₋₆ alkyl, —O—C₁₋₆ alkyl, and —S—C₁₋₆ alkyl;
R₃ and R₄ are independently selected from the group consisting of —H, —OH, —F, —Cl, —Br, —I, —C₁₋₆ alkyl, —O—C₁₋₆ alkyl, and —S—C₁₋₆ alkyl;
R₅ and R₆ are independently selected from group consisting of —H, —OH, —F, —Cl, —Br, —I, —C₁₋₆ alkyl, —O—C₁₋₆ alkyl, and —S—C₁₋₆ alkyl;
R₇ is selected from the group consisting of —H, -D, OH, CH₂═, —CH₃, CH₃CH₂—, (CH₃)₂CH—, (CH₃)₃C—, —CH₂Ph, and substituted —CH₂Ph;
R₈-R₁₀ are independently selected from group consisting of —H, —OH, —F, —Cl, —Br,
—I, —NO₂, —SO₃H, —SO₂NH₂, —NH₂, —N₃, —NH═NH, —C₁₋₆ alkyl, —O—C₁₋₆ alkyl, and —S—C₁₋₆ alkyl; and
R₁₁ is selected from the group consisting of —H, -D, C₁-C₁₀ alkyl, C₃-C₁₀ cycloalkyl, adamantyl,

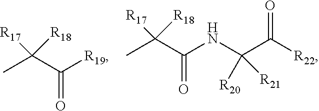

substituted or unsubstituted aryl, substituted or unsubstituted C₇-C₂₀ alkyl aryl, wherein the aryl is:

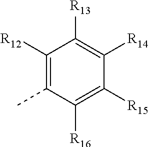

wherein:
R₁₂ and R₁₆ are independently selected from the group consisting of —H, -D, halogen, C₁-C₃ alkyl, C₁-C₃ alkoxy, —CF₃, and —C(═O)—OR₂₃, wherein R₂₃ is selected from the group consisting of H, D, halogen, C₁-C₄ alkyl, and C₁-C₃ alkoxy;
R₁₃, R₁₄ and R₁₅ are independently selected from the group consisting of —H, -D, halogen, —OMe, C₁-C₃ alkyl, C₁-C₃ alkoxy, —CF₃, and —C(═O)—OR₂₃, wherein R₂₃ is selected from the group consisting of —H, -D, halogen, C₁-C₄ alkyl, and C₁-C₃ alkoxy;
R₁₇, R₁₈, R₂₀, and R₂₁ are independently selected from —H and —CH₃; and
R₁₉ and R₂₂ are independently selected from the group consisting of phenyl, dimethoxyphenyl, and aryl.

The targeting ligand of the second bispecific adapter, or pharmaceutically acceptable salt or hydrate thereof, of the combination can comprise a radical of a PSMA ligand. The linker of the first bispecific adaptor of the combination can comprise or consists essentially of PEG.

The targeting ligand can be or comprise PSMAL1 or DUPA.

The targeting ligand of the second bispecific adaptor of the combination is a radical of a folate or a functional fragment or analog thereof.

The folate can be folate, dihydrofolate tetrahydrofolate, 5, 10-methylene tetrahydrofolate (5,10-MTHF), 5-methyltetrahydrofolate (5-MTHF), or raltitrexed.

The PSMA ligand can be DUPA and the linker can comprise or consists essentially of PEG or a PEG derivative such as, optionally: PEG₃ to PEG₁₂ and, optionally, PEG₆; PEG₃ to PEG₁₆ and, optionally, PEG₄ to PEG₁₅ or PEG₃ to PEG₁₂; PEG₁₂, PEG₁₅, PEG₁₆, or PEG₁₈; PEG₄ to PEG₁₆ and, optionally, PEG₁₆; PEG₃ to PEG₁₅ and, optionally, PEG₁₅; PEG₃ to PEG₈ and, optionally, PEG₆.

The combination can be used with an anti-fluorescein CAR-T cell in the treatment of cancer. The combination can be used with an anti-fluorescein CAR-T cell in the treatment of FAP-expressing cancer. The combination can be used with an anti-fluorescein CAR-T cell in the treatment of PSMA-expressing cancer. The combination can be used with an anti-fluorescein CAR-T cell in the treatment of folate-expressing cancer. The first and second bispecific adapters of the combination can be formulated in separate pharmaceutical compositions.

Further provided is a method of treating FAP-expressing (e.g., FAPα-expressing) cancer in a subject, which method comprises administering to the subject cancer-treatment effective amounts of (i) anti-fluorescein (e.g., fluorescein, FITC, or NHS-fluorescein) CAR-T cells or a pharmaceutical composition comprising the same and a pharmaceutically acceptable carrier or excipient, and (ii) a herein-described bispecific adapter or a pharmaceutical composition comprising the same and a pharmaceutically acceptable carrier or excipient, whereupon the subject is treated for cancer.

The CAR can have a recognition region and the recognition region is a single chain fragment variable (scFv) region of an anti-fluorescein (e.g., fluorescein, FITC, or NHS-fluorescein) antibody. In certain embodiments, the CAR has a co-stimulation domain and the co-stimulation domain is CD28, CD137 (4-1BB), CD134 (OX40), or CD278 (ICOS). The CAR can have an activation signaling domain and the activation signaling domain is a T cell CD3ζ chain or an Fc receptor γ.

In the methods hereof, (i) and (ii) can be administered simultaneously or sequentially, in either order, by the same or different routes. Further, optionally, (i) and (ii) of the method can be administered intravenously.

In certain embodiments, a method of treating cancer in a subject is provided, the method comprising administering to the subject cancer-treatment effective amounts of (i) anti-fluorescein CAR-T cells or a pharmaceutical composition comprising anti-fluorescein CAR-T cells and a pharmaceutically acceptable carrier or excipient; and (ii) any bispecific adapter described herein, a pharmaceutical composition described herein, or any combination described herein; whereupon the subject is treated for cancer.

The CAR of the method can comprise: a recognition region comprising a single chain fragment variable (scFv) region of an anti-fluorescein antibody; a co-stimulation domain and the co-stimulation domain is CD28, CD137 (4-1BB), CD134 (OX40), or CD278 (ICOS); and/or an activation signaling domain that is a T cell CD3ζ chain or an Fc receptor γ.

The fluorescein of the bispecific adapter of the method can bind the anti-fluorescein CAR-T cell with affinity upon exposure thereto, and the targeting ligand of the bispecific adapter links the bound anti-fluorescein CAR-T cell to a targeted cancer cell or cancer-associated fibroblast (CAF) upon the targeted ligand of the bispecific adapter binding a receptor on such targeted cancer cell or CAF with affinity. The receptor on the targeted cancer cell or CAF can be an overexpressed FAP, an over-expressed PSMA, and/or a FR.

In certain embodiments, (i) and (ii) of the method are administered simultaneously or sequentially, in either order, by the same or different routes. Step (ii) of the method can comprise any of the combinations described herein and the first and second bispecific adapters can be administered to the subject simultaneously by the same or different routes.

In certain embodiments, step (ii) of the method comprises any of the combinations described herein and the first and second bispecific adapters are administered to the subject sequentially, in either order, by the same or different routes.

Steps (i) and (ii) of the method can each be administered intravenously, for example.

The cancer can be a FAP-expressing cancer and at least one bispecific adapter of step (ii) can comprise a radical of a FAP ligand.

In certain embodiments of the method, the cancer is a PSMA-expressing cancer and at least one bispecific adapter of step (ii) of the method comprises a radical of a PSMA ligand.

The cancer can be a FR-expressing cancer and step (ii) of the method can comprise any combination described herein.

A method of treating a FAP-expressing cancer in a subject is also provided. Such method can comprise administering to the subject cancer-treatment effective amounts of (i) anti-fluorescein CAR-T cells or a pharmaceutical composition comprising anti-fluorescein CAR-T cells and a pharmaceutically acceptable carrier or excipient; and (ii) any of the bispecific adapters or pharmaceutical compositions described herein, whereupon the subject is treated for cancer.

The CAR can have a recognition region and the recognition region is a scFv region of an anti-fluorescein antibody. The CAR can comprise: a co-stimulation domain and the co-stimulation domain is CD28, CD137 (4-1BB), CD134 (OX40), or CD278 (ICOS); and/or an activation signaling domain and the activation signaling domain is a T cell CD3 chain or an Fc receptor γ.

A method of treating cancer in a subject is also provided, such method comprising administering to the subject cancer-treatment effective amounts of: (i) anti-fluorescein CAR-T cells or a pharmaceutical composition comprising anti-fluorescein CAR-T cells and a pharmaceutically acceptable carrier or excipient; and (ii) any of the combinations described herein; whereupon the subject is treated for cancer. The CAR can comprise: a recognition region comprising a scFv region of an anti-fluorescein antibody; a co-stimulation domain and the co-stimulation domain is CD28, CD137 (4-1BB), CD134 (OX40), or CD278 (ICOS); and/or an activation signaling domain that is a T cell CD3ζ chain or an Fc receptor γ.

In certain embodiments of the method, steps (i) and (ii) are administered simultaneously or sequentially, in either order, by the same or different routes. The first and second bispecific adapters of the combination can be administered to the subject simultaneously by the same or different routes. The first and second bispecific adapters of the combination can be administered to the subject sequentially, in either order, by the same or different routes. In certain embodiments of the method, steps (i) and (ii) are each administered intravenously.

The methods hereof can further comprise imaging the cancer in the subject. Imaging the cancer can comprise imaging by optical imaging, positron emission tomography (PET), or single photon emission computed tomography (SPECT).

The cancer can be ovarian cancer, endometrial cancer, breast cancer, glioma such as, optionally, stage 3-4 glioma, or clear cell renal cell carcinoma such as, optionally, stage 3-4 clear cell renal cell carcinoma.

A method for enhancing CAR-T cell activation is also provided. In certain embodiments, such a method comprises: providing a bispecific adapter hereof, a pharmaceutical composition hereof, or a combination hereof; and exposing anti-fluorescein CAR-T cells or a pharmaceutical composition comprising anti-fluorescein CAR-T cells and a pharmaceutically acceptable carrier or excipient to the bispecific adaptor, pharmaceutical composition or combination; wherein the CAR-T cell experiences enhanced activation following exposure as compared to a CAR-T cell not exposed to the bispecific adapter. The anti-fluorescein CAR-T cells can be in systemic circulation in a subject when exposed to the bispecific adaptor.

Still further provided is a kit. The kit can comprise (i) an above-described bispecific adapter or a pharmaceutical composition comprising same and a pharmaceutically acceptable carrier or excipient, and (ii) anti-fluorescein an anti-fluorescein (e.g., fluorescein, FITC, or NHS-fluorescein) CAR-T cells or a pharmaceutical composition comprising same and a pharmaceutically acceptable carrier or excipient.

In certain embodiments, the kit comprises: (i) at least one dosage unit of any of the bispecific adapters described herein, a pharmaceutical composition described herein, or any of the combinations described herein; and (ii) at least one dosage unit of an anti-fluorescein CAR-T cells or a pharmaceutical composition comprising anti-fluorescein CAR-T cells and a pharmaceutically acceptable carrier or excipient; wherein (i) and (ii) are optionally in separate containers.

### DESCRIPTION OF THE DRAWINGS

The above and other objects, features, and advantages of the present invention will become more apparent when taken in conjunction with the following description and drawings, wherein:

FIG. 1 shows graphs of fibroblast activation protein (FAP)-fluorescein isothiocyanate (FITC), concentration (nmol/L) vs. FITC mean fluorescence intensity (MFI) for FAP5 K_{D} on human FAP (left) and murine FAP (right).

FIG. 2A is a graph of ligand vs. MFI (allophycocyanin (APC)), which shows FITC exposure. FAP-FITC coated cells were stained with anti-FITC antibody that has an APC signal to measure the APC MFI, which represents FITC exposure.

FIG. 2B is a graph of ligand vs. FITC (MFI), which shows Bₘₐₓ on MDA-MB231-hFAP.

FIG. 3 is a graph of FAP5-FITC concentration (nmol/L) vs. FITC (MFI), which shows the results of a binding affinity assay.

FIG. 4A is a graph of FAP5-FITC concentration (nM) vs. % lysis.

FIG. 4B is a graph of FAP5-FITC concentration (nM) vs. interferon gamma (IFNγ) (pg/ml).

FIG. 5A is a graph of days after CAR-T injection vs. tumor volume (mm³) for grouped tumor size.

FIG. 5B is a graph of days after CAR-T injection vs. tumor volume (mm³) for single tumor disease control.

FIG. 5C is a graph of days after CAR-T injection vs. tumor volume (mm³) for single tumor CAR-T only.

FIG. 5D is a graph of days after CAR-T injection vs. tumor volume (mm³) for single tumor FAP5-PEG₁₆-FITC.

FIG. 5E is a graph of days after CAR-T injection vs. tumor volume (mm³) for single tumor FAP8-PEG₁₅-FITC.

FIG. 5F is a bar graph of control and treatment groups vs. % hCD3+ cells/total live cells.

FIG. 6A are graphs of FAP-FITC concentration (nM) vs. MFI (FITC), which show the binding affinity of FAP8-FITC with PEG linkers of different length to hFAP.

FIG. 6B is a graph of FAP-FITC concentration (nM) vs. MFI (FITC), which shows the binding affinity of FAP8-FITC with PEG linkers of different length to mFAP.

FIG. 6C is a graph of FITC showing the results normalized to mode (non-staining vs. FAP8-PEG₈-FITC, FAP8-PEG₁₂-FITC, and FAP8-PEG₁₅-FITC).

FIG. 7A is a graph of anti-FITC APC showing the results normalized to mode (non-staining vs. aFITC-APC, FAP8-PEG₈-FITC, FAP8-PEG₁₂-FITC, and -PEG₁₅-FITC).

FIG. 7B is a graph showing FITC exposure of FAP8-FITC with PEG linkers of different length by aFITC-APC antibody (geometric mean).

FIG. 7C is a graph of days after CAR-T injection vs. tumor volume (mm³).

FIG. 7D is a bar graph of control and treatment groups vs. % hCD3+ cells/total live cells.

FIG. 8A are graphs of hours vs. mCh+ surface area (μm²), which show MDA-hFAP killing by FAP8-FITC adapters with PEG linkers of different lengths under adapter co-culture conditions.

FIG. 8B is a graph of FAP-FITC concentration (nM) vs. % total lysis at 68 hours of co-culture, which shows MDA-hFAP killing by 4M5.3 for FAP8-FITC adapters with PEG linkers of different lengths.

FIG. 8C is a graph of FAP-FITC concentration (nM) vs. IFNγ (pg/ml), which shows cytokine release at 68 hours.

FIG. 8D are graphs of hours vs. mCh+ surface area (μm²), which show MDA-hFAP killing by FAP8-FITC adapters with PEG linkers of different lengths under adapter washed conditions.

FIG. 8E is a graph of FAP-FITC concentration (nM) vs. % total lysis, which shows MDA-hFAP killing by 4M5.3 CAR-T cells and FAP8-FITC adapters with PEG linkers of different lengths under washed conditions at 68 hours.

FIG. 8F is a graph of FAP-FITC concentration (nM) vs. IFNγ (pg/ml), which shows cytokine release at 68 hours.

FIG. 9A is a graph of FAP-FITC concentration (nm) vs. MFI (FITC), which shows the comparison binding of FAP8-PEG₁₅-FITC and FAP5-PEG₁₆-FITC to MDA-MB-231 cells over-expressing hFAP.

FIG. 9B is a graph of FAP-FITC concentration (nmole/L) vs. MFI (FITC), which shows the comparison binding of FAP8-PEG₁₅-FITC and FAP5-PEG₁₆-FITC to MDA-MB-231 cells over-expressing mFAP.

FIG. 10A is a graph of anti-FITC APC showing the results normalized to mode (non-staining, aFITC-APC only, FAP8-PEG₁₅-FITC, and FAP5-PEG₁₆-FITC).

FIG. 10B is a graph showing FITC exposure of FAP8-FITC with PEG linkers of different length by aFITC-APC antibody (geometric mean).

FIG. 11 is a graph of time after incubation (hrs) vs. MFI (APC), which compares dissociation of FAP8-PEG₁₆-FITC, FAP8-PEG₁₂-FITC, FAP8-PEG₁₅-FITC, and FAP5-PEG₁₆-FITC.

FIG. 12A are graphs of hours vs. mCh+ surface area (μm²), which show MDA-hFAP killing by FAP8-PEG₁₅-FITC and FAP5-PEG₁₆-FITC adapters under adapter co-culture and adapter washed conditions.

FIG. 12B are graphs of FAP-FITC concentration (nM) vs. % total lysis (left) or IFNγ (pg/ml) (right) for FAP8-PEG₁₅-FITC and FAP5-PEG₁₆-FITC under adapter co-culture and adapter washed conditions.

FIG. 13A is a bar graph of control and treatment groups vs. % hCD3+ cells/total live cells.

FIG. 13B is a bar graph of control and treatment groups vs. IFNγ (pg/ml).

FIG. 14 is a graph of days post CAR T-cell injection vs. weight change (%).

FIG. 15A is a bar graph of FAP-FITC adapters with increasing PEG linker lengths vs. MFI (anti-FITC APC).

FIG. 15B is a bar graph of FAP-FITC adapters with increasing PEG linker lengths vs. FITC (MFI).

FIG. 16A is a graph of FAP-FITC concentration (nmol/L) vs. % total lysis.

FIG. 16B is a graph of FAP-FITC concentration (nmol/L) vs. IFNγ (pg/ml).

FIG. 17A is a graph of FAP-FITC concentration (nmol/L) vs. % total lysis.

FIG. 17B is a graph of FAP-FITC concentration (nmol/L) vs. IFNγ (pg/ml).

FIG. 18A is a graph of FAP-FITC concentration (nmol/L) vs. % total lysis.

FIG. 18B is a graph of FAP-FITC concentration (nmol/L) vs. IFNγ (pg/ml).

FIG. 19A is a graph of days post CAR T-cell injection vs. tumor volume (mm³).

FIG. 19B is a graph of days post CAR T-cell injection vs. tumor volume (mm³).

FIG. 19C is a graph of days post CAR T-cell injection vs. tumor volume (mm³).

FIG. 19D is a graph of days post CAR T-cell injection vs. tumor volume (mm³).

FIG. 19E is a graph of days post CAR T-cell injection vs. tumor volume (mm³).

FIG. 19F is a graph of days post CAR T-cell injection vs. tumor volume (mm³).

FIG. 20A is a graph of control and treatment groups vs. IFNγ (pg/ml).

FIG. 20B is a graph of control and treatment groups vs. CAR+ count/μl of blood.

FIG. 21A is a graph of control and treatment groups vs. IFNγ (pg/ml).

FIG. 21B is a graph of control and treatment groups vs. CAR+ count/μl of blood.

FIG. 22A is a graph of control and treatment groups vs. IFNγ (pg/ml).

FIG. 22B is a graph of control and treatment groups vs. CAR+ count/μl of blood.

FIG. 23A is a graph of control and treatment groups vs. IFNγ (pg/ml).

FIG. 23B is a graph of control and treatment groups vs. CAR+ count/μl of blood.

FIG. 24 is a graph of control and treatment groups vs. CAR+ cells/50,000 live cells.

FIG. 25 shows bispecific adapter structures.

FIG. 26A is a graph of days after CAR T injection vs. tumor volume (mm³), which compares FAP5-PEG₁₆-FITC and FAP8-PEG₁₅-FITC.

FIG. 26B is a graph of days after CAR T injection vs. tumor volume (mm³) for single tumor disease control.

FIG. 26C is a graph of days after CAR T injection vs. tumor volume (mm³) for single tumor CAR T only.

FIG. 26D is a graph of days after CAR T injection vs. tumor volume (mm³) for single tumor FAP5-PEG₁₆-FITC.

FIG. 26E is a graph of days after CAR T injection vs. tumor volume (mm³) for single tumor FAP8-PEG₁₅-FITC.

FIG. 27 is a graph of treatment group vs. % hCD3+ cells/total live cells.

FIG. 28 shows chemical structures of DUPA-FITC (2-[3-(1,3-dicarboxypropyl)ureido]pentanedioic acid (DUPA) linked to fluorescein isothiocyanate (FITC)) with different PEG linkers and the chemical structure of (((S)-5-amino-1-carboxypentyl)carbamoyl)-L-glutamic acid (PSMAL1).

FIG. 29 is a graph of [FL-DUPA](nM) vs. mean fluorescence intensity (MFI) of FITC, which shows the effects of linker length on binding affinity to PSMA.

FIG. 30 is a graph of cell line vs. MFI of APC-anti-PSMA.

FIG. 31A is a graph of DUPA-FITC with different PEG linkers in MDA-PSMA cells vs. MFI of FITC.

FIG. 31B is a graph of DUPA-FITC with different PEG linkers in MDA-PSMA cells vs. MFI of APC-anti-FITC.

FIG. 32A is a graph of [DUPA-FITC](nm) vs. lysis (%), which shows FITC-PEG-DUPA-mediated cytotoxicity of anti-FITC CAR-T cells on HOS-143b-PSMA cells.

FIG. 32B is a graph of [DUPA-FITC](nm) vs. IFNγ (pg/ml), which shows FITC-PEG-DUPA-mediated IFNγ release from anti-FITC CAR-T cells when cocultured with HOS-PSMA cells.

FIG. 32C is a graph of [DUPA-FITC](nm) vs. lysis (%), which shows FITC-PEG-DUPA-mediated cytotoxicity of anti-FITC CAR-T cells on LNCap cells.

FIG. 32D is a graph of [DUPA-FITC](nm) vs. IFNγ (pg/ml), which shows FITC-PEG-DUPA-mediated IFNγ release from anti-FITC CAR-T cells when cocultured with LNCap cells.

FIG. 32E is a graph of [DUPA-FITC](nm) vs. lysis (%), which shows FITC-PEG-DUPA-mediated cytotoxicity of anti-FITC CAR-T cells on 22Rv1 cells.

FIG. 32F is a graph of [DUPA-FITC](nm) vs. IFNγ (pg/ml), which shows FITC-PEG-DUPA-mediated IFNγ release from anti-FITC CAR-T cells when cocultured with 22Rv1 cells.

FIG. 33A shows the protocol used in Example 33.

FIG. 33B is a graph of hours post-injection vs. mean rad (photons/s/cm²/sr).

FIG. 34A shows the timeline and dosing schedule of the in vivo study of Example 8 to test DUPA-FITC with different PEG linkers.

FIG. 34B is a graph of days post-CAR-T injection vs. tumor volume (mm³).

FIG. 34C is a graph of days post-CAR-T injection vs. body weight change (%).

FIG. 35 is a graph of [PSMA ligand-FITC](nm) vs. DUPA-PEG₆-FITC.

FIG. 36A shows confocal images.

FIG. 36B shows flow cytometry data.

FIG. 36C shows flow cytometry data.

FIG. 37A shows [PSMA ligand-FITC](nM) vs. lysis (%).

FIG. 37B shows [PSMA ligand-FITC](nM) vs. IFNγ (pg/ml).

FIG. 38 shows the protocol used in Example 38 and fluorescent images at indicated time points of MDA-PSMA tumor-bearing mice intravenously injected with the indicated adapters at 500 nmol/kg.

FIG. 39 shows the protocol used in Example 39 and total FITC and surface FITC for MDA-MB-231 PSMA tumors for the indicated adapters 48-hours post-injection. Also shown are the results 144-hours post-injection with PSMAL1-PEG₆-FITC.

FIG. 40A shows the protocol used in Example 14.

FIG. 40B shows a graph of days post-CAR-T injection vs. tumor volume (mm³).

FIG. 40C shows a graph of days post-CAR-T injection vs. body weight change (%).

FIG. 41A shows the protocol used in in Example 15.

FIG. 41B shows a graph of days post-CAR-T injection vs. tumor volume (mm³).

FIG. 41C a graph of days post-CAR-T injection vs. body weight change (%).

FIG. 42A is a graphical representation of a TagCAR lentiviral particle, which contains cocal glycoprotein and a multi-domain fusion protein composed of an anti-CD3 scFV sandwiched between costimulatory proteins.

FIG. 42B is representative flow plots one hour following incubation of peripheral blood mononuclear cells with TagCAR lentiviral particles, where anti-cocal antibody is used to detect TagCAR lentiviral particle binding to cells.

FIG. 42C shows the mean (+/−SEM) percent and gMFI of cocal on circulating immune cell subsets following incubation of peripheral blood mononuclear cells (PBMCs) with TagCAR lentiviral particles.

FIG. 42D shows the mean (+/−SEM) of percent CD25+(activation) and TagCAR+(transduction) of CD3+ T cells at day 3 and 7 post transduction of peripheral blood mononuclear cells with TagCAR lentiviral particles, respectively.

FIG. 43A is a graphical representation of a competition assay to define a FITC ligand/TagCAR interaction, where TagCAR T cells are incubated with saturating levels of FL-AF647, which is then competed off by increasing concentrations of FITC ligand.

FIG. 43B shows FL-AF647 mean fluorescent intensity (MFI) on TagCAR+ T cells in the presence of increasing concentrations of PSMAL1-PEG₆-FITC or the antigen only control, sodium fluorescein (NaFL).

FIG. 43D is a graph showing the mean calculated inhibitor constant (Ki) NaFL and PSMAL1-PEG₆-FITC (triplicates=TagCAR T cells derived from different donors).

FIG. 44A is a graphical representation of using anti-fluorescein antibody to detect surface fluorescein antigen (SurfaceTag) on MDA-MB-231 tumor cells.

FIG. 44B shows SurfaceTag levels (APC MFI) following incubation of PSMA-overexpressing (PSMA⁺) or wildtype (PSMA⁻) MDA-MB-231 tumor cells with PSMAL1-PEG₆-FITC.

FIG. 45A is a graph showing the mean (+/−SEM) normalized fold tumor cell growth in the presence of TagCAR T cells with PSMAL1-PEG₆-FITC over time.

FIG. 45B shows the mean (+/−SEM) cytokines (left, IFNg; right, IL-2) levels in culture supernatants 24 hours following the addition of TagCAR T cells and at different concentrations of PSMAL1-PEG₆-FITC to PSMA-overexpressing MDA-MB-231 tumor cells.

FIG. 45C shows the mean (+/−SEM) percent of TagCAR+ cells after 88 hours of coculture with PSMA-overexpressing MDA-MB-231 tumor cells and different concentrations of PSMAL1-PEG₆-FITC.

FIG. 45D shows the percent CD25+ of TagCAR+ and TagCAR− CD3+ T cells after 88 hours of coculture with PSMA-overexpressing MDA-MB-231 tumor cells and different concentrations of PSMAL1-PEG₆-FITC.

FIG. 46A shows a series of graphs of day vs. tumor volume (mm³) for grouped tumor size (4M5.3 group), single tumor disease control, single tumor 4M5.3 CAR-T cell only, and single tumor 4M5.3 and FAP5-PEG₈-FITC.

FIG. 46B shows a series of graphs of day vs. tumor volume (mm³) for group tumor size (E2 group), single tumor disease control, single tumor E2 CAR-T cell only, and single tumor E2 and FAP5-PEG₈-FITC.

FIG. 46C is a graph of day vs. weight change (%), which shows mice weight over the course of treatment.

FIG. 47A is a graph of FAP5-PEG₁₆-FITC concentration vs. % total lysis, which shows the flow cytometry results over 24 hours coculture of in vitro killing of MDA-MB-231-hFAP cells by 4M5.3 CAR-T cells (E:T=1:1).

FIG. 47B is a graph of hours vs. AU, which shows the Incucyte results of an in vitro killing assay of MDA-MB-231-hFAP cells by 4M5.3 CAR-T cells over 68 hours coculture (E:T=1:1).

FIG. 48A is a graph of day vs. tumor volume (mm³), which shows the effect of treatment on tumor size.

FIG. 48B is a graph of day vs. weight change (%), which shows the effect of treatment on body weights of mice.

FIG. 48C shows graphs of treatment vs. hCD3+ count/μL of blood and treatment vs. hIFNγ (pg/ml).

FIG. 48D shows a graph of treatment vs. hCD3+/live cell (%).

FIG. 48E shows a diagram of an experimental design, a graph of days post-CAR-T injection vs. tumor volume (mm³), a graph of treatment vs. hCD3+ T-cells/total tumor cell (%), and a graph of treatment vs. hIFNγ (pg/mL).

FIG. 48F shows immunohistochemistry (IHC) slides, where CAFs were stained using anti-mouse alpha smooth muscle actin (a marker for CAFs), and the cancer cells were stained with anti-mouse Ki67 as a proliferation marker of cancer cells.

FIG. 49A shows a dosing schedule for combination therapy with FAP5-PEG₈-FITC and DUPA-PEG₆-FITC.

FIG. 49B is a graph of days post-CAR-T injection vs. tumor volume (mm³), which shows HOS-PSMA4+ tumor growth.

FIG. 49C is a graph of treatment vs. human T-cell counts/μl, which shows human T-cell counts in blood.

FIG. 49D are graphs of treatment vs. human CD3+ T-cells/live cells (%), which shows human T-cell counts in HOS-PSMA tumors (left) and h-IFNγ from mice blood (right).

FIG. 50 shows a dosing schedule for combination therapy with FAP5-PEG₈-FITC and DUPA-PEG₆-FITC, graphs of days post-CAR-T injection vs. tumor size (mm³) for DUPA-PEG₆-FITC alone and DUPA-PEG₆-FITC in combination with FAP5-FITC, and a graph of treatment vs. human T-cell count/μL blood, which shows human T-cell counts in blood at day 29.

FIG. 51A shows the timeline and dosing schedule of an in vivo study to test Aza-PEG₆-FITC and orthoCAL-PEG₆-FITC in combination with EC17.

FIG. 51B is a graph of days post-CAR-T cell injection vs. tumor volume (mm³), which shows tumor growth curves of different treatment groups. Aza-PEG₆-FITC in combination with EC17 significantly inhibited the growth of KB tumors. OrthoCAL-PEG₆-FITC in combination with EC17 also showed slightly better inhibition of the growth of KB tumors.

FIG. 51C is a graph of days post-CAR-T cell injection vs. body weight change (%), which shows body weight changes of mice in different treatment groups. Aza-PEG₆-FITC and EC17 induced body weight loss in mice.

FIG. 52A shows the timeline and dosing schedule of an in vivo study to test different CAIX bispecific adapters.

FIG. 52B is a graph of days post-CAR-T cell injection vs. tumor volume (mm³), which shows tumor growth curves of different treatment groups. Aza-PEG₆-FITC in combination with FAP8-PEG₁₈-FITC slightly inhibited the growth of KB tumors. The efficacy was similar to the combination of EC17 and FAP8-PEG₁₈-FITC. OrthoCAL-PEG₆-FITC in combination with FAP8-PEG₁₈-FITC showed better efficacy on inhibiting the growth of KB tumors.

FIG. 52C is a graph of days post-CAR-T cell injection vs. body weight change (%), which shows body weight changes of mice in different treatment groups. All the combinations did not induce significant body weight loss in the treated mice.

FIG. 53A shows the timeline and dosing schedule of an in vivo study.

FIG. 53B is a graph of days post-CAR-T cell injection vs. tumor volume (mm³), which shows tumor growth curves of different treatment groups. Aza-PEG₆-FITC in combination with FAP8-PEG₁₈-FITC and EC17 has similar efficacy as orthoCAL-PEG6-FITC in combination with FAP8-PEG₁₈-FITC and EC17. They both showed slightly better efficacy than the combination of EC17 and FAP8-PEG₁₈-FITC on inhibiting the growth of KB tumors.

FIG. 53C is a graph of days post-CAR-T cell injection vs. body weight change (%), which shows body weight changes of mice in different treatment groups. Mice injected with the combination of Aza-PEG₆-FITC, EC17 and FAP8-PEG₁₈-FITC showed body weight loss in the treatment. The body weight loss could be due to cytokine release from expanded CAR-T cells. The toxicity can be minimized by optimizing the dosing of the adapter.

FIG. 54A is a graph of FAP-FITC concentration (nmol/L) vs. MFI (FITC).

FIG. 54B is a graph of FAP-FITC concentration (nmol/L) vs. MFI (FITC).

FIG. 54C are IHC images showing KB tumor had less CAR-T cell infiltration than the MDA-MB231 tumor (top row) and that the KB tumor contains more mFAP⁺ CAF (bottom row), possibly restricting CAR-T cell infiltration.

FIG. 55A is a graph of FAP-FITC concentration (nmol/L) vs. % total lysis.

FIG. 55B is a graph of FAP-FITC concentration (nmol/L) vs. % total lysis.

FIG. 56A is a graph of days post-CAR T cell injection vs. tumor volume (mm³).

FIG. 56B is a graph of days post-CAR T cell injection vs. tumor volume (mm³).

FIG. 56C is a graph of days post-CAR T cell injection vs. tumor volume (mm³).

FIG. 56D is a graph of days post-CAR T cell injection vs. tumor volume (mm³).

FIG. 56E is a graph of days post-CAR T cell injection vs. tumor volume (mm³).

FIG. 57A is a graph of treatment group vs. IFNγ (pg/ml).

FIG. 57B is a graph of treatment group vs. CAR+ count/μl of blood.

FIG. 58A is a graph of treatment group vs. IFNγ (pg/ml).

FIG. 58B is a graph of treatment group vs. CAR+ count/μl of blood.

FIG. 59 is a graph of treatment group vs. CAR+ cells/50,000 live cells.

FIG. 60 shows IHC images of hCD3 staining of the treatment groups, with an increase of T cell infiltration observed after FAP-FITC treatment.

### DETAILED DESCRIPTION

The present disclosure is predicated, at least in part, on the discovery that cancer-associated fibroblasts (CAFs) can affect the efficacy of chimeric antigen receptor (CAR)-T cells in the treatment of solid tumors. For example, KB tumors (human epithelial carcinoma) and MDA-MB-231 tumors (human invasive ductile carcinoma) were implanted in NOD scid gamma (NSG) mice. Both groups of mice were treated with anti-fluorescein isothiocyanate (FITC) CAR-T and EC17. Both tumors were then harvested from the mice, fixed with 10% formalin overnight, and rinsed with 70% ethanol for tumor fixation. The tumor cells were then sent for immunohistochemistry (IHC) staining for either anti-human CD3 antibody to detect human CAR-T cells or anti-mouse fibroblast activation protein (FAP) antibody to detect mouse FAP+ CAFs. Anti-human CD3 antibody staining indicated CAR-T cells infiltrated the MDA-MB-231 tumor but did not infiltrate the KB tumor. Anti-mouse FAP antibody staining indicated significant infiltration of FAP+ CAFs in the KB tumor and substantially less infiltration of FAP+ CAFs in the MDA-MB-231 tumor.

In view of the above, provided is a bispecific adapter or a pharmaceutically acceptable salt or hydrate thereof for use with anti-fluorescein (e.g., fluorescein, FITC, or N-hydroxysuccinimide (NHS)-fluorescein) CAR-T cells in the treatment of FAP-expressing (e.g., FAPα- or FAPβ-expressing) or a prostate-specific membrane antigen (PSMA)-expressing cancer.

In certain embodiments, the bispecific adapter comprises the following structure:

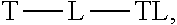

or is a pharmaceutically acceptable salt or hydrate thereof, wherein:
F comprises a CAR-T cell targeting moiety such as, for example, fluorescein. FITC, or NHS-fluorescein,
L comprises a linker, and
TL comprises a cancer targeting or cancer-associated cell targeting ligand comprising a FAP ligand, a PSMA ligand, or a radical of either of the foregoing.

The use of bispecific adapters can enable the use of a single CAR-T cell, i.e., a “universal” CAR-T cell, that displays, for example, a molecule on its surface that binds fluorescein. When the universal CAR-T cell, such as one displaying a molecule on its surface that binds fluorescein, is used in conjunction with a bispecific adapter, such as one comprising FITC connected (e.g., by a linker and/or spacer) to a molecule, which binds a cell-surface receptor on CAFs and tumor cells, the T-cell can kill the CAFs and tumor cells to which it is bound.

This approach can reduce the cost of producing CAR-T cells that can bind different cancers that express different cell-surface receptors. Instead, the universal CAR-T cell can bind to different types of cancers by changing the part of the bispecific adapter that binds a cell-surface receptor on a tumor cell. Accordingly, the bispecific adapters can improve the immune response effected by CAR-T cells bound to tumor cells and inhibit the effect of CAFs.

### CAR T-Cell Targeting Moiety

The CAR T-cell targeting moiety of the bispecific adapter can be fluorescein, FITC, NHS-fluorescein, or any other moiety that a CAR can be engineered to recognize and bind with specificity.

“Bind with specificity,” “binds with high affinity,” or “specifically” or “selectively” binds, when referring to a ligand/receptor, a recognition region/targeting moiety, an antibody/antigen, or other binding pair indicates a binding reaction that is determinative of the presence of the protein in a heterogeneous population of proteins and other biologics. Thus, under designated conditions, a specified ligand or recognition region binds to a particular receptor (e.g., one present on a cancer cell or CAR T-cell) or targeting moiety, respectively, and does not bind in a significant amount to other proteins present in the sample (e.g., those associated with normal, healthy cells). Specific binding or binding with high affinity can also mean, for example, that the binding compound, ligand, antibody, or binding composition derived from the antigen-binding site of an antibody binds to its target with an affinity that is often at least 25% greater, more often at least 50% greater, most often at least 100% (2-fold) greater, normally at least ten times greater, more normally at least 20-times greater, and most normally at least 100-times greater than the affinity with any other binding compound. In a typical embodiment, a molecule that specifically binds a target will have an affinity that is at least about 10⁶ liters/mol (K_{D}=10⁻⁶ M), and preferably at least about 10 liters/mol, as determined, for example, by Scatchard analysis.

### Targeting Ligands

As noted above, the bispecific adapters can comprise a cancer-targeting or cancer-associated cell-targeting ligand comprising a FAP ligand, a PSMA ligand, a folate receptor (FR), or a radical of any of the foregoing. When administered, the targeting ligand targets the bispecific adapter compound to a cancer or tumor of interest or a cancer-associated cell of interest. In some embodiments, the targeting moieties (in their free form, a radical thereof) do not bind with uptake receptors on non-targeted cells.

Generally, tumors can comprise infiltrating immune and inflammatory cells such as cancer-associated fibroblasts (CAFs), extracellular matrix (ECM) proteins, T cells, tumor-associated macrophages (TAMs), myeloid-suppressor cells, blood and lymphatic vasculature, etc., which aid in the growth and development of the tumor by growth factor secretion, immunosuppression, metastasis, resistance, etc. CAFs are one of the major types of cells present in the tumor stroma and perform several critical roles to promote tumor growth. These functions include ECM production, remodeling, and cytokine secretion, which can lead to angiogenesis to promote tumor growth, signaling factor secretion to increase chemoresistance, denser tumor stroma to provide a physical blockade against immune cells, and enhanced cell motility to direct metastasis. In some instances, such processes parallel the behavior of pathogenic fibroblasts in fibrotic diseases.

In some instances, a prevalent marker of CAFs is fibroblast activation protein alpha (FAPα). FAPα is a serine protease (primarily) found on the cell surface of activated fibroblasts in diseased cells and tissue, such as in fibrotic disease, inflammatory disease, and/or cancer (e.g., fibrosis, rheumatoid arthritis, wound healing, and cancer). FAP is expressed on the surfaces of CAFs and has been proven to correlate with poor patient prognosis in multiple solid tumors. In addition, virtually every human solid cancer over-expresses FAP.

For example, more than 90% of epithelial carcinomas show FAPα expression in immunohistochemical (IHC) staining. Additional FAPα expression has been found in a subset of primary glioma cell cultures and TAMs. Recently, FAPα expression has been detected in at least 28 different types of human cancers. However, FAPα expression is very low or nonexistent in the majority of healthy adult tissues. Therefore, because the expression is restricted to the surfaces of diseased cells, such as carcinomas, FAPα is uniquely qualified as a receptor for selectively delivering pharmacotherapeutics to tumors via ligand-targeting.

The bispecific adapters hereof can comprise a FAP-targeting ligand (or a radical thereof) attached to a linker, wherein the linker is further attached to a CAR-targeting moiety. FAP is a type II membrane bound serine protease that cleaves proline-amino acid peptide bonds and can be expressed on CAFs and on myofibroblasts that produce collagen. In certain embodiments, the bispecific adapter can target CAR T-cells coupled with the CAR-targeting moiety of the bispecific adaptor to a FAP-expressing cancer or fibrotic or inflammatory disease. In certain embodiments, this improved FAP ligand scaffold can additionally be used with albumin-binding moieties to achieve the targeted delivery of radiolabeled and other functional groups. In certain embodiments, the FAP ligand is a high affinity FAP ligand that comprises a triazole moiety (or a derivative thereof) introduced into a scaffold of the ligand. In certain embodiments, the FAP ligand is a high-affinity FAP ligand that comprises a triazole moiety (or a derivative thereof) and a phenyl ring introduced into a scaffold of the ligand (e.g., an isoindoline ring scaffold). Unless otherwise specified, “high affinity” or “higher affinity” with respect to a ligand's affinity for a target means a ligand that has a Schrodinger molecular docking score of at least about −8.0 kcal/mol. In certain embodiments, the high affinity FAP ligand has an improved affinity for FAP as compared to a ligand without a triazole moiety introduced therein.

The targeting moiety can be, for example, a radical of FAPα ligand with a molecular weight less than about 10,000, less than 7,500, less than 5,000, less than 2,500, less than 1,000, less than 760, less than 500; from about 500 to about 10,000 g/mol, about 1,000 to about 7,500 g/mol, about 750 g/mol to about 1,500 g/mol, about 1,000, to about 5,000 g/mol or about 500 to about 2,500 g/mol.

The targeting ligand can bind to an activated fibroblast expressing FAP (e.g., FAPα or FAPβ) where such activated fibroblast is involved in cancer. In certain embodiments, the targeting ligand can have a binding affinity to a FAP (e.g., FAPα) in the range between about 1 nM to about 25 nM, such as 1 nM to about 25 nM or about 1 nM to 25 nM.

In certain embodiments, the FAP ligand is FAP5 having or comprising the structure of:

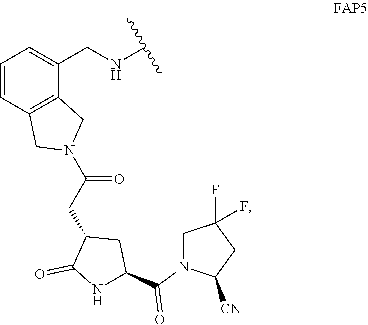 [SMILES: *NCc1cccc2c1CN(C(=O)C[C@@H]1C[C@@H](C(=O)N3CC(F)(F)C[C@H]3C#N)NC1=O)C2]

wherein

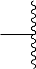

is the point of attachment to the linker of the adaptor.

In certain embodiments, the FAP ligand or radical thereof is FAP8 having or comprising the structure of

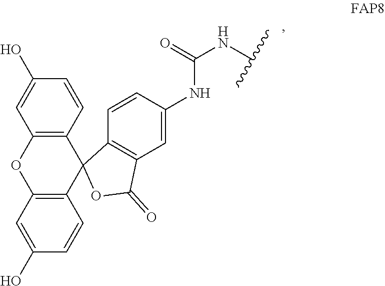 [SMILES: *NC(=O)Nc1ccc2c(c1)C(=O)OC21c2ccc(O)cc2Oc2cc(O)ccc21]

wherein

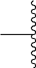

is the point of attachment to the linker of the adaptor.

In certain embodiments, the FAP ligand or radical thereof can be or comprise a FAP5 ligand or radical having a structure represented by the formula I-B:

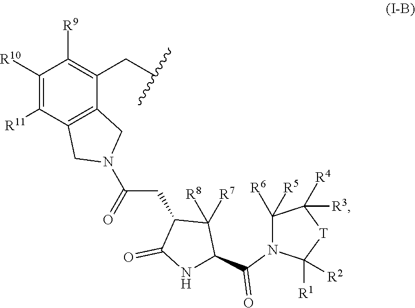

wherein:

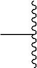

is the point of attachment to the linker of the adaptor;
T is substituted or unsubstituted methylene (—CH₂—), substituted or unsubstituted amino (—NH—), —O—, or —S—;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F and 5-tetrazolyl;
R³ and R⁴ are each independently selected from the group consisting of —H, —OH, F. Cl, Br, I, —C₁₋₆alkyl, —O—C₁₋₆alkyl, and —S—C₁₋₆alkyl;
R⁵, R⁶, R⁷, and R⁸ are each independently selected from the group consisting of H, alkyl and halo; and
R⁹, R¹⁰, and R¹¹ are each independently selected from the group consisting of H, —C₁₋₆alkyl, —O—C₁₋₆alkyl, —S—C₁₋₆ alkyl, F, Cl, Br and I.

The FAP ligand or radical thereof can be or comprise a FAP5 ligand or radical having a structure represented by the formula I-C:

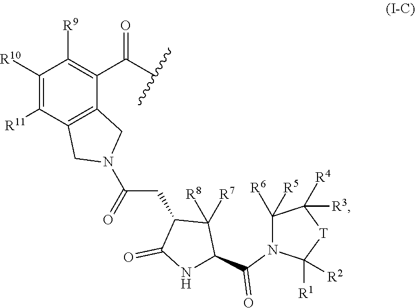

wherein:

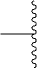

is a point of attachment to the linker;
T is substituted or unsubstituted methylene (—CH₂—), substituted or unsubstituted amino (—NH—), —O—, or —S—;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, and 5-tetrazolyl;
R³ and R⁴ are each independently selected from the group consisting of —H, —OH, F. Cl, Br, I, —C₁₋₆alkyl, —O—C₁₋₆alkyl, and —S—C₁₋₆alkyl;
R⁵, R⁶, R⁷, and R⁸ are each independently selected from the group consisting of H, alkyl and halo; and
R⁹, R¹⁰, and R¹¹ are each independently selected from the group consisting of H, —C₁₋₆alkyl, —O—C₁₋₆alkyl, —S—C₁₋₆ alkyl, F, Cl, Br and I.

In certain embodiments, the FAP ligand or radical thereof can be or comprise a FAP8 ligand or radical having the structure:

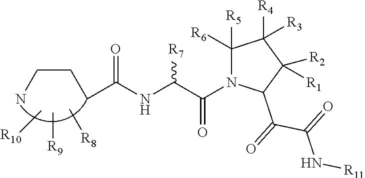

wherein:

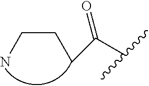 [SMILES: *C(=O)CCCN]

represents a functionalized 5- to 10-membered N-containing aromatic or non-aromatic mono- or bi-cyclic heterocycle, which optionally further comprises 1-3 heteroatoms independently selected from O, N, and S;
R₁ and R₂ are independently selected from the group consisting of —H, -D, —OH, —F, —Cl, —Br, —I, —C₁₋₆ alkyl, —O—C₁₋₆ alkyl, and —S—C₁₋₆ alkyl;
R₃ and R₄ are independently selected from the group consisting of —H, —OH, —F, —Cl, —Br, —I, —C₁₋₆ alkyl, —O—C₁₋₆ alkyl, and —S—C₁₋₆ alkyl;
R₅ and R₆ are independently selected from the group consisting of —H, —OH, —F, —Cl, —Br, —I, —C₁₋₆ alkyl, —O—C₁₋₆ alkyl, and —S—C₁₋₆ alkyl;
R₇ is selected from the group consisting of —H, -D, OH, CH₂═, —CH₃, CH₃CH₂—, (CH₃)₂CH—, (CH₃)₃C—, —CH₂Ph, and substituted —CH₂Ph;
R₈-R₁₀ are independently selected from the group consisting of —H, —OH, —F, —Cl, —Br,
—I, —NO₂, —SO₃H, —SO₂NH₂, —NH₂, —N₃, —NH═NH, —C₁₋₆ alkyl, —O—C₁₋₆ alkyl, and —S—C₁₋₆ alkyl; and
R₁₁ is selected from the group consisting of —H, -D, C₁-C₁₀ alkyl, C₃-C₁₀ cycloalkyl, adamantyl,

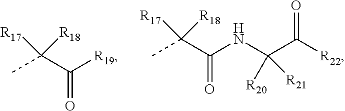

substituted or unsubstituted aryl, substituted or unsubstituted C₇-C₂₀ alkyl aryl, wherein the aryl is:

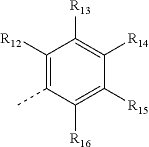

wherein:
R₁₂ and R₁₆ are independently selected from the group consisting of —H, -D, halogen, C₁-C₃ alkyl, C₁-C₃ alkoxy, —CF₃, and —C(═O)—OR₂₃, wherein R₂₃ is selected from the group consisting of H, D, halogen, C₁-C₄ alkyl, and C₁-C₃ alkoxy;
R₁₃, R₁₄ and R₁₅ are independently selected from the group consisting of —H, -D, halogen, —OMe, C₁-C₃ alkyl. C₁-C₃ alkoxy, —CF₃, and —C(═O)—OR₂₃, wherein R₂₃ is selected from the group consisting of —H, -D, halogen, C₁-C₄ alkyl, and C₁-C₃ alkoxy;
R₁₇, R₁₈, R₂₀, and R₂₁ are independently selected from —H and —CH₃; and
R₁₉ and R₂₂ are independently selected from the group consisting of phenyl, dimethoxyphenyl, and aryl.

In certain embodiments, the targeting ligand of the bispecific adapter comprises a PSMA ligand or radical thereof. PSMA is expressed in tumor neovasculature of multiple cancers including ovarian cancer (100%), endometrial cancer (100%), breast cancer (60%), stage 3-4 gliomas (100%), and stage 3-4 clear cell renal cell carcinoma (100%). PSMA is also over-expressed in prostate cancer but has little expression in normal tissue. Though PSMA is expressed in the brain, that expression is minimal, and most ligands of PSMA are polar and not capable of penetrating the blood brain barrier. As such. PSMA can be a valuable targeting ligand in the present context.

PSMA is a type H, cell-surface membrane-bound glycoprotein with ~110 kD molecular weight, including an intracellular segment (amino acids 1-18), a transmembrane domain (amino acids 19-43), and an extensive extracellular domain (amino acids 44-750). While the functions of the intracellular segment and the transmembrane domains are currently believed to be insignificant, the extracellular domain is involved in several distinct activities. PSMA plays a role in the central nervous system, where it metabolizes N-acetyl-aspartyl glutamate (NAAG) into glutamic and N-acetyl aspartic acid. Accordingly, it is also sometimes referred to as an N-acetyl alpha linked acidic dipeptidase (NAALADase). PSMA is also sometimes referred to as a folate hydrolase 1 (FOLH I) or glutamate carboxypeptidase (GCP II) due to its role in the proximal small intestine where it removes T-linked glutamate from poly-γ-glutamated folate and α-linked glutamate from peptides and small molecules.

Unlike many other membrane-bound proteins, PSMA undergoes rapid internalization into the cell in a similar fashion to cell surface-bound receptors like vitamin receptors. PSMA is internalized through clathrin-coated pits and subsequently can either recycle to the cell surface or go to lysosomes. It has been suggested that the dimer and monomer form of PSMA are inter-convertible, though direct evidence of the interconversion is being debated. Even so, only the dimer of PSMA possesses enzymatic activity, and the monomer does not.

Though the activity of the PSMA on the cell surface of the prostate cells remains under investigation, PSMA is a viable target for the selective and/or specific delivery of CAR T-cells to PSMA-expressing cells.

In certain embodiments, the targeting ligand of the bispecific adapter comprises a PSMA ligand or radical thereof. The PSMA can be or can comprise (((S)-5-amino-1-carboxypentyl)carbamoyl)-L-glutamic acid (PSMAL1). The PSMA can be or can comprise 2-[3-(1,3-dicarboxypropyl)ureido]pentanedioic acid (DUPA) or a derivative thereof (see, e.g., International Patent Application Publication No. WO 2015/057852, which describes DUPA derivatives and which is hereby incorporated by reference for its teachings regarding the same). When administered, a bispecific adapter compound comprising a PSMA ligand or radical thereof can target a PSMA-expressing cancer in a subject.

The bispecific adapter can be specifically designed and synthesized to achieve a particular binding affinity for PSMA. For example, when PSMAL1-PEG₆-FITC was compared with DUPA-PEG₆-FITC in vitro and in vivo, PSMAL1-PEG₆-FITC demonstrated higher binding affinity to PSMA and longer retention time in PSMA-positive cells. Further, PSMAL1-PEG₆-FITC demonstrated higher efficacy in mediating anti-FITC CAR-T cell function at low concentrations. PSMAL1-PEG₆-FITC also demonstrated efficacy in mediating eradication of tumors expressing low levels of PSMA by anti-FITC CAR-T cell.

In certain embodiments, the targeting ligand of the bispecific adapter comprises a folate or radical thereof. “Folate” refers to a FR-binding molecule (e.g., FRα or FRβ) including, for example, folic acid and analogs and derivatives of folic acid such as, without limitation, folinic acid, pteroylpolyglutamic acid, pteroyl-D-glutamic acid, and FR-binding pteridines such as tetrahydropterins, dihydrofolates, tetrahydrofolates, and their deaza and dideaza analogs. The folate of the adapter comprising fluorescein-linker-folate can be folate, dihydrofolate tetrahydrofolate, 5, 10-methylene tetrahydrofolate (5,10-MTHF), 5-methyltetrahydrofolate (5-MTHF), or raltitrexed (binds FRα but not FRβ).

The terms “deaza” and “dideaza” analogs refer to the art-recognized analogs having a carbon atom substituted for one or two nitrogen atoms in the naturally occurring folic acid structure, or analog or derivative thereof. For example, the deaza analogs may include the 1-deaza, 3-deaza. 5-deaza, 8-deaza, and 10-deaza analogs of folate, folinic acid, pteropolyglutamic acid, and folate receptor-binding pteridines such as tetrahydropterins, dihydrofolates, and tetrahydrofolates. The dideaza analogs include, for example, 1,5-dideaza, 5,10-dideaza, 8,10-dideaza, and 5,8-dideaza analogs of folate. Other folates useful as complex-forming ligands are the folate receptor-binding analogs pemetrexed, proguanil, pyrimethamine, trimethoprim, pralatrexate, raltitrexed, aminopterin, amethopterin (also known as methotrexate). N¹⁰-methylfolate, 2-deamino-dydroxyfolate, deaza analogs such as 1-deazamethopterin or 3-deazamethopterin, and 3′,5′-dichloro-4-amino-4-deoxy-N¹⁰-methylpteroylglutamic acid (dichloromethotrexate).

Folic acid and the foregoing analogs and/or derivatives are also termed “a folate.” “the folate,” or “folates” reflecting their ability to bind to FRs, such as FRα or FRβ. Such molecules, when conjugated with exogenous molecules, are effective to enhance transmembrane transport, such as via folate-mediated endocytosis. The foregoing can be used in the fluorescein-linker-folate bispecific adapters.

FR alpha (FRα) is over-expressed in approximately 90% of ovarian cancer, 70% of endometrial cancer, 80% of triple-negative breast cancer, 80% of non-small cell lung cancer, and 65% of renal cell cancer. FR beta (FRβ) is over-expressed on immunosuppressive myeloid-derived suppressor cells (MDSCs) and tumor-associated macrophages (TAMs) in the tumor microenvironment (TME).

Immunohistochemistry (IHC) staining of solid tumors demonstrates the limited efficacy of CAR-T cells in solid tumors may be due to CAFs. KB tumor cells and MDA-MB-231 tumor cells were implanted in NOD scid gamma (NSG) mice. Both mice were treated with anti-FITC CAR-T and EC17 (a folate-FITC conjugate). Both tumors were then harvested from the mice, fixed with 10% formalin overnight, and rinsed with 70% ethanol for tumor fixation. Then, tumor cells were sent for IHC staining for either anti-human CD3 antibody to detect human CAR-T cells or anti-mouse FAP antibody to detect mouse FAP+ CAFs.

Anti-human CD3 antibody staining indicated CAR-T cells infiltrated the MDA-MB-231 tumor but did not infiltrate the KB tumor. Anti-mouse FAP antibody staining indicated significant infiltration of FAP+ CAFs in the KB tumor and substantially less infiltration of FAP+ CAFs in the MDA-MB-231 tumor.

KB tumor cells (i.e., a cell line that creates an immunologically “cold” FR-expressing solid tumor) were administered to NSG mice and the CAR T cell's toxicity was quantitated in the presence of one or two bispecific adapters. While administration of the universal CAR-T cells followed by intravenous injection of an FR-targeting bispecific adaptor achieved significant anti-tumor efficacy, co-injection of a FAP-targeted bispecific adaptor measurably enhanced this efficacy without apparent toxicity. Analyses of tumor masses over the course of the therapy further revealed that co-administration of the FAP-targeted bispecific adaptor not only promoted CAF elimination, but also enhanced CAR-T cell infiltration and activation.

Accordingly, in certain embodiments, the bispecific adapter can comprise a fluorescein conjugated to a radical of a FR ligand (e.g., via a linker). Such a fluorescein-linker-folate bispecific adapter can be used in combination with other bispecific adapters hereof (e.g., fluorescein-L-PSMA and/or fluorescein-L-FAP) for the treatment of cancer in a subject.

The fluorescein-linker-folate bispecific adapter can include a ligand (or radical thereof) having a structure of formula V or a functional fragment or analog thereof:

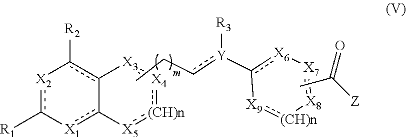

wherein:
X₁, X₂, X₃, X₄, X₅, X₆, X₇, X₈, and X₉ are each independently nitrogen (N), NH, CH, CH₂, oxygen (O), or sulfur (S);
Y is C, CH, CH₂, N, NH, O, or S;
Z is glutamic acid, valine, or a substrate;
R₁ and R₂ are each independently NH₂, OH, SH, CH₃, or H;
R₃ is H or an alkyl;
m and n are each independently 0, 1, or between 0 and 1; and
[symbol] is representative of either a single or double bond C—C.

In a further aspect, by way of nonlimiting example, the ligand (or radical thereof) of formula V has a structure of VI (or a functional fragment or analog thereof):

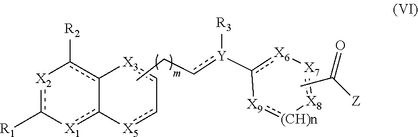

wherein
X₁, X₂, X₃, X₅, X₆, X₇, X₈, and X₉ are each independently N, NH, CH, CH₂, O, or S;
Y is C, CH, CH₂, N, NH, O, or S;
Z is glutamic acid, valine, or a substrate;
R₁ and R₂ are each independently NH₂, OH, SH, CH₃, or H;
R₃ is H or an alkyl;
m and n are each independently 0, 1, or between 0 and 1; and
[symbol] is representative of either a single or double bond C—C.

Another specific ligand (or radical thereof) of formula V (or a functional fragment or analog thereof) can have a structure of formula VII:

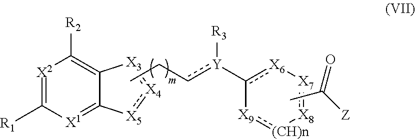

wherein
X₁, X₂, X₃, X₄, X₅, X₆, X₇, X₈, and X₉ are each independently N, NH, CH, CH₂, O, or S;
Y is C, CH, CH₂, N, NH, O, or S;
Z is glutamic acid, valine, or a substrate;
R₁ and R₂ are each independently NH₂, OH, SH, CH₃, or H;
R₃ is H or an alkyl;
m and n are each independently 0, 1, or between 0 and 1; and
[symbol] is representative of either a single or double bond C—C.

In certain embodiments, a ligand (or radical thereof) of formula VI can have the structure of formula VIII or a functional fragment or analog thereof:

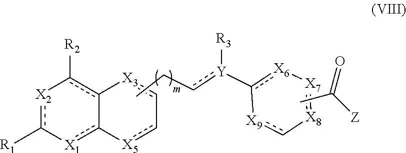

wherein
X₁, X₂, X₃, X₅, X₆, X₇, X₈, and X₉ are each independently N, NH, CH, CH₂, O, or S;
Y is C, CH, CH₂, N, NH, O, or S;
Z is glutamic acid, valine, or a substrate;
R₁ and R₂ are each independently NH₂, OH, SH, CH₃, or H;
R₃ is H or an alkyl;
m is 0, 1, or between 0 and 1; and
[symbol] is representative of either a single or double bond C—C.

A ligand (or radical thereof) of formula VI can have the structure of formula IX (or a functional fragment or analog thereof):

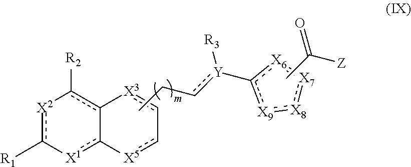

wherein
X₁, X₂, X₃, X₅, X₆, X₇, X₈, and X₉ are each independently N, NH, CH, CH₂, O, or S;
Y is C, CH, CH₂, N, NH, O, or S;
Z is glutamic acid, valine, or a substrate;
R₁ and R₂ are each independently NH₂, OH, SH, CH₃, or H;
R₃ is H or an alkyl;
in is 0, 1, or between 0 and 1; and
[symbol] is representative of either a single or double bond C—C.

A ligand (or radical thereof) of formula VII can have the structure of formula X or XI (or a functional fragment or analog of either):

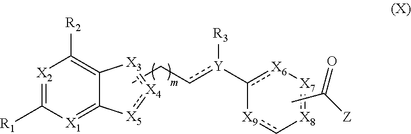

wherein
X₁, X₂, X₃, X₄, X₅, X₆, X₇, X₈, and X₉ are each independently N, NH, CH, CH₂, O, or S;
Y is C, CH, CH₂, N, NH, O, or S;
Z is glutamic acid, valine, or a substrate;
R₁ and R₂ are each independently NH₂, OH, SH, CH₃, or H;
R₃ is H or an alkyl;
m is 0, 1, or between 0 and 1; and
[symbol] is representative of either a single or double bond C—C;
   or

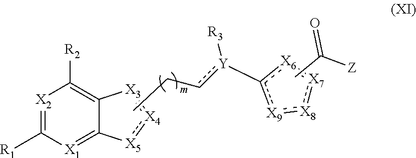

wherein
X₁, X₂, X₃, X₄, X₅, X₆, X₇, X₈, and X₉ are each independently N, NH, CH, CH₂, O, or S;
Y is C, CH, CH₂, N, NH, O, or S;
Z is glutamic acid, valine, or a substrate;
R₁ and R₂ are each independently NH₂, OH, SH, CH₃, or H;
R₃ is H or an alkyl;
m is 0, 1, or between 0 and 1; and
[symbol] is representative of either a single or double bond C—C.

Table 1 provides non-limiting examples of additional embodiments of a targeting ligand comprising a FR-targeting ligand (e.g., or radicals thereof) having the structure of formula VIII.

TABLE 1
Formula VIII

| Ligand | Structure |
|---|---|
| a | 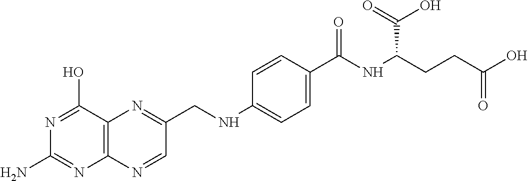 [SMILES: Nc1nc(O)c2nc(CNc3ccc(C(=O)N[C@@H](CCC(=O)O)C(=O)O)cc3)cnc2n1] |
| b | 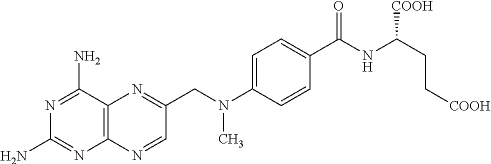 [SMILES: CN(Cc1cnc2nc(N)nc(N)c2n1)c1ccc(C(=O)N[C@@H](CCC(=O)O)C(=O)O)cc1] |
| c | 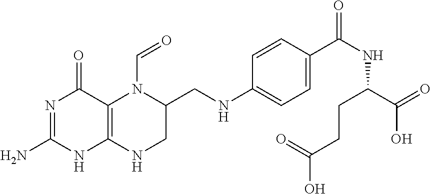 [SMILES: Nc1nc(=O)c2c([nH]1)NCC(CNc1ccc(C(=O)N[C@@H](CCC(=O)O)C(=O)O)cc1)N2C=O] |
| d | 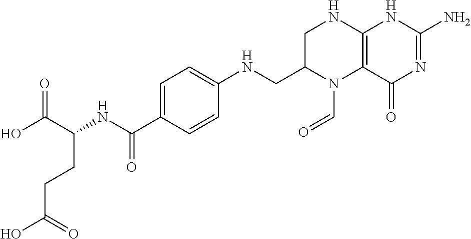 [SMILES: Nc1nc(=O)c2c([nH]1)NCC(CNc1ccc(C(=O)N[C@H](CCC(=O)O)C(=O)O)cc1)N2C=O] |
| e | 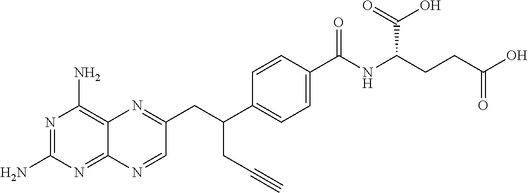 [SMILES: C#CCC(Cc1cnc2nc(N)nc(N)c2n1)c1ccc(C(=O)N[C@@H](CCC(=O)O)C(=O)O)cc1] |
| f | 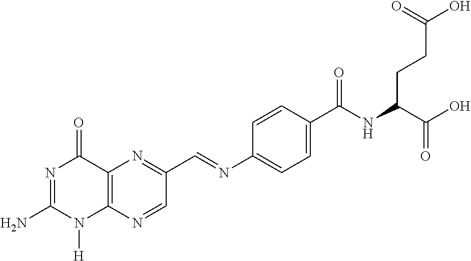 |
| g | 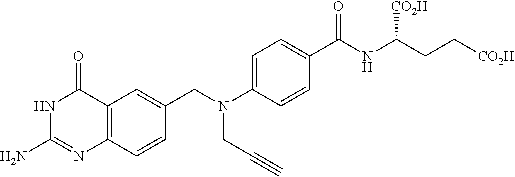 [SMILES: C#CCN(Cc1ccc2nc(N)[nH]c(=O)c2c1)c1ccc(C(=O)N[C@@H](CCC(=O)O)C(=O)O)cc1] |
| h | 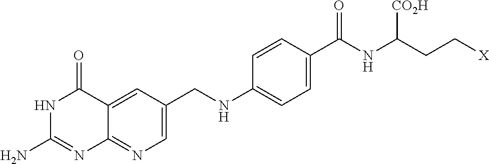 |
| | wherein X is PO(OH)₂, CH₂NH₂, or SO₂OH |
| i | 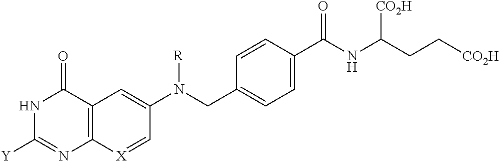 |
| | wherein |
| | X is N or CH; |
| | Y is NH₂, H, or CH₃; and |
| | R is H, CH₃, or CHO |
| j | 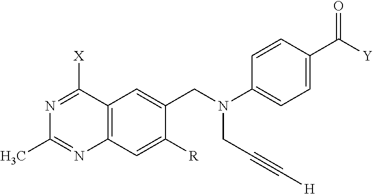 |
| | wherein |
| | X is OH or OCH₃; |
| | R is H or CH₃; and |
| | Y is glutamic acid, valine, or a substrate. |

Table 2 provides non-limiting examples of additional embodiments of a targeting ligand of the bispecific adapter comprising a FR-targeting ligand (e.g., or radicals thereof) having the structure of formula IX.

TABLE 2
Formula IX

| Ligand | Structure |
|---|---|
| aa | 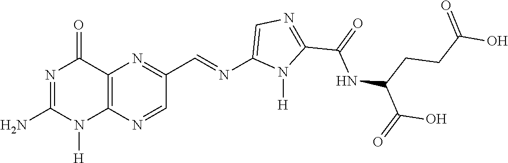 |
| bb | 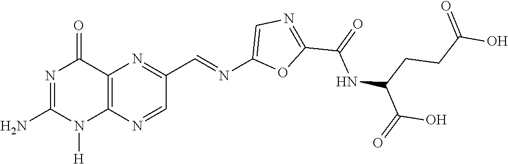 |
| cc | 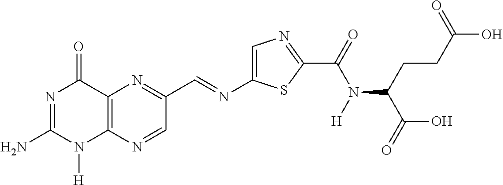 |
| dd | 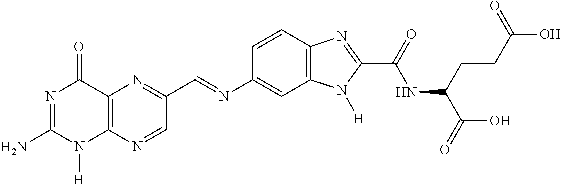 |
| ee | 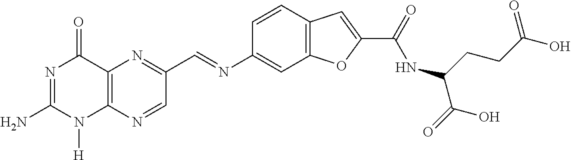 |
| ff | 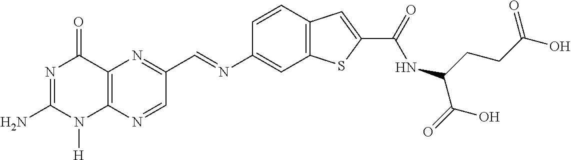 |

Table 3 provides non-limiting examples of additional embodiments of a targeting ligand of the bispecific adapter hereof comprising a FR-targeting ligand (or radical thereof) having the structure of formula X′.

TABLE 3
Formula X′

| Ligand | Structure |
|---|---|
| aaa | 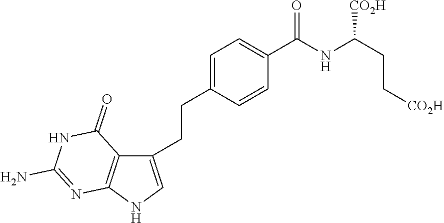 [SMILES: Nc1nc2[nH]cc(CCc3ccc(C(=O)N[C@@H](CCC(=O)O)C(=O)O)cc3)c2c(=O)[nH]1] |
| bbb | 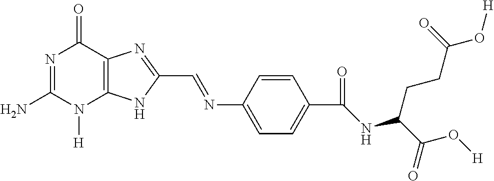 |
| ccc | 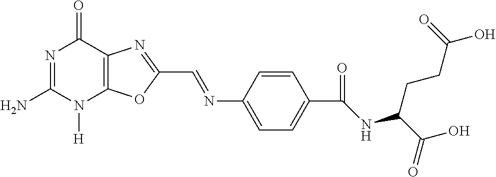 |
| ddd | 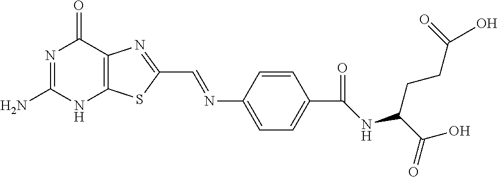 |

Instead of a folate, the targeting ligand (e.g., a radical thereof) can be one or more nonclassical antifolate analogs such as, for example, pyrido[2,3-d]pyrimidine or similar analogs (or radicals thereof) having the formulae (e.g., radicals of the formulae) set forth in Table 4 below (or an analog or functional fragment thereof).

TABLE 4
Nonclassical antifolate analogs

| Ligand | Formula |
|---|---|
| aaaa | 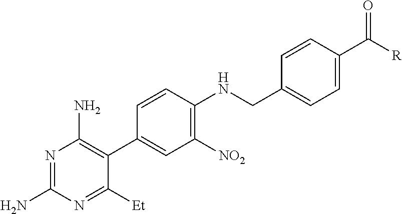 |
| | wherein R is NH₂, NHMe, NHCH(CO₂Et)(CH₂)₂CO₂Et, |
| | 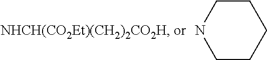 |
| bbbb | 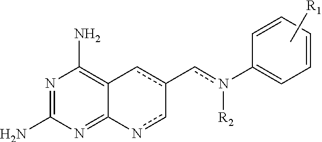 |
| | wherein |
| | R₁ is 3,4,5-(OCH₃)₃, 3,4-(OCH₃)₂, or 4-OCH₃; |
| | R₂ is an H, an alkyl chain or CHO; and [symbol] |
| | is representative of either a single or double bond C—C. |
| cccc | 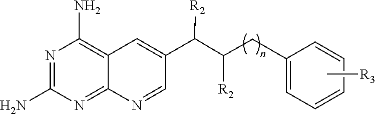 |
| | wherein |
| | n is 0 or 1; |
| | R₁ and R₂ are each independently an H or an alky; and |
| | R₃ is an H, 3′,4′,5′-Ome, 2′,3′,4′-Ome, 2′,4′,5′-Ome, |
| | 2′,4′,6′-Ome, 3′,4′-Ome, 3′,5′-Ome, 2′,5′-Ome, 2′,3′-C₄H₄, |
| | 4′-OMe,2′,3′-C₄H₄, 6′-OMe, 2′,3′-C₄H₄, 4′-O—C₆H₅ or 4′- |
| | CONH-L-glutamic acid. |
| dddd | 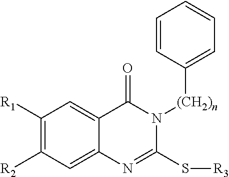 |
| | wherein |
| | n is 0 or 1; |
| | R₁ is CH₃, chloride (Cl) or OCH₃; |
| | R₂ is H or OCH₃; and |
| | R₃ is one of the following: |
| | 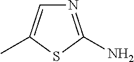 |
| | 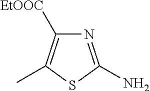 |
| | 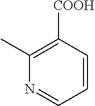 |
| | 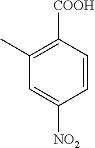 |
| eeee | 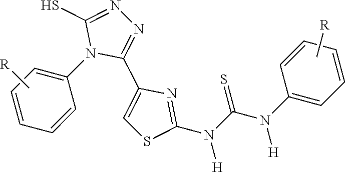 |
| | wherein R is H, 4-Cl, 2-CH₃O, 4-CH₃O, 2,4-(CH₃O)₂, 4- |
| | CH₃, or 4-C₆H₅O. |
| ffff | 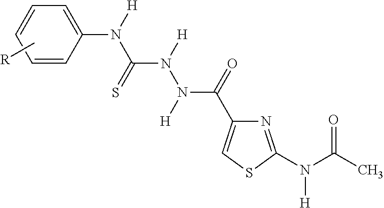 |
| | herein R is H, 4-Cl, 2-CH₃O, 4-CH₃O, 2,4-(CH₃O)₂, 4- |
| | CH₃, or 4-C₆H₅O. |
| gggg | 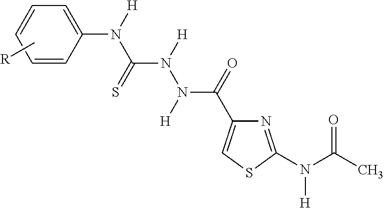 |
| | wherein R is H, 4-Cl, 2-CH₃O, 4-CH₃O, 2,4-(CH₃O)₂, 4- |
| | CH₃, or 4-C₆H₅O. |
| hhhh | 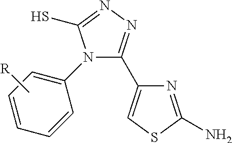 |
| | wherein R is H, 4-Cl, 2-CH₃O, 4-CH₃O, 2,4-(CH₃O)₂, 4- |
| | CH₃, or 4-C₆H₅O. |
| iiii | 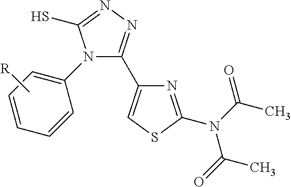 |
| | wherein R is H, 4-Cl, 2-CH₃O, 4-CH₃O, 2,4-(CH₃O)_{2=l , 4-} |
| | CH₃, or 4-C₆H₅O. |
| jjjj | 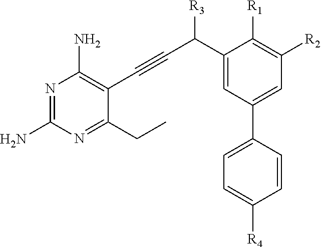 |
| | wherein |
| | R₁ and R₂ are each independently H or OMe; |
| | R₃ is H or an alky; and |
| | R₄ is o-COOH, m-COOH or p-COOH. |
| kkkk | 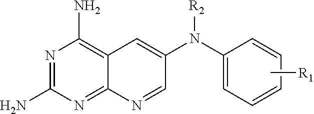 |
| | wherein |
| | R₁ is H, 2′-Ome, 4′-OMe or 2′,5′-diOMe, 3′,4′,5′-triOMe, |
| | 4′-Me, 4′-i-Pr, 3′,4′-(CH₄H₄), 2′,3′-(C₄H₄), 4′-NO₂, 2′,5′-diF |
| | or 3′,4′,5′--triF; and |
| | R₂ is H or an alkyl. |

### Linkers

The linkers of the bispecific adapters hereof are disposed between the targeting ligand (e.g., a radical thereof) and the CAR T-cell targeting moiety (for example, comprising fluorescein, FITC, or NHS-fluorescein). The linker can be any suitable linker.

As used herein, the term “linker” includes a chain of atoms that is bio-functionally adapted to form a chemical bond and connects the CAR 2-cell targeting moiety and the cancer- or CAF-targeting ligand to form a conjugate. Illustratively, the chain of atoms can include carbon, nitrogen, oxygen, sulfur, silicon (Si), and phosphorus (P), such as C, N, O, S, and P, or C, N, O, and S.

The linker can comprise a wide variety of links, such as in the range from about 2 to about 100 atoms in the contiguous backbone. The linker can comprise a releasable form of PEG, a non-releasable form of PEG, polyproline, a hydrophilic amino acid, a sugar, an unnatural peptidoglycan, polyvinylpyrrolidone, or a triblock copolymer comprising a central hydrophobic block of polypropylene glycol flanked on each side by a hydrophilic block of PEG.

The linker can comprise PEG or a PEG derivative. The linker can be (PEG)₃.

The linker can be non-releasable, i.e., non-labile. However, in some embodiments, it may be desirable for a linker in a bispecific adapter to be releasable. i.e., labile, such as, for example, photocleavable, acid-labile, base-labile, or enzyme-cleavable. The term “releasable” in the context of a linker means a linker that includes at least one bond that can be easily broken (e.g., chemically or enzymatically hydrolyzed) under physiological conditions, such as, for example, by reducing agent-labile, pH-labile, acid-labile, base-labile, oxidatively labile, metabolically labile, biochemically labile, enzyme-labile, or a p-aminobenzylic-based multivalent releasable bond. It is appreciated that the physiological conditions resulting in bond breaking do not necessarily include a biological or metabolic process and instead can include a standard chemical reaction, such as a hydrolysis reaction for example, at physiological pH or as a result of compartmentalization into a cellular organelle such as an endosome having a lower pH than cytosolic pH. A cleavable bond can connect two adjacent atoms within the releasable linker and/or connect other linker portions or the targeting moiety and/or CAR T-cell targeting moiety, as described herein, for example, at either or both ends of the releasable linker. In some instances, the releasable linker is broken into two or more fragments. In some instances, the releasable linker is separated from the CAR T-cell targeting moiety.

In some embodiments, the linker is formed such that the CAR T-cell targeting ligand is cleaved from the cancer or CAF-targeting moiety only after sufficient time has passed for the bispecific adapter to circulate within a subject's systemic circulation following administration (e.g., to allow time to be captured and internalized by the targeted cell and/or receptor). In some embodiments, the time period for the release will vary (e.g., from subject to subject (e.g., based on a variety of factors)). In some embodiments, a releasable linker can be engineered such that it will not cleave/release until at least 24 hours post administration or even over a period of a week. In some embodiments, the bispecific adapter can safely pass through the subject's system and any amount not captured by the targeted cells (e.g., those expressing FRα, FRβ, or PSMA, for example) can be excreted.

In contrast, the term “non-releasable” in the context of a linker means a linker that includes at least one bond that is not easily or quickly broken under physiological conditions. In some embodiments, a non-releasable linker comprises a backbone that is stable under physiological conditions (e.g., the backbone is not susceptible to hydrolysis (e.g., aqueous hydrolysis or enzymatic hydrolysis)). In some embodiments, a composition provided herein comprising a non-releasable linker does not release any component of the bispecific adapter (e.g., a cancer- or CAF-targeting ligand or a CAR T-cell targeting ligand). In some embodiments, the non-releasable linker lacks a disulfide bond (e.g., S—S) or an ester in the backbone. In some embodiments, the composition comprises a cancer- or CAF-targeting ligand or a CAR T-cell targeting ligand connected by a backbone that is substantially stable for the entire duration of the bispecific adapter's circulation (e.g., during endocytosis into the target cell endosome). The non-releasable linker can comprise: an amide, ester, ether, amine, and/or thioether (e.g., thio-maleimide). While specific examples are provided herein, it will be understood that any molecule(s) can be used in the non-releasable linker provided that at least one bond that is not easily or quickly broken under physiological conditions is formed.

Perhaps more specifically, a non-releasable linker comprises a linker that, at a neutral pH, for example, less than ten percent (10%) (e.g., less than 5%, less than 4%, less than 3%, less than 2%, less than 1%, less than 0.1%, less than 0.01%, or less than 0.001%) will hydrolyze in an aqueous (e.g., buffered (e.g., phosphate buffer) solution) within a period of time (e.g., 24 hours). In some embodiments, where a non-releasable linker is employed, less than about ten percent (10%), and preferably less than five percent (5%) or none, of the bispecific adapter administered releases a moiety to which it is connected (e.g., in systemic circulation prior to uptake by the targeted cells/tissue).

In some embodiments, a cancer- or CAF-targeting ligand does not cleave from the or a CAR T-cell targeting ligand of the bispecific adapter in vivo. In some embodiments, this is advantageous as it allows for the bispecific adapter to bind and deliver a CAR T-cell to a targeted cancer cell or CAF.

The length of a linker can be selected to optimize linker-imposed separation of molecules on the targeted cell surface, which in turn can facilitate uptake of a bound CAR T-cell into the targeted cell (e.g., when the bispecific adapter is administered). The linker can have a chain length of at least about 5 nm. In certain embodiments, each linker is approximately 5 nm to 15 nm in length. In some embodiments, the linker is at least about 7 nm in length. In certain embodiments, each linker is approximately 7 nm in length and flexible. In certain embodiments, each linker is approximately 7-10 nm in length. In some embodiments, the linker is at least about 14 nm in length. In some embodiments, the linker is about 15 nm in length. In some embodiments, the linker is between about 7 nm and about 31 nm in length (such as, about 7 to 31, 7 to about 31, or 7 to 31), between about 7 nm and about 24 nm in length (such as, about 7 to 24, 7 to about 24, or 7 to 24), or between about 7 nm and about 20 nm in length (such as, about 7 to 20, 7 to about 20, or 7 to 20). In some embodiments, the linker is between about 14 nm and about 31 nm in length (such as, about 14 to 31, 14 to about 31, or 14 to 31), between about 14 nm and about 24 nm in length (such as, about 14 to 24, 14 to about 24, or 14 to 24), or between about 14 nm and about 20 nm in length (such as, about 14 to 20, 14 to about 20, or 14 to 20). In some embodiments, the linker has a chain length of at least 7 nm, at least 14 nm, at least 20 nm, at least 25 nm, at least 30 nm, or at least 40 nm; or from 5 nm to 15 nm, 5 nm to 10 nm, 7 nm to 10 nm, 5 nm to 20 nm, 10 nm to 40 nm, or 25 nm to 100 nm. In certain embodiments, the length of each linker is selected to facilitate micro-clustering of bound molecules on a cell surface to provide at or about 7-10 nm of separation therebetween (such as about 7 nm to about 10 nm, 7 nm to about 10 nm, about 7 nm to 10 nm, or 7 nm to 10 nm). The ranges specified in this paragraph are inclusive of the stated end points and all 1 nm increments encompassed within the stated ranges.

A linker can comprise at least one carbon-carbon bond and/or at least one amide bond. The linker can comprise one or more L- or D-configurations, natural or unnatural amino acids, or a combination of any of the foregoing.

In certain embodiments, a linker is a group comprising one or more covalently connected structural units.

The linker can further be engineered to optimize biodistribution, bioavailability, and PK/PD (e.g., of the bispecific adapter) and/or to increase uptake (e.g., of the bispecific adapter) as previously described into the targeted tissue pursuant to methodologies commonly known in the art or hereinafter developed such as through PEGylation and the like.

In some embodiments, linkers may comprise one or more spacers (e.g., to facilitate a particular release time, facilitate an increase in uptake into a targeted tissue, and/or optimize biodistribution, bioavailability, and/or PK/PD of a bispecific adapter provided herein). A spacer may comprise one or more alkyl chains, PEGs, peptides, sugars, peptidoglycans, clickable linkers (e.g., triazoles), rigid linkers such as poly prolines and poly piperidines, and the like.

In some embodiments, linkers of the bispecific adapter comprise PEG, a PEG derivative, or any other linker known in the art or hereinafter developed that can achieve the purpose set forth herein. In some embodiments, the linker is repeated n times, where n is a positive integer. For example, and without limitation, n may be any integer selected from a range of 1-16, 1-32, 1-64, or 1-96. The number of repeats in the linker (i.e., n) can be selected to achieve the desired functionality, size, and/or potency of the compound and/or in view of the desired application. In some embodiments, the linker comprises one or more spacers (e.g., which may also be used to specifically design characteristics of the bispecific adapter).

In certain embodiments, the linker comprises, consists of, or consists essentially of PEG₃
PEG₁₈. In certain embodiments, the linker comprises, consists of, or consists essentially of PEG₃
PEG₁₂. In certain embodiments, the linker comprises, consists of, or consists essentially of PEG₃
PEG₈. In certain embodiments, the linker comprises, consists of, or consists essentially of PEG₅
PEG₁₅. In certain embodiments, the linker comprises, consists of, or consists essentially of PEG₆
PEG₁₄. In certain embodiments, the linker comprises, consists of, or consists essentially of PEG₇
PEG₁₃. In certain embodiments, the linker comprises, consists of, or consists essentially of PEG₈
PEG₁₂. In certain embodiments, the linker comprises, consists of, or consists essentially of PEG₉
PEG₁₂. In certain embodiments, the linker comprises, consists of, or consists essentially of PEG₄
PEG₁₅. In certain embodiments, the linker comprises, consists of, or consists essentially of PEG₄
PEG₁₆. In certain embodiments, the linker comprises, consists of, or consists essentially of PEG₃
PEG₁₁. In certain embodiments, the linker comprises, consists of, or consists essentially of PEG₃
PEG₁₂. In certain embodiments, the linker comprises, consists of, or consists essentially of PEG₃
PEG₁₅. The linker can comprise (or consist essentially of or consist of) PEG₄ to PEG₁₆, such as PEG₄, PEG₅, PEG₆, PEG₇, PEG₈, PEG₉, PEG₁₀, PEG₁₁, PEG₁₂, PEG₁₃, PEG₁₄, PEG₁₅, or PEG₁₆. All ranges stated in this paragraph are inclusive of the stated end points.

The linker can be or comprise (or consist essentially of or consist of) PEG₆. In certain embodiments, the linker comprises, consists of or consists essentially of PEG₁₀. In certain embodiments, the linker comprises, consists of, or consists essentially of PEG₁₂. In certain embodiments, the linker comprises, consists of, or consists essentially of PEG₁₅. In certain embodiments, the linker comprises, consists of, or consists essentially of PEG₁₆.

In some embodiments, the linker is a hydrolyzable linker. In some embodiments, the linker is a non-hydrolyzable linker. In some embodiments, the linker is an optionally substituted heteroalkyl. In some embodiments, the linker is a substituted heteroalkyl comprising at least one substituent selected from the group consisting of alkyl, hydroxyl, oxo, PEG, carboxylate, and halo. In some embodiments, the linker comprises a spacer (e.g., as described elsewhere herein).

In some embodiments, the linker is substituted heteroalkyl with at least one disulfide bond in the backbone thereof. In some embodiments, the linker is a peptide with at least one disulfide bond in the backbone thereof.

In some embodiments, the linker comprises —CONH—CH(COOH)—CH₂—S—S—CH₂—CRₐR_{b}—O—CO—, —CONH—CH(COOH)CRₐR_{b}—O—CO—, —C(O)NHCH(COOH)(CH₂)₂—CONH—CH(COOH)CRₐR_{b}—O—CO— or —C(O)NHCH(COOH)(CH₂)₂—CONH—CH(COOH)—CH₂—S—S—CH₂—CRₐR_{b}—O—CO—, wherein Rₐ and R_{b} are independently H, alkyl, or heteroalkyl (e.g., PEG).

In some embodiments, the linker comprises a structure of

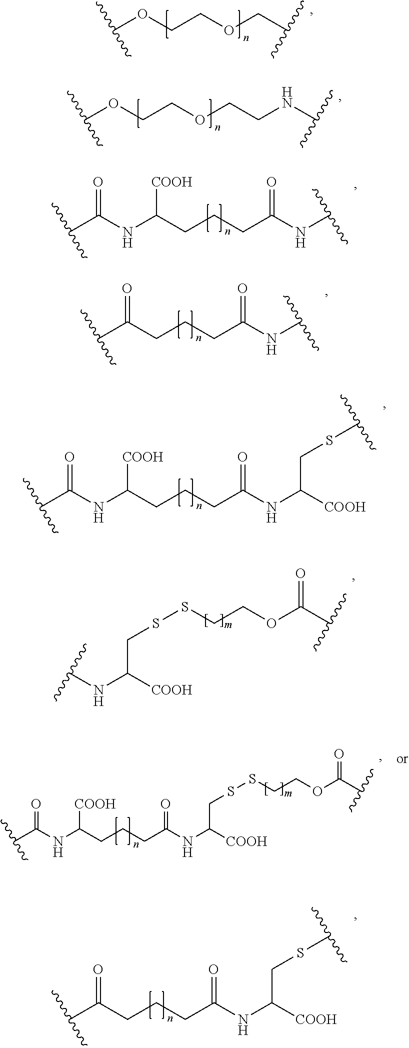

wherein n or m (where applicable) is 0 to 10.

In some embodiments, the linker comprises a structure of:

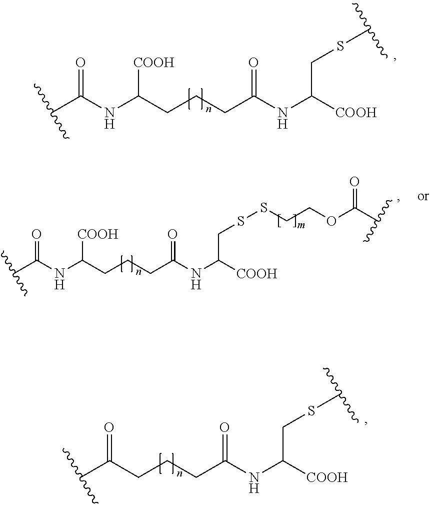

wherein n and in are each independently 0 to 10.

In some embodiments, the linker comprises a structure of:

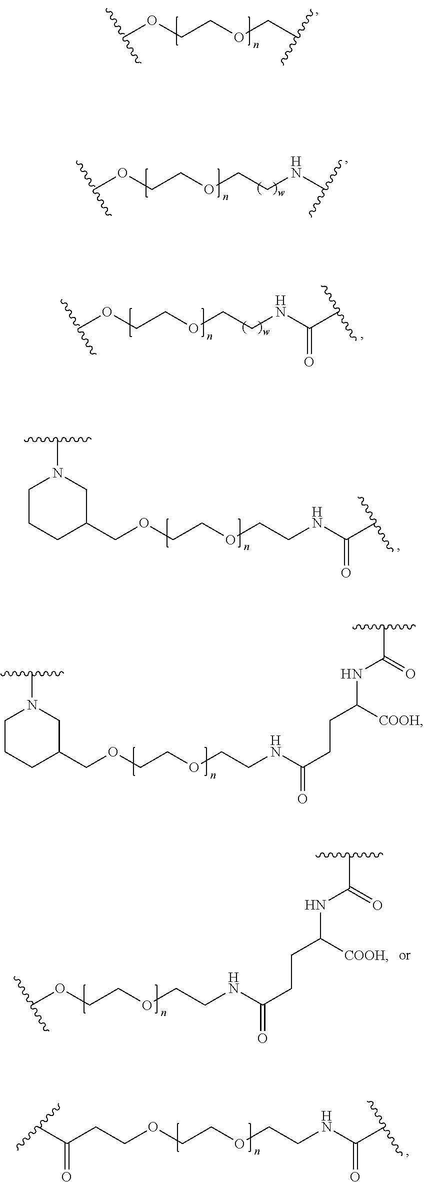

wherein n is 1 to 32. In at least one exemplary embodiment, n is 1 to 30 and w is 0 to 5 (where applicable).

In some embodiments, the linker comprises the structure of:

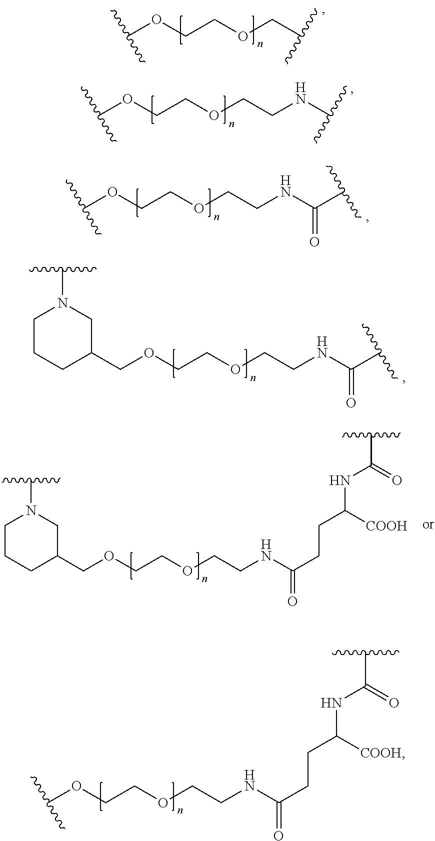

wherein n is 1 to 16.

In certain embodiments, the linker can comprise the structure of:

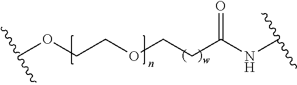

wherein n is 1 to 30 and w is 0 to 5.

For further discussion of linkers relevant to the present disclosure and, in particular related to folate and fluorescein-linker-folate adapters, see, e.g., International Patent Application Publication No. WO 2020/033129, which is hereby incorporated by reference for its teachings regarding same.

### Bispecific Adapters

The bispecific adapter, or pharmaceutically acceptable salt or hydrate thereof, can be for use with an anti-fluorescein CAR-T cell in the treatment of a FAP-expressing (e.g., FAPα-expressing) cancer and can comprise a fluorescein-linker-FAP ligand. The adapter, or pharmaceutically acceptable salt or hydrate thereof, can comprise fluorescein-linker-FAP ligand (e.g., fluorescein=fluorescein, FITC, or NHS-fluorescein), wherein the FAP ligand is:

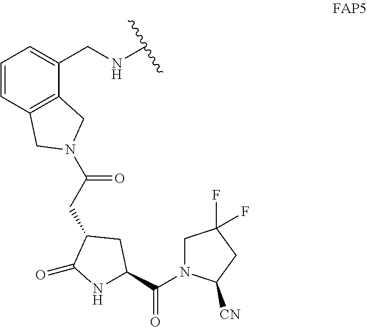 [SMILES: *NCc1cccc2c1CN(C(=O)C[C@@H]1C[C@@H](C(=O)N3CC(F)(F)C[C@H]3C#N)NC1=O)C2]

and the linker comprises (or consists essentially of or consists of) PEG. The linker can comprise (or consist essentially of or consist of) PEG₄ to PEG₁₆, such as PEG₄, PEG₅, PEG₆, PEG₇, PEG₈, PEG₉, PEG₁₀, PEG₁₁, PEG₁₂, PEG₁₃, PEG₁₄, PEG₁₅, or PEG₁₆. The linker can comprise (or consist essentially of or consist of) PEG₆. The linker can comprise (or consist essentially of or consist of) PEG₁₆. The FAP ligand can have a structure represented by the formula I-B:

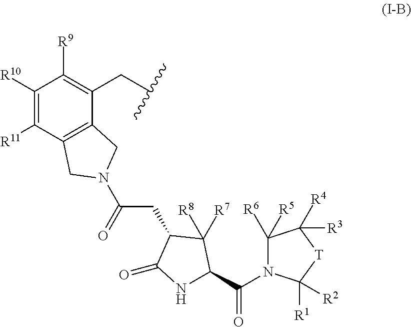

wherein:

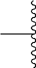

is the point of attachment to the linker of the adaptor;
T is substituted or unsubstituted methylene (—CH₂—), substituted or unsubstituted amino (—NH—), —O—, or —S—;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F and 5-tetrazolyl;
R³ and R⁴ are each independently selected from the group consisting of —H, —OH, F, Cl, Br, I, —C₁₋₆alkyl, —O—C₁₋₆alkyl, and —S—C₁₋₆ alkyl;
R⁵, R⁶, R⁷, and R⁸ are each independently selected from the group consisting of H, alkyl and halo; and
R⁹, R¹⁰, an R¹¹ are each independently selected from the group consisting of H, —C₁₋₆alkyl, —O—C₁₋₆alkyl, —S—C₁₋₆ alkyl, F, Cl, Br and I.

In certain embodiments, the FAP ligand (or radical thereof) of the bispecific adapter can have a structure represented by the formula I-C:

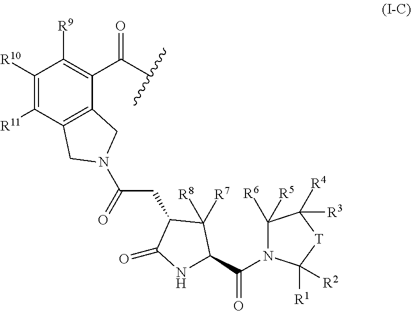

wherein:

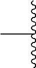

is the point of attachment to the linker of the adaptor;
T is substituted or unsubstituted methylene (—CH₂—), substituted or unsubstituted amino (—NH—), —O—, or —S—;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, and 5-tetrazolyl;
R³ and R⁴ are each independently selected from the group consisting of —H, —OH, F, Cl, Br, I, —C₁₋₆alkyl, —O—C₁₋₆alkyl, and —S—C₁₋₆alkyl;
R⁵, R⁶, R⁷, and R⁸ are each independently selected from group consisting of H, alkyl and halo; and
R⁹, R¹⁰, and R¹¹ are each independently selected from group consisting of H, —C₁₋₆alkyl, —O—C₁₋₆alkyl, —S—C₁₋₆ alkyl, F, Cl, Br and L.

In certain embodiments, the bispecific adapter, or pharmaceutically acceptable salt or hydrate thereof, is for use with anti-fluorescein (e.g., fluorescein, FITC, or NHS-fluorescein) CAR-T cells in the treatment of FAP-expressing (e.g., FAPα-expressing) cancer, which adapter comprises fluorescein-linker-FAP ligand (e.g., fluorescein=fluorescein, FITC, or NHS-fluorescein), wherein the FAP ligand (or radical thereof) is a FAP8 ligand having a structure of:

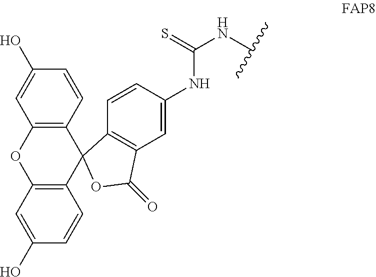 [SMILES: *NC(=S)Nc1ccc2c(c1)C(=O)OC21c2ccc(O)cc2Oc2cc(O)ccc21]

and the linker comprises (or consists essentially of or consists of) PEG. The linker can comprise (or consist essentially of or consist of) PEG₃ to PEG₁₅, such as PEG₃, PEG₄, PEG₅, PEG₆, PEG₇, PEG₈, PEG₉, PEG₁₀, PEG₁₁, PEG₁₂, PEG₁₃, PEG₁₄, or PEG₁₅. The linker can comprise (or consist essentially of or consist of) PEG₁₅. The linker can comprise (or consist essentially of or consist of) PEG₁₆.

The FAP ligand (or radical thereof) can have the structure:

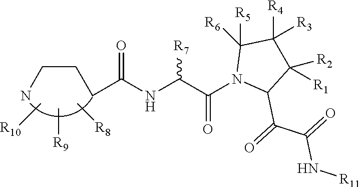

wherein:

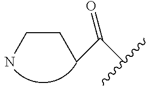 [SMILES: *C(=O)CCCN]

represents a functionalized 5- to 10-membered N-containing aromatic or non-aromatic mono- or bi-cyclic heterocycle, which optionally further comprises 1-3 heteroatoms selected from O, N. and S;
R₁ and R₂ are independently selected from the group consisting of —H, -D, —OH, —F, —Cl, —Br, —I, —C₁₋₆ alkyl, —O—C₁₋₆ alkyl, and —S—C₁₋₆ alkyl;
R₃ and R₄ are independently selected from the group consisting of —H, —OH, —F, —Cl, —Br, —I, —C₁₋₆ alkyl, —O—C₁₋₆ alkyl, and —S—C₁₋₆ alkyl;
R₅ and R₆ are independently selected from the group consisting of —H, —OH, —F, —Cl, —Br, —I, —C₁₋₆ alkyl, —O—C₁₋₆ alkyl, and —S—C₁₋₆ alkyl;
R₇ is selected from the group consisting of —H, -D, OH, CH₂═, —CH₃, CH₃CH₂—, (CH₃)₂CH—(CH₃)₃C—, —CH₂Ph. and substituted —CH₂Ph;
R₈-R₁₀ are independently selected from the group consisting of —H, —OH, —F, —Cl, —Br, —I, —NO₂, —SO₃H, —SO₂NH₂, —NH₂, —N₃, —NH═NH, —C₁₋₆ alkyl, —O—C₁₋₆ alkyl, and —S—C₁₋₆ alkyl; and R₁₁ is selected from the group consisting of —H, -D, C₁-C₁₀ alkyl, C₃-C₁₀ cycloalkyl, adamantyl,

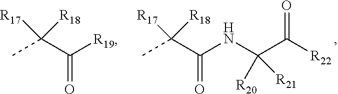

substituted or unsubstituted aryl, substituted or unsubstituted C₇-C₂₀ alkyl aryl, wherein the aryl is:

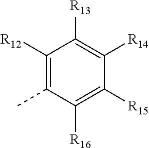

wherein:
R₁₂ and R₁₆ are independently selected from the group consisting of —H, -D, halogen, C₁-C₃ alkyl, C₁-C₃ alkoxy, —CF₃, and —C(═O)—OR₂₃, wherein R₂₃ is selected from the group consisting of H, D, halogen, C₁-C₄ alkyl, and C₁-C₃ alkoxy;
R₁₃, R₁₄ and R₁₅ are independently selected from the group consisting of —H, -D, halogen, —OMe, C₁-C₃ alkyl. C₁-C₃ alkoxy, —CF₃, and —C(═O)—OR₂₃, wherein R₂₃ is selected from the group consisting of —H, -D, halogen, C₁-C₄ alkyl, and C₁-C₃ alkoxy;
R₁₇, R₁₈, R₂₀, and R₂₁ are independently selected from —H and —CH₃; and
R₁₉ and R₂₂ are independently selected from the group consisting of phenyl, dimethoxyphenyl, and aryl.

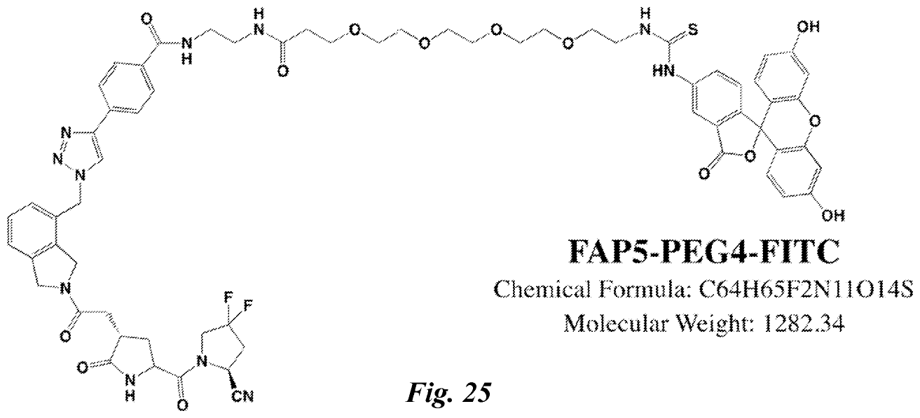

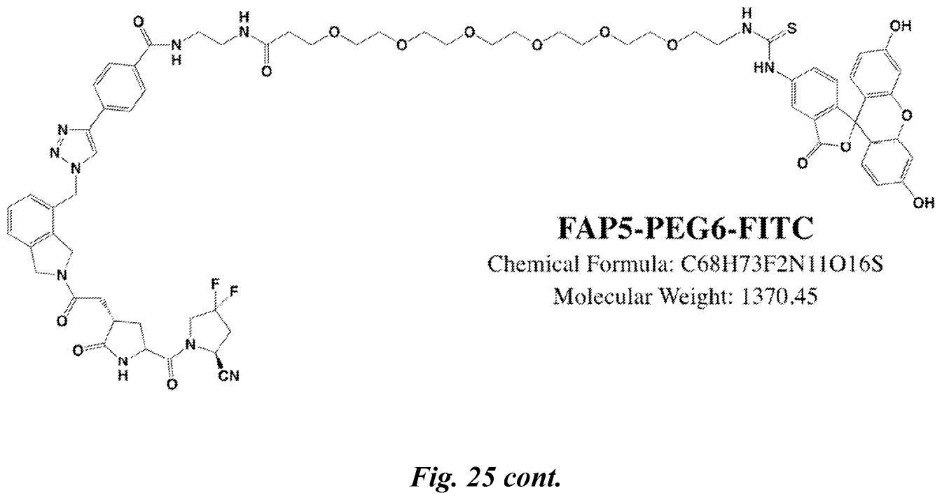

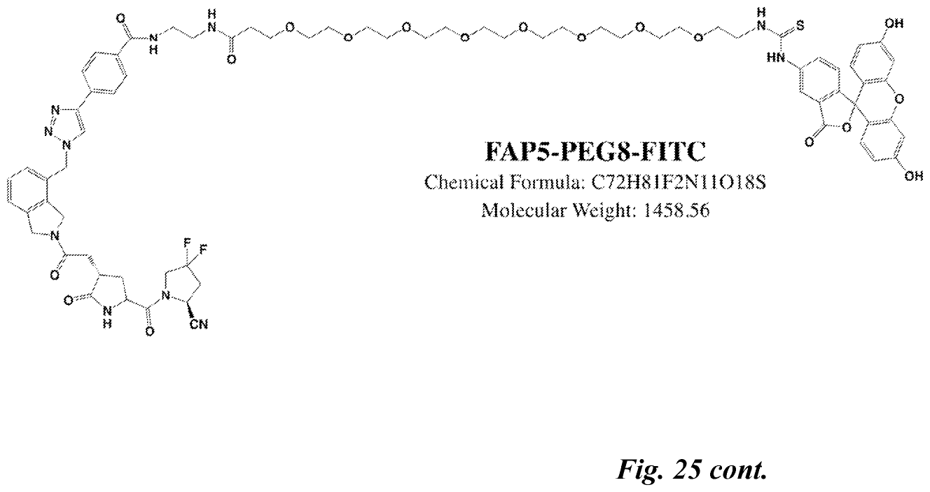

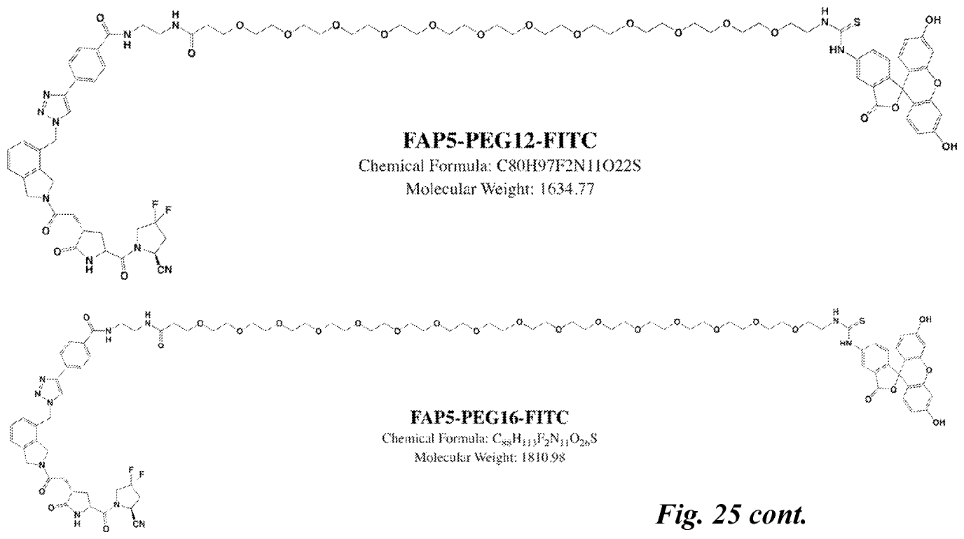

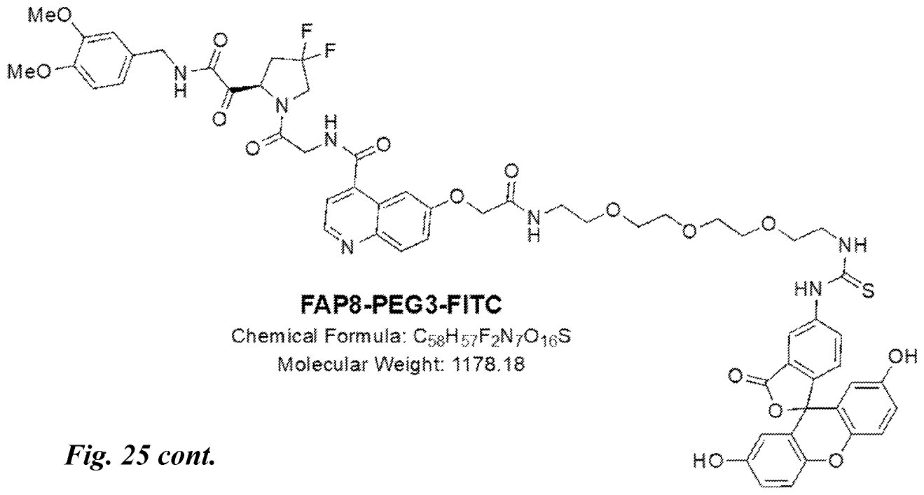

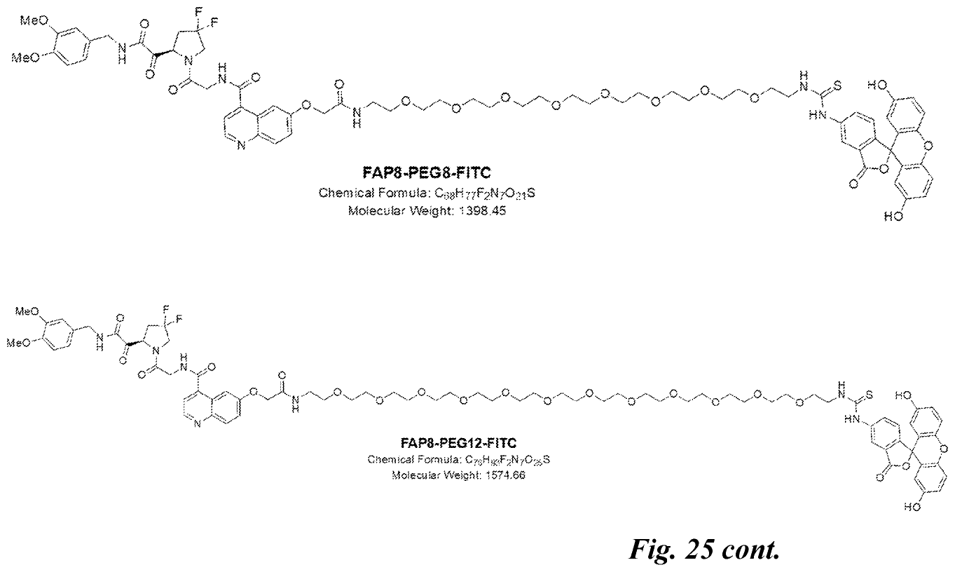

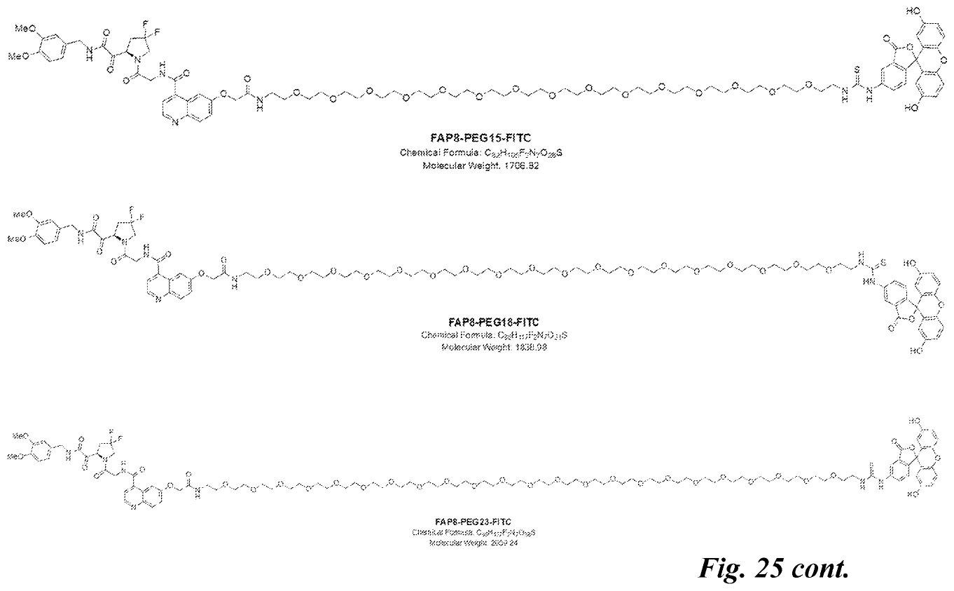

The bispecific adapter can have a structure of the formulae shown in FIG. 25.

The bispecific adapter can comprise a fluorescein-linker-PSMA ligand. In certain embodiments, the bispecific adapter is for use with an anti-fluorescein CAR-T cell in the treatment of a PSMA-expressing cancer. In certain embodiments, the PSMA ligand (or radical thereof) is or comprises DUPA. In certain embodiments, the fluorescein of the adapter comprises FITC and the PSMA ligand (or radical thereof) is or comprises DUPA such that the bispecific adaptor comprises FITC-DUPA conjugated with a PEG linker, such as PEG₃, PEG₆, PEG₈ or PEG₁₂. Such adaptors were designed and synthesized to control the distance between a FITC-binding CAR-T cell (chimeric antigen receptor-T cell) and a PSMA-expressing tumor cell. Compared to a DUPA-FITC conjugate without any linker, incorporation of a linker and increasing the length of the linker can improve anti-FITC CAR-T cell function. In certain embodiments, an optimal linker length can be obtained with PEG₆.

The bispecific adapter, or a pharmaceutically acceptable salt or hydrate thereof, can comprise fluorescein-linker-PSMA ligand (e.g., fluorescein=fluorescein. FITC, or NHS-fluorescein), wherein the PSMA ligand is PSMAL1 (i.e., radical thereof), and the linker comprises (or consists essentially of or consists of) PEG. The linker can comprise (or consists essentially of or consists of) PEG₃ to PEG₁₂, such as PEG₃, PEG₄, PEG₅, PEG₆, PEG₇, PEG₈, PEG₉, PEG₁₀, PEG₁₁, or PEG₁₂. The linker can comprise (or consists essentially of or consists of) PEG₆. The bispecific adapter can have the structure:

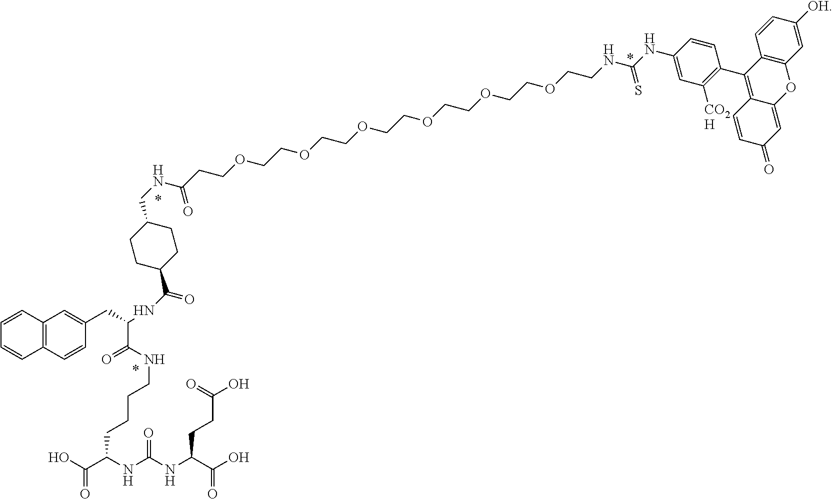 [SMILES: O=C(O)CC[C@H](NC(=O)N[C@@H](CCCCNC(=O)[C@H](Cc1ccc2ccccc2c1)NC(=O)[C@H]1CC[C@H](CNC(=O)CCOCCOCCOCCOCCOCCOCCNC(=S)Nc2ccc(-c3c4ccc(=O)cc-4oc4cc(O)ccc34)c(C(=O)O)c2)CC1)C(=O)O)C(=O)O]

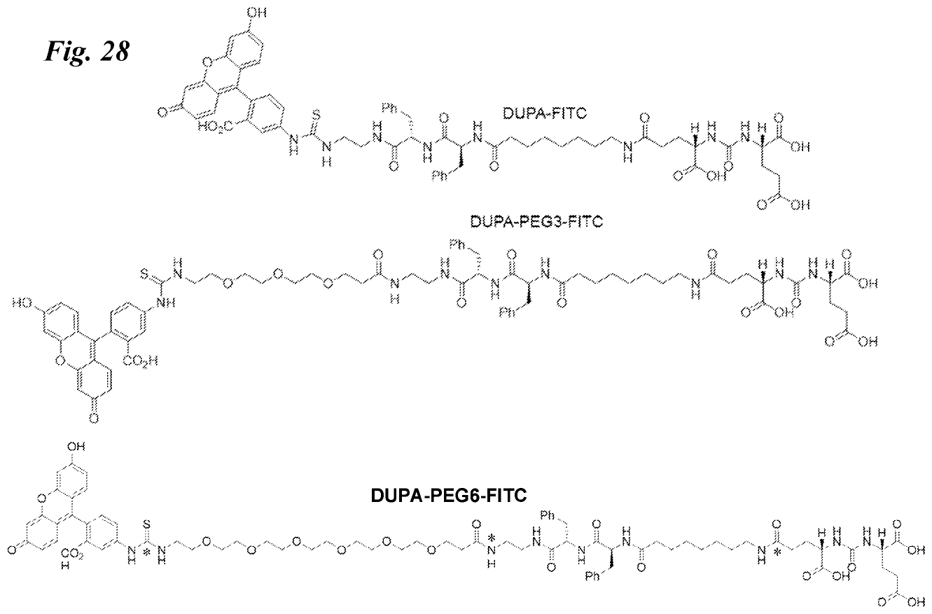

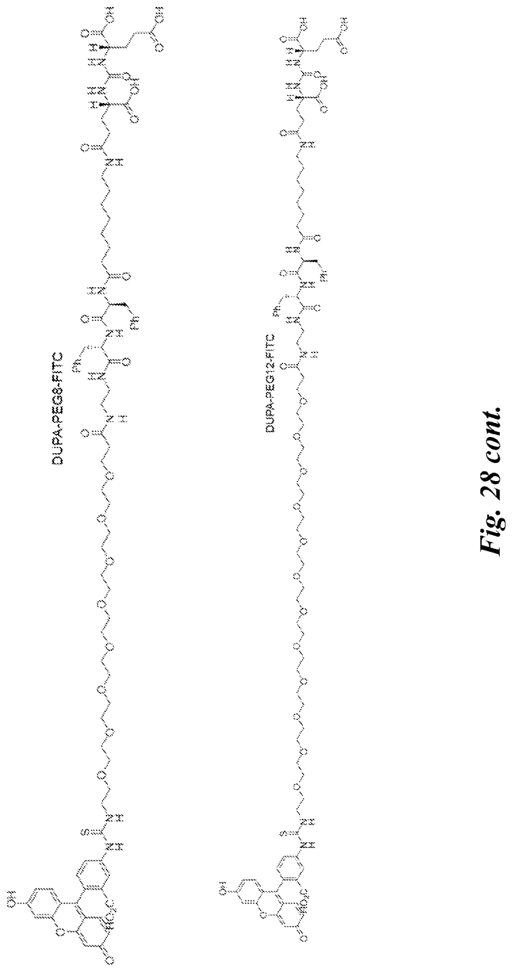

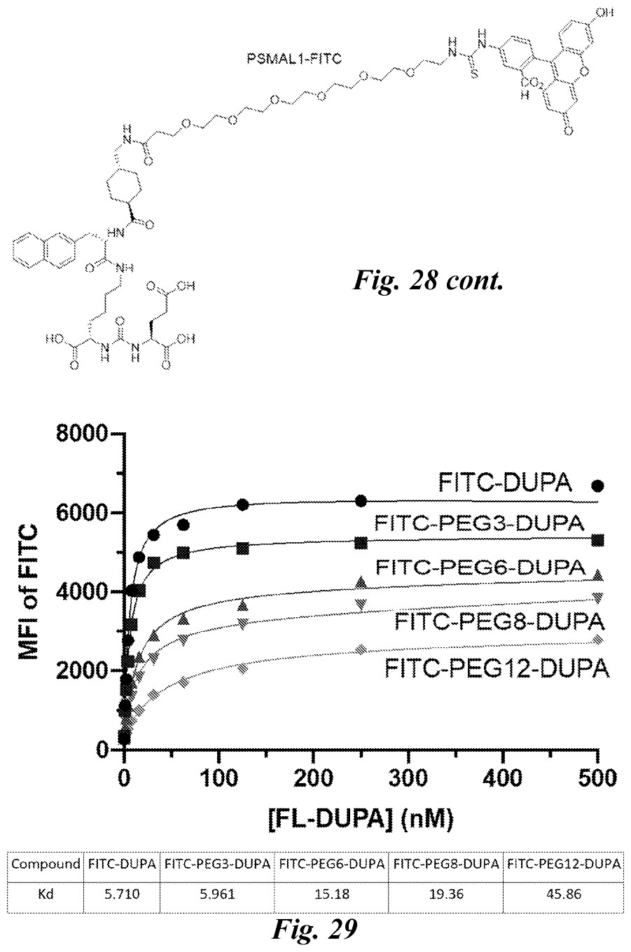

The bispecific adapter can be for use with an anti-fluorescein CAR-T cell in the treatment of a PSMA-expressing cancer, and can comprise a fluorescein-linker-PSMA ligand, wherein the PSMA ligand (or radical thereof) is or comprises DUPA or a DUPA derivative and the linker comprises (or consists essentially of or consists of) PEG₃ to PEG₁₁, and wherein the adapter can be a pharmaceutically acceptable salt or hydrate thereof. The linker can comprise (or consists essentially of or consists of) PEG₃ to PEG₁₂, such as PEG₃, PEG₄, PEG₅, PEG₆, PEG₇, PEG₈, PEG₉, PEG₁₀, PEG₁₁, or PEG₁₂. The linker can comprise (or consists essentially of or consists of) PEG₆. The bispecific adapter can have a structure of the formulae shown in FIG. 28.

The bispecific adapter may contain one or more chiral centers or may otherwise be capable of existing as multiple stereoisomers. Accordingly, various embodiments of the bispecific adapter may include pure stereoisomers, as well as mixtures of stereoisomers, such as enantiomers, diastereomers, and enantiomerically or diastereomerically enriched mixtures. The bispecific adapter can be capable of existing as geometric isomers, such as pure geometric isomers or mixtures of geometric isomers.

The bispecific adapter compounds can be synthesized in accordance with methods known in the art. Various methods of synthesis are exemplified herein.

### Salts and Hydrates

The bispecific adapters hereof can be presented as a pharmaceutically acceptable salt. A “pharmaceutically acceptable salt” of a bispecific adapter refers to those salts whose counter ions can be used in pharmaceuticals. Such salts include (i) acid addition salts, which can be obtained by reaction of the free base of the parent compound with inorganic acids, such as hydrochloric acid, hydrobromic acid, nitric acid, phosphoric acid, sulfuric acid, perchloric acid, and the like, or with organic acids, such as acetic acid, oxalic acid, (D) or (L) malic acid, maleic acid, methane sulfonic acid, ethane sulfonic acid, p-toluene sulfonic acid, salicylic acid, tartaric acid, citric acid, succinic acid, malonic acid, and the like, and (ii) salts formed when an acidic proton present in the parent compound either is replaced by a metal ion, e.g., an alkali metal ion, an alkaline earth ion, or an aluminum ion, or coordinates with an organic base, such as ethanolamine, diethanolamine, triethanolamine, trimethamine, N-methyl glucamine, and the like. Pharmaceutically acceptable salts are well-known to those skilled in the art, and any such pharmaceutically acceptable salt is contemplated herein.

In various embodiments, suitable basic salts are formed from bases which form non-toxic salts. Illustrative examples include arginine, benzathine, calcium, choline, diethylamine, diolamine, glycine, lysine, magnesium, meglumine, olamine, potassium, sodium, tromethamine, and zinc salts. Hemisalts of acids and bases also may be formed, e.g., hemisulphate and hemicalcium salts.

Pharmaceutically acceptable salts can be synthesized from the parent bispecific adapter compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company. Easton, Pa., 1985, the disclosure of which is hereby incorporated by reference.

The bispecific adapter, or pharmaceutically acceptable salt thereof, may exist in unsolved forms as well as solvated forms, including hydrated forms. Solvated forms can be equivalent to unsolvated forms. In each embodiment hereof, it will be understood that the formulae include and represent not only all pharmaceutically acceptable salts of the bispecific adapters, but also include any and all hydrates and/or solvates of the compound formulae or salts thereof. The term “solvate” means a compound, or a salt thereof, that further includes a stoichiometric or non-stoichiometric amount of solvent bound by non-covalent intermolecular forces. Where the solvent is water, the solvate is a hydrate.

Certain functional groups, such as the hydroxy, amino, and like, can form complexes and/or coordination conjugates with water and/or various solvents. Accordingly, the formulae are to be understood to include and represent those various hydrates and/or solvates. Non-hydrates and/or non-solvates of the bispecific adapters are also included.

### Pharmaceutical Compositions

In view of the above, also provided is a composition (e.g., a pharmaceutical composition) for the treatment of cancer comprising at least one bispecific adapter and a pharmaceutically acceptable carrier or excipient. “Pharmaceutically acceptable carrier” includes any of the standard pharmaceutical carriers, such as, but not limited to, a buffering agent, a preserving agent, an anesthetic agent, a solubilizing agent, an isotonic agent, a wetting agent, and a stabilizer. The term also encompasses any of the agents approved by a regulatory agency, such as the U.S. Food and Drug Administration, or listed in the U.S. Pharmacopeia for use in animals (e.g., mammals, such as humans). The carrier can be a phosphate-buffered saline solution, water, or an emulsion such as an oil/water or water/oil emulsion.

In certain embodiments, the pharmaceutical composition comprises a combination of two or more types of bispecific adapters hereof. For example, and without limitation, the pharmaceutical composition can comprise (i) a combination of a first set of bispecific adapters comprising or consisting of fluorescein-linker-FAP and a second set of bispecific adapters comprising or consisting of fluorescein-linker-FR; and (ii) a pharmaceutically acceptable carrier or excipient. In certain embodiments, the pharmaceutical composition can comprise (i) a combination of a first set of bispecific adapters comprising or consisting of fluorescein-linker-FAP and a second set of bispecific adapters comprising or consisting of fluorescein-linker-PSMA; and (ii) a pharmaceutically acceptable carrier or excipient.

Further provided is a combination of separate pharmaceutical compositions for the treatment of cancer. The combination can comprise (i) a pharmaceutical composition comprising a bispecific adapter comprising fluorescein-linker-folate and a pharmaceutically acceptable carrier or excipient and (ii) a pharmaceutical composition comprising a bispecific adapter comprising fluorescein-linker-FAP ligand and a pharmaceutically acceptable carrier or excipient.

Still further provided is a combination of separate pharmaceutical compositions for the treatment of cancer comprising (i) a pharmaceutical composition comprising a bispecific adapter comprising fluorescein-linker-PSMA ligand and a pharmaceutically acceptable carrier or excipient and (ii) a pharmaceutical composition comprising a bispecific adapter comprising fluorescein-linker-FAP ligand and a pharmaceutically acceptable carrier or excipient.

Also provided is a pharmaceutical composition for use in the treatment of FAP-expressing cancer comprising a bispecific adapter comprising fluorescein-linker-FAP and a pharmaceutically acceptable carrier or excipient. In certain embodiments, a pharmaceutical composition for use in the treatment of a PSMA-expressing cancer is provided. There, the pharmaceutical composition can comprise a bispecific adapter comprising fluorescein-linker-PSMA and a pharmaceutically acceptable carrier or excipient. In certain embodiments, a pharmaceutical composition for use in the treatment of FR-expressing cancer (e.g., FRα or FRβ) is provided, such composition comprising a bispecific adapter comprising fluorescein-linker-FR and a pharmaceutically acceptable carrier or excipient.

The bispecific adapters can be formulated as pharmaceutical compositions and administered to a mammalian host, such as a human patient, in a variety of forms adapted to the chosen route of administration. For example, the pharmaceutical composition can be formulated for and administered via oral or parenteral, intravenous, intraarterial, intraperitoneal, intrathecal, epidural, intracerebroventricular, intraurethral, intrasternal, intracranial, intratumoral, intramuscular, topical, inhalation and/or subcutaneous routes. Indeed, in at least one embodiment, a bispecific adapter and/or composition as described herein can be administered directly into the blood stream, into muscle, or into an internal organ.

For example, in at least one embodiment, the present bispecific adapters can be systemically administered (orally, for example) in combination with a pharmaceutically acceptable vehicle such as an inert diluent or an assimilable edible carrier. For oral therapeutic administration, the bispecific adapter can be combined with one or more excipients and used in the form of ingestible tablets, buccal tablets, troches, capsules, elixirs, suspensions, syrups, wafers, and the like. The percentage of the compositions and preparations may vary and may be between about 1 to about 99% weight of the active ingredient(s) and a binder, excipients, a disintegrating agent, a lubricant, and/or a sweetening agent (as are known in the art). The amount of active compound in such therapeutically useful compositions is such that an effective dosage level will be obtained.

The bispecific adapters and pharmaceutical compositions hereof can be formulated as parenteral formulations. Parenteral formulations are typically aqueous solutions, which can contain carriers or excipients such as salts, carbohydrates, and buffering agents (preferably at a pH of from 3 to 9), but they can be more suitably formulated as a sterile, non-aqueous solution or as a dried from to be used in conjunction with a suitable vehicle such as sterile, pyrogen-free water or sterile saline. Preparation under sterile conditions, by lyophilization to produce a sterile, lyophilized powder for a parenteral formulation, can be accomplished using methods well-known in the art. The solubility of the bispecific adapter, or a pharmaceutically acceptable salt thereof, for parenteral formulation can be increased by the use of appropriate formulation techniques, such as the incorporation of solubility-enhancing agents.

The bispecific adapters/compositions can also be administered via infusion or injection (e.g., using needle (including microneedle) injectors and/or needle-free injectors). Solutions of the composition can be aqueous, optionally mixed with a nontoxic surfactant and/or can contain carriers or excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water or phosphate-buffered saline (PBS). For example, dispersions can be prepared in glycerol, liquid PEGs, triacetin, and mixtures thereof and in oils. Under ordinary conditions of storage and use, these preparations can further contain a preservative to prevent the growth of microorganisms.

The pharmaceutical dosage forms suitable for injection or infusion can include sterile aqueous solutions or dispersions or sterile powders comprising the active ingredients that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example and without limitation, water, ethanol, a polyol (e.g., glycerol, propylene glycol, liquid PEG(s), and the like), vegetable oils, nontoxic glyceryl esters, and/or suitable mixtures thereof. In at least one embodiment, the proper fluidity can be maintained by the formation of liposomes, by the maintenance of the required particle size in the case of dispersions or by the use of surfactants. The action of microorganisms can be prevented by the addition of various antibacterial and antifungal agents such as parabens, chlorobutanol, phenol, sorbic acid, thimerosal, and the like. In certain cases, it will be desirable to include one or more isotonic agents such as sugars, buffers, or sodium chloride. Prolonged absorption of the injectable compositions can be brought about by the incorporation of agents formulated to delay absorption, for example, aluminum monostearate and gelatin.

Sterile injectable solutions can be prepared by incorporating the bispecific adapter(s) and/or composition in the required amount of the appropriate solvent with one or more of the other ingredients set forth above, as required, followed by filter sterilization. In the case of sterile powders for the preparation of sterile injectable solutions, the preferred methods of preparations are vacuum drying and freeze-drying techniques, which yield a powder of the active ingredient plus any additional desired ingredient present in the previously sterile-filtered solutions.

For topical administration, it may be desirable to administer the bispecific adapters to the skin as compositions or formulations in combination with a dermatologically acceptable carrier, which may be a solid or a liquid. For example, in certain embodiments, solid carriers may include finely divided solids such as talc, clay, microcrystalline cellulose, silica, alumina and the like. Similarly, useful liquid carriers may comprise water, alcohols or glycols or water-alcohol/glycol blends, in which the present compounds can be dissolved or dispersed at effective levels, optionally with the aid of non-toxic surfactants. Additionally or alternatively, adjuvants such as fragrances and antimicrobial agents can be added to optimize the properties for a given use. The resultant liquid compositions can be applied from absorbent pads, used to impregnate bandages and/or other dressings, sprayed onto the targeted area using pump-type or aerosol sprayers, or simply applied directly to a desired area of the subject.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like for application directly to the skin of the subject.

The amount of the bispecific adapter (or pharmaceutically acceptable salt thereof) to be administered to a subject can vary significantly, depending on the cancer being treated, the route of administration, and tissue distribution. As used herein, the terms “therapeutically effective,” “therapeutically effective dose,” “therapeutically effective amount,” “prophylactically effective amount,” or “prophylactically effective dose” mean (unless specifically stated otherwise) a quantity of a bispecific adapter which, when administered either one time or over the course of a treatment cycle affects the health, wellbeing or mortality of a subject (e.g., and without limitation, delays the onset of and/or reduces the severity of one or more of the symptoms associated with a cancer). Useful dosages of the bispecific adapters can be determined by comparing their in vitro activity, and the in vivo activity in animal models. Methods of the extrapolation of effective dosages in mice and other animals to human subjects are known in the art. Indeed, the dosage of the bispecific adapter can vary significantly depending on the condition of the host subject, the cancer being treated, how advanced the pathology is, the route of administration of the bispecific adapter and tissue distribution, and the possibility of co-usage of other therapeutic treatments (such as radiation therapy or additional drugs in combination therapies such as, for example CAR T-cell therapy). The amount of the composition required for use in treatment (e.g., the therapeutically or prophylactically effective amount or dose) will vary not only with the particular application, but also with the salt selected (if applicable) and the characteristics of the subject (such as, for example, age, condition, sex, the subject's body surface area and/or mass, tolerance to drugs) and will ultimately be at the discretion of the attendant physician, clinician, or otherwise.

The amount to be administered to a subject can range, for example, from about 0.05 mg to about 30 mg, about 0.05 mg to about 25 mg, about 0.05 mg to about 20 mg, about 0.05 mg to about 15 mg, about 0.05 mg to about 10 mg, about 0.05 mg to about 9 mg, about 0.05 mg to about 8 mg, about 0.05 mg to about 7 mg, about 0.05 mg to about 6 mg, about 0.05 mg to about 5 mg, about 0.05 mg to about 4 mg, about 0.05 mg to about 3 mg, about 0.05 mg to about 2 mg, about 0.05 mg to about 1 mg, about 0.05 mg to about 0.5 mg, about 0.05 mg to about 0.4 mg, about 0.05 mg to about 0.3 mg, about 0.05 mg to about 0.2 mg, about 0.05 mg to about 0.1 mg, about 0.01 mg to about 20 mg, about 0.3 mg to about 10 mg, about 0.1 mg to about 20 mg, or about 0.8 mg to about 3 mg. The ordinarily skilled artisan will readily appreciate that the dose may vary within the various ranges provided above based on the factors pointed out above and may be at the treating physician's discretion.

Therapeutically effective or prophylactically effective amounts or doses can range, for example, from about 0.05 mg/kg of patient body weight to about 30.0 mg/kg of patient body weight, or from about 0.01 mg/kg of patient body weight to about 5.0 mg/kg of patient body weight, including but not limited to 0.01 mg/kg, 0.02 mg/kg. 0.03 mg/kg, 0.04 mg/kg, 0.05 mg/kg, 0.1 mg/kg. 0.2 mg/kg. 0.3 mg/kg. 0.4 mg/kg. 0.5 mg/kg. 1.0 mg/kg, 1.5 mg/kg, 2.0 mg/kg, 2.5 mg/kg, 3.0 mg/kg, 3.5 mg/kg, 4.0 mg/kg, 4.5 mg/kg, and 5.0 mg/kg, all of which are kg of patient body weight. The total therapeutically or prophylactically effective amount of the bispecific adaptor can be administered in single or divided doses and may, at the practitioner's discretion, fall outside of the typical range given herein.

In another embodiment, the bispecific adaptor can be administered in a therapeutically or prophylactically effective amount of from about 0.5 g/m² to about 500 mg/m², from about 0.5 g/m² to about 300 mg/m², or from about 100 g/m² to about 200 mg/m². In other embodiments, the amounts can be from about 0.5 mg/m² to about 500 mg/m², from about 0.5 mg/m² to about 300 mg/m², from about 0.5 mg/m² to about 200 mg/m², from about 0.5 mg/m² to about 100 mg/m², from about 0.5 mg/m² to about 50 mg/m², from about 0.5 mg/m² to about 600 mg/m², from about 0.5 mg/m² to about 6.0 mg/m², from about 0.5 mg/m² to about 4.0 mg/m², or from about 0.5 mg/m² to about 2.0 mg/m². The total amount can be administered in single or divided doses and may, at the physician's discretion, fall outside of the typical range given herein. These amounts are based on meters of body surface area. All ranges specified in this paragraph are inclusive of the stated end points and include all 0.5 g/m² increments encompassed in each specified range.

In other embodiments, the amount of the bispecific adapter (or pharmaceutically acceptable salt or hydrate thereof) to be administered to a subject can range, for example, from about 50 nmol/kg to about 3,000 nmol/kg of subject body weight, about 50 nmol/kg to about 2,000 nmol/kg, about 50 nmol/kg to about 1,000 nmol/kg, about 50 nmol/kg to about 900 nmol/kg, about 50 nmol/kg to about 800 nmol/kg, about 50 nmol/kg to about 700 nmol/kg, about 50 nmol/kg to about 600 nmol/kg, about 50 nmol/kg to about 500 nmol/kg, about 50 nmol/kg to about 400 nmol/kg, about 50 nmol/kg to about 300 nmol/kg, about 50 nmol/kg to about 200 nmol/kg, about 50 nmol/kg to about 100 nmol/kg, about 100 nmol/kg to about 300 nmol/kg, about 100 nmol/kg to about 500 nmol/kg, about 100 nmol/kg to about 1,000 nmol/kg, or about 100 nmol/kg to about 2,000 nmol/kg of subject body weight. In other embodiments, the dose can be about 100 nmol/kg, about 150 nmol/kg, about 200 nmol/kg, about 250 nmol/kg, about 300 nmol/kg, about 350 nmol/kg, about 400 nmol/kg, about 450 nmol/kg, about 500 nmol/kg, about 600 nmol/kg, about 700 nmol/kg, about 800 nmol/kg, about 900 nmol/kg, about 1,000 nmol/kg, about 2,000 nmol/kg, or about 3,000 nmol/kg of subject body weight. In other embodiments, between about 20 μg/kg to about 3 mg/kg of subject body weight can be administered. The amount can be between about 0.2 mg/kg to about 0.4 mg/kg of subject body weight or about 50 μg/kg subject body weight. All ranges specified in this paragraph are inclusive of the stated end points and include all 1 nmol/kg or 10 μg/kg increments, as applicable, encompassed in each specified range.

### Uses and Methods

Further provided is a method of treating cancer in a subject, which method comprises administering to the subject cancer-treatment effective amounts of (i) anti-fluorescein (e.g., fluorescein, FITC, or NHS-fluorescein) CAR-T cells or a pharmaceutical composition comprising same and a pharmaceutically acceptable carrier or excipient and (ii) a bispecific adapter or a pharmaceutical composition comprising same and a pharmaceutically acceptable carrier or excipient, whereupon the subject is treated for cancer.

The terms “treat,” “treating,” “treated,” and “treatment” refer to therapeutic treatment. Such treatment can have a prophylactic effect. Cancer is treated when the symptoms or signs of cancer are ameliorated, such as a reduction in the size of a tumor, complete or partial elimination of a tumor, stabilization of cancer such as by inhibiting the progression of cancer (e.g., increase in the size of a tumor or increase in the number of tumors, such as due to metastasis), or any other effect on the cancer that a physician would consider to constitute therapeutic (or prophylactic) treatment.

The term “subject,” as used herein, means an animal, such as a mammal, and in particular a human. In veterinary applications, the subject can be a laboratory, an agricultural, a domestic, or a wild animal. Examples of such animals include, but are not limited to, a rodent, a rabbit, a monkey, a chimpanzee, a dog, a cat, a cow, a horse, a pig, a sheep, a goat, a bear, a panda, a lion, a tiger, a leopard, an elephant, a zebra, a giraffe, a gorilla, a dolphin, or a whale.

The anti-fluorescein (e.g., fluorescein, FITC, or NHS-fluorescein) CAR-T cells are T cells (alternatively, NK cells can be used) engineered to express a CAR that recognizes and binds to fluorescein (e.g., fluorescein, FITC, or NHS-fluorescein) in the bispecific adapter.

The CAR is a fusion protein comprising at least three domains, which include (i) a recognition region (e.g., a single-chain fragment variable (scFv) region of an antibody), which recognizes and binds to fluorescein (e.g., fluorescein, FITC, or NHS-fluorescein) with specificity, (ii) a co-stimulation domain, which enhances the proliferation and survival of the T lymphocytes, and (iii) an activation signaling domain, which generates a cytotoxic T lymphocyte activation signal.

scFv regions of antibodies that bind fluorescein (e.g., in FITC) can be used and prepared from (i) an antibody known in the art that binds to fluorescein (e.g., fluorescein, FITC, or NHS-fluorescein), (ii) a newly prepared anti-fluorescein antibody, or (iii) sequence variants derived from the scFv regions of such antibodies. e.g., scFv regions having at least about 80%, at least about 90%, at least about 95%, at least about 97%, at least about 98%, at least about 99%, or at least about 99.5% sequence identity to the amino acid sequence of the scFv region from which they are derived. The binding portion of the CAR can be, for example, an scFv of an antibody, an Fab, Fv, Fc, or (Fab′)₂ fragment.

“Percent (%) sequence identity” with respect to a reference to a polypeptide sequence is defined as the percentage of amino acid or nucleic acid residues, respectively, in a candidate sequence that are identical with the residues in the reference sequence, after aligning the sequences and introducing gaps, if necessary, to achieve the maximum percent sequence identity and not considering any conservative substitutions as part of the sequence identity. Alignment for purposes of determining percent sequence identity can be achieved in various ways that are within the skill of the art, for instance, using publicly available computer software. For example, determination of percent identity or similarity between sequences can be done, for example, by using the GAP program (Genetics Computer Group, software; now available via Accelrys online), and alignments can be done using, for example, the ClustalW algorithm (VNTI software, InforMax Inc.). Further, a sequence database can be searched using the nucleic acid or amino acid sequence of interest. Algorithms for database searching are typically based on the BLAST software (Altschul et al., 1990), but those skilled in the art can determine appropriate parameters for aligning sequences, including any algorithms needed to achieve maximal alignment over the full length of the sequences being compared. In some embodiments, the percent identity can be determined along the full length of the nucleic acid or amino acid sequence.

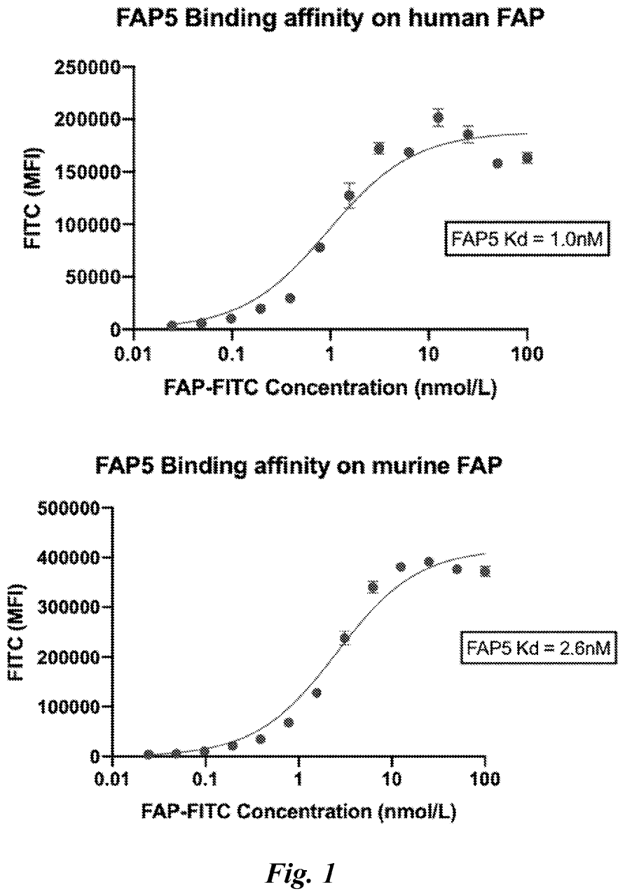

In various embodiments, the CAR has a recognition region and the recognition region is a scFv region of an anti-fluorescein antibody, which can bind fluorescein. FITC, or NHS-fluorescein (see, e.g., the E2 anti-fluorescein antibody described in Vaughan et al., Nature Biotechnol 14(3): 309-314 (1996), and the exemplary CAR construct, which expresses a CAR comprising the E2 anti-fluorescein antibody, shown in FIG. 1 and described on page 66, line 16, through page 69, line 12, of International Patent Application Publication No. WO 2019/144091, both of which are hereby incorporated by reference for their teachings regarding same). The CAR has a co-stimulation domain and the co-stimulation domain can be CD28 (cluster of differentiation 28), CD2 (cluster of differentiation 2), CD137 (cluster of differentiation 137; 4-1BB), a member of the tumor necrosis factor (TNF) family, CD134 (cluster of differentiation 134; OX40), a member of the TNF receptor (TNFR) super family of receptors, CD27 (cluster of differentiation 27), CD30 (cluster of differentiation 30), CD150 (cluster of differentiation 150), DAP10, NKG2D, CD278 (cluster of differentiation 278; ICOS), a CD28-superfamily co-stimulatory molecule expressed on activated T cells, signaling lymphocytic activation molecule (SLAM)-related receptor family (such as 2B4), or any combination thereof. Sequence variants of the aforementioned co-stimulation domains, which have the same or similar activity as the domain on which they are modeled, also can be used without adversely impacting the method. The CAR has an activation signaling domain, and the activation signaling domain can be a T cell CD3 chain, CD3 delta receptor protein, mbl receptor protein, B29 receptor protein, or an Fc receptor γ. Sequence variants of the aforementioned activation signaling domains, which have the same or similar activity as the domain on which they are modeled, also can be used without adversely impacting the method. Such co-stimulation domains and variants of such co-stimulation domains and activation signaling domains can have at least about 80%, at least about 90%, at least about 95%, at least about 97%, at least about 98%, at least about 99%, or at least about 99.5% sequence identity to the amino acid sequence of the domain from which they are derived.

In an embodiment of a CAR comprising an E2 anti-fluorescein antibody fragment, the CAR comprises an IgG4 hinge domain and a CD28 transmembrane domain. The co-stimulation domain is CD137 (4-1BB), and the activation signaling domain is CD3ζ.

In certain embodiments, a CAR comprises an scFv of an anti-fluorescein antibody as a recognition region, a CD137 (4-1BB) co-stimulation domain, and CD3ζ as an activation signaling domain.

Constructs encoding CARs are prepared using genetic engineering techniques. Such techniques are described, for example, in Sambrook et al., Molecular Cloning: A Laboratory Manual, 3^{rd} ed., Cold Spring Harbor Laboratory Press (2001), which is hereby incorporated by reference. By way of example, a plasmid or viral expression vector (e.g., a lentiviral vector, a retroviral vector, sleeping beauty, and piggyback (transposon/transposase systems that include a non-viral-mediated CAR gene delivery system)) can be prepared to encode a fusion protein comprising a recognition region, one or more co-stimulation domains, and an activation signaling domain in frame and linked in a 5′ to 3′ direction. Other arrangements can be acceptable and can include a recognition region, an activation signaling domain, and one or more co-stimulation domains. The placement of the recognition region in the fusion protein will generally be such that display of the region on the exterior of the cell is achieved. The CAR can also include additional elements, such as a signal peptide to ensure proper export of the fusion protein to the cell surface, a transmembrane domain to ensure the fusion protein is maintained as an integral membrane protein, and a hinge domain that imparts flexibility to the recognition region and allows strong binding to the CAR-targeting moiety.

T lymphocytes (e.g., cytotoxic T lymphocytes) can be genetically engineered to express CAR constructs by transfecting a population of the T lymphocytes with an expression vector encoding the CAR construct. Suitable methods for preparing a transduced population of T lymphocytes expressing a selected CAR construct are well-known to the skilled artisan and are described in Sambrook et al. (2001), supra.

The T lymphocytes can be autologous, although heterologous cells can be used, such as when the patient being treated has received high-dose chemotherapy or radiation treatment to destroy the patient's immune system. In various embodiments, allogeneic cells can be used.

T lymphocytes can be obtained from a patient by means well-known in the art. For example, T cells can be obtained by collecting peripheral blood from the patient, subjecting the blood to Ficoll density gradient centrifugation, and then using a negative T cell isolation kit (such as EasySep™ T Cell Isolation Kit) to isolate a population of cytotoxic T cells from the peripheral blood. In various embodiments, the population of cytotoxic T lymphocytes need not be pure and may contain other cells, such as other T cells, monocytes, macrophages, natural killer cells, and B cells. The population of cells being collected can comprise at least about 90% of the selected cell type, such as at least about 91%, 92%, 93%, 94%, 95%, 96%, 97%, 98%, 99% or 100% of the selected cell type.

After the T lymphocytes are obtained, the cells can be cultured under conditions that promote the activation of the cells. The culture conditions can be such that the cells can be administered to a patient without concern for reactivity against components of the culture medium. For example, the culture conditions may not include bovine serum products, such as bovine serum albumin (BSA). Activation can be achieved by introducing known activators into the culture medium, such as anti-CD3 antibodies in the case of cytotoxic T cells. Other suitable activators include anti-CD28 antibodies. The population of lymphocytes can be cultured under conditions promoting activation for about 1 to about 4 days. The appropriate level of activation can be determined by cell size, proliferation rate, or activation markers determined by flow cytometry.

After the population of cytotoxic T lymphocytes has been cultured under conditions promoting activation, the cells can be transfected with an expression vector encoding a CAR. After transfection, the cells can be immediately administered to the patient or the cells can be cultured for at least about 1, 2, 3, 4, 5, 6, 7, 8, 9, 10, 11, 12, 13, 14, 15, 16, 17, 18 or more days, or between about 5 and about 12 days, between about 6 and about 13 days, between about 7 and about 14 days, or between about 8 and about 15 days, for example, to allow time for the cells to recover from the transfection. Suitable culture conditions can be similar to the conditions under which the cells were cultured for activation, either with or without the agent that was used to promote activation.

When the cells have been transfected and activated, a composition comprising the CAR-T cells can be prepared and administered to the subject. Culture media that lack any animal products, such as BSA, can be used. Tissue culture conditions typically used in the art can be used to avoid contamination with bacteria, fungi, and mycoplasma. Cells can be pelleted, washed, and resuspended in a pharmaceutically acceptable carrier, diluent, or excipient.

Exemplary compositions comprising CAR-T cells include compositions comprising the cells in sterile 290 mOsm saline, infusible cryomedia (containing Plasma-Lyte A, dextrose, sodium chloride, human serum albumin (HSA), and dimethylsulfoxide (DMSO)) in 0.9% NaCl with 2% HSA, or in any other sterile 290 mOsm infusible material. Depending on the identity of the culture medium, the CAR-T cells can be administered in the culture medium as the composition or concentrated and resuspended in the culture medium before administration.

The CAR-T cell composition can be administered to the subject by any suitable means, such as parenteral administration, e.g., intradermally, subcutaneously, intramuscularly, intraperitoneally, intravenously, or intrathecally.

The total number of CAR-T cells and the concentration of the cells in the composition administered to the subject will vary depending on a number of factors including the type of CAR-T cells being used, the binding specificity of the CAR, the identity of the CAR-targeting moiety (in the examples herein. FITC), and the identity small molecule ligand/targeting ligand of the bispecific adapter (e.g., PSMAL1, DUPA, a FR ligand, and/or a FAP ligand), the identity of the cancer, the location of the cancer in the subject, the means used to administer the compositions to the subject, and the health, age and weight of the subject being treated. Suitable compositions comprising transduced CAR-T cells include those having a volume of between about 5 ml and about 200 ml, containing from about 1×10⁵ to about 1×10¹⁵ transduced CAR-T cells. Typical compositions comprise a volume of between about 10 ml and about 125 ml and contain from about 1×10⁷ to about 1×10¹⁰ CAR-T cells. An exemplary composition comprises about 1×10⁹ CAR-T cells in a volume of about 100 ml. A single dose or multiple doses of the CAR-T cells can be administered to the subject. Compositions can comprise about 1 million (M), 2M, 3M, 4M, 5M, 6M, 7M, 8M, 9M, 10M, 11M, 12M, 12.5M, 13M, 14M or 15M CAR-T cells, such as per kg of patient body weight. When the CAR-T cell composition is administered by injection into the subject's bloodstream, the CAR-T cells in the subject's bloodstream are at least 5%, 7%, 10%, 11%, 12%, 13%, 14%, or 15% of the subject's total T cells in the subject's bloodstream by about four weeks after injection, at least 20%, 25%, 30%, 35%, 40%, or 50% of the subject's total T cells in the subject's bloodstream by about two weeks after injection, or at least 85%, 90% or 95% of the subject's total T cells by about one week after injection.

The bispecific adapter (or pharmaceutically acceptable salt or hydrate thereof) or pharmaceutical composition comprising same, or a combination thereof and the anti-fluorescein CAR-T cells or pharmaceutical composition comprising the anti-fluorescein CAR-T cells can be administered to the patient using any suitable method known in the art. The terms “administer,” “administering,” “administered.” and “administration” refer to methods of introducing the bispecific adapter (or a pharmaceutically acceptable salt or hydrate thereof) or a pharmaceutical composition comprising the bispecific adapter (or a pharmaceutically acceptable salt or hydrate thereof) and methods of introducing the anti-fluorescein CAR-T cells or a pharmaceutical composition comprising the anti-fluorescein CAR-T cells. Examples of suitable routes of administration include, but are not limited to, oral, intravenous, intramuscular, subcutaneous, and transdermal. The components can be administered directly into the blood stream, into muscle, or into an internal organ. Suitable routes for parenteral administration include, but are not limited to, intravenous, intra-arterial, intraperitoneal, intrathecal, epidural, intracerebroventricular, intraurethral, intrasternal, intracranial, intratumoral, intramuscular, and subcutaneous. Use can be made of needle injectors, including microneedles, needle-free injectors, and infusions. The aforementioned components can be administered in unit dosage forms and/or formulations containing conventional non-toxic pharmaceutically acceptable carriers or excipients (or vehicles or adjuvants).

In the method, the anti-fluorescein CAR-T cells (or pharmaceutical composition comprising anti-fluorescein CAR-T cells and a pharmaceutically acceptable carrier or excipient) and the bispecific adapter, a pharmaceutical composition, or combination can be administered simultaneously or sequentially, in either order, by the same or different routes. When administered simultaneously by the same route, the formulations can be the same or differ. In various embodiments, the bispecific adapter can be administered to the subject after the CAR-T cells. The timing between the administration of CAR-T cells and the administration of the bi-specific adapter can vary widely depending on factors that include the type of CAR-T cells being used, the binding specificity of the CAR, the identity of the CAR-targeting moiety (in the examples herein, a fluorescein) and the small molecule ligand/targeting moiety of the bispecific adapter (in the examples herein, a PSMA ligand, a FAP ligand, a FR ligand, or a combination of PSMA and FAP ligands, or a combination of FAP ligands and FR ligands), the identity of the cancer, the location in the subject of the cancer, the means used to administer to the subject the CAR-T cells and the bispecific adapter, as well as the health, age, and weight of the patient.

The bispecific adapter(s) can be administered before or after the CAR-T cells, such as within about 3, 6, 9, 12, 15, 18, 21 or 24 hours, or within about 0.5, 1, 1.5, 2, 2.5, 3, 4, 5, 6, 7, 8, 9, 10 or more days. The rate of tumor lysis can be regulated by adjusting the rate of administration of the bispecific adapter, for example (e.g., as a function of dosing schedule, such as continuous, once daily, twice daily, thrice daily, once weekly, twice weekly, or thrice weekly). By “continuous” is meant for at least one hour, at least four hours, at least six hours, at least eight hours, at least 10 hours, at least 12 hours, or at least 24 hours, or a regimen of daily or weekly administration, such as once/day twice/day, thrice/day, every other day, once/week, twice/week, thrice/week or any other suitable regimen. In the method, (i) and (ii) can be administered intravenously. The cancer can be ovarian cancer, endometrial cancer, breast cancer, glioma (e.g., stage 3-4 glioma), or clear cell renal cell carcinoma (e.g., stage 3-4 clear cell renal cell carcinoma).

Such combination therapy methods can be performed using any engineered cell that is suitable for the treatment of cancer and can include using more than one of these types of agents. In certain embodiments, the engineered cells used in this combination therapy are CAR T-cells and can also (or alternatively) comprise engineered stem cells and other cells.

The engineered cells used in combination with the inventive bispecific adapters or compositions can be any CAR T cells, stem cells or other engineered cell or combination thereof. Various adoptive cell therapies (also termed cellular immunotherapy) are known in the art for use in the treatment of cancer, and T-cell immunotherapy, in particular, has received much attention. Some non-limiting examples of such therapies include engineered T cell receptor (TCR) therapy, CAR T cell therapy, and natural killer (NK) cell therapy.

In certain approaches, administering both the bispecific adaptor compounds and the engineered cellular therapy results in a greater than additive inhibition of growth of the cancer.

Where multiple therapeutics and/or therapies are co-administered, dosages may be adjusted accordingly, as is recognized in the pertinent art. “Co-administration” and combination therapy are not limited to simultaneous administration, but also include treatment regimens in which a targeted bispecific adaptor is administered at least once during a course of treatment that involves administering a cellular therapy to a subject.

Cytokine release syndrome (CRS) can be controlled by varying the dose of the bispecific adapter. See, e.g., International Patent Application Publication No. WO 2017/177149.

The methods of treating cancer hereof can comprise administering any of the bispecific adapters to the patient and administering any of the above-described engineered cell compositions or engineered cell therapy to the patient.

In certain embodiments, a method of treating cancer in a subject is provided. The method comprises administering to the subject cancer-treatment effective amounts of: (i) anti-fluorescein CAR-T cells or a pharmaceutical composition comprising anti-fluorescein CAR-T cells and a pharmaceutically acceptable carrier or excipient; and (ii) any bispecific adapter, any pharmaceutical composition comprising same, or any combination of bispecific adapters. Steps (i) and (ii) can be administered simultaneously or sequentially, in either order, by the same or different routes.

The anti-fluorescein CAR-T cells can comprise any CAR T-cells described herein or suitable for use as described. In certain embodiments, anti-fluorescein CAR-T cells comprise a recognition region comprising a scFv region of an anti-fluorescein antibody; a co-stimulation domain, and the co-stimulation domain is CD28, CD137 (4-1BB), CD134 (OX40), or CD278 (ICOS); and/or an activation signaling domain that is a T cell CD3ζ chain or an Fc receptor γ.

Where the method administers a combination, the combination can comprise first and second bispecific adapters, which can be administered to the subject simultaneously by the same or different routes. Alternatively, the first and second bispecific adapters can be administered to the subject sequentially, in either order, by the same or different routes.

In certain embodiments, both steps (i) and (ii) of the method are administered intravenously.

The fluorescein of the bispecific adapter can bind the anti-fluorescein CAR-T cell with affinity upon exposure thereto, and the targeting ligand of the bispecific adapter can link the bound anti-fluorescein CAR-T cell to a targeted cancer cell or CAF upon the targeted ligand of the bispecific adapter binding a receptor on such targeted cancer cell or CAF with affinity. In this manner, the compounds, compositions, and combinations hereof facilitate enhanced efficacy of CAR-T cell therapy.

In certain embodiments, the receptor on the targeted cancer cell or CAF is an overexpressed FAP, an over-expressed PSMA, and/or a FR (e.g., an over-expressed FR). The cancer can be a FAP-expressing cancer, and at least one bispecific adapter of (ii) can comprise a radical of a FAP ligand. The cancer can be a PSMA-expressing cancer, and at least one bispecific adapter of (ii) can comprise a radical of a PSMA ligand. In certain embodiments, for example where the method administers a combination hereof, the cancer is a FR-expressing cancer and (ii) can comprise a combination hereof comprising a folate.

Also provided are methods for treating FAP-expressing cancer in a subject. In certain embodiments, the method comprises administering to the subject cancer-treatment effective amounts of (i) anti-fluorescein CAR-T cells or a pharmaceutical composition comprising anti-fluorescein CAR-T cells and a pharmaceutically acceptable carrier or excipient; and (ii) any bispecific adapter or a pharmaceutical composition comprising a bispecific adapter and a pharmaceutically acceptable carrier or excipient. As noted above, the CAR can have a recognition region, and the recognition region can be a scFv region of an anti-fluorescein antibody. In certain embodiments, the CAR comprises: a co-stimulation domain, and the co-stimulation domain can be CD28, CD137 (4-1BB), CD134 (OX40), or CD278 (ICOS); and/or an activation signaling domain, and the activation signaling domain can be a T cell CD3ζ chain or an Fc receptor γ.

In certain embodiments, a method of treating cancer in a subject comprises administering to the subject cancer-treatment effective amounts of (i) anti-fluorescein chimeric antigen receptor (CAR)-T cells or a pharmaceutical composition comprising anti-fluorescein CAR-T cells and a pharmaceutically acceptable carrier or excipient; and (ii) any combination of bispecific adapters. Here, the CAR can comprise: a recognition region comprising a scFv region of an anti-fluorescein antibody; a co-stimulation domain and the co-stimulation domain can be CD28, CD137 (4-1BB), CD134 (OX40), or CD278 (ICOS); and/or an activation signaling domain that is a T cell CD3ζ chain or an Fc receptor γ. Steps (i) and (ii) can be administered simultaneously or sequentially, in either order, by the same or different routes. In certain embodiments, the first and second bispecific adapters of the combination are administered to the subject simultaneously by the same or different routes. In certain embodiments, the first and second bispecific adapters of the combination are administered to the subject sequentially, in either order, by the same or different routes. (i) and (ii) can each be administered intravenously.

The methods hereof can further comprise imaging the cancer in the subject. Imaging the cancer can comprise imaging by optical imaging, positron emission tomography (PET), or single photon emission computed tomography (SPECT), for example.

In the methods the cancer can additionally be imaged prior to administration to the subject of the bispecific adapter, or the pharmaceutically acceptable salts or hydrates thereof, or the engineered cell composition (e.g., a CAR-expressing cytotoxic lymphocyte composition or a CAR-NK cell composition). The cancer additionally, or alternatively, can be imaged during or after administration to assess metastasis, for example, and the efficacy of treatment. For example, imaging can occur by PET imaging, magnetic resonance imaging (MRI), or SPECT/computed tomography (CT) imaging. The imaging method can be any suitable imaging method known in the art.

The cancer can be any cancer. “Cancer” has its plain and ordinary meaning when read in light of the specification and can include, but is not limited to, a group of diseases involving abnormal cell growth with the potential to invade or spread (i.e., metastasize) to other parts of the body. Examples include, but are not limited to, a cancer of the brain, thyroid, lung, pancreas, kidney, stomach, gastrointestinal stroma, endometrium, breast, cervix, ovary, colon, prostate, leukemias, lymphomas, other blood-related cancers, or head and neck cancer. In certain embodiments, the cancer being treated is a tumor. In certain embodiments, the cancer is malignant. In certain embodiments, the cancer is ovarian cancer, endometrial cancer, breast cancer, glioma such as, optionally, stage 3-4 glioma, or clear cell renal cell carcinoma such as, optionally, stage 3-4 clear cell renal cell carcinoma.

In some aspects of these embodiments, the cancer is a folate receptor-expressing cancer, for example and without limitation, an FR α-expressing cancer. In other embodiments, the cancer is an FR β-expressing cancer. In certain embodiments, the cancer is a FAP-expressing cancer. In certain embodiments, the cancer is a PSMA-expressing cancer.

In some embodiments, the cancer is imaged prior to administration of (i) and (ii) to the subject. Imaging can be done by PET, MRI or SPECT/CT.

In certain embodiments, a use of a bispecific adaptor, a pharmaceutically acceptable salt, hydrate, or solvate of the bispecific adaptor, or a composition thereof in the manufacture of a medicament for the treatment of cancer in a subject is provided. The bispecific adaptor can be any compound or conjugate hereof. The medicament can be for use in combination with administration of an engineered cell therapy to the subject such as, for example, CAR T-cell therapy, wherein the CAR T-cells express anti-fluorescein.

Still further, a method for enhancing CAR-T cell activation is provided. The method can comprise providing a bispecific adapter hereof, a pharmaceutical composition hereof, or a combination hereof (e.g., a therapeutically effective amount of any of the foregoing); and exposing anti-fluorescein CAR-T cells or a pharmaceutical composition comprising anti-fluorescein CAR-T cells and a pharmaceutically acceptable carrier or excipient to the bispecific adaptor(s), pharmaceutical composition, or combination; wherein the CAR-T cell experiences enhanced activation following exposure as compared to a CAR-T cell not exposed to the bispecific adapter. The anti-fluorescein CAR-T cells can be in systemic circulation in a subject when exposed to the bispecific adaptor, pharmaceutical composition, or combination, for example. Alternatively, the anti-fluorescein CAR-T cells can be exposed in vitro to the bispecific adaptor, pharmaceutical composition, or combination.

### Kits

Still further provided is a kit. The kit can comprise (i) a bispecific adapter, or a pharmaceutical composition comprising the same and a pharmaceutically acceptable carrier or excipient, or a combination thereof, and (ii) anti-fluorescein CAR-T cells (e.g., anti-FITC CAR-T cells) or a pharmaceutical composition comprising the same and a pharmaceutically acceptable carrier or excipient.

In certain embodiments, the bispecific adaptor(s), pharmaceutical composition, or combination and the CAR-T cells are stored in separate containers. In certain embodiments where the kit comprises a combination hereof, the first and second bispecific adapters are stored in separate containers.

### General

Those skilled in the art will recognize that numerous modifications can be made to the specific implementations described above. The implementations should not be limited to the particular limitations described. Other implementations may be possible.

While the bispecific adapters and pharmaceutical compositions are illustrated and described in detail in the foregoing description, the same is to be considered as illustrative and not restrictive in character, it being understood that only certain embodiments have been shown and described and that all changes and modifications that come within the spirit of the invention are desired to be protected.

It is intended that that the scope of the present bispecific adapters, compositions, and methods are defined by the following claims. However, this disclosure may be practiced otherwise than is specifically explained and illustrated without departing from its spirit or scope. Those skilled in the art will understand that various alternatives to the embodiments described herein can be employed in practicing the claims without departing from the spirit and scope as defined in the following claims.

Any use of section headings is intended to aid reading of the document and is not to be interpreted as limiting. Further, information that is relevant to a section heading may occur within or outside of that particular section.

All publications, patents, patent application publications, journal articles, textbooks, and other publications referred to in this document are indicative of the level of skill of those in the art to which the disclosure pertains. All such publications are incorporated herein by reference to the same extent as if each individual publication were specifically and individually indicated to be incorporated by reference. In the event of inconsistent usages between this document and those documents so incorporated by reference, the usage in the incorporated reference should be considered supplementary to that of this document; for irreconcilable inconsistencies, the usage in this document controls.

Various techniques and mechanisms will sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note. Consequently, a connection does not necessarily mean a direct, unimpeded connection unless otherwise noted.

### Certain Definitions

As used herein, the following terms and phrases shall have the meanings set forth below. Unless defined otherwise, all technical and scientific terms used herein have the same meaning as commonly understood to one of ordinary skill in the art.

The term “about” or “approximately” means within an acceptable range for the particular value as determined by one of ordinary skill in the art, which will depend in part on how the value is measured or determined. e.g., the limitations of the measurement system. For example. “about” can mean a range of up to 20%, preferably up to 10%, more preferably up to 5%, and more preferably still up to 1% of a given value. By way of further example, “about” or “approximately” can mean within 90%, within 95%, 99%, 99.5%, 99.9%, 99.99%, or at least about 99.999% or more of a stated value or of a stated limit of a range. Alternatively, particularly with respect to biological systems or processes, the term can mean within an order of magnitude, preferably within 5-fold, and more preferably within 2-fold, of a value. Unless otherwise stated, the term “about” means within an acceptable error range for the particular value, such as ±1-20%, preferably ±1-10% and more preferably ±1-5%.

Where a range of values is provided, it is understood that each intervening value, between the upper and lower limit of that range and any other stated or intervening value in that stated range, is encompassed. The upper and lower limits of these smaller ranges may independently be included in the smaller ranges, and are also encompassed, subject to any specifically excluded limit in the stated range. Where the stated range includes one or both limits, ranges excluding either or both of those limits are also included.

A phrase referring to “at least one of” a list of items refers to any combination of those items, including single members. As an example, “at least one of a, b, or c” is intended to cover: a, b, c, a-b, a-c, b-c, and a-b-c.

The terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation.

The term “or” is used to refer to a nonexclusive “or” unless otherwise indicated. In addition, it is to be understood that the phraseology or terminology employed herein, and not otherwise defined, is for the purpose of description only and not of limitation.

The terms and expressions employed are used as terms of description and not of limitation. Where certain terms are defined and are otherwise described or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings may be used in the “Detailed Description,” such use is solely for ease of reference and is not intended to limit any disclosure made in one section to that section only; rather, any disclosure made under one subheading is intended to constitute a disclosure under each and every other subheading.

It is recognized that various modifications are possible within the scope of the claimed invention. Thus, although the present invention has been specifically disclosed in the context of preferred embodiments and optional features, those skilled in the art may resort to modifications and variations of the concepts disclosed herein. Such modifications and variations are considered within the scope of the invention as claimed herein.

### EXAMPLES

The following examples serve to illustrate the present disclosure. The examples are not intended to limit the scope of the claimed invention in any way.

### Example 1

### Synthesis of FAP8-PEG₅-FITC Conjugate

The FAP8-PEG₃-FITC conjugate can be synthesized according to Scheme 1.

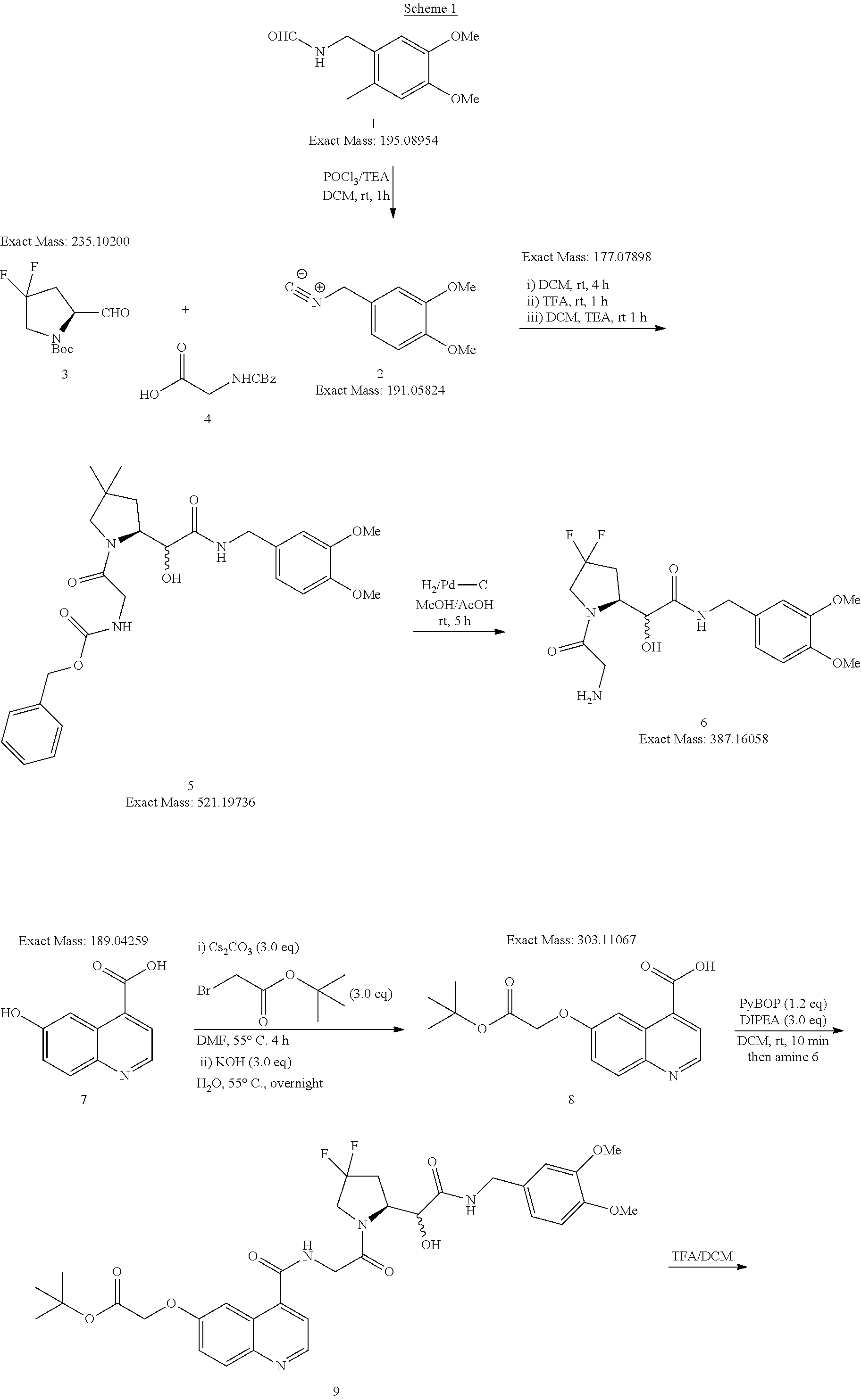

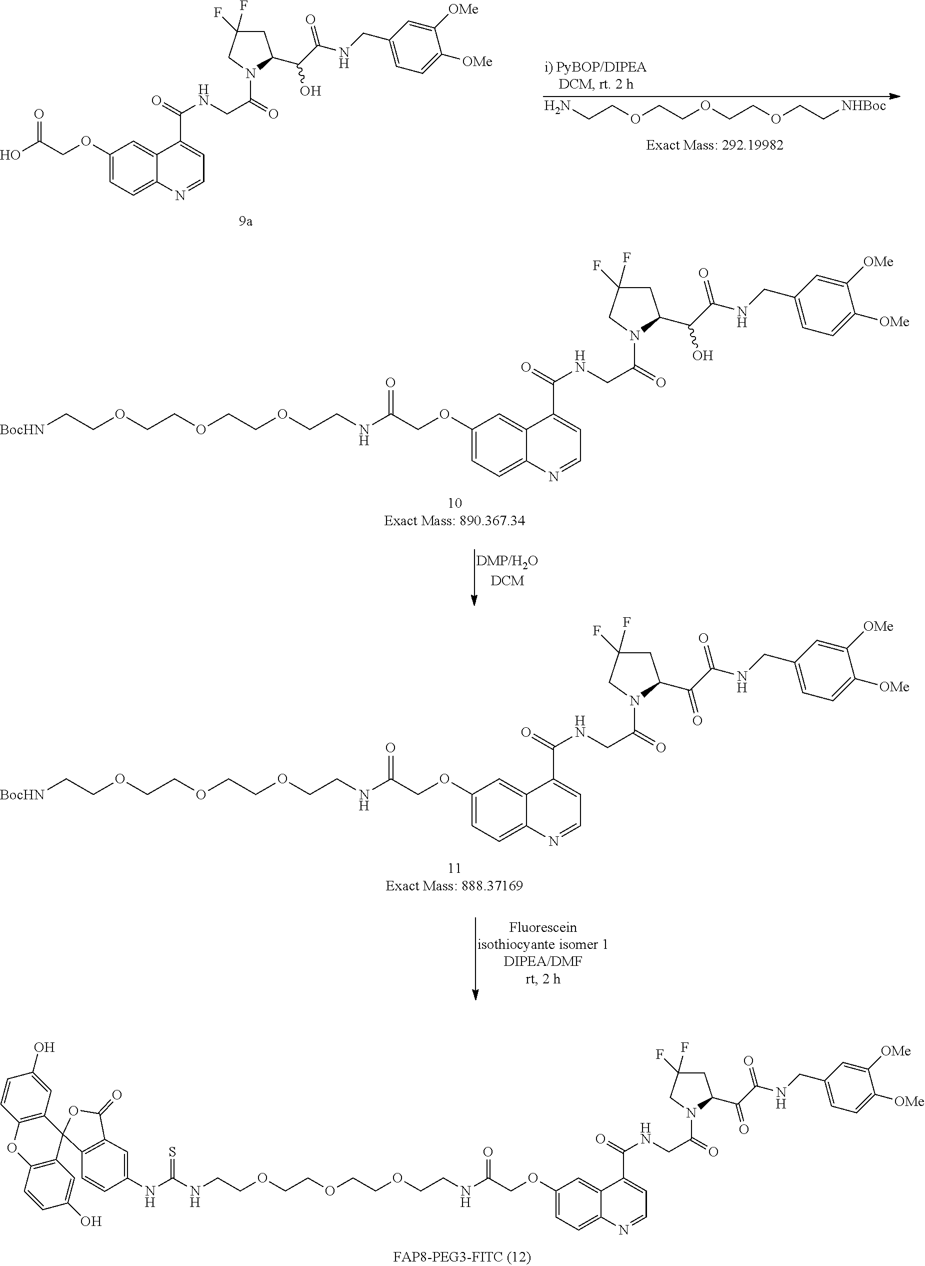 [SMILES: COc1ccc(CNC(=O)C(O)[C@@H]2CC(F)(F)CN2C(=O)CNC(=O)c2ccnc3ccc(OCC(=O)O)cc23)cc1OC ; CC(C)(C)OC(=O)NCCOCCOCCOCCN ; COc1ccc(CNC(=O)C(O)[C@@H]2CC(F)(F)CN2C(=O)CNC(=O)c2ccnc3ccc(OCC(=O)NCCOCCOCCOCCNC(=O)OC(C)(C)C)cc23)cc1OC ; COc1ccc(CNC(=O)C(=O)[C@@H]2CC(F)(F)CN2C(=O)CNC(=O)c2ccnc3ccc(OCC(=O)NCCOCCOCCOCCNC(=O)OC(C)(C)C)cc23)cc1OC ; COc1ccc(CNC(=O)C(=O)[C@@H]2CC(F)(F)CN2C(=O)CNC(=O)c2ccnc3ccc(OCC(=O)NCCOCCOCCOCCNC(=S)Nc4ccc5c(c4)C(=O)OC54c5cc(O)ccc5Oc5ccc(O)cc54)cc23)cc1OC]

To a stirred solution compound 1 (1.0 g, 5.1 mmol) in dry methylene chloride (DCM) (3.0 mL) at 0° C. was added Et₃N (5.0 eq), followed by POCl3 (1.5 eq), and the reaction mixture was allowed to continue at same temperature for 1 hour. Progress of the reaction was monitored by thin layer chromatography (TLC). After completion of starting materials as indicated on TLC plates, the reaction mixture was further diluted with DCM then absorbed on silica-gel cartridge and purified using ethylacetate+hexanes as mobile phase to provide the desired compound 2 (750 mg, 81%) as light yellow liquid.

Synthesis of compounds (5 and 6): To a mixture of N-Boc-L-prolinal (1 eq. 200 mg, 0.851 mmol), N-protected gly cine (1 eq, 161 mg, 0.851 mmol) and isocyanide (1 eq, 162 mg. 0.851 mmol.) were dissolved in anhydrous DCM (10 mL) and stirred for 4 hours. After complete conversion of starting materials (as monitored by liquid chromatography-mass spectrometry (LC-MS)), trifluoroacetic acid (3.0 mL) was added to the reaction mixture and stirring was continued at room temperature for 1 hour. The volatiles were evaporated under reduced pressure. The oily residue was redissolved in anhydrous DCM (10 mL) and cooled down to 0° C. with ice bath. Triethylamine (5.0 mL) was added dropwise, and the stirring continued until full conversion (monitored by LC-MS, different retention time with same mass), which usually took less than 2 hours. The liquids were evaporated under reduced pressure, the mixture was redissolved in DCM and washed 3 times with water. Organic phase was washed with brine, dried over sodium sulfate and the solvent was evaporated under reduced pressure and obtained crude residue was purified by using combiflash with hexanes+ethyl acetate as mobile phase provided desired α-hydroxyamide 5 brown color solid. The product was then used in the next step by dissolving in MeOH+AcOH (10 mL, 1:1) and added 10% Pd—C(100 mg for 1 g of starting material), then stirred under hydrogen atmosphere for 6 hours. The reaction mixture was filtered thorough celite pad and filtrate was evaporated under reduced pressure to obtain crude residue. The crude residue was azeotrope with EtOH, which was purified by combiflash using methanol (MeOH)+DCM to give the amine 6 as white color solid.

Synthesis of compound (8): Cs₂CO₃ (2.65 gm, 7.93 mmol) followed by tertiary-butyl bromo acetate (7.93 mmol) was added to a solution of compound 7 (500 mg, 2.64 mmol) in DMF (10. mL). The reaction mixture was stirred at 55° C. for 4 hours, then (KOH 3 eq with respect to dialkylated product)+H₂O (5.0 mL) were added to the same reaction mixture and stirring was continued there for additional 12 hours. The progress of the reaction was monitored by LC-MS, and the reaction mixture was carefully neutralized with IN HCl and extracted multiple times with EtOAc. Combined organic extracts were evaporated under reduced pressure, and crude residue was obtained and purified by combiflash using a EtOAc+Hexanes system to provide the desired compound 8 as white solid (650 mg 81%).

Synthesis of compound (9): PyBOP (411 mg, 0.792 mmol) and DIPEA (0.22 mL, 1.32 mmol) were added to a stirred solution of compound 8 (200 mg. 0.66 mmol) in anhydrous CH₂Cl₂ (10.0 mL) and stirred there for 10 minutes. Thereafter, amine 6 (0.66 mmol) was added to the reaction mixture and stirring continued there for an additional 2 hours. The reaction mixture was diluted with water, then extracted into DCM (2×20 mL). The combined organic extracts were dried over anhydrous sodium sulphate, filtered and the filtrate was evaporated under reduced pressure to obtain crude residue. The crude residue was purified by combiflash using EtOAc+Hexanes for 15 minutes, followed by MeOH+DCM as mobile phase for 25 minutes to provide compound 9 as a white solid.

Synthesis of compound (10): Trifluoroacetic acid (TFA) (2.0 mL) was added to a solution of compound 10 (200.0 mg, 0.297 mmol) in DCM (5 mL) and stirred at room temperature for 2 hours. The reaction mixture was then was evaporated under reduced pressure and dried under vacuum, to result in crude residue which was purified by combiflash using EtOAc+Hexanes for 15 minutes, followed by MeOH+DCM for 25 minutes to provide the acid compound 9a as white solid. The acid compound 9a (1.0 eq) was then dissolved in DCM, followed by the addition of PyBOP (1.2 eq)+N,N-Diisopropylethylamine (DIPEA) (2.0 eq). After 10 minutes of stirring, BocNH(PEG)₃NH₂ (1.2 eq) was added to the reaction mixture and the stirring continued there for an additional 2 hours. Work up and purification followed the same procedure as described above to provide compound 10 as a white solid. Finally, water (10. eq) was added to compound 10 (1.0 eq) after it was redissolved in DCM followed by Dess-Martin periodinane (DMP) (3.0 eq), and the solution was stirred at room temperature overnight. The reaction mixture was further diluted with water and extracted into DCM (2×30 mL).

The combined organic extracts were dried over anhydrous sodium sulphate and filtered. The filtrate was evaporated under reduced pressure to obtain a crude residue, which was purified by combiflash using MeOH+DCM as mobile phase to provide the desired keto compound 11 as a white solid.

Synthesis of compound 12 (FAP8-PEG₃-FITC): TFA (0.5 mL) was added to a stirred solution of compound 11 (100 mg. 0.1126 mmol) in DCM (1.0 mL). Stirring continued there for 30 minutes. The reaction mixture was then evaporated under reduced pressure to provide the amine 12 as a brown color gummy solid, and this amine was used for further steps without purification.

To a stirred solution of amine 12 (20 mg, 0.0253 mmol) in DMF (500 ul), DIPEA (0.11 mmol) followed by Fluorescein isothiocyanate isomer 1 (1.2 eq) were added and the reaction mixture was continuously stirred at room temperature for an additional 2 hours. The reaction mixture was diluted with water and purified by ultra high performance liquid chromatography (U-HPLC) using (A=Ammonium acetate (10 mM, P^{II}=7.5), B=Acetonitrile) for 60 minutes using the 5-35 method to obtain the desired fractions. These fractions were quickly freezed using liquid nitrogen and lyophilized for 48 hours to provide the desired compound FAP8-PEG3-FITC (13) as a yellow solid.

### Example 2

### Synthesis of FAP5-PEG₈-FITC Conjugate

The FAP5-PEG₈-FITC conjugate can be synthesized according to Scheme 2.

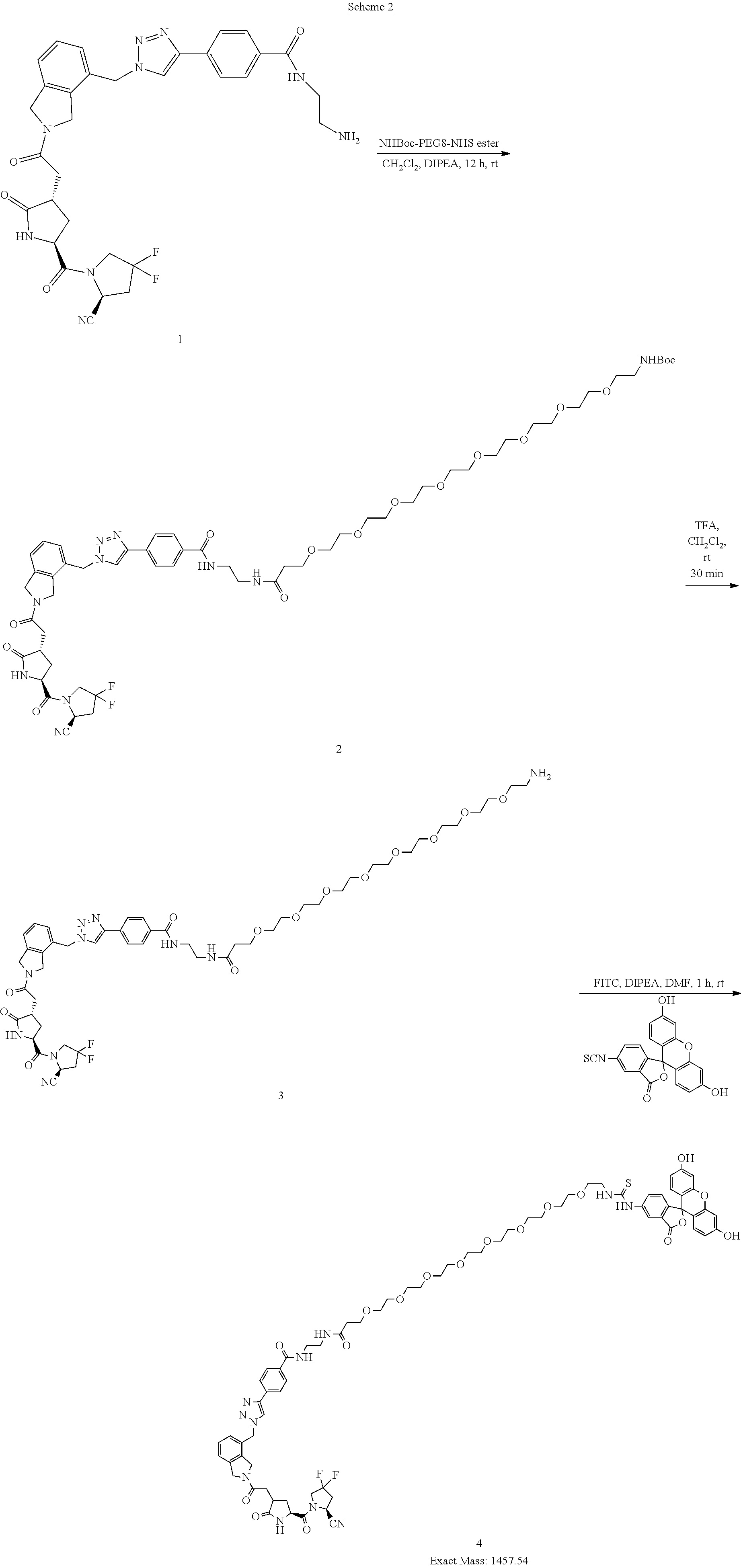

Fluorescein isothiocyanate (FITC) (13.65 mg, 0.035 mmol, 0.95 eq) and DIPEA (13.0 μL, 0.074 mmol, 2 eq) were added to a stirred solution of compound 3 (40 mg, 0.037 mmol, 1 eq) in dry DMF (1.0 mL) and the mixture was continuously stirred for 1 hour at room temperature under N₂. After completion of the reaction (confirmed by LC-MS), the solvent was removed and crude was loaded on celite, which was purified by combi flash (column: 15.5 g HP C18 Aq, flow rate. 40 ml/min, wave length 254 nm, eluent: A=20 mM ammonium acetate buffer (pH=7), B=acetonitrile), solvent gradients 0% B to 100% in 60 minutes to provide FAP5-PEG₈-FITC as a yellow solid. LC-MS (m/z): [M+H] found: 1458.4.

¹H NMR (500 MHz, DMSO) δ 10.18 (s, 1H), 8.69 (d, J=9.5 Hz, 1H), 8.54-8.51 (m, 1H), 8.24 (s, 2H), 8.05-8.01 (m, 2H), 7.97-7.74 (m, 5H). 7.73 (d, J=8.0 Hz, 1H). 7.36-7.31 (m. 2H), 7.23-7.11 (m. 2H), 6.60 (d. J=9.0 Hz. 4H). 6.52 (d. J=8.5 Hz, 2H). 5.66 (d, J=7.0 Hz, 2H), 5.12-5.05 (m, 1H), 4.96 (s, 1H), 4.85 (d, J=6.0 Hz, 1H), 4.65 (d, J=8.0 Hz, 2H), 4.32-4.28 (m, 1H), 4.19-4.06 (m, 2H), 3.66 (s, 2H), 3.59-3.53 (m, 7H), 3.51-3.39 (m, 26H), 3.24-3.16 (m, 3H), 3.00-2.60 (m, 5H), 2.46-2.42 (m. 1H), 2.38-2.27 (m, 3H), 2.14-2.04 (m, 1H).

¹³C NMR (126 MHz, DMSO) δ 181.96, 179.73, 178.88, 171.87, 170.87, 169.85, 169.80, 169.60, 168.99, 166.34, 152.75, 146.48, 146.43, 141.76, 138.30, 137.55, 136.78, 136.48, 135.85, 134.08, 133.56, 131.19, 130.94, 129.63, 12.10, 128.80, 128.69, 128.44, 128.34, 127.95, 127.75, 127.11, 125.31, 123.64, 123.44, 122.83, 122.86, 118.10, 110.51, 102.73, 70.22, 70.10, 69.97, 68.89, 67.23, 52.32, 51.15, 44.79, 44.14, 38.70, 36.65, 36.45, 35.47, 35.40, 31.78, 31.73.

### Example 3

### Synthesis of FAP5-PEG₆-FITC Conjugate

The FAP5-PEG₆-FITC conjugate can be synthesized according to Scheme 3.

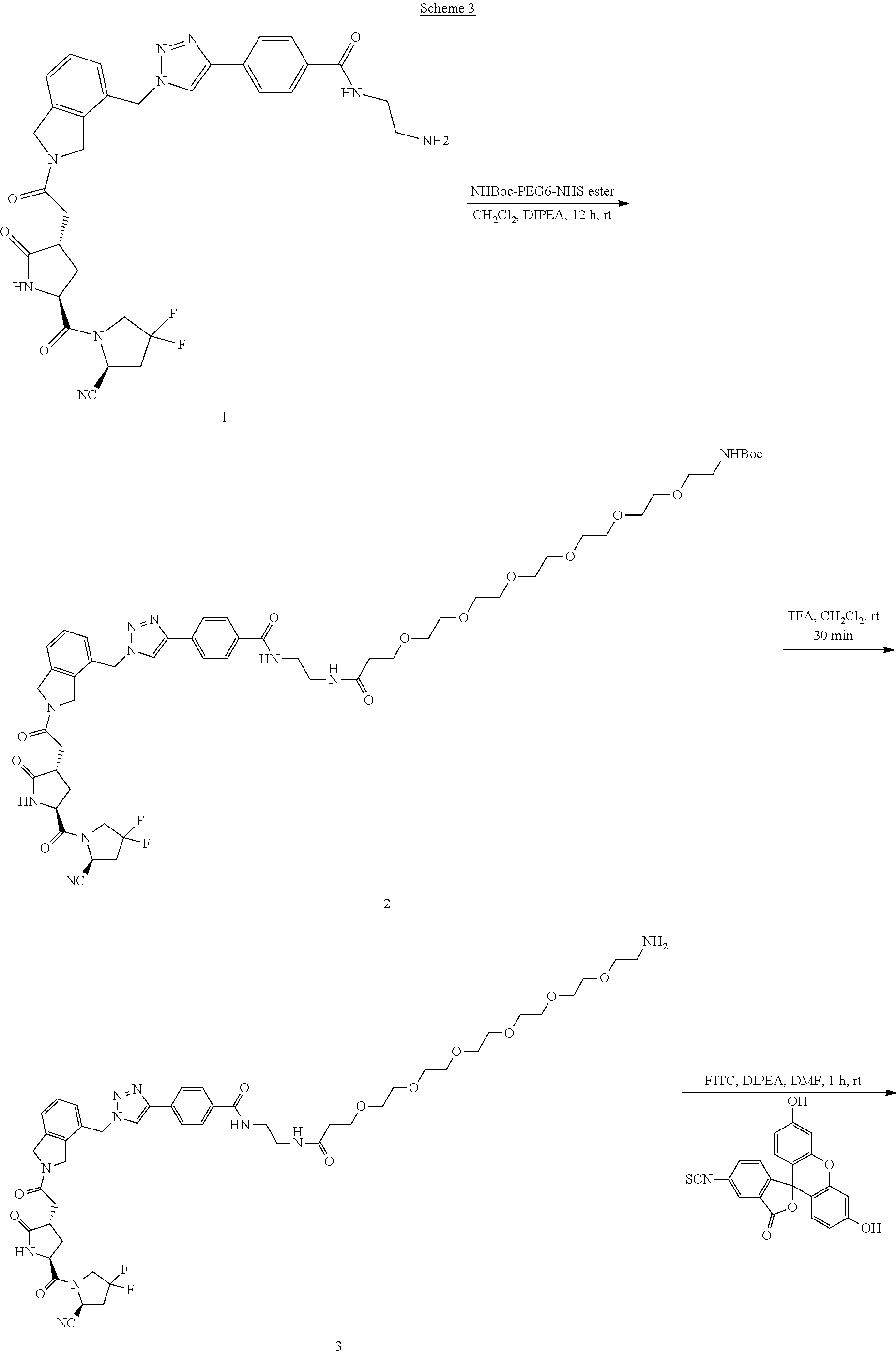

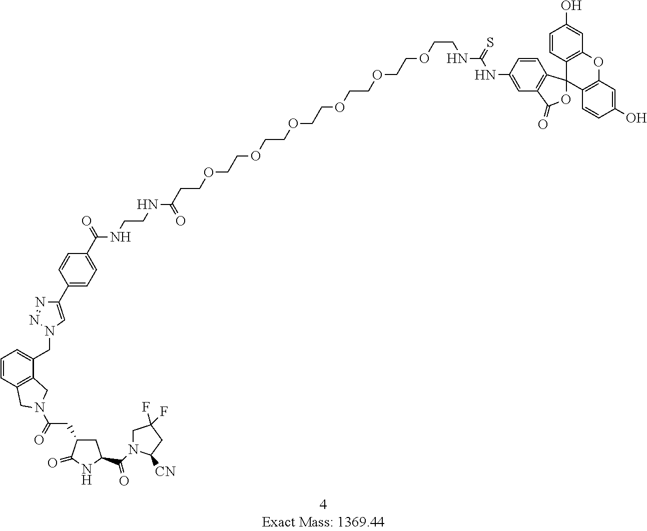 [SMILES: N#C[C@@H]1CC(F)(F)CN1C(=O)[C@@H]1C[C@@H](CC(=O)N2Cc3cccc(Cn4cc(-c5ccc(C(=O)NCCNC(=O)CCOCCOCCOCCOCCOCCOCCNC(=S)Nc6ccc7c(c6)C(=O)OC76c7ccc(O)cc7Oc7cc(O)ccc76)cc5)nn4)c3C2)C(=O)N1]

FITC (5.96 mg, 0.014 mmol, 0.95 eq) and DIPEA (5.32 μL, 0.03 mmol, 2 eq) were added to a stirred solution of compound 3 (15 mg, 0.015 mmol, 1 eq) in dry DMF (1.0 mL) and stirring continued at room temperature for 1 hour under N₂ gas. After completion of reaction (confirmed by LC-MS), the solvent was removed and crude was loaded on celite, and purified by combi flash (column: 15.5 g HP C18 Aq, flow rate: 40 ml/min, wave length: 254 nm, eluent: A=20 mM ammonium acetate buffer (pH=7), B=acetonitrile), solvent gradients 0% B to 100% in 60 minutes to provide FAP5-PEG₆-FITC as a yellow solid (Compound 4). LC-MS (m/z): [M+H] found: 1370.4.

¹H NMR (500 MHz, DMSO-d₆) δ 10.14 (s, 1H), 8.72 (d, J=9.5 Hz, 1H), 8.55-8.51 (m, 1H), 8.26 (s, 2H), 8.06-8.02 (m, 2H), 7.94-7.90 (m, 5H), 7.76 (d, J=8.0 Hz, 1H), 7.36-7.33 (m, 2H), 7.25-7.15 (m, 2H), 6.63 (d, J=9.0 Hz, 4H), 6.55 (d, J=8.5 Hz, 2H), 5.68 (d, J=7.0 Hz, 2H), 5.15-5.07 (m, 1H), 4.98 (s, 1H), 4.88 (d, J=6.0 Hz, 1H), 4.69 (d, J=8.0 Hz, 2H), 4.34-4.30 (m, 1H), 4.21-4.08 (m, 2H), 3.68 (br-s, 2H), 3.60-3.54 (m, 7H), 3.50-3.46 (m, 18H), 3.245-3.19 (m, 3H), 2.89-2.71 (m, 5H), 2.47-2.41 (m, 1H), 2.37-2.27 (m, 3H), 2.15-2.07 (m, 1H).

¹³C NMR (126 MHz, DMSO-d₆) δ 180.99, 178.91, 178.85, 171.87, 170.86, 170.83, 169.85, 169.78, 168.98, 166.33, 152.72, 146.48, 146.41, 141.71, 138.30, 137.55, 136.49, 135.86, 134.08, 133.57, 133.15, 131.18, 130.93, 129.64, 128.79, 128.69, 128.58, 128.34, 128.25, 127.94, 127.75, 127.12, 125.34, 123.63, 123.43, 122.86, 122.62, 118.10, 110.48, 102.73, 70.22, 70.10, 69.96, 67.23, 52.31, 51.98, 51.42, 44.76, 44.14, 38.70, 36.84, 36.65, 36.45, 35.48, 35.31, 31.79, 31.67.

### Example 4

### Synthesis of FAP5-PEG₄-FITC Conjugate

The FAP5-PEG₄-FITC conjugate can be synthesized according to Scheme 4.

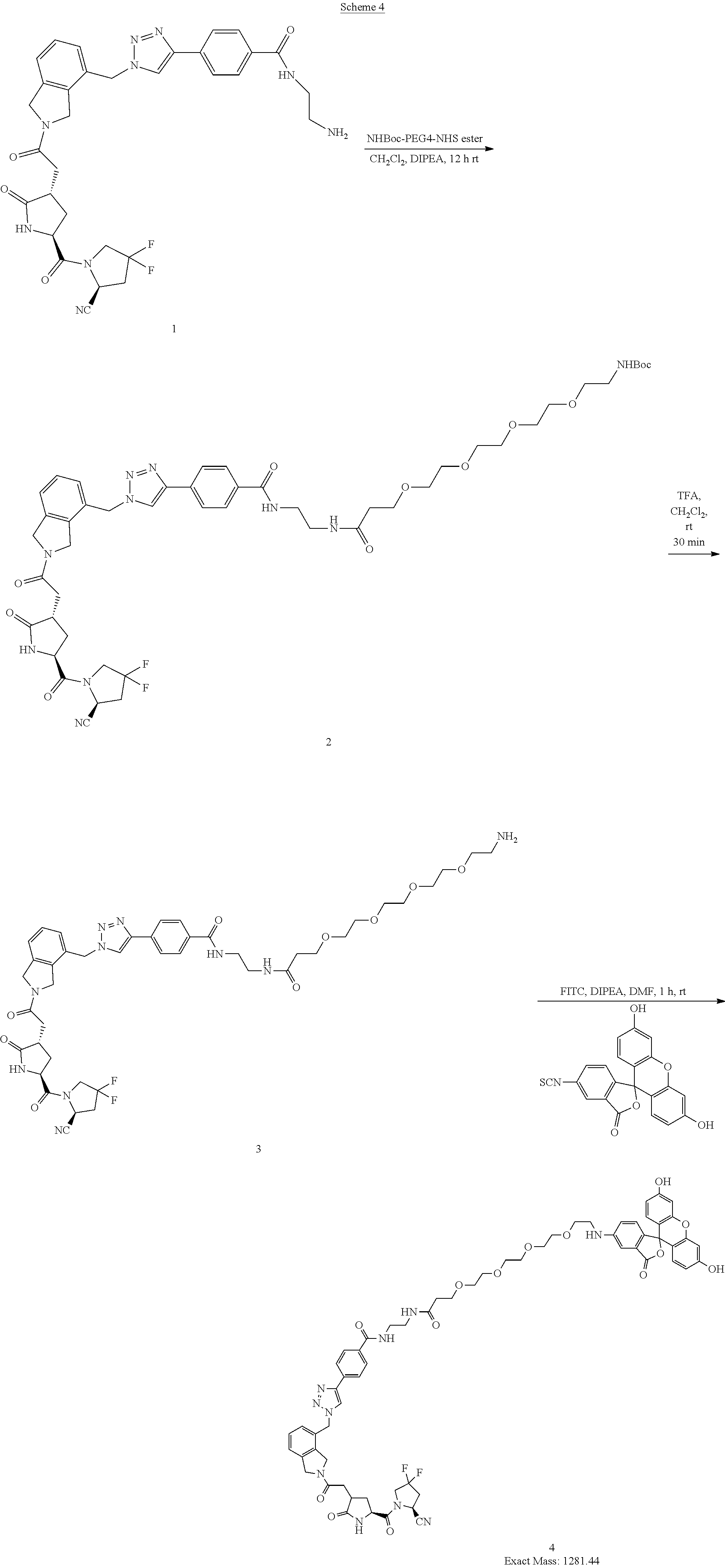

FITC (5.96 mg, 0.018 mmol, 0.95 eq) and DIPEA (6.6 μL, 0.038 mmol, 2 eq) were added to a stirred solution of compound 3 (17 mg, 0.019 mmol, 1 eq) in dry DMF (1.0 mL) and stirring was continued for 1 hour at room temperature under N₂ gas. After completion of the reaction (confirmed by LC-MS), the solvent was removed and crude was loaded on celite, and purified by combi flash (column: 15.5 g HP C18 Aq, flow rate: 40 ml/min, wave length 254 nm, eluent: A=20 mM ammonium acetate buffer (pH=7), B=acetonitrile), solvent gradients 0% B to 100% in 60 minutes to provide FAP5-PEG₄-FITC as a yellow solid (Compound 4 above). LC-MS (m/z): [M+H] found: 1282.4.

¹H NMR (500 MHz, DMSO-d₆) δ 10.18 9 s, 1H), 8.69 (d, J=9.5 Hz, 1H). 8.54-8.49 (m, 1H), 8.25 (s, 2H), 8.04-7.98 (m, 2H), 7.93-7.87 (m, 5H), 7.72 (d, J=8.0 Hz, 1H), 7.34-7.30 (m, 2H), 7.22-7.13 (m, 2H), 6.62-6.59 (m, 4H), 6.53 (d, J=8.5, 2H), 5.66 (d, J=7.0 Hz, 2H), 5.10-5.07 (m, 1H), 4.97 (s, 11H), 4.85 (d, J=6.5 Hz, 1H), 4.67 (d, J=8.0 Hz, 2H), 4.32-4.29 (m. 1H), 4.20-4.04 (m. 2H), 3.66 (br-s, 2H), 3.59-3.51 (m, 8H), 3.49-3.42 (m, 9H), 3.23-3.17 (m, 3H), 2.88-2.70 (m, 5H). 2.46-2.38 (m. 1H), 2.31-2.24 (m. 1H), 2.11-2.07 (m, 1H).

¹³C NMR (126 MHz, DMSO-d₆) δ 181.01, 178.91, 178.87, 171.88, 170.86, 169.85, 169.80, 169.01, 166.34, 152.69, 146.48, 146.43, 141.80, 138.30, 137.56, 136.49, 135.86, 134.09, 133.56, 133.11, 131.20, 130.94, 129.62, 128.80, 128.69, 128.57, 128.34, 128.21, 127.95, 127.76, 127.15, 125.32, 123.64, 123.45, 122.84, 122.61, 118.11, 110.45, 102.73, 70.22, 70.12, 69.97, 67.23, 52.32, 51.98, 51.43, 50.95, 44.76, 44.12, 38.71, 36.86, 36.65, 36.45, 35.48, 35.31, 31.67.

### Example 5

### 5 FAP-FITC Conjugate has Binding Affinity (K_{D}) of <10 nM Towards Human FAP and Murine FAP

To assess binding affinity. MDA-MB-231 cells (~0.1 million cells/well) over-expressing human fibroblast activation protein (hFAP) or murine fibroblast activation protein (mFAP) were suspended in complete RPMI medium in 96-well plates. Complete RPMI was prepared by supplementing RPMI 1640 (Gibco #21870076) with 10% fetal bovine serum (FBS; bio-techne #S11150H), 1% streptomycin/penicillin (Corning #30002CI), and 1% L-glutamine (ATCC #302214).

FAP-FITC conjugates were diluted with phosphate-buffered saline (PBS; Sigma #D8537). Their concentrations were confirmed by measuring absorbance at 495 nm with a nanodrop spectrophotometer. FAP-FITC was then added to the MDA-MB-231 cells at different final concentrations (in triplicate).

Cells were incubated with FAP5-FITC at room temperature for 40 minutes, washed three time with PBS containing 2% FBS, and subjected to flow cytometry for measurement of mean fluorescence intensity on the FITC channel. The K_{D} of different FAP-FITC conjugates was calculated via Prism 8.

The results are shown in FIG. 1. As shown in FIG. 1, FAP5-FITC binds hFAP and mFAP with a K_{D}<10 nM.

### Example 6

### 6 FAP-FITC was Retained on Cell Surface ~24 Hours

MDA-MB231-hFAP cells (~0.1 million cells/well) were seeded in complete RPMI in confocal chambers (Thermo Scientific #155382) overnight. FAP5-PEG₄-FITC (25 nM) was added to the cells, and the mixture was incubated for one hour in the cell incubator at 37° C. and 5% CO₂. Cells were washed with PBS containing 2% FBS twice to remove free FAP5-PEG₄-FITC, and incubation was continued in complete RPMI for 0 min, 1 hour, 2 hours, 4 hours, 24 hours or 48 hours. Cells were then examined at the different timepoints using confocal microscopy.

FAP5-FITC was retained on the surfaces of cells for ~24 hours.

### Example 7

### Optimization of Polyethylene Glycol Length for FAP5-FITC Conjugates

The protein structure of hFAP protein (PDB 1z68) was examined using Pymol. FAP5-PEG₄-FITC with hFAP proteins was molecularly modeled to examine the depth of the FAP binding pocket relative to FAP5-PEG₄-FITC. Molecular modeling indicated a deep binding pocket in hFAP, which typically requires a long PEG length for better FITC exposure.

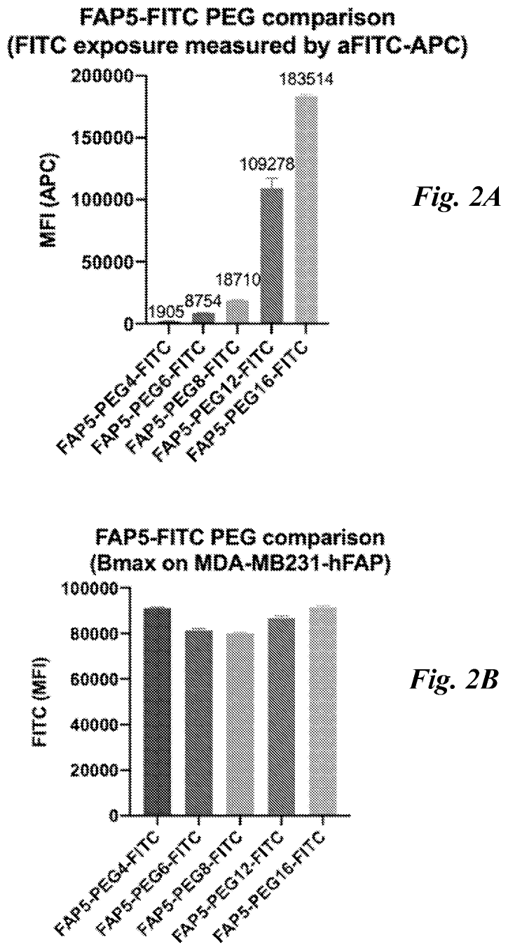

MDA-MB-231 cells (~100,000 cells/well) over-expressing hFAP were suspended in complete RPMI medium in 96-well plates. Cells were incubated with 100 nM of FAP5-PEG₄-FITC, FAP5-PEG₆-FITC, FAP5-PEG₈-FITC, FAP5-PEG₁₂-FITC, or FAP5-PEG₁₆-FITC at room temperature for 40 minutes. Cells were then washed twice, and either incubated with anti-FITC antibody (APC) at 4° C. for 30 minutes (for FITC exposure) prior to flow cytometry or immediately subjected to flow cytometry. The results are shown in FIGS. 2A and 2B. FIG. 2A shows that increasing PEG linker length for FAP5-FITC allowed better FITC exposure, while FIG. 2B shows that increasing PEG linker length for FAP5-FITC did not change the Bₘₐₓ of the bispecific adapters.

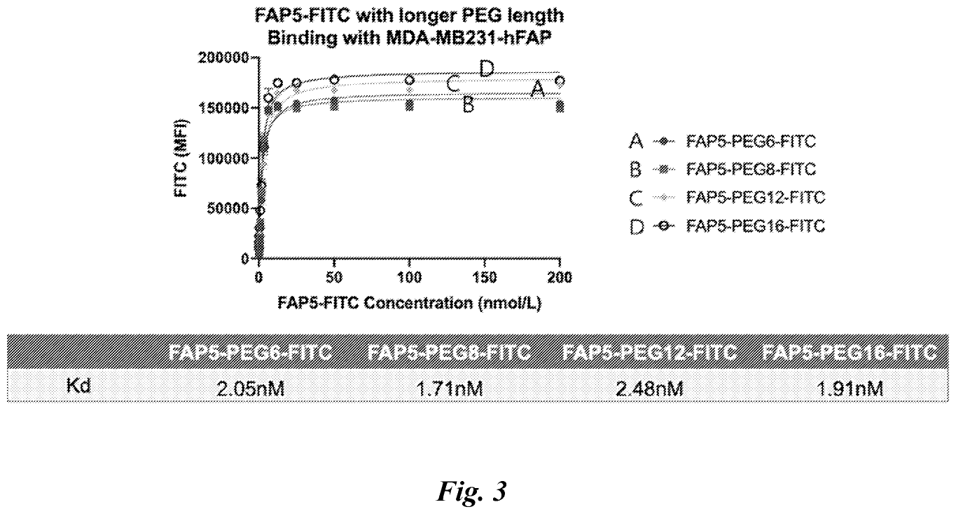

The effect of PEG linker length on binding affinity was also examined. MDA-MB-231 cells (~100,000 cells/well) over-expressing hFAP or mFAP were suspended in complete RPMI medium in 96-well plates. Concentrations of FAP5-PEG₆-FITC, FAP5-PEG₈-FITC. FAP5-PEG₁₂-FITC, or FAP5-PEG₁₆-FITC were confirmed by measuring absorbance with a nanodrop spectrophotometer. The bispecific adapters were separately added to MDA-MB-231 cells at different final concentrations (in triplicate). Cells were incubated with the adapters at room temperature for 40 minutes, washed three times with PBS+2% FBS, spun at 300 g for 5 minutes, and were subjected to flow cytometry for mean fluorescence intensity measurement. The results are shown in FIG. 3. FIG. 3 shows that FAP5-FITC with different PEG linker lengths did not change the K_{D} of the compound to hFAP.

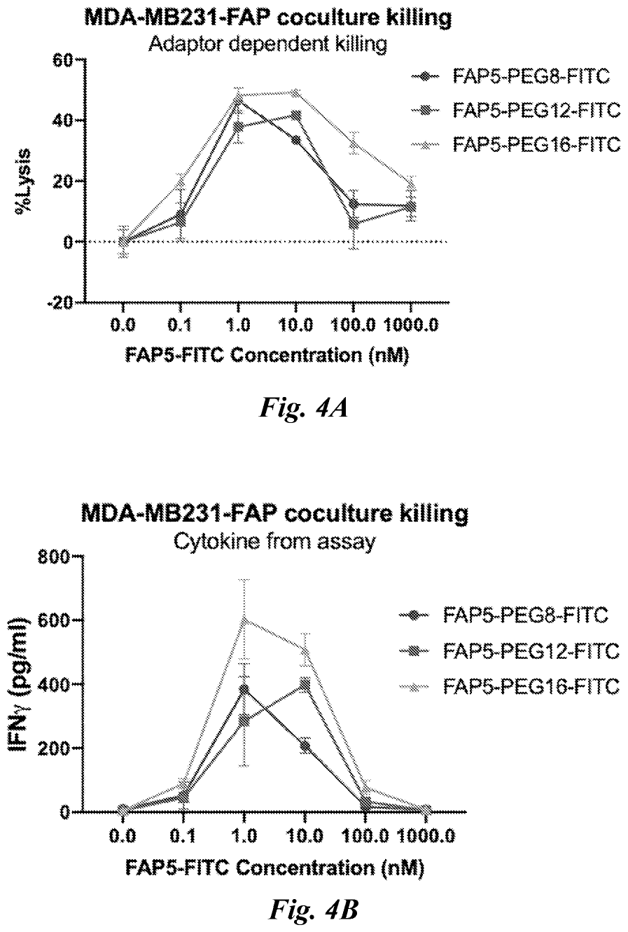

The killing efficacy mediated by FAP5-FITC with different PEG linker lengths was also examined. MDA-MB-231 cells (~7.000 cells/well) were seeded on 96-well plates overnight. On day 18, 4M5.3 CAR-T cells were added to the target cells at 1:4 effector:target cell ratio. FAP5-PEG₈-FITC, FAP5-PEG₁₂-FITC, or FAP5-PEG₁₆-FITC was either co-incubated with the cells for 24 hours or aspirated and washed away with PBS after one hour of incubation (0 nM, 0.1 nM, 1 nM, 10 nM. 100 nM, or 1,000 nM, all in triplicate) at room temperature. Live cells were determined by mCherry positive cells. Percent killing was determined by (1−(live cells)/(live cells in tumor cell only well))*100%. The results are shown in FIGS. 4A and 4B.

As shown in FIG. 4A, a FAP5-FITC with a longer linker (e.g., PEG₁₆) was more effective at killing tumor cells. As shown in FIG. 4B, a FAP5-FITC with a longer linker (e.g., PEG₁₆) was more effective at activating CAR-T cells.

### Example 8

### FAP8-FITC is Better than FAP5-FITC with Similar PEG Length Due to Better Serum Stability

MDA-MB-231-hFAP cells were subcutaneously injected into NSG mice and allowed to grow to 150 mm³. At that point, E2 (anti-fluorescein antibody) CAR-T cells (8 million) were injected intravenously, and FAP8-PEG₈-FITC, FAP8-PEG₁₂-FITC, FAP8-PEG₁₅-FITC, or FAP5-PEG₁₆-FITC was injected three times per week at 500 nmol/kg. The following six groups of mice were used:
Group 1 (n=6): Disease control group (tumor cells only).
Group 2 (n=6): E2 CAR-T cells only group (PBS three times/week),
Group 3 (n=6): FAP8-PEG₈-FITC group,
Group 4 (n=7): FAP8-PEG₁₂-FITC group,
Group 5 (n=7): FAP8-PEG₁₅-FITC group, and
Group 6 (n=6): FAP5-PEG₁₆-FITC group.

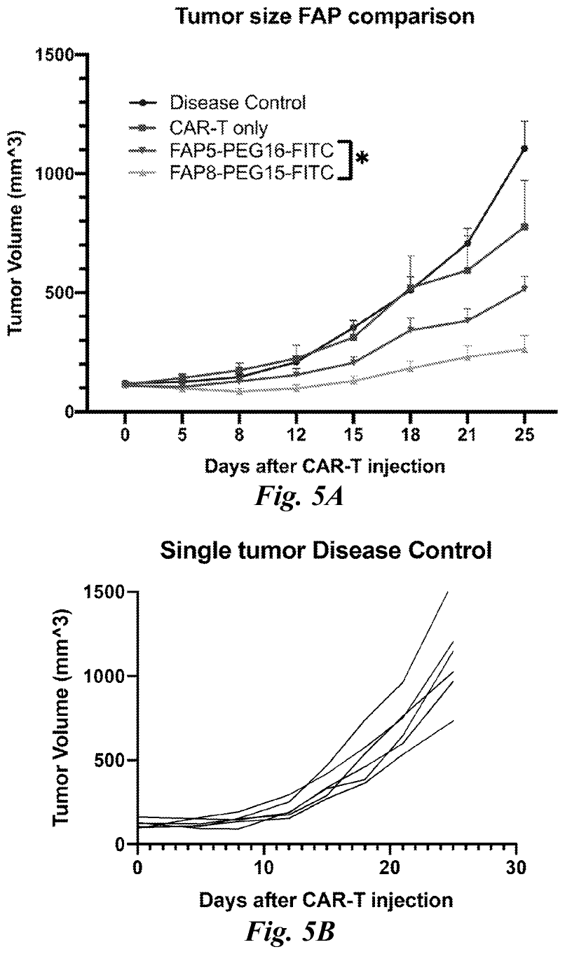

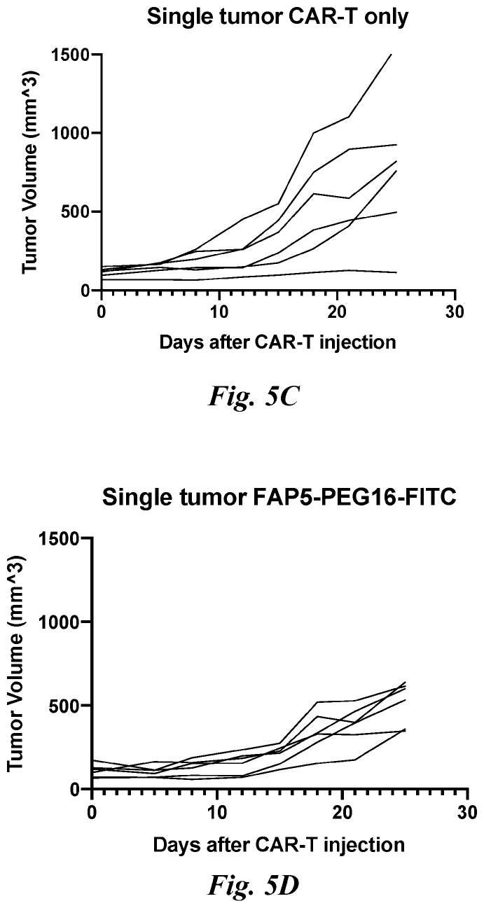

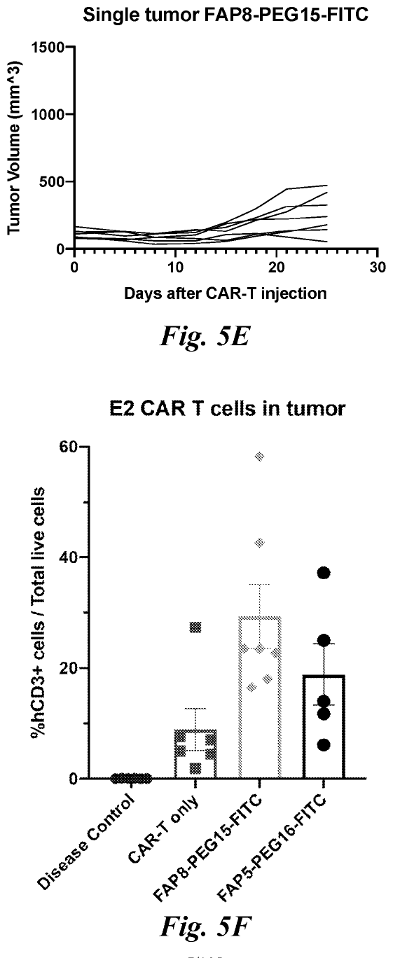

As shown in FIGS. 5A-5E, FAP5-PEG₁₆-FITC and FAP8-PEG₁₅-FITC work similarly in vitro, FAP8-PEG₁₅-FITC has better serum stability and mediates better engagement with CAR T-cells in vivo. As shown in FIG. 5F, more CAR T-cells infiltrated tumors when treated with FAP8-PEG₁₅-FITC than with FAP5-PEG₁₆-FITC. Tumors were harvested from mice at endpoint and digested with human tumor dissociation kit (Miltenyi) with 50% enzyme R to enhance lymphocyte recovery. Cells were then stained with Zombie violet and Fc blocker on ice for 30 minutes, followed by two washes, staining with anti-hCD3 antibody on ice for 30 minutes, three washes, and flow cytometry.

### Example 9

### FAP8-FITC with Optimal PEG Linker Length

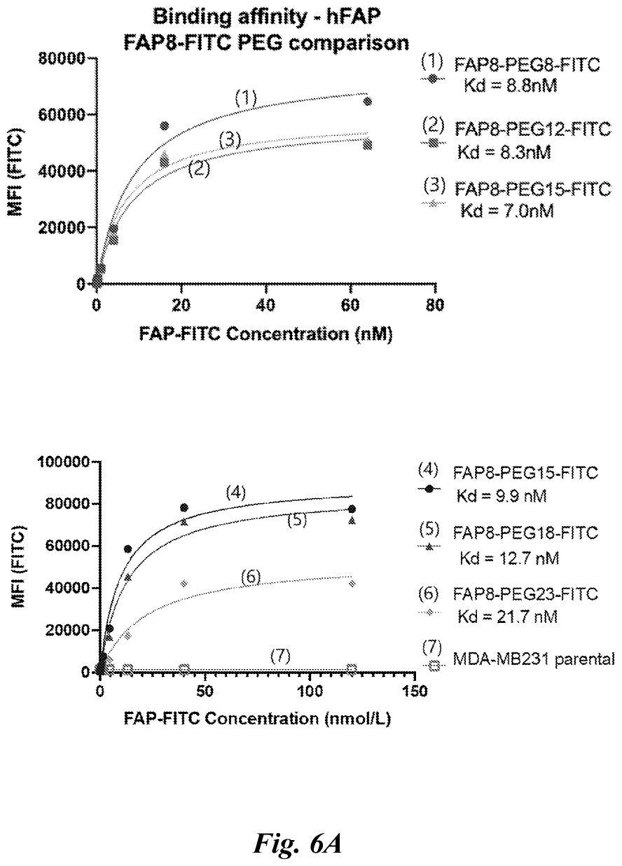

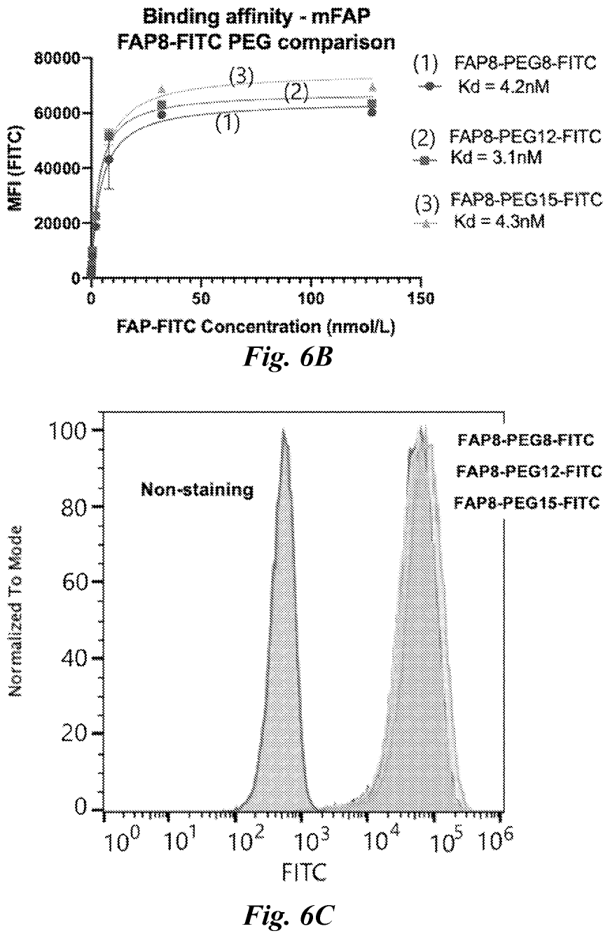

MDA-MB-231 cells (~100,000 cells/well) over-expressing hFAP or mFAP were suspended in complete RPMI medium in 96-well plates. FAP8-FITC adapters with increasing PEG linker lengths were diluted with PBS. Their concentrations were confirmed by measuring absorbance with a nanodrop spectrophotometer. The FAP-FITC bispecific adapters with different PEG linker lengths were then separately added to MDA-MB-231 cells at different final concentrations (in triplicate). Cells were incubated with the adapters at room temperature for 40 minutes, washed three times with PBS and 2% FBS, spun at 300 g for five minutes, and subjected to flow cytometry for MFI. The results are shown in FIGS. 6A-6C. As shown in FIGS. 6A-6C, FAP8-FITC binds to FAP+ cells with high specificity. A decrease in Kd was observed with increasing linker length.

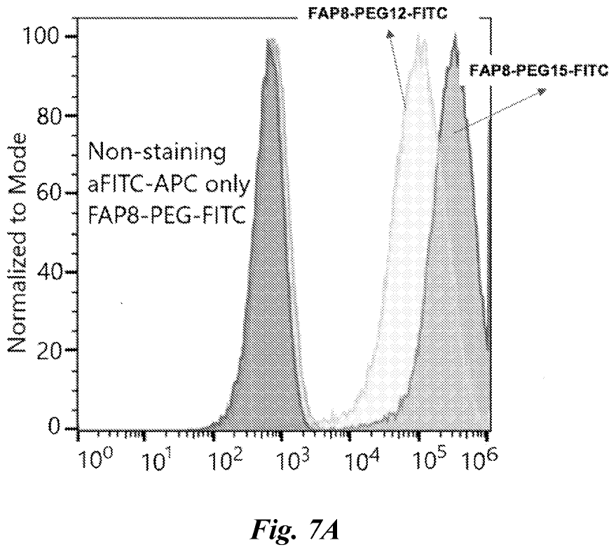

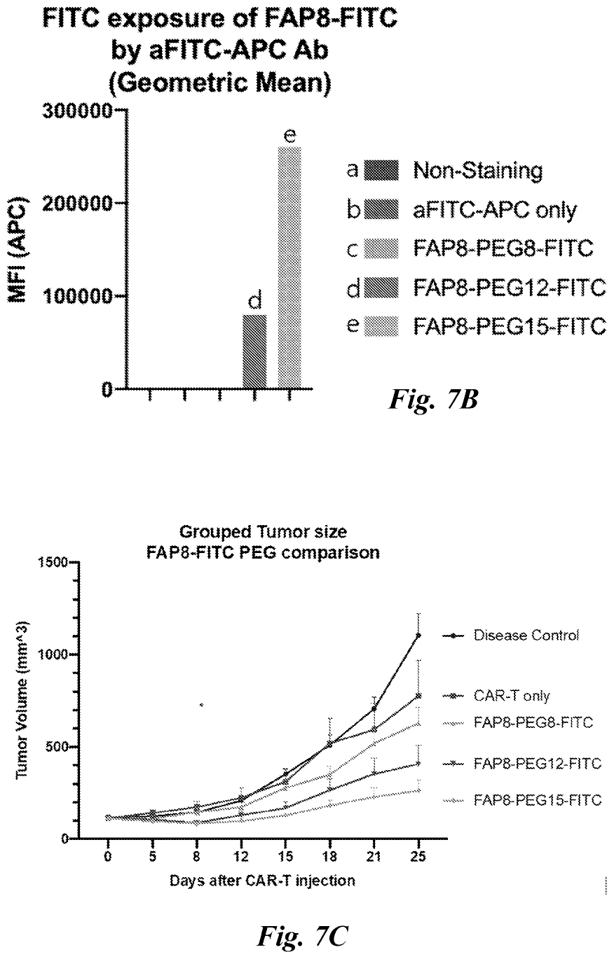

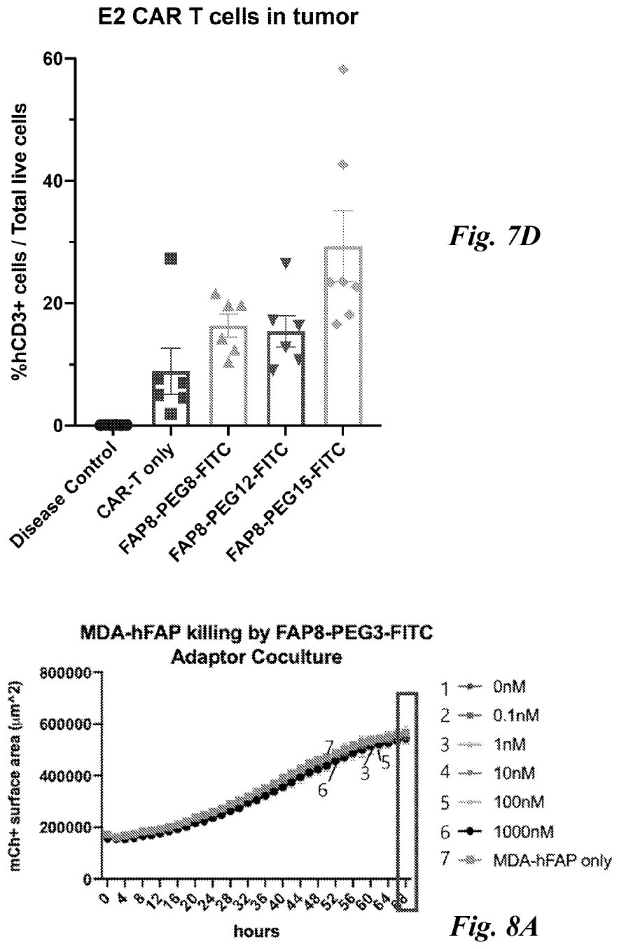

MDA-MB231-FAP cells were suspended in 1.5 mL Eppendorf tubes (~0.1 million cells/tube). Cells were incubated with 100 nM of FAP8-PEG₈-FITC, FAP8-PEG₁₂-FITC, or FAP8-PEG₁₅-FITC at room temperature for 40 minutes. Cells were then washed twice, incubated with anti-FITC antibody (APC) at 4° C. for 30 minutes (for FITC exposure), and then were subjected to flow cytometry. The results are shown in FIGS. 7A-7B. As shown in FIGS. 7A-7B, increasing the length of the PEG linker allowed better exposure, with FAP8-PEG₁₅-FITC showing the best FITC exposure. As shown in FIGS. 7C-7D, increasing the length of the PEG linker allows better engagement with CAR T-cells, thereby enhancing tumor elimination.

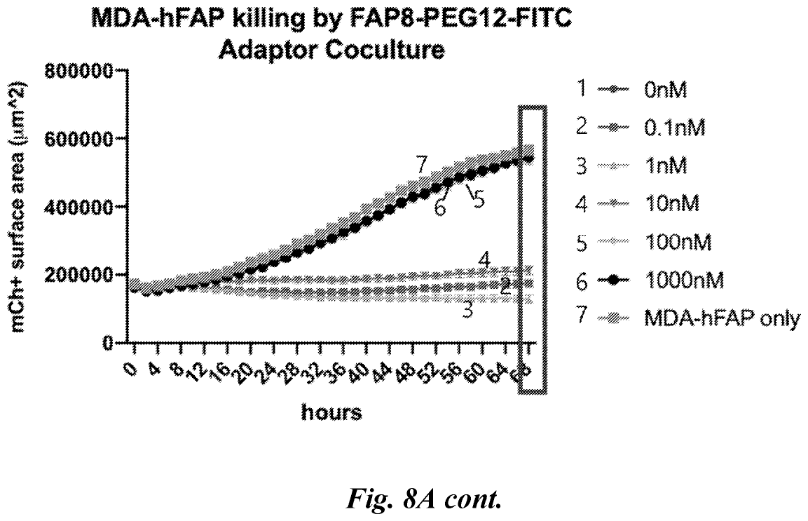

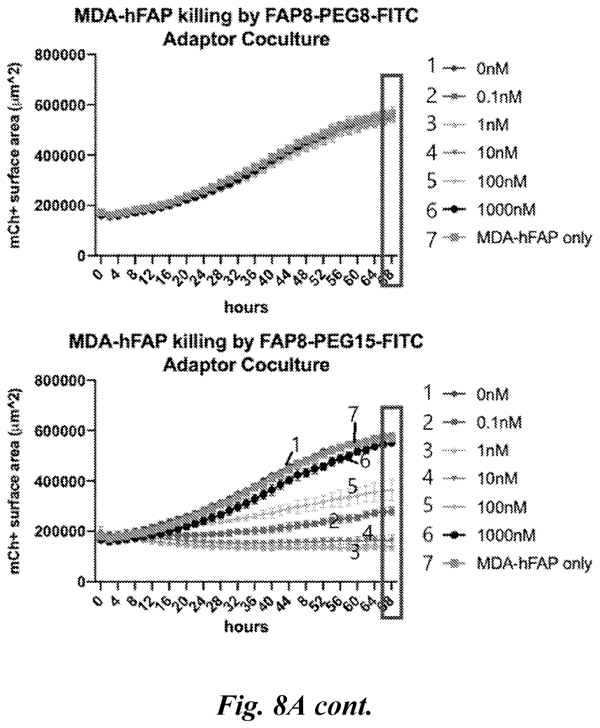

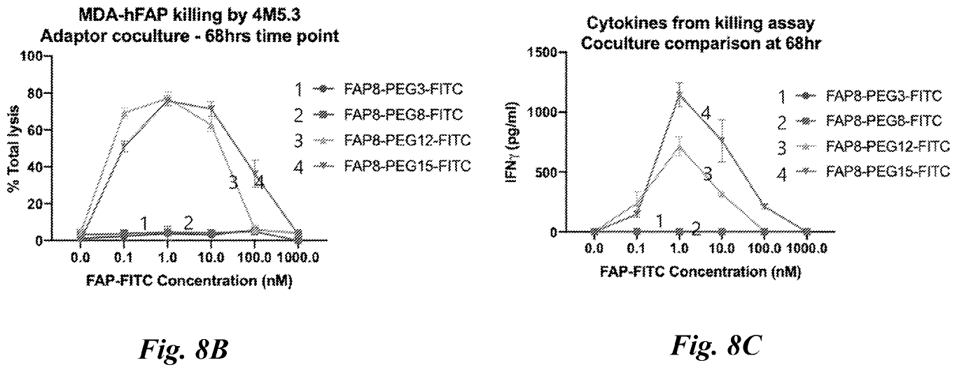

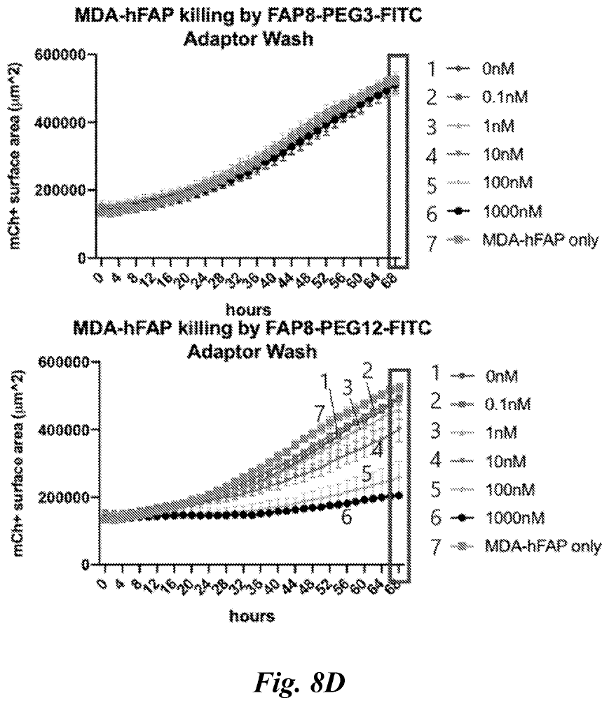

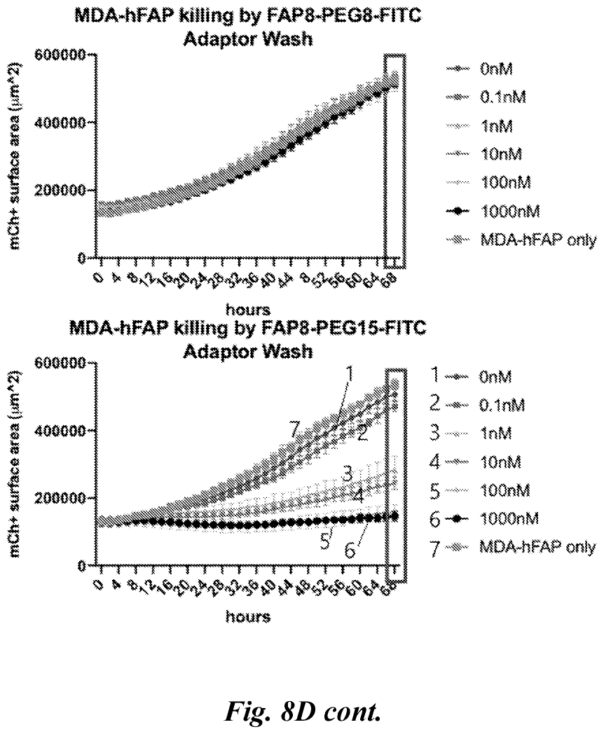

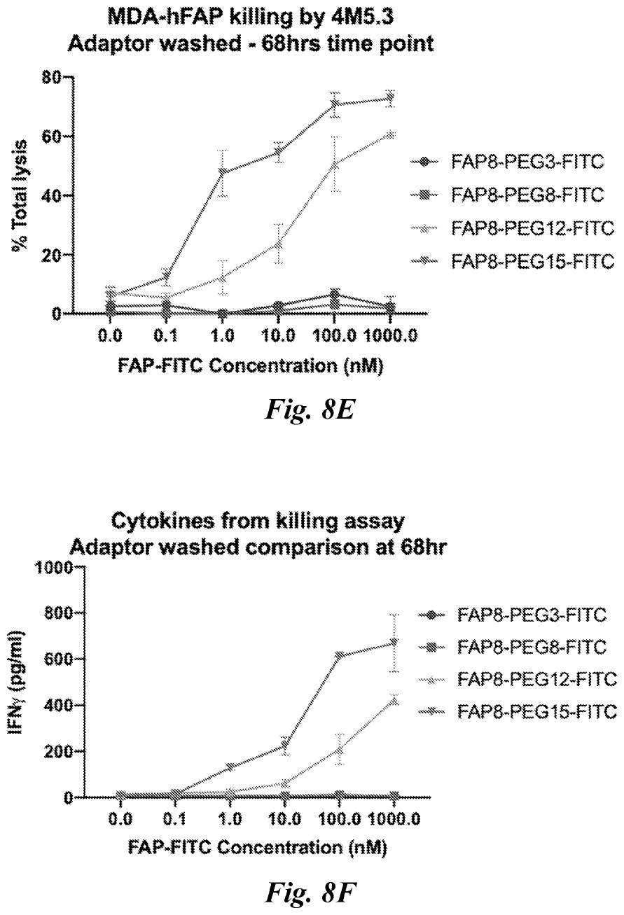

MDA-MB-231-FAP-mCh cells were seeded on 96 wells overnight (~5,000 cells/well). FAP8-PEG₃-FITC, FAP8-PEG₈-FITC, FAP8-PEG₁₂-FITC, and FAP8-PEG₁₅-FITC were separately added to the target cells at different concentrations (0 nM, 0.1 nM, 1 nM, 10 nM, 100 nM, or 1,000 nM, all in triplicate). FAP5-PEG₈-FITC was either co-incubated with the cells or aspirated and washed away with complete RPMI after 1 hour room temperature incubation. At day 19, 4M5.3 CAR-T cells were added to the target cells at 1:3 effector:target cell ratio and then co-incubated for 68 hours. Incucyte pictures were taken every two hours to measure total mCherry+ surface area. The results are shown in FIGS. 8A-8F. As shown in FIGS. 8A-8F, increasing the length of the PEG linker in FAP8-FITC increased killing efficacy, with FAP8-PEG₁₅-FITC showing the best killing efficacy and CAR-T activation in vitro.

### Example 10

### FAP8-PEG₁₅-FITC is Comparable to FAP5-PEG₁₆-FITC in Killing Efficacy but Provides Significantly Better FITC Exposure In Vitro

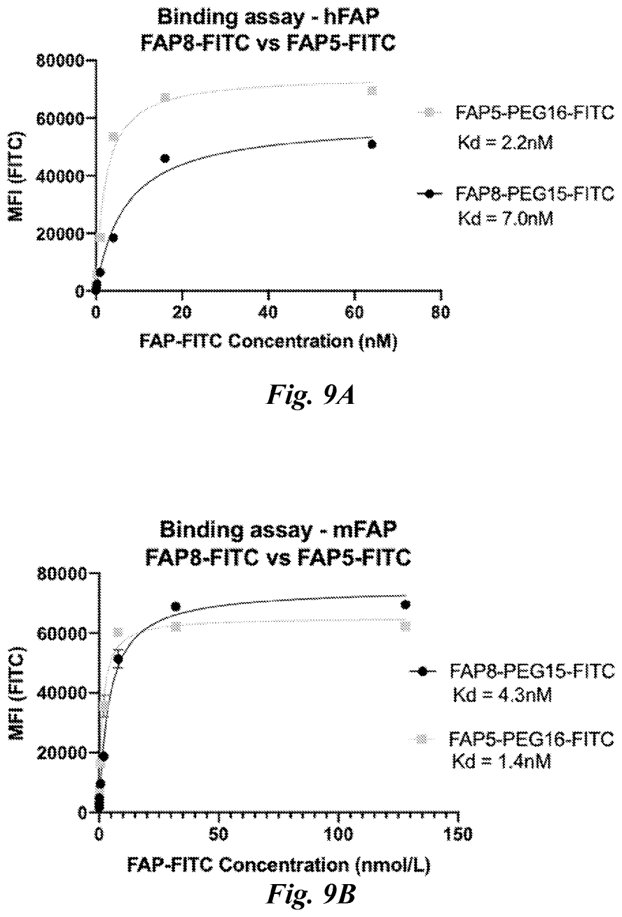

MDA-MB-231 cells over-expressing hFAP or mFAP were suspended in complete RPMI 96-well plates (~100,000 cells/well). Concentrations of FAP8-PEG₁₅-FITC and FAP5-PEG₁₆-FITC were confirmed measure absorbance with a nanodrop spectrometer. The adapters were added to the MDA-MB-231 cells at different final concentrations (in triplicate). Cells were incubated with the compounds at room temperature for 40 mins. washed three times with PBS and 2% FBS, spun @300 g for five minutes, and subjected to flow cytometry for MFI measurement. The results are shown in FIGS. 9A and 9B. As shown in FIGS. 9A and 9B, the binding affinity of FAP8-PEG₁₅-FITC is comparable to, but not quite as good as, the binding affinity of FAP5-PEG₁₆-FITC on hFAP- and mFAP-overexpressing MDA-MB-231 cells.

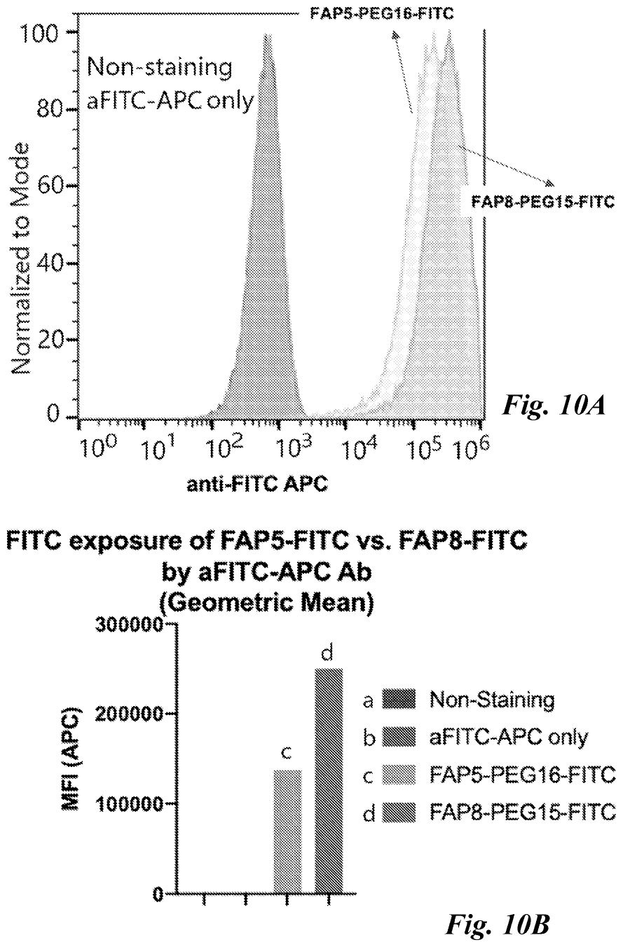

MDA-MB-231 cells over-expressing FAP were suspended in 1.5 mL Eppendorf tubes (~0.1 million cells/tube). Cells were incubated with 100 nM of FAP5-PEG₁₆-FITC or FAP8-PEG₁₅-FITC at room temperature for 40 minutes. Cells were then washed twice, incubated with anti-FITC antibody (APC) at 4° C. for 30 minutes, and subjected to flow cytometry. The results are shown in FIGS. 10A and 10B. As shown in FIGS. 10A and 10B, FAP8-PEG₁₅-FITC mediates more FITC exposure than FAP5-PEG₁₆-FITC.

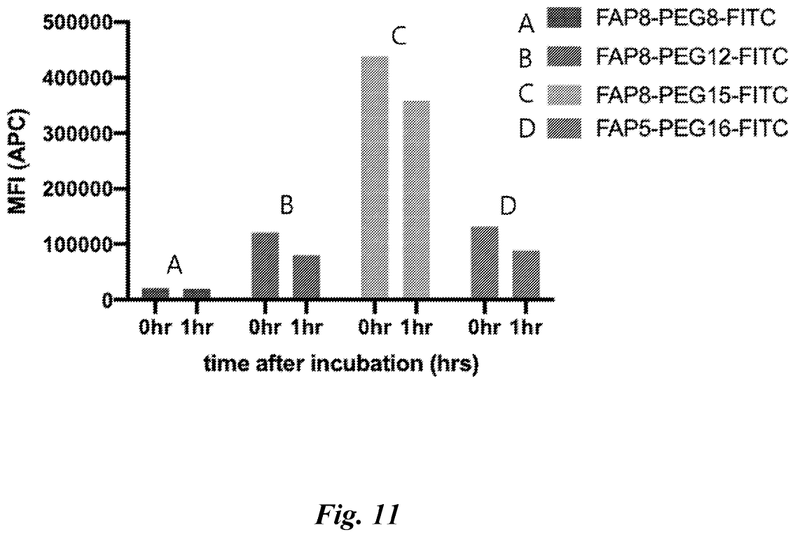

MDA-MB-231 cells over-expressing FAP were suspended in 1.5 mL Eppendorf tubes (~0.2 million cells/tube). Cells were incubated with 100 nM of FAP8-PEG₈-FITC, FAP8-PEG₁₂-FITC, FAP8-PEG₁₅-FITC, or FAP5-PEG₁₆-FITC at room temperature for 40 minutes. Cells were then washed twice, incubated at 37° C. for zero minutes or one hour, then incubated with anti-FITC antibody (APC) at 4° C. for 30 minutes, and subjected to flow cytometry. The results are shown in FIG. 11. As shown in FIG. 11, FAP8-PEG₁₅-FITC adapter had less dissociation (i.e., internalization) from the FAP protein compared to FAP5-PEG₁₆-FITC, resulting in better FITC exposure.

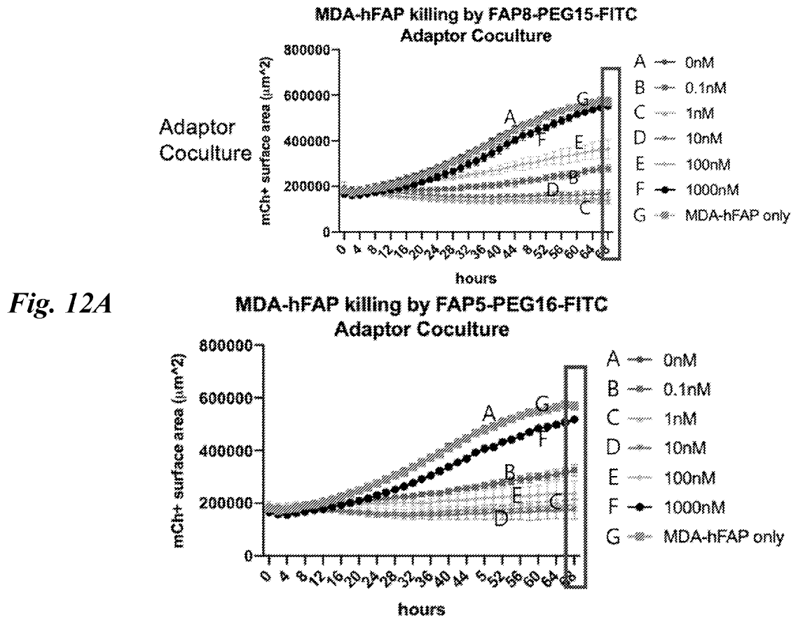

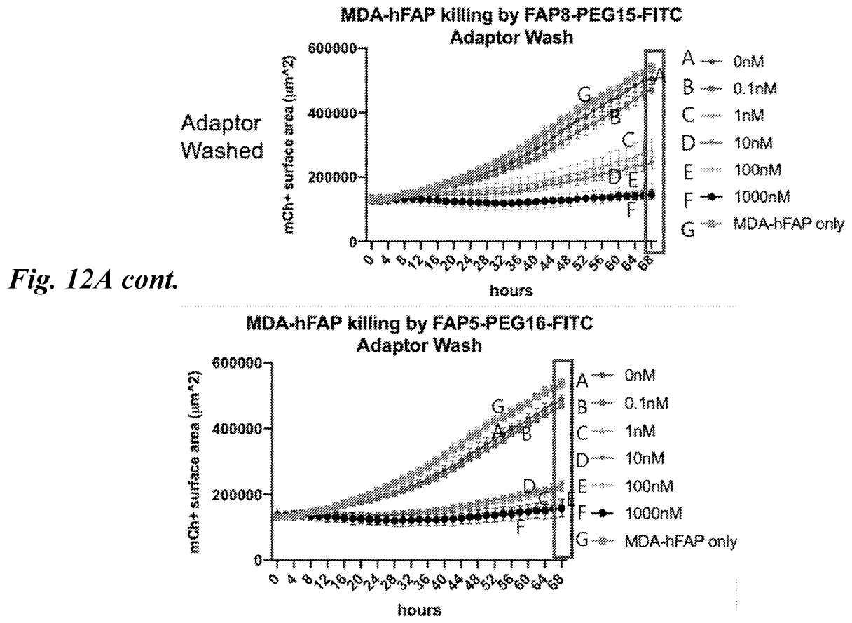

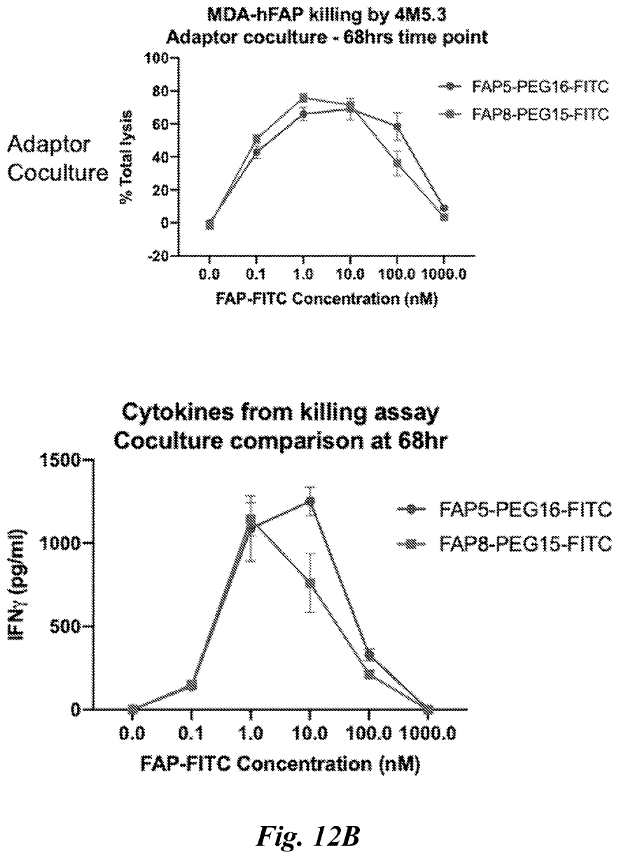

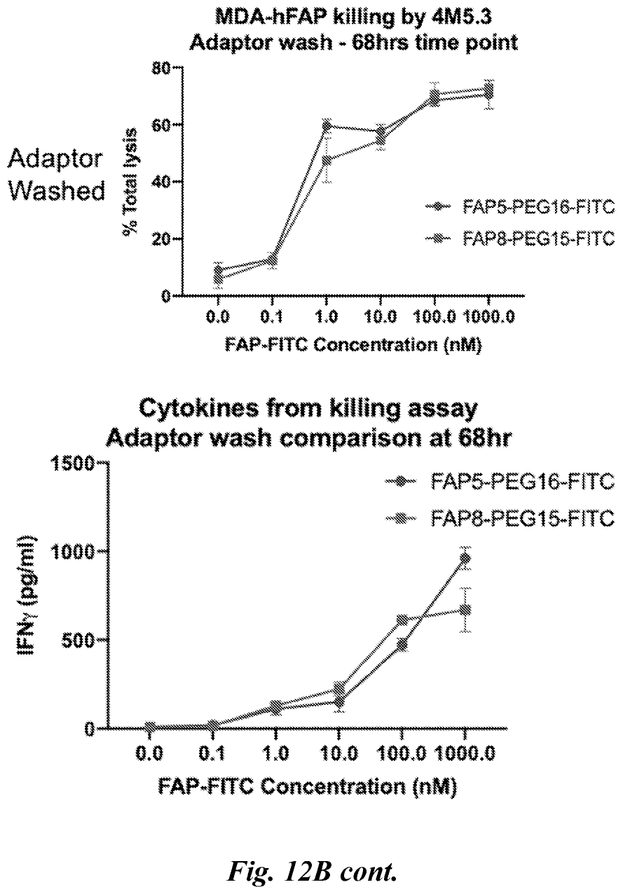

MDA-MB-23a-FAP-mCh cells were seeded in 96-well plates (~5,000 cells/well) overnight. FAP8-PEG₁₅-FITC and FAP5-PEG₁₆-FITC adapters were added to the target cells at different concentrations (0 nM, 0.1 nM, 1 nM, 10 nM. 100 nM, or 1,000 nM, all in triplicate). FAP5-PEG₁₆-FITC and FAP8-PEG₁₅-FITC were either co-incubated with the cells or aspirated and washed away with complete RPMI after one hour incubation at room temperature. 4M5.3 CAR-T at D19 were added to the target cells at 1:3 E:T ratio and then co-incubated for 68 hours. Incucyte pictures were taken every two hours to measure total mCherry+ surface area. The results are shown in FIGS. 12A-12B. As shown in FIGS. 12A-12B, FAP8-PEG₁₅-FITC was similar to FAP5-PEG₁₆-FITC in efficacy in vitro under adapter co-culture and adapter washed conditions.

### Example 11

### CAR T-Cell Proliferation and Activation in Blood

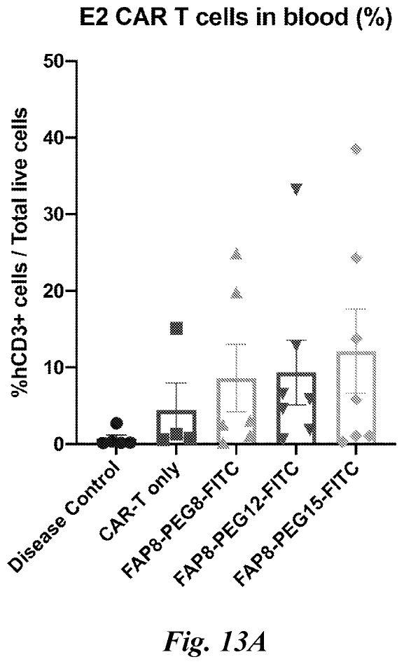

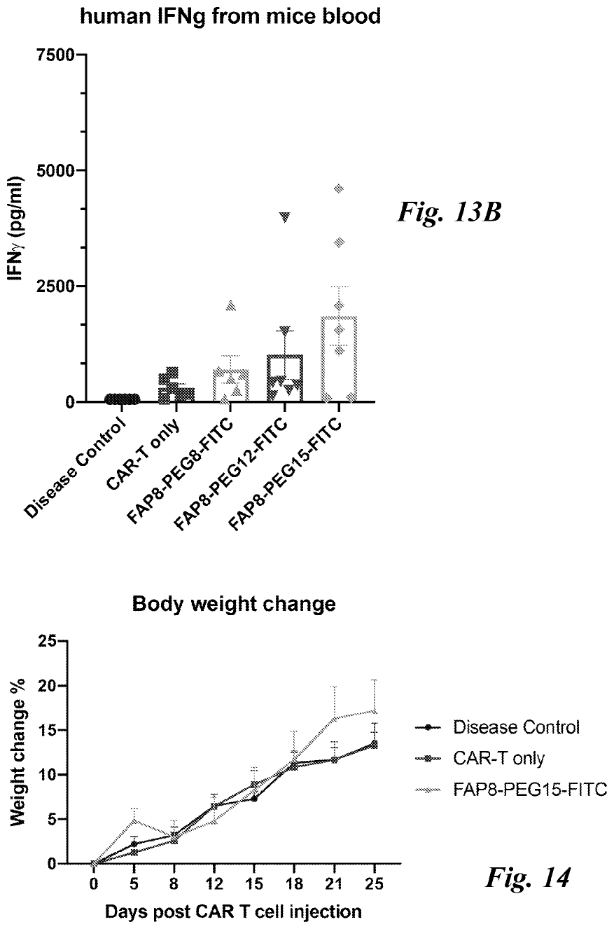

Blood samples were harvested from mice hearts at the endpoint of treatment. Part of the blood was incubated with red blood cell lysis buffer for 30 minutes on ice and washed three times. The rest of the blood was centrifuged at 1,000 g for 10 minutes to collect serum for human IFNγ ELISA analysis. Cells were then stained with Zombie violet and Fc blocker on ice for 30 minutes, washed twice, stained for anti-hCD3 on ice for 30 minutes, washed thrice, and subjected to flow cytometry analysis. The results are shown in FIGS. 13A-13B. As shown in FIGS. 13A-13B. FAP8-FITC adapters with longer PEG linkers result in better CAR T-cell proliferation and activation.

### Example 12

### FAP8-PEG₁₅-FITC Treatment Did not Lead to Toxicity in Mice

Disease control mice were injected with 500 nmols/kg FAP8-PEG₁₅-FITC or phosphate-buffered saline 24 hours prior to imaging using Spectral Ami Optical Imaging System. Tumors and all major organs were dissected for imaging of FITC signal.

Mice body weights were measured twice per week and compared to initial body weight before any treatment or tumor implantation. Data indicate that FAP8-PEG₁₅-FITC specifically localizes to tumor sites and does not bind to cells in major organs. As shown in FIG. 14, body weight analysis indicates that FAP8-PEG₁₅-FITC treatment did not lead to toxicity in mice.

### Example 13

### Bmax and FITC Exposure of FAP8-FITC Adapter with PEG₈-PEG₂₃ Linker Lengths

MDA-MB231-FAP cells were suspended in 1.5 mL Eppendorf tubes (~0.1 million cells/tube). Cells were incubated with 100 nM of FAP8-FITC adapters with PEG₈-PEG₂₃ linker lengths (n=3) at room temperature for 1 hour. Cells were then washed twice and either incubated with anti-FITC antibody (APC) at 4° C. for 30 minutes (for FITC exposure) or subjected to flow cytometry (for Bmax).

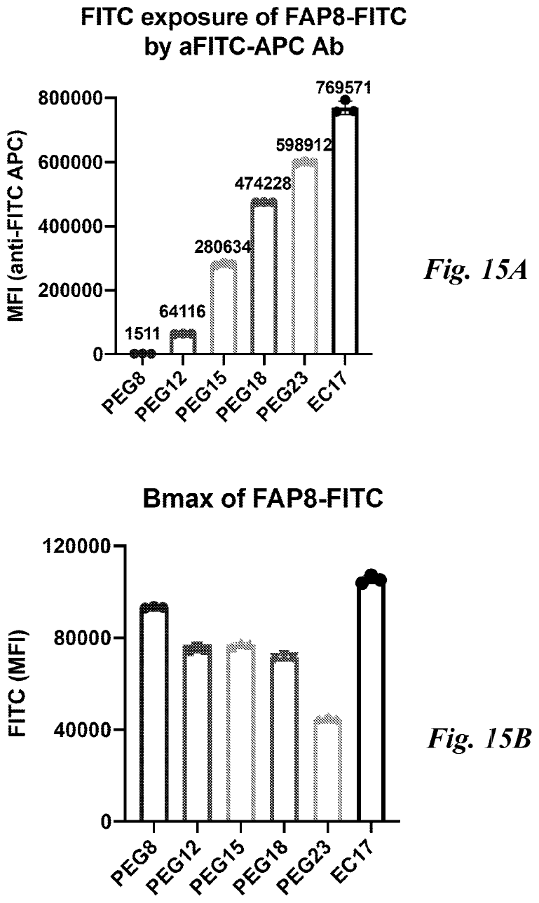

The results are shown in FIGS. 15A-15B. As shown in FIG. 15A, FITC exposure from the FAP binding pocket increased with increasing PEG linker length. As shown in FIG. 15B, Bmax gradually decreased with increasing PEG linker length.

### Example 14

### Comparison of FAP8-FITC with PEG₈-PEG₂₃ Linker Lengths in In Vitro Coculture Killing Assay with 4M5.3 CAR-T

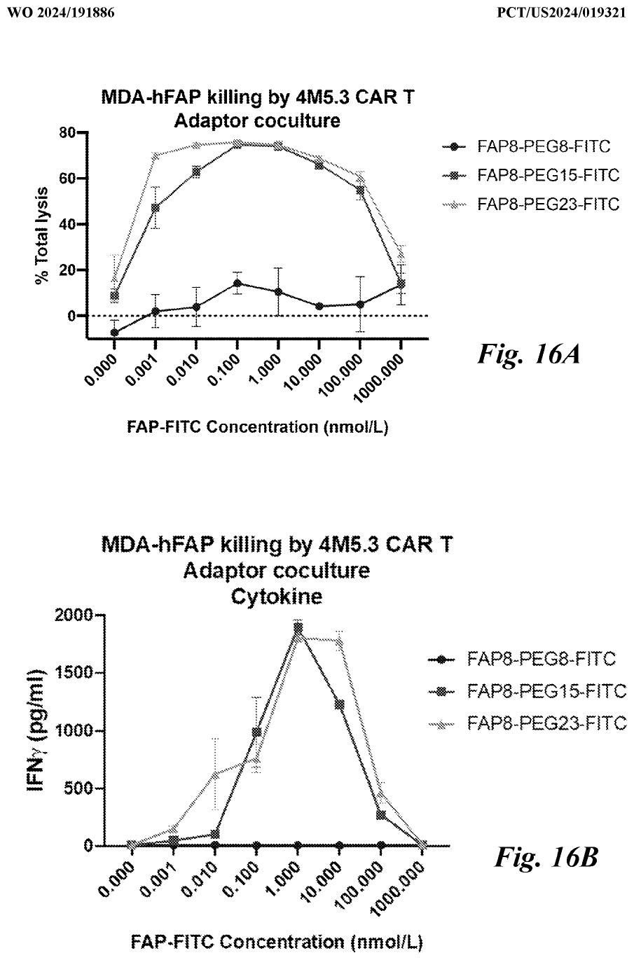

MDA-MB231-FAP-mCh cells (~7,000 cells/well) were seeded on 96-well plates overnight. FAP8-FITC with PEG₈-PEG₂₃ linker lengths was added to the target cells at different concentration (0 nM to 1,000 nM; n=3). The adapters were either co-incubated with the cells or aspirated and replaced with complete RMI after 1 hour of incubation at room temperature. 4M5.3 CAR-T was added to the target cells at 1:3 E:T ratio at around day 20 and then allowed to co-incubate for 48 hours. Live cells were measured by mCherry+ cells via flow cytometry. The results are shown in FIGS. 16A-16B.

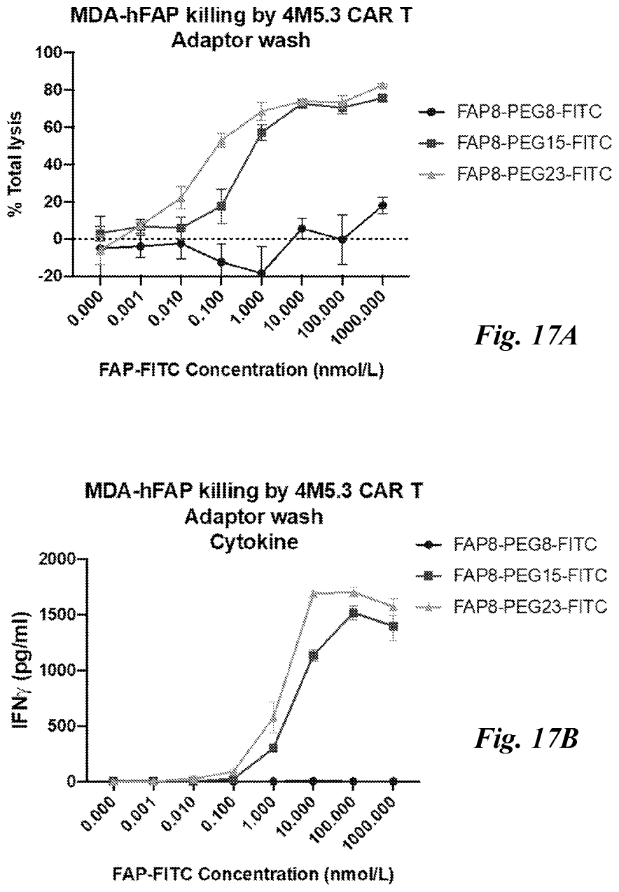

As shown in FIGS. 16A-16B, better killing and T-cell activation was observed with 4M5.3 CAR-T and FAP8-PEG₂₃-FITC. The same results were observed with adaptor washed condition in the in vitro killing assay as shown in FIGS. 17A-17B.

### Example 15

### Comparison of FAP8-FITC with PEG₈-PEG₂₃ Linker Lengths in In Vitro Coculture Killing Assay with E2 CAR-T

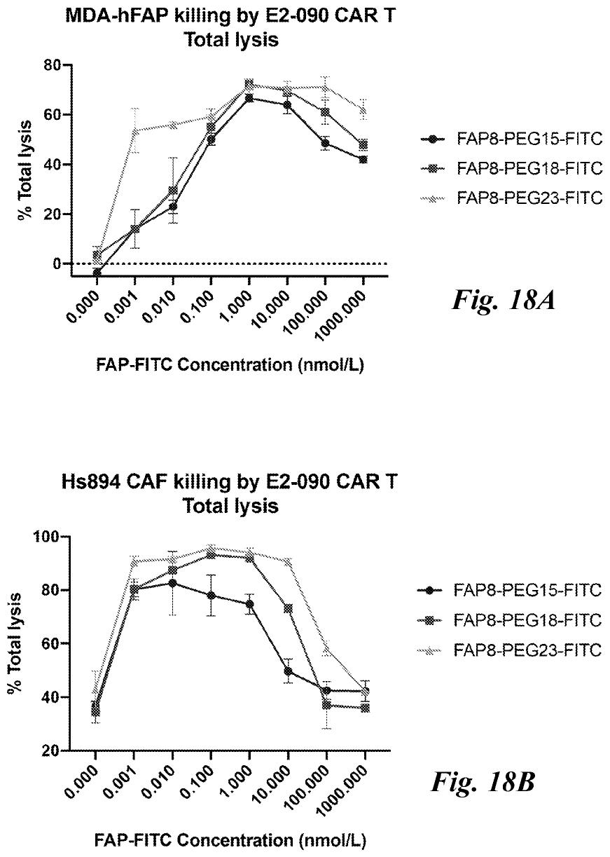

MDA-hFAP cells or Hs894 CAFs were seeded on 96-well plates overnight. FAP8-FITC with PEG₈-PEG₂₃ linker lengths was added to the target cells at different concentration (0 nM to 1,000 nM; n=3). E2 CAR-T was added to the target cells at 1:3 E:T ratio for MDA-hFAP and 2:1 E:T ratio for CAFs and then allowed to co-incubate for 48 hours. Live cells were measured by CellTrace+ cells via flow cytometry. The results are shown in FIGS. 18A-18B.

As shown in FIGS. 18A-18B, the efficacy of killing in vitro increased with increasing PEG linker length but with minor differences.

### Example 16

### The Effect of FAP8-FITC with PEG₈-PEG₂₃ Linker Lengths in Combination with EC17 on KB Tumor In Vivo

The KB tumor is FAP− and FR+, and the CAFs are FAP+. KB tumors were implanted in NSG mice, and the tumors were allowed to grow to approximately 50 mm³. The mice groups were as follows:
Group 1 (n=5) (FAP-FITC adaptor only): FAP8-FITC only (no CAR-T)
Group 2 (n=6) (CAR T only): E2 CAR-T and PBS
Group 3 (n=6) (FAP-FITC only): E2 CAR-T and FAP-FITC
Group 4 (n=6) (EC17 only): E2 CAR-T and EC17
Groups 5, 6, 7 and 8 (n=6) (FAP-FITC and EC17): E2 CAR-T, FAP8-PEG₈₋₂₃-FITC and EC17.

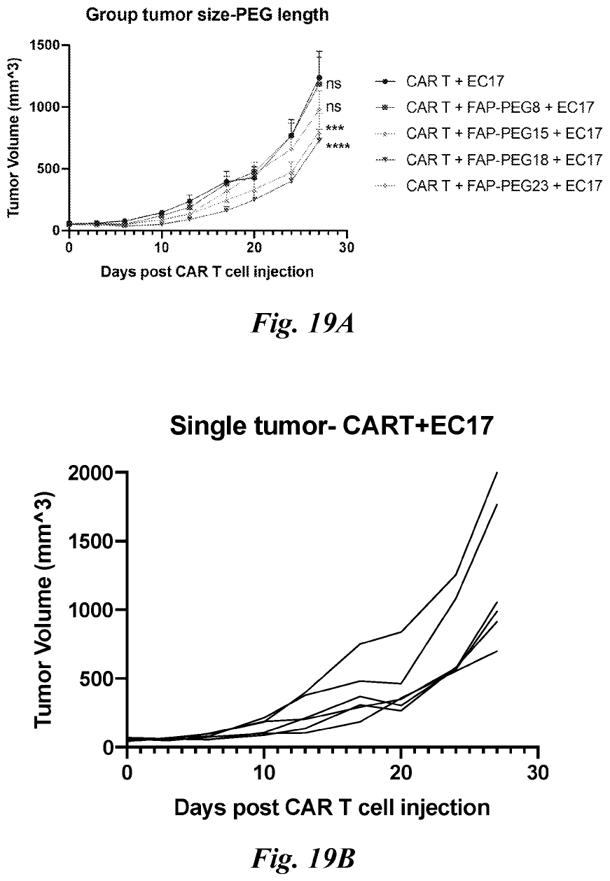

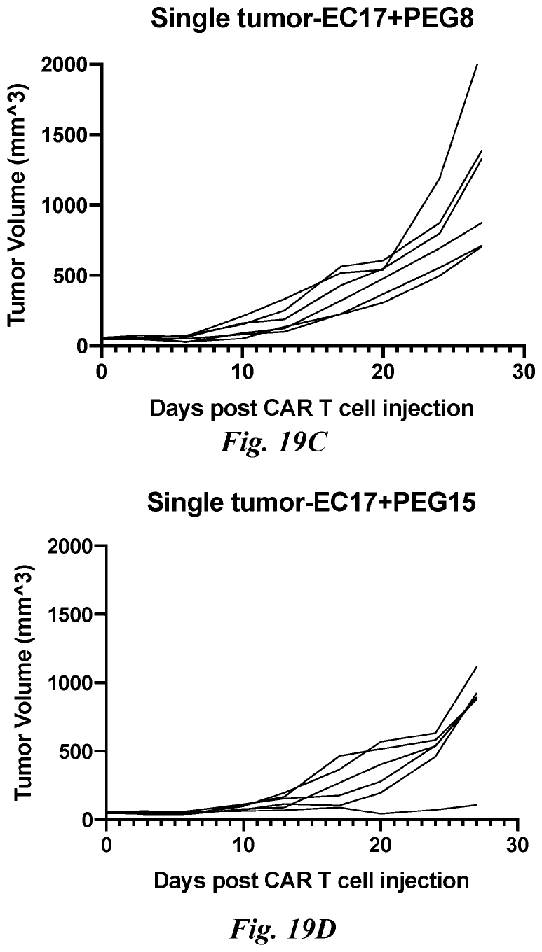

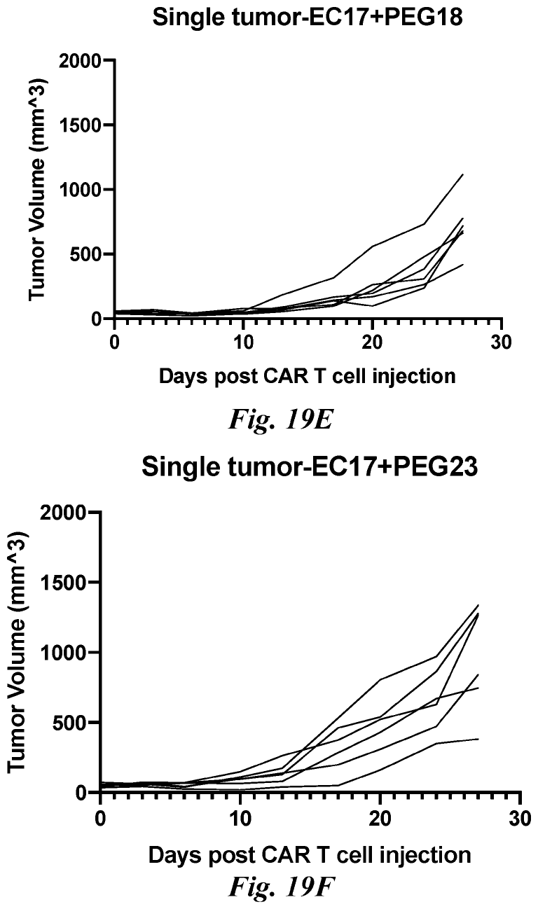

Mice were intravenously injected with 10×10⁶ E2 CAR-T (VPN404) with FAP8-FITC and/or EC17. Tumors were harvested with the disease control tumor reached 1,500 mm³ or when tumors were eliminated in the treatment groups. The results are shown in FIGS. 19A-19F.

As shown in FIGS. 19A-19F, FAP8-FITC with a PEG₁₅ linker mediated better regression in KB tumors.

### Example 17

### FAP8-PEG₁₅-FITC and FAP8-PEG₁₈-FITC Showed Similar Cytokine Release and CAR T Count at Study Midpoint

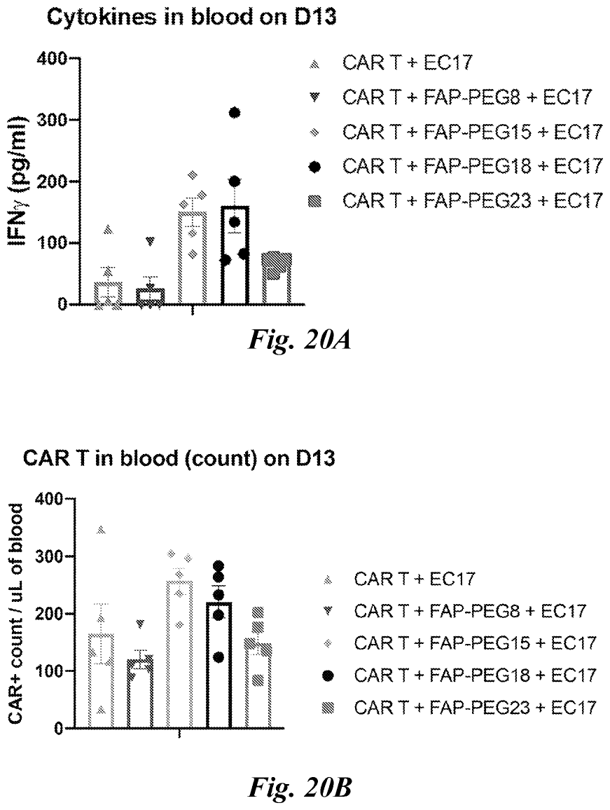

Blood was harvested from mice tail veins at study midpoint. Part of the blood was incubated with red blood cell (RBC) lysis buffer for 30 minutes on ice and washed three times. The rest of the blood was centrifuged at 1,000 g for 10 minutes to collect serum for human IFNγ enzyme-linked immunosorbent assay (ELISA) analysis. Cells were then stained with Zombie violet and Fc blocker on ice for 30 minutes, washed twice, stained for anti-hCD3 on ice for 30 minutes, washed thrice, and subjected to flow cytometry analysis. The results are shown in FIGS. 20A-20B.

As shown in FIGS. 20A-20B, similar cytokine release and CAR T-cell count were realized at study midpoint with FAP8-PEG₁₅-FITC and FAP8-PEG₁₅-FITC.

### Example 18

### FAP8-PEG₁₅-FITC Showed Slightly Better Cytokine Release and CAR T Count than FAP8-PEG₁₅-FITC at Study Endpoint

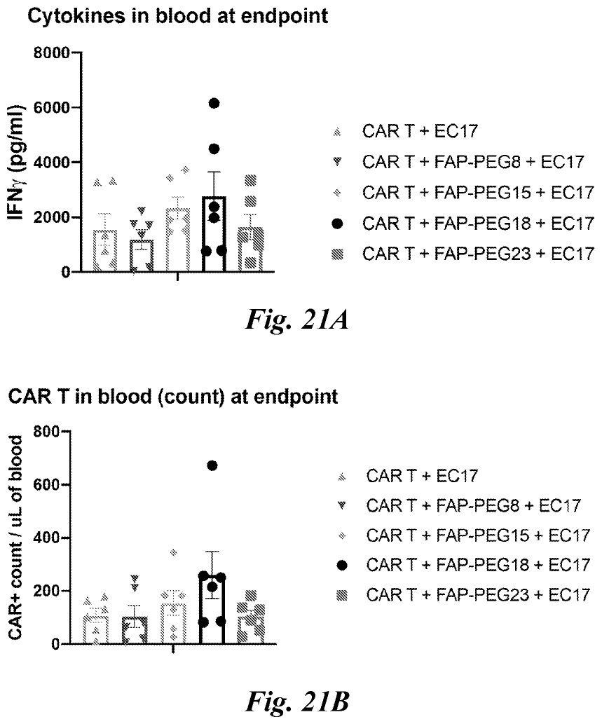

Blood was harvested from mice tail veins at study endpoint. Part of the blood was incubated with RBC lysis buffer for 30 minutes on ice and washed three times. The rest of the blood was centrifuged at 1.000 g for 10 minutes to collect serum for human IFNγ ELISA analysis. Cells were then stained with Zombie violet and Fc blocker on ice for 30 minutes, washed twice, stained for anti-hCD3 on ice for 30 minutes, washed thrice, and subjected to flow cytometry analysis. The results are shown in FIG. 21A-21B.

As shown in FIGS. 21A-21B, FAP8-PEG₁₈-FITC showed slightly better cytokine release and CAR T count than FAP8-PEG₁₅-FITC at study endpoint.

### Example 19

### Combination Therapy Yields Better CAR T Proliferation and Activation at Study Midpoint

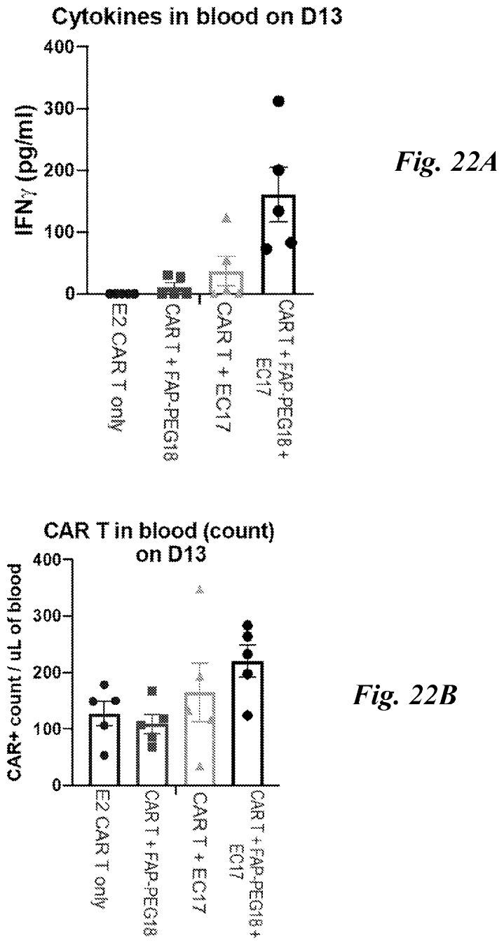

Blood was harvested from mice tail veins at study midpoint. Part of the blood was incubated with RBC lysis buffer for 30 minutes on ice and washed three times. The rest of the blood was centrifuged at 1,000 g for 10 minutes to collect serum for human IFNγ ELISA analysis. Cells were then stained with Zombie violet and Fc blocker on ice for 30 minutes, washed twice, stained for anti-hCD3 on ice for 30 minutes, washed thrice, and subjected to flow cytometry analysis. The results are shown in FIGS. 22A-22B.

As shown in FIGS. 22A-22B, FAP8-PEG₁₅-FITC and FAP8-PEG₁₈-FITC showed similar cytokine release and CAR T count at study midpoint.

### Example 20

### Combination Therapy Yields Better CAR T Proliferation and Activation at Study Endpoint

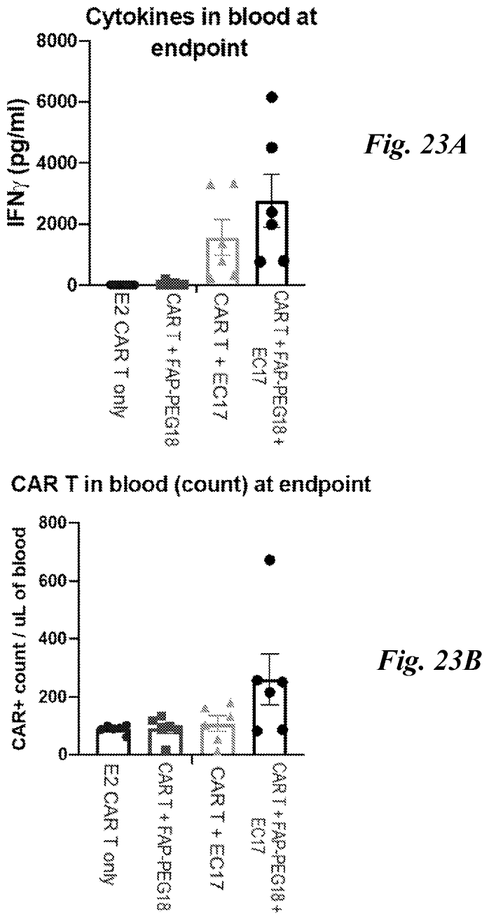

Blood was harvested from mice tail veins at study endpoint. Part of the blood was incubated with RBC lysis buffer for 30 minutes on ice and washed three times. The rest of the blood was centrifuged at 1,000 g for 10 minutes to collect serum for human IFNγ ELISA analysis. Cells were then stained with Zombie violet and Fe blocker on ice for 30 minutes, washed twice, stained for anti-hCD3 on ice for 30 minutes, washed thrice, and subjected to flow cytometry analysis. The results are shown in FIGS. 23A-23B.

As shown in FIGS. 23A-23B, FAP8-PEG₁₈-FITC with EC17 showed better cytokine release and CAR T count than EC17 without FAP8-PEG₁₈-FITC at study endpoint.

### Example 21

### Combination Therapy Yields Better CAR T Infiltration in KB Tumor at Study Endpoint

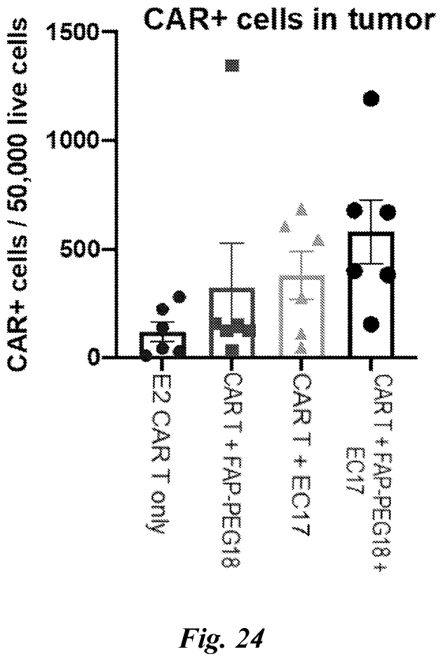

Tumors were harvested from mice at endpoint and digested with human tumor dissociation kit (Miltenyi) with 50% enzyme R to enhance lymphocyte recover. Cells were then stained with Zombie violet and Fc blocker on ice for 30 minutes, washed twice, stained for anti-E2 CAR antibody on ice for 30 minutes, washed thrice, and subjected to flow cytometry analysis. The results are shown in FIG. 24.

As shown in FIG. 24, more CAR T cell infiltration was observed with the combination therapy of FAP8-FITC and EC17.

When FAP8-FITC (500 nmols/kg) was injected 24 hours before imaging in disease control mice (KB tumor), FAP8-PEG₂-FITC showed weaker retention than FAP8-PEG₁₅-FITC and FAP8-PEG₁₈-FITC.

### Example 22

### Immunohistochemistry of KB Tumor and MDA-MB231 Tumor

To characterize and compare infiltrating FAP+ CAFs in cold KB tumors and hot MDA-MB-231 tumors, KB tumors or MDA-MB-231 tumors were implanted on NOD scid gamma (NSG) mice, and the mice were treated with the universal anti-FITC CAR-T and EC17 (folate-fluorescein). Both tumors were then harvested from the mice, fixed with 10% formalin overnight, and rinsed with 70% ethanol for tumor fixation. Then, the tumor cells were sent for IHC staining for either anti-human CD3 antibody to detect human CAR-T cells or anti-mouse FAP antibody to detect mouse FAP+CAFs.

Results from the immunohistochemical (IHC) staining demonstrated a lack of CAR-T cell infiltration in the immunologically cold KB tumor but not in the hot MDA-MB-231 tumor. Elevated infiltration of FAP+CAFs was found in cold KB tumor, forming a physical barrier around the tumor, but the infiltrating CAFs were significantly less on the hot MDA-MB-231 tumor. It was hypothesized that these infiltrating fibroblasts are one of the reasons for an immunologically cold solid tumor.

### Example 23

### Confocal Microscopy of FAP8-FITC Specific Binding

To confirm the adaptor specificity of FAP8-FITC in FAP-expressing cells, MDA-MB231-hFAP (human FAP), MDA-MB231-mFAP (murine FAP) and parental MDA-MB231 cells (no FAP expression) were seeded on confocal chambers overnight (~0.1 million cells/well). FAP8-PEG₁₈-FITC (50 nM) was added to the cells, and the mixture was incubated for one hour at room temperature. Cells were washed with PBS+2% FBS twice and examined under confocal microscopy immediately. FAP8-FITC specifically bound to hFAP and mFAP.

### Example 24

### In Vivo Study to Determine Optimal Linker of FAP8-FITC in Mice

FAP8-FITC with different PEG linkers were tested in mice to determine the optimal linker. The efficacy of FAP8-PEG_{8, 12, 15}-FITC and FAP5-PEG₁₆-FITC were also compared. MDA-MB231-hFAP (5 million cells) were injected per NSG mouse subcutaneously. E2 CAR T-Cells (8 million) were injected into each mouse when tumor size reached ~100 mm³. FAP8-FITC was injected via tail vein three times per week at 500 nmol/kg. The mice groups were as follows:
Group 1 (n=6) (disease control); only tumor cells were injected
Group 2 (n=6) (CAR T only); CAR T-cells (8×10⁶ cells) were injected: FAP-FITC was injected three times per week
Group 3 (n=6) (FAP8-PEG₈-FITC treatment); CAR T-cells (8×10⁶ cells) were injected; FAP-FITC was injected three times per week
Group 4 (n=7) (FAP8-PEG₁₂-FITC treatment); CAR T-cells (8×10⁶ cells) were injected; FAP-FITC was injected three times per week
Group 5 (n=7) (FAP8-PEG₁₅-FITC treatment); CAR T-cells (8×10⁶ cells) were injected; FAP-FITC was injected three times per week
Group 6 (n=6) (FAP5-PEG₁₆-FITC treatment); CAR T-cells (8×10⁶ cells) were injected; FAP-FITC was injected three times per week

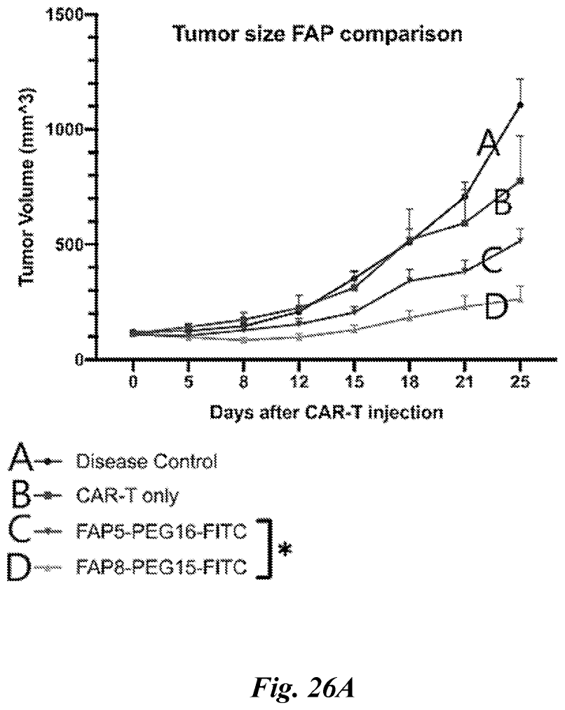

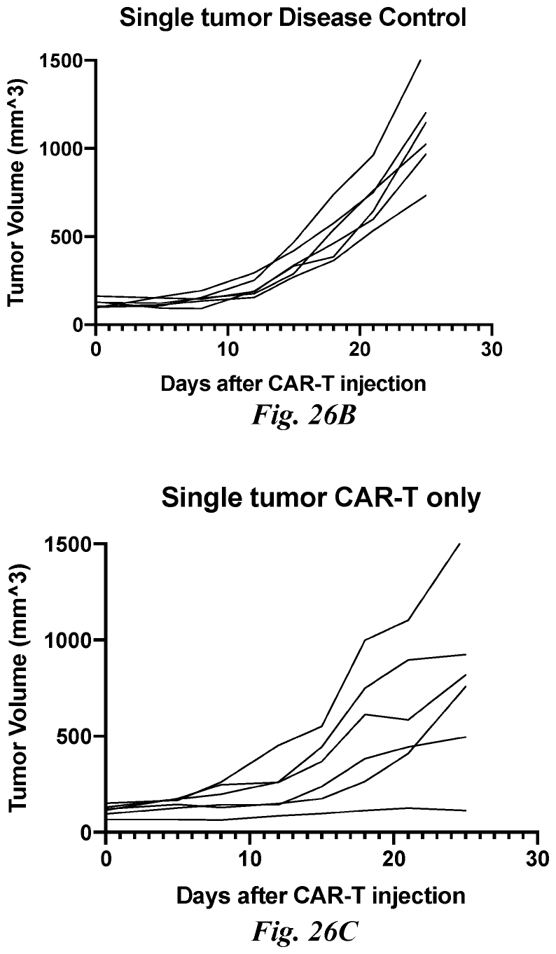

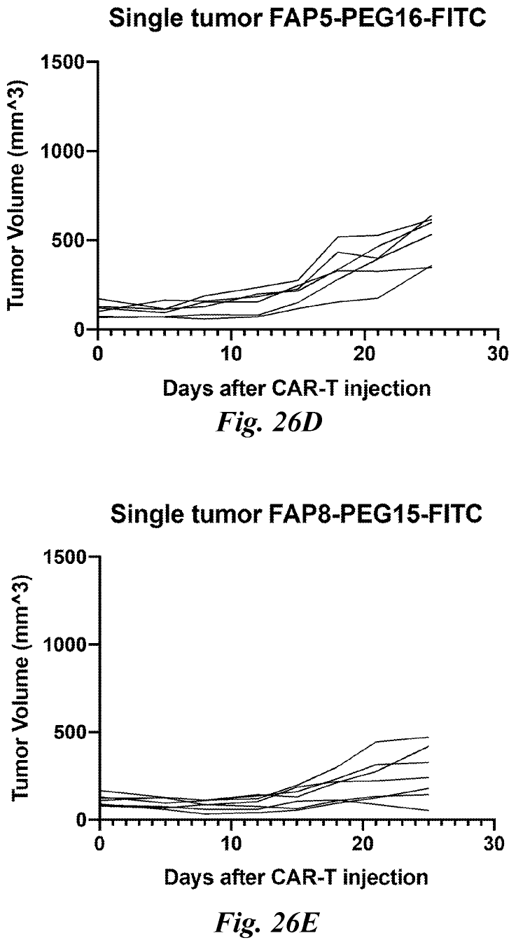

Although FAP5-FITC works similarly to FAP8-FITC in vitro, FAP8-FITC, which had better serum stability, seemed to mediate better engagement with CAR T-cells in vivo. See FIGS. 26A-26E.

### Example 25

### CAR T-Cell Tumor Infiltration

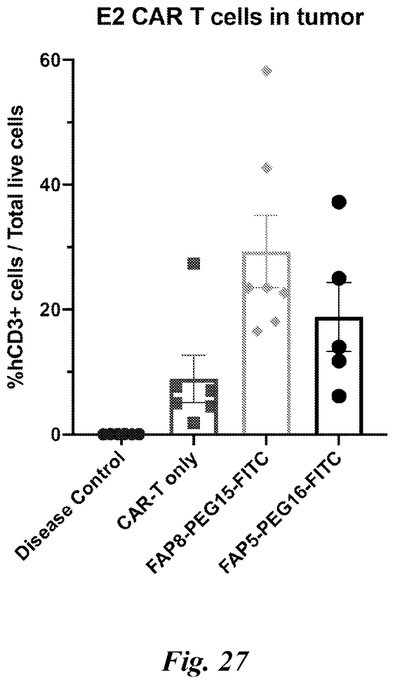

Tumors were harvested from mice at endpoint, digested with human tumor dissociation kit (Miltenyi) with 50% Enzyme R to enhance lymphocytes recovery. Cells were then stained with Zombie violet and Fc blocker on ice for 30 minutes and then washed twice. Cells were then stained with anti-hCD3 antibody on ice for 30 minutes and then washed thrice. Afterwards, cells were subjected to flow cytometry. More CAR T cell infiltration was observed with treatment of FAP8-FITC than with treatment of FAP5-FITC. See FIG. 27.

### Example 26

### FAP8-FITC Tumor Retention Analysis Via Imaging

FAP8-FITC (500 nmols/kg) was injected 24 hours before imaging in disease control mice (KB tumor). FAP8-FITC with PEG₂₃ linker showed weaker retention in the tumor, while FAP8-FITC with PEG₁₅ or PEG₁₈ linker showed good retention.

### Example 27

### Synthesis of DUPA-PEG₆-FITC

2-[3-(3-Benzyloxycarbonyl-1-tert-butoxycarbonyl-propyl)-ureido]pentanedioic Acid Di tert-butyl Ester (2). TEA (1.0 mL, 8.19 mmol) was added to a solution of L-glutamate di-tert-butyl ester hydrochloride (1.0 g, 3.39 mmol) and triphosgene (329.8 mg, 1.12 mmol) in dichloromethane (DCM) (25.0 mL) at −65° C. (Scheme 5). After the mixture was stirred for 2 hours at −65° C. under argon, a solution of L-Glu(OBn)-OtBu (1.2 g. 3.72 mmol) and TEA (600 μL, 4.91 mmol) in DCM (5.0 mL) was added. The reaction mixture was allowed to come to room temperature over a period of 1 hour and stirred at room temperature overnight.

The reaction was quenched with 1 M HCl, and the organic layer was washed with brine and dried over Na₂SO₄. The crude product was purified using flash chromatography (hexane:EtOAc; 1:1) to yield 2 (1.76 g, 90.2%) as a colorless oil. LCMS (m/z): (M+H)⁺ calcd for C₃₀H₄₇N₂O₉=579.33; found=579.30.

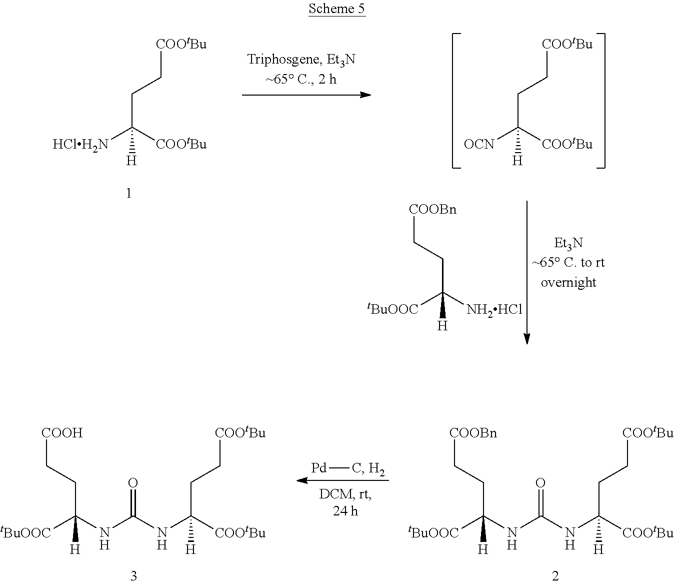

2-[3-(1,3-Bis-tert-butoxycarbonyl-propyl)-ureido]pentanedioic Acid 1-tert-Butyl Ester (3). 10% Pd/C was added to a solution of 2 (1.00 g, 432) in DCM. The reaction mixture was hydrogenated at 1 atm for 24 hours at room temperature. Pd/C was filtered through a Celite pad and washed with DCM. The crude product was purified using flash chromatography (hexane:EtOAc; 40:60) to yield 3 (80.2%) as a colorless oil. LCMS (m/z): (M+H)⁺ calcd for C₂₃H₄₁N₂O₉=489.28; found=489.28.

Solid Phase synthesis of 2-[3-(1,3-dicarboxypropyl)ureido]pentanedioic acid (DUPA) conjugate. The DUPA conjugate was synthesized by solid phase methodology as follows (see Scheme 5). 1,2-Diaminoethane trityl resin, polymer-bound 200-400 mesh (500 mg, 0.400 mM) was swollen with DCM (8 mL) by bubbling argon for 10 minutes, draining DCM, bubbling dimethylformamide (DMF, 8 mL) for 10 minutes, and then draining. The resin was washed with DMF (3×8 mL). After swelling the resin in DMF, a solution of Fmoc-Phen-OH (2.0 equiv), PyBOP (2.0 equiv) and DIPEA (5.0 equiv) in DMF (8.0 mL) was added (Scheme 6). Argon was bubbled for 5 hours, and resin was washed with DMF (3×8 mL). A solution of 20% piperidine in DMF (3×8 mL) was added to the resin, argon was bubbled for 15 minutes for each addition, and resin was washed with DMF (3×8 mL). The above sequence was repeated for three more coupling steps with Fmoc-Phen OH (2.0), 8-aminooctanoic acid (1.5 eq) and DUPA(OtBu)-OH 3 (1.5 eq) as shown in Scheme 7. Final compound was cleaved from the resin using a trifluoroacetic acid (TFA):H₂O:triisopropylsilane cocktail (95:2.5:2.5) and concentrated under vacuum. The concentrated product was precipitated in diethyl ether and dried under vacuum. The crude product was dissolved in a minimum amount of DMF and was purified using a C-18 column (reverse phase) [solvent gradient: 0% B to 80% B in 55 min run; A=A=milli Q water (TFA) pH=3; B=acetonitrile (ACN)]. Pure fraction was collected, froze at −80° C. for three hours, and lyophilized to yield conjugate 4. HRMS (ESI) (m/z): (M+H)⁺ calcd for C₃₉H₅₆N₇O₁₁=798.40, found, 798.40.

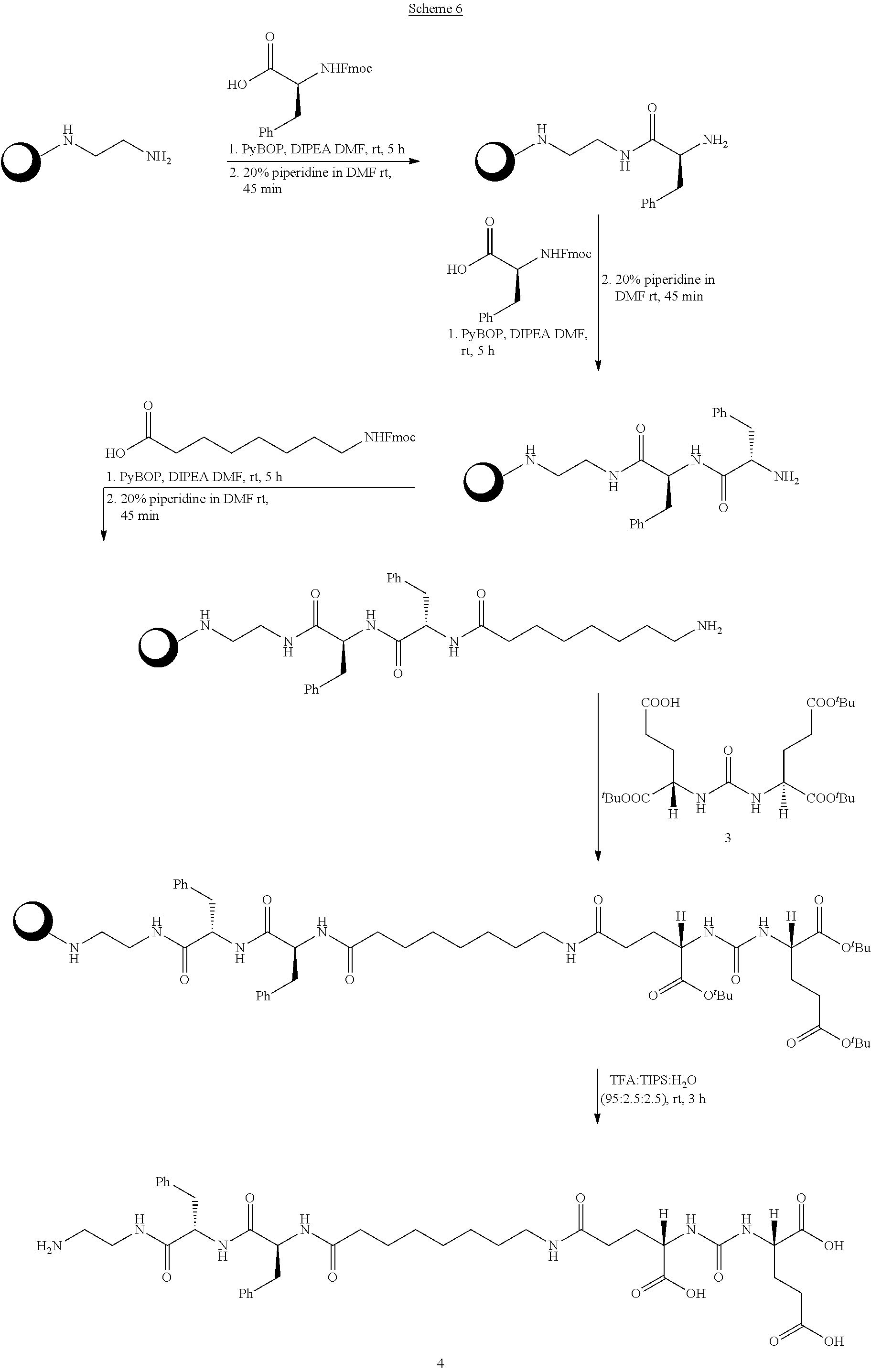

DUPA-PEG₆ NHFmoc (6). To a stirred solution of the DUPA conjugate 4 (0.100 g, 1 equiv) and PEG₆ NH ester 5 (0.092 g 1.1 equiv) in DMF (1.5 mL) was added DIPEA (0.044 mL, 2.0 equiv) at room temperature under argon, and the solution was stirred for 3 hours as mentioned in Scheme 7. The reaction was monitored by LCMS and purified by C-18 column reverse phase (mobile phase: A=milli Q water (TFA) pH=3, B=ACN: method: 0% B to 90% B in 50 min). The pure fractions were pooled and freeze-dried, furnishing the DUPA-PEG₆ NHFmoc 6.

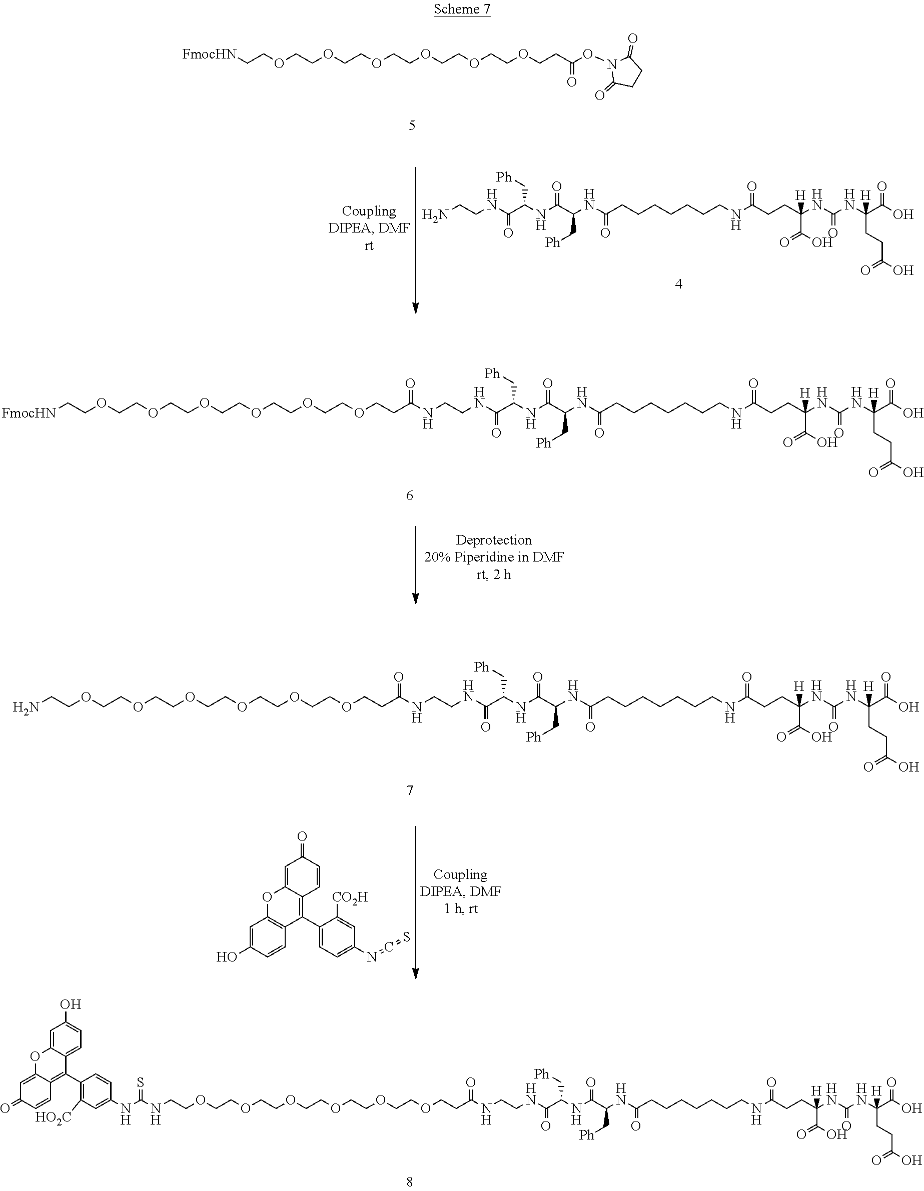

DUPA-PEG₆ NH₂ (7). DUPA-PEG₆ NHFmoc (6) was dissolved 20% piperidine in DMF (1.0 mL) at room temperature and stirred for 2 hours under argon (Scheme 7). The reaction was monitored by LCMS and purified by C-18 column reverse phase (mobile phase: A=milli Q water (TFA) pH=3, B=ACN; method: 0% B to 90 B in 50 min). The pure fractions were pooled and freeze-dried, furnishing the DUPA-PEG₆ NH₂ 7.

DUPA-PEG₆-FITC (8). DUPA-PEG₆ NH₂ 7 (20 mg, 1.0 equv) was dissolved in DMF (1.0 mL) under argon atmosphere, after which FITC (1.2 equiv) was added under dark condition (Scheme 7). After stirring for 5 minutes, DIPEA (4.0 equiv) was added to the solution, and reaction was monitored with LCMS (reaction completed within 2.0 hours). The crude product was purified by C-18 column reverse phase (mobile phase: A=milli Q water (TFA) pH=3, B=ACN; method: 0% B to 90% B in 60 min). Pure fraction was collected, froze at −80° C. for three hours, and lyophilized to yield DUPA-PEG₆-FITC conjugate 8. LCMS (ESI) (m/z): (M+H)⁺ calcd for C₇₅H₉₅N₉O₂₅S+H=1522.6 found (m/2): (M+H)⁺ 762.4 and (m/3): (M+H)⁺ 508.4.

### Example 28

### Synthesis of PSMAL1-PEG₆-FITC

PSMAL1-PEG₆-FITC conjugate was synthesized by solid phase methodology as follows (see Schemes below):

DIPEA (1.3 mL, 10 equiv, 7.5 mmol) was added to a solution of L-glutamate di-tert-butyl ester hydrochloride (1.33 g, 6.00 equiv, 4.50 mmol) and triphosgene (0.445 g, 2.00 equiv, 1.5 mmol) in DCM (15.0 mL) at −65° C. (Scheme 8). After stirring for 1 hour at −65° C. under argon, the solution was warmed to room temperature and stirred for 1 hour at room temperature to produce corresponding isocyanate intermediate in situ.

H-L-LYS(ALLOC)-2-Cl-trityl resin (1.0 g, 1.0 equiv, 0.75 mM) was swollen with dry DCM (8 mL) by bubbling argon for 10 minutes in a peptide vessel. Then DCM was drained, and the resin was washed twice with dry DCM and then drained. Immediately after swelling the resin in DCM, the in situ-generated isocyanate was transferred to a peptide vessel under argon atmosphere. Argon was bubbled overnight (16 hours) at room temperature, and the isocyanate was washed with DCM. The completion of the reaction was confirmed by Kaiser Test.

After that Tetrakis(triphenylphosphine)palladium(0) (100 mg) and 0.800 ml of morpholine were dissolved in 8 mL of dry DCM in a round bottom flask under argon atmosphere, was then transferred to a peptide vessel, and argon was bubbled for 3 hours at room temperature to remove allyloxy protecting group. After deprotection, the resin was washed three times with dry DCM. Again, the completion of the reaction was confirmed by Kaiser Test. Further resin was washed with twice with DMF and thrice with sodium diethyldithiocarbamate (0.03 M in DMF) to remove residual Pd catalyst. Finally, the resin was washed thrice with DMF and DCM.

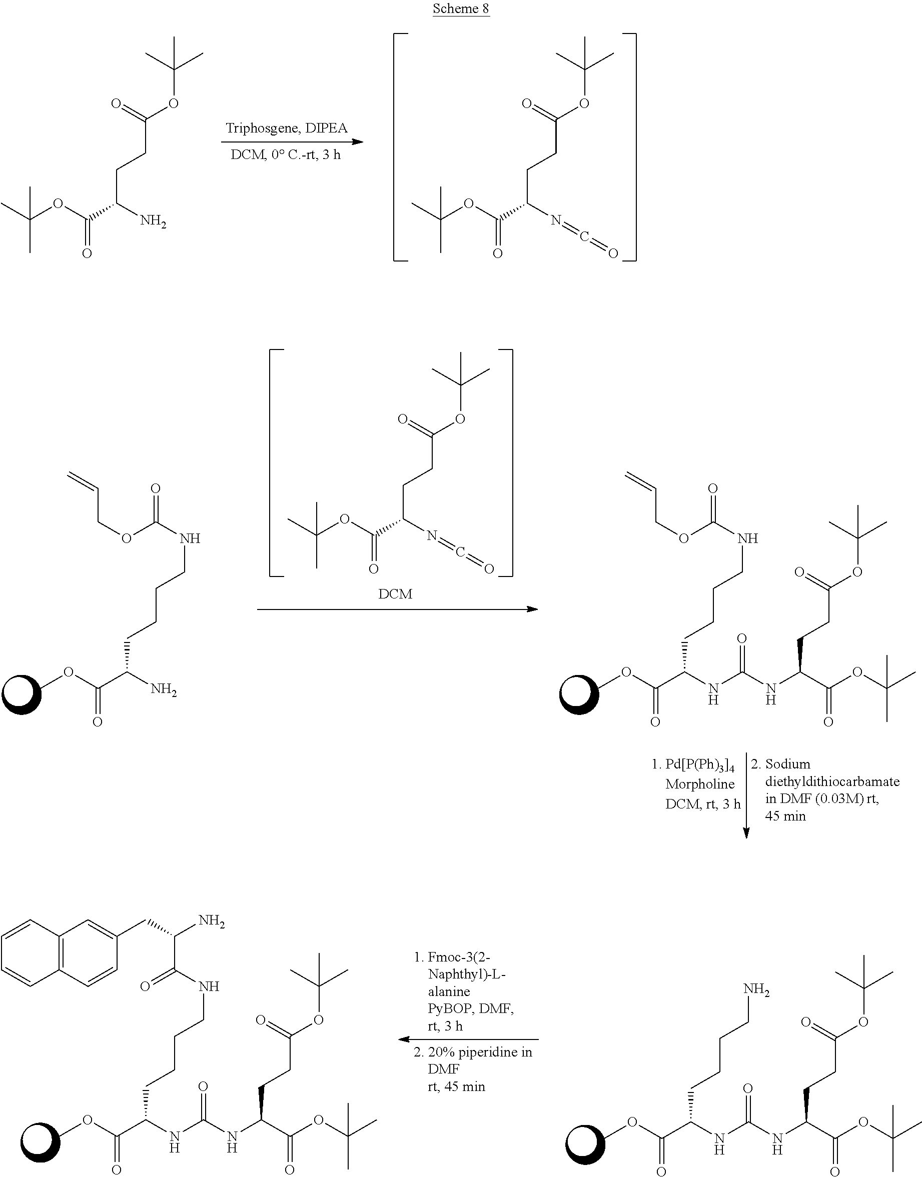

Immediately, the resin was swelled in DMF for further coupling, and a solution of Fmoc-3(2-naphthyl)-L-alanine (2.0 equiv), PyBOP (2.0 equiv) and DIPEA (5.0 equiv) in DMF (8.0 mL) was added (Scheme 8). Argon was bubbled for 5 hours, and resin was washed with DMF (3×8 mL). A solution of 20% piperidine in DMF (3×8 mL) was added to the resin, argon was bubbled for 15 minutes for each addition, and resin was washed with DMF (3×8 mL). The above sequence was repeated for two more coupling steps with N-Fmoc-tranexamic acid (2.0), Fmoc-N-amido-PEG₆-acid (2.0 eq) as shown in Scheme 9. Final compound was cleaved from the resin using a trifluoroacetic acid (TFA):H₂O:triisopropylsilane cocktail (95:2.5:2.5) and concentrated under vacuum. The concentrated product was precipitated in diethyl ether and dried under vacuum. The crude product was dissolved in a minimum amount of DMF and was purified using C-18 column (reverse phase) [solvent gradient: 0% B to 80% B in 55 min run; A=milli Q water (TFA pH=3); B=acetonitrile (ACN)]. Pure fraction was collected, froze at −80° C. for three hours, and lyophilized to yield conjugate PSMAL1-PEG₆-NH₂ intermediate. HRMS (ESI) (m/z): (M+H) calcd for C₄₈H₇₅N₆O₁₆=991.52, found, 991.5.

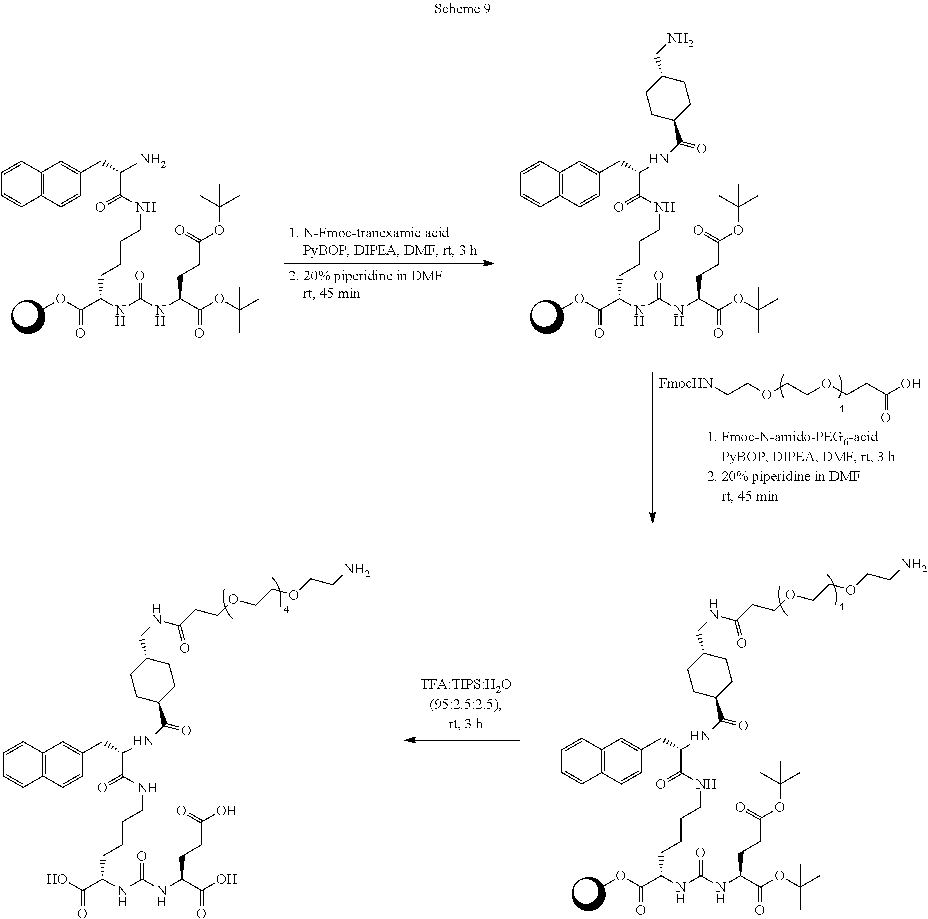

Synthesis of PSMAL1-PEG₆-FITC Conjugate. PSMAL1-PEG₆-NH₂ (10 mg, 1.0 equv) was dissolved in DMF (1.0 mL) under argon atmosphere, after which FITC (1.2 equiv) was added under dark conditions (scheme 10). After stirring for 5 minutes, DIPEA (4.0 equiv) was added to the solution, and the reaction was monitored with LCMS (reaction completed within 1.0 to 2 hrs). The crude product was purified by C-18 column reverse phase (mobile phase: A=milli Q water (TFA, pH=3), B=ACN; method: 0% B to 90% B in 60 minutes). Pure fraction was collected and froze at −80° C. for three hours and lyophilized to yield PSMAL1-PEG₆-FITC conjugate 8. LCMS (ESI) (n/z): (M+H)⁺ calcd for C₆₉H₈₆N₇O₂₁S+H=1380.56 found (m/2): (M+H)⁺ 690.9 and (m/3): (M+H)⁺ 461.0

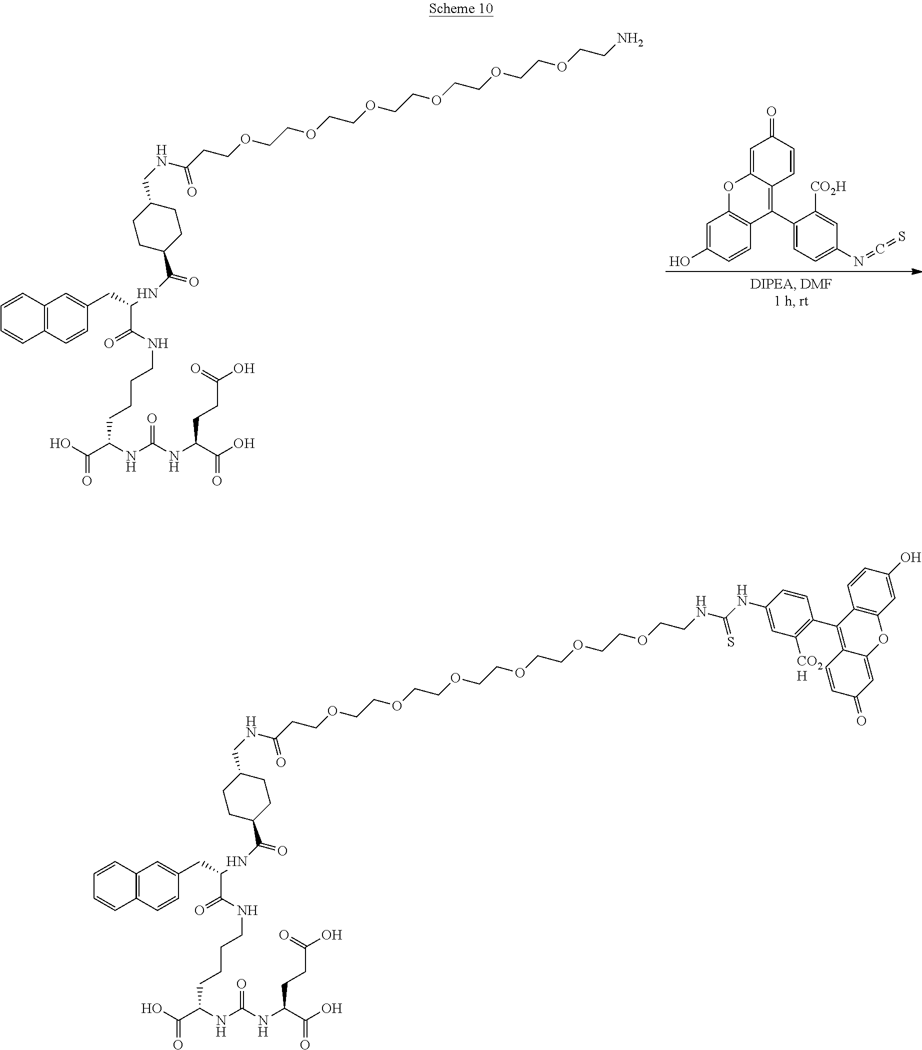 [SMILES: NCCOCCOCCOCCOCCOCCOCCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1 ; O=C(O)c1cc(N=C=S)ccc1-c1c2ccc(=O)cc-2oc2cc(O)ccc12 ; O=C(O)CC[C@H](NC(=O)N[C@@H](CCCCNC(=O)[C@H](Cc1ccc2ccccc2c1)NC(=O)[C@H]1CC[C@H](CNC(=O)CCOCCOCCOCCOCCOCCOCCNC(=S)Nc2ccc(-c3c4ccc(=O)cc-4oc4cc(O)ccc34)c(C(=O)O)c2)CC1)C(=O)O)C(=O)O]

### Example 29

### Effect of Linker Length on Binding Affinity to PSMA

MDA-PSMA cells (0.2 million) were incubated with 2-fold serial diluted adapters starting from 500 nM in complete RPMI medium (RPMI+10% FBS) for 1 hour at room temperature. Cells were washed twice with 2% FBS in PBS and resuspended in the same buffer. Fluorescent intensity of fluorescein isothiocyanate (FITC) was analyzed by flow cytometer. Results are shown in FIG. 29.

As shown in FIG. 29, as the length of the linker between DUPA and FITC increased, the binding affinity and the total binding of DUPA-FITC adapters decreased.

### Example 30

### PSMA Expression Levels in Various Cell Lines Used

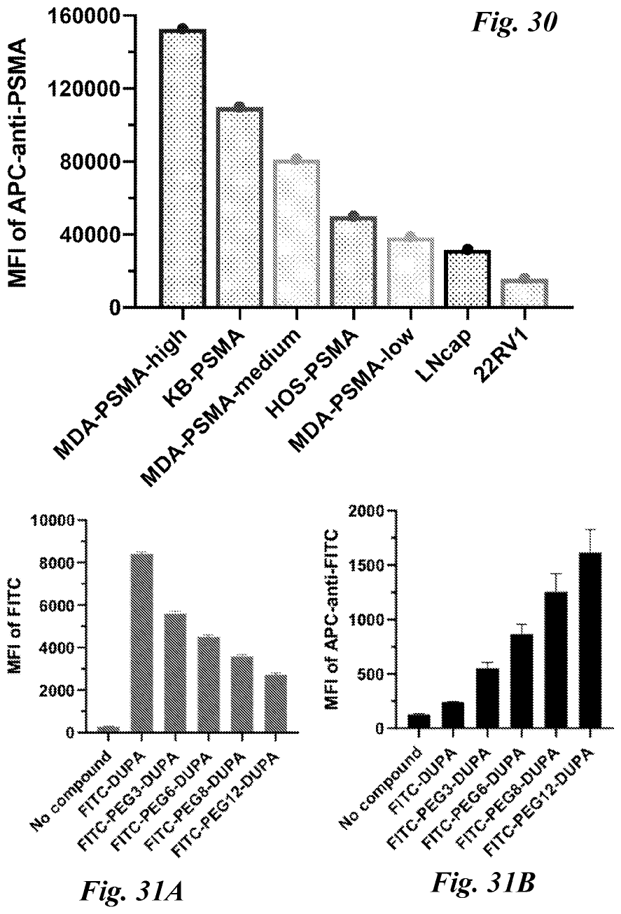

Cells (0.2 million) were stained with APC-anti-PSMA antibody on ice for 30 minutes and washed twice with 2% FBS in PBS. Fluorescent intensity of APC-anti-PSMA was analyzed by flow cytometry. Results are shown in FIG. 30, which shows the level of expression of PSMA in the indicated cell lines.

### Example 31

### Length of PEG Linker Affects Binding and Surface Exposure of FITC Moiety on Target Cells

MDA-PSMA cells (0.2 million) were incubated with 1 μM of DUPA-FITC conjugates with different PEG linkers in complete RPMI medium (RPMI+10% FBS) for 1 hour at room temperature. Free compounds were washed away, and fluorescent intensity of FITC was analyzed by flow cytometer. To analyze the surface exposure of FITC moiety, the stained cells were incubated with APC-anti-FITC on ice for 30 minutes. Then the cells were washed, and the fluorescent intensity of APC was analyzed by flow cytometer. Results are shown in FIGS. 31A-31B.

As shown in FIG. 31A, the total binding of DUPA-FITC decreases as the linker length increases. As shown in FIG. 31B, the surface exposure of FITC increases as the linker length increases.

### Example 32

### Increasing Linker Length Up to PEG₆ Increases Activation of Anti-HTC CAR-T Cells

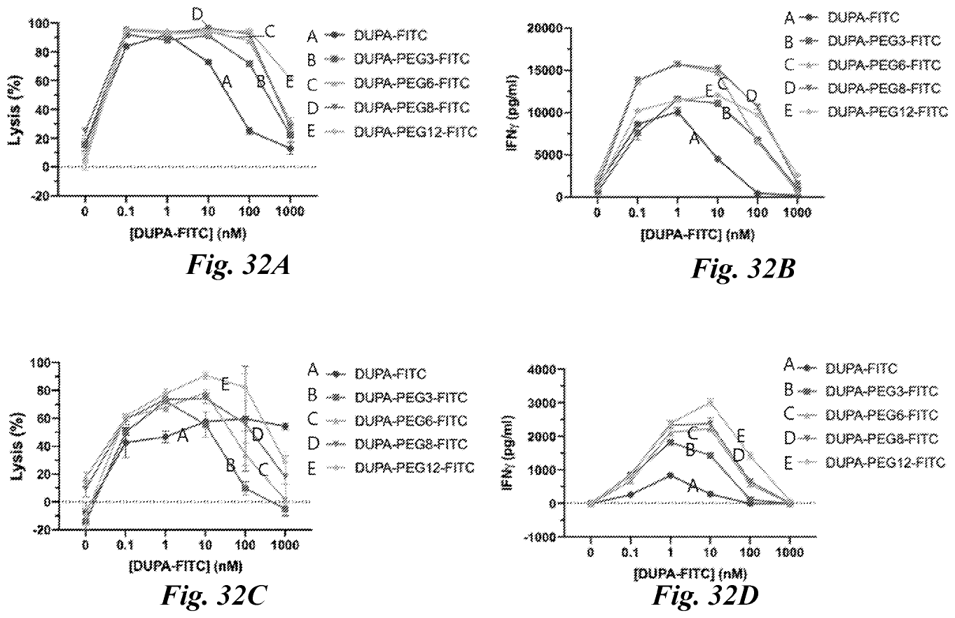

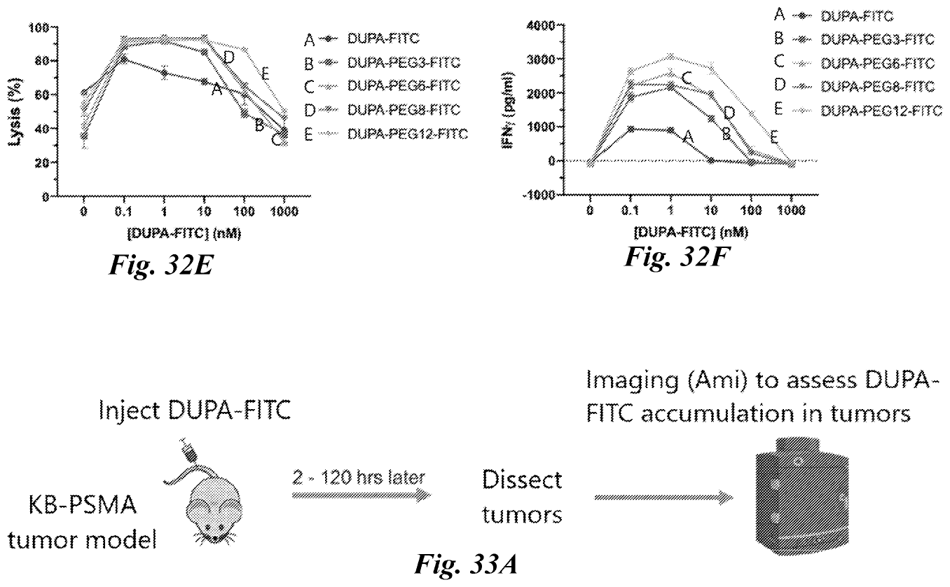

Anti-FITC CAR-T cells were incubated with equal number of target cells in the presence of FITC-PEG-DUPA at different concentrations. The number of target cells was determined at the end of the co-culture, and the cytotoxicity was calculated using the formula: [(number of untreated cells−number of treated cells)/number of untreated cells]*100%. Secretion of IFNγ from CAR-T cells in the co-culture medium was analyzed by ELISA using a human IFNγ ELISA kit (Biolegend). Results are shown in FIGS. 32A-32F. As shown in the figures, increasing linker length in the bispecific adapter increased the activation of anti-FITC CAR-T cells. When the linker length reached PEG₆, the CAR-T cell reached the highest activation level. Longer linker lengths did not dramatically improve the activation of CAR-T cells.

### Example 33

### Time Course of DUPA-FITC Accumulation in KB-PSMA Tumors

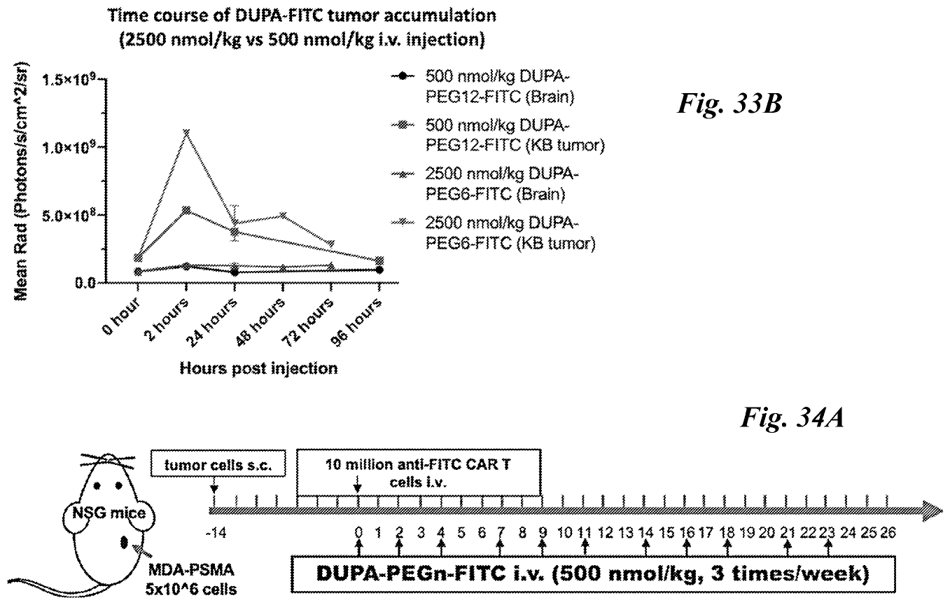

KB-PSMA tumor bearing mice were injected with indicated doses of DUPA-PEG₆-FITC or DUPA-PEG₁₂-FITC. The mice were euthanized at desired time points, and the tumors were dissected. FITC fluorescent images of the tumors were taken in the Spectral Ami imaging system, and the mean rads of the images were analyzed by the Aura software. The results are shown in FIG. 33B. As shown in FIG. 33B, peak accumulation of DUPA-PEG₆-FITC and DUPA-PEG₁₂-FITC in KB-PSMA tumors occurred about two hours after injection and almost complete clearance occurred at about 96 hours.

### Example 34

### DUPA-FITC with Different PEG Linkers Mediates Eradication of MDA-PSMA Tumors in Mice without Causing Loss of Body Weight

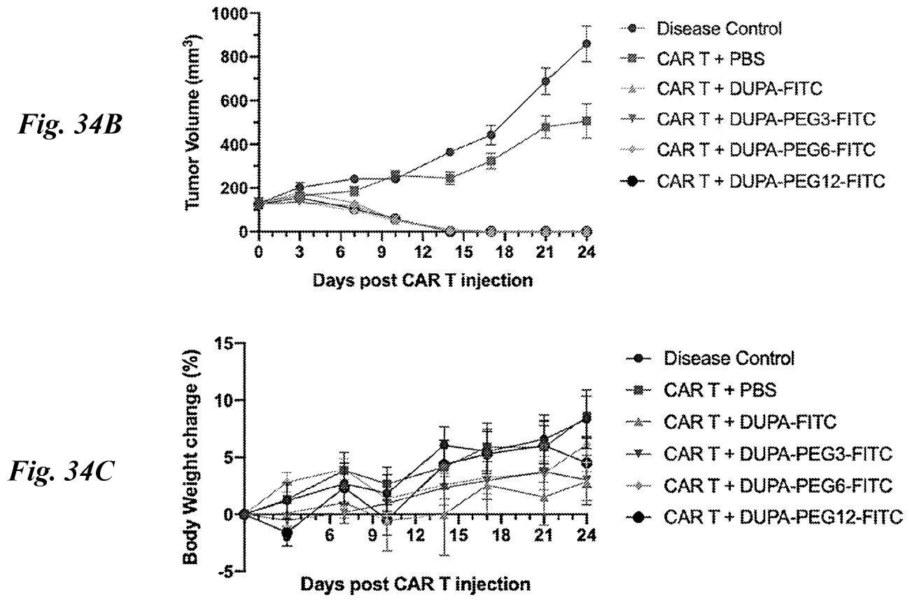

MDA-PSMA cells (high PSMA level; 5 million cells) were implanted into each NSG mouse by subcutaneous injection. When the tumor volumes reached about 100 mm, the treatment groups were injected with 10 million anti-FITC CAR T-cells and bispecific adapters (no linker, PEG₃, PEG₆, or PEG₁₂) as shown in FIG. 34A. The tumor volume and body weight were monitored regularly. Tumor volume was calculated using the formula: (length*width²)/2. Results are shown in FIGS. 34B and 34C. As shown in FIG. 34B, all the DUPA-FITC adapters with different PEG linkers successfully mediated the eradication of tumors. As shown in FIG. 34C, all the DUPA-FITC adapters tested did not cause body weight loss during treatment.

### Example 35

### PSMAL1-PEG₆-FITC has Higher Binding Affinity and Higher Total Binding than DUPA-PEG₆-FITC

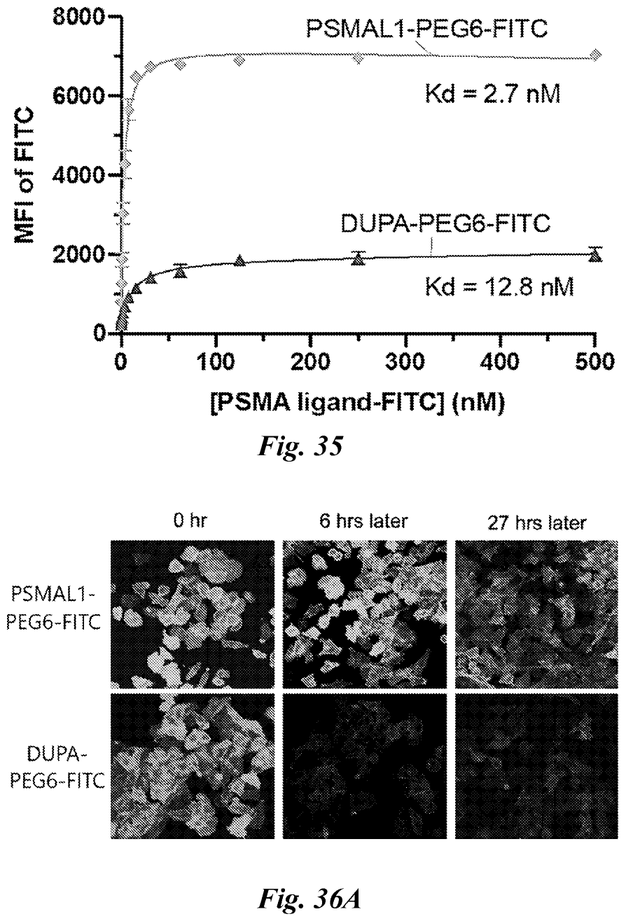

MDA-PSMA cells (0.2 million) were incubated with 2-fold serial diluted adapters starting from 500 nM in complete RPMI medium (RPMI+10% FBS) for 1 hour at room temperature. Cells were washed twice with 2% FBS in PBS and resuspended in the same buffer. Fluorescent intensity of FITC was analyzed by flow cytometer. Results are shown in FIG. 35.

As shown in FIG. 35, the binding affinity of PSMAL1-PEG₆-FITC is five-fold higher than the binding affinity of DUPA-PEG₆-FITC to MDA-PSMA cells.

### Example 36

### PSMAL1-PEG₆-FITC has Better Retention in PSMA⁺ Cells than DUPA-PEG₆-FITC

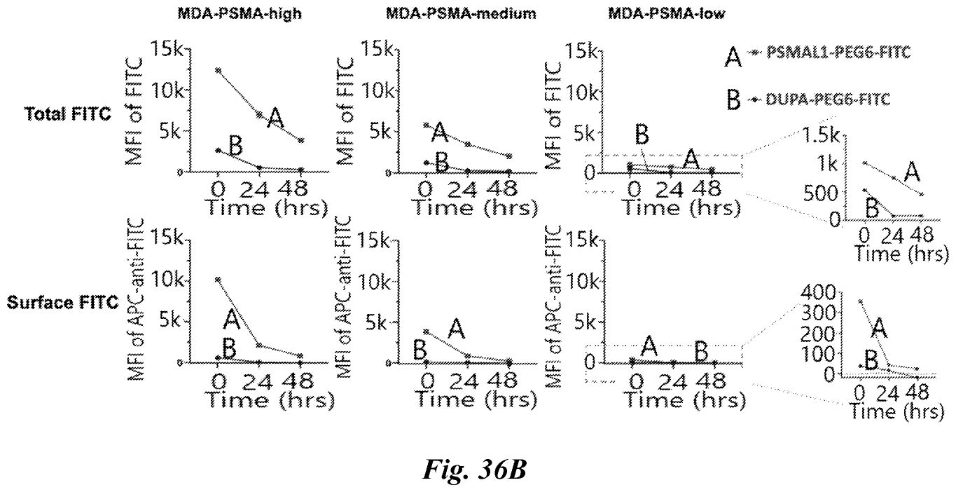

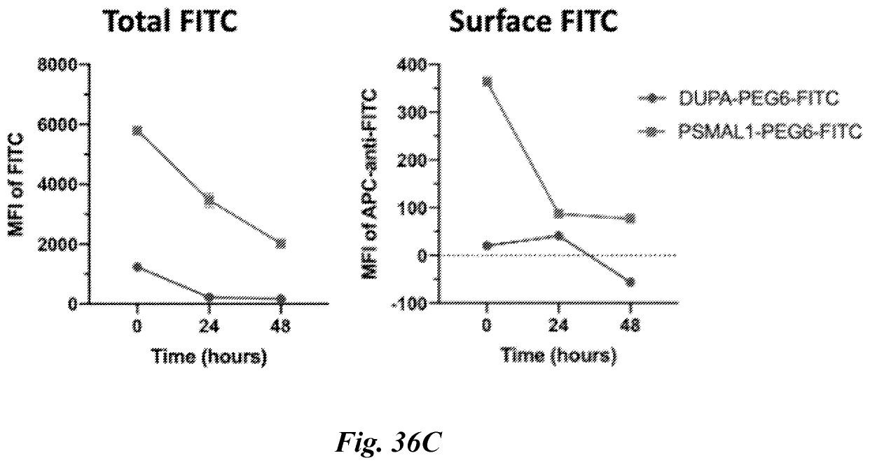

MDA-PSMA cells or 22Rv1 cells were incubated with 1 μM of DUPA-PEG₆-FITC or PSMAL1-PEG₆-FITC for one hour at room temperature. Unbound compounds were washed away, and the cells were cultured in RPMI containing 10% FBS at 37° C. with 5% CO₂. At indicated time points, the cells were imaged under a confocal microscope to analyze the intensity and location of FITC. To quantify the intensity of FITC and surface-bound FITC, cells stained with DUPA-PEG₆-FITC and PSMAL1-PEG₆-FITC were analyzed by flow cytometry at indicated time points to analyze the FITC fluorescence intensity. To analyze the surface-bound FITC, the cells were stained with APC-anti-FITC antibody for 30 minutes on ice at different time points. The fluorescence intensity of APC was analyzed by flow cytometer. Results are shown in FIGS. 36A-36C. As shown in FIG. 36A, PSMAL1-PEG₆-FITC stayed longer in MDA-MB-231-PSMA cells than DUPA-PEG₆-FITC. FIG. 36B shows PSMAL1-PEG₆-FITC stayed longer in MDA-PSMA cells expressing different levels of PSMA than DUPA-PEG₆-FITC. As shown in FIG. 36C, PSMAL1-PEG₆-FITC stayed longer in 22Rv1 cells, which naturally express low levels of PSMA, than DUPA-PEG₆-FITC.

### Example 37

### Efficacy of Anti-FITC CAR-T Cells Mediated by PSMAL1-PEG₆-FITC is Higher than DUPA-PEG₆-FITC at Low Concentrations

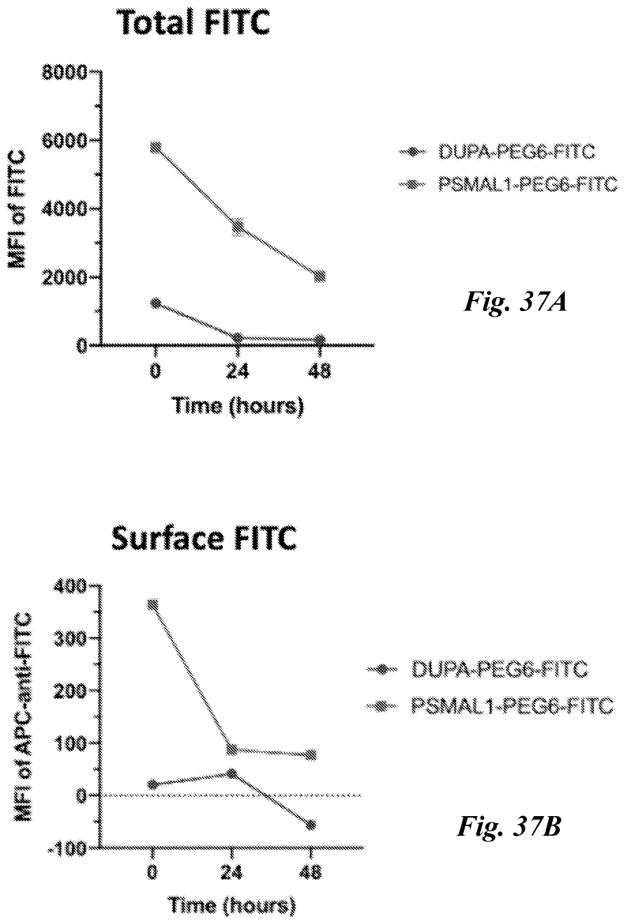

Equal number of 22Rv1 and anti-FITC CAR T cells were co-cultured in 96-well plates with DUPA-PEG₆-FITC or PSMAL1-PEG₆-FITC at different concentrations for 48 hours. The number of 22Rv1 cells was counted at the end of the co-culture. The cytotoxicity of anti-FITC CAR T cells was calculated using the formula: (number of untreated 22Rv1 cells−number of treated 22Rv1 cells)/number of untreated 22Rv1 cells*100%. Concentrations of IFNγ in the co-culture medium were analyzed by ELISA using human IFNγ ELISA kit (Biolegend). Results are shown in FIGS. 37A-37B. As shown in FIG. 37A, lysis of 22Rv1 cells by anti-FITC CAR-T cells mediated by PSMAL1-PEG₆-FITC was much higher than DUPA-PEG₆-FITC at low concentration. As shown in FIG. 37B, IFNγ secreted by anti-FITC CAR-T cells when co-cultured with 22Rv1 cells in the presence of PSMAL1-PEG₆-FITC was higher than DUPA-PEG₆-FITC at low concentrations.

### Example 38

### PSMAL1-PEG₆-FITC has Better Retention in MDA-MB-231-PSM4 Tumors than DUPA-PEG₆-FITC and DUPA-FITC

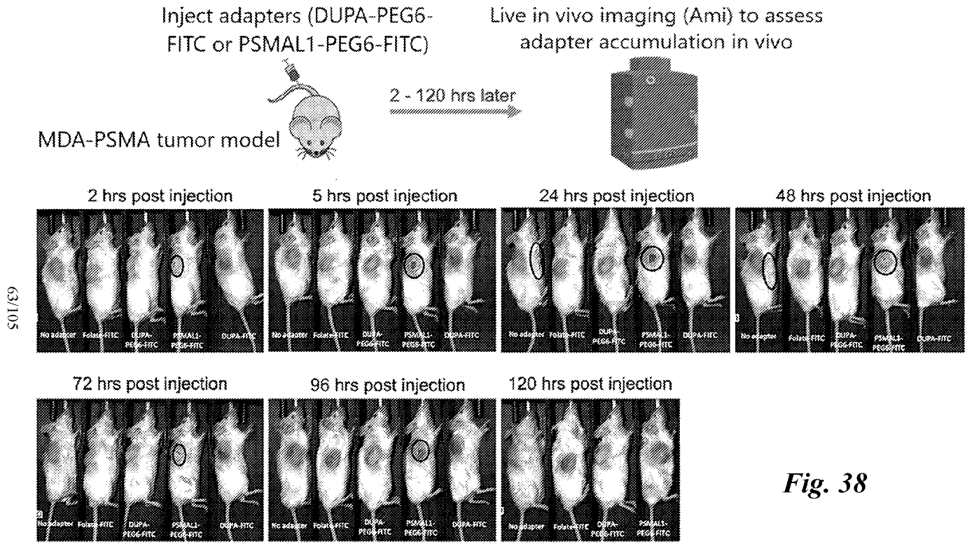

MDA-PSMA tumor bearing mice were intravenously injected with the indicated adapters at 500 nmol/kg. Fluorescent images of the mice were taken in the Spectral Ami imaging system at the indicated time points after injection. Results are shown in FIG. 38.

### Example 39

### PSMAL1-PEG₆-FITC has Longer Retention on MDA-MB-231-PSMA Tumor Cell Surfaces In Vivo

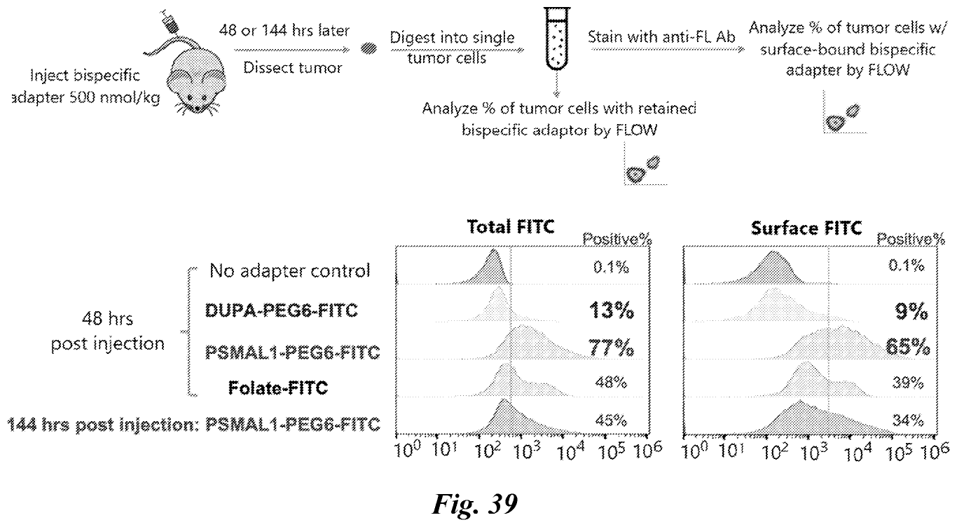

MDA-PSMA tumor bearing mice were injected with DUPA-PEG₆-FITC, PSMAL1-PEG6-FITC or folate-FITC as control. The mice were euthanized at the indicated time points after injection. Tumors were dissected and dissociated into single cells. Fluorescent intensity of FITC indicating the total retention of the adapters in the tumor cells were analyzed by flow cytometry. To analyze the surface retention of the adapters on the tumor cell surface, the dissociated tumor cells were stained with APC-anti-FITC antibody, and the fluorescent intensity of APC was analyzed by flow cytometry. Results are shown in FIG. 39. After injection (48 hours post-injection), 77% of the tumor cells still had PSMAL1-PEG₆-FITC in the cells, and 65% of the cells retained PSMAL1-PEG₆-FITC on the surface. In contrast, only 13% of the tumor cells had DUPA-PEG₆-FITC, and 9% of the cells retained DUPA-PEG₆-FITC on the surface. At 144 hours post-injection, 45% of the tumor cells retained PSMAL1-PEG₆-FITC, and 34% of the cells retained PSMAL1-PEG₆-FITC on the surface.

### Example 40

### PSMAL1-PEG₆-FITC and DUPA-PEG₆-FITC can Mediate Eradication of MDA-PSMA Tumors Expressing High Levels of PSMA

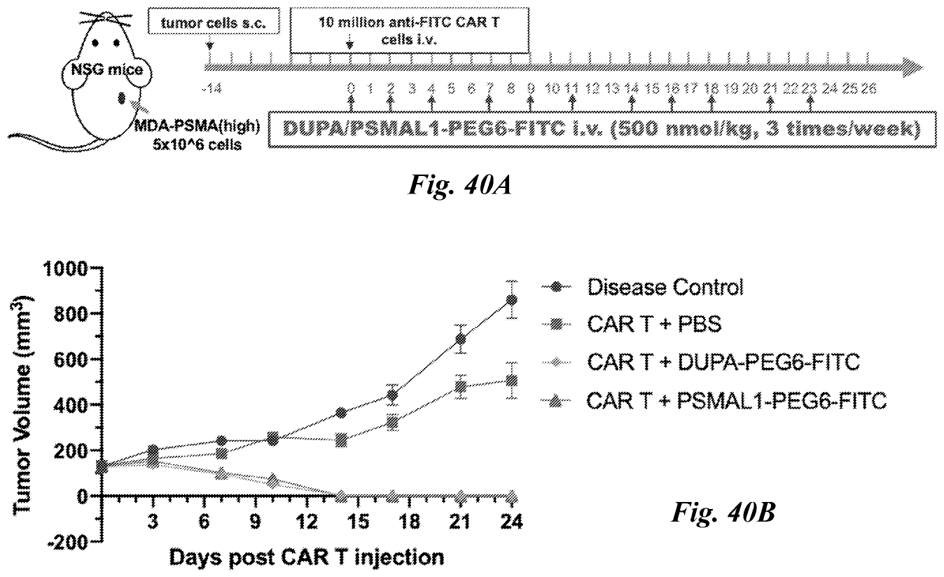

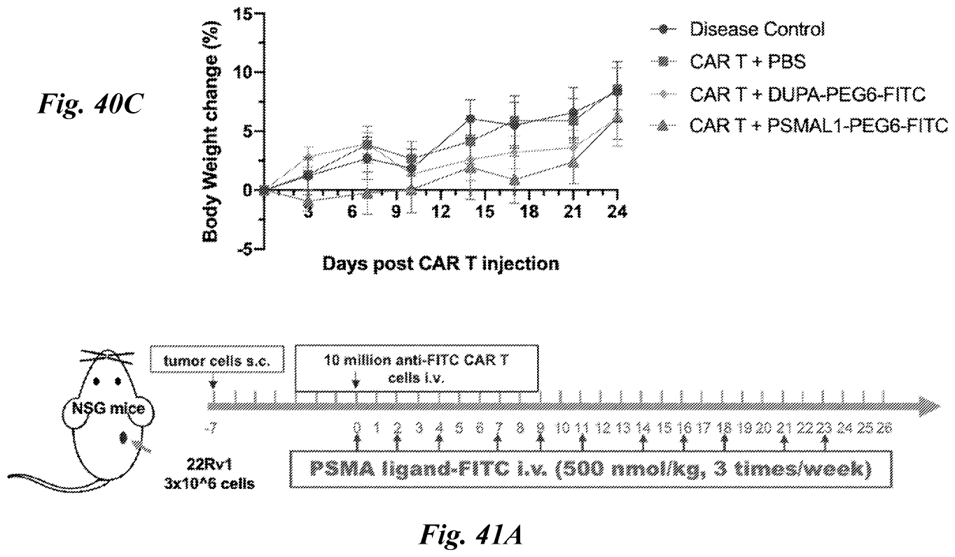

MDA-PSMA cells (5 million) were implanted into each NSG mouse by subcutaneous injection. When the tumor volumes reached about 100 mm³, the mice were injected with 10 million anti-FITC CAR T-cells and corresponding adapters. The volumes of the tumors and the body weights of the mice were monitored regularly. Tumor volume was calculated using the formula: (length*width²)/2. Results are shown in FIGS. 40A-40C. Both DUPA-PEG₆-FITC and PSMAL1-PEG₆-FITC have good efficacy on eliminating MDA-PSMA tumors expressing high levels of PSMA, and no body weight loss was observed in the mice during the treatment.

### Example 41

### PSMAL1-PEG₆-FITC Significantly Inhibited the Growth of 22Rv1 Tumor

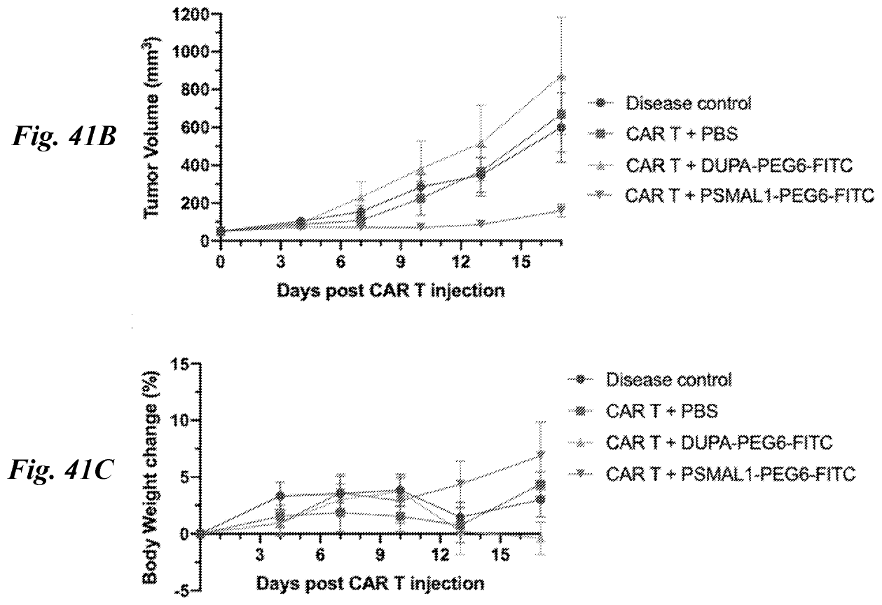

22Rv1 cells (3 million) were implanted in NSG mice by subcutaneous injection. When the tumor volumes reached about 50 mm³, the mice were injected with 10 million anti-FITC CAR T cells and the indicated adapters. The volumes of the tumors and the body weights of the mice were monitored regularly. Tumor volume was calculated using the formula: (length*width²)/2. Results are shown in FIGS. 41A-41C. PSMAL1-PEG₆-FITC and anti-FITC CAR-T cells inhibited the growth of 22Rv1 tumor significantly, while DUPA-PEG₆-FITC didn't show much efficacy. No obvious body weight loss was observed in any of the treatment groups during treatment.

### Example 42

### Efficacy of PSMAL1-PEG₆-FITC Binding to TagCAR T Cells Generated Using a Lentiviral Vector

This study was carried out to assess the efficacy of a combination of PSMAL1-PEG₆-FITC administered with T cells engineered in vivo with lentiviral vectors encoding a universal anti-fluorescein CAR (TagCAR). Such methods may be advantageous to other costly or toxic autologous methods.

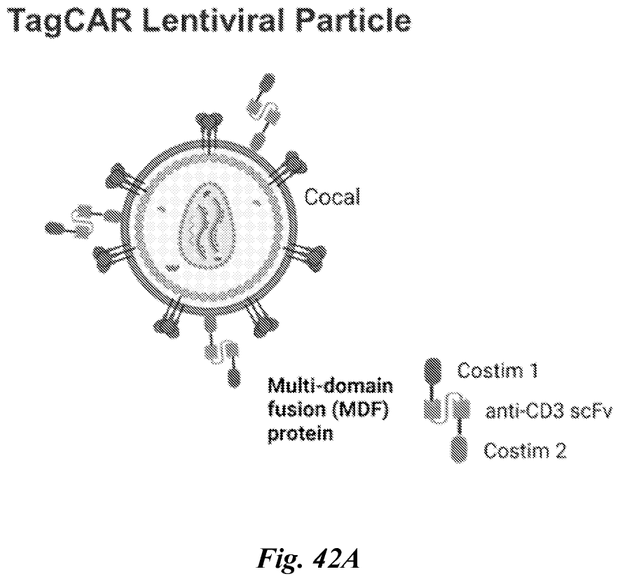

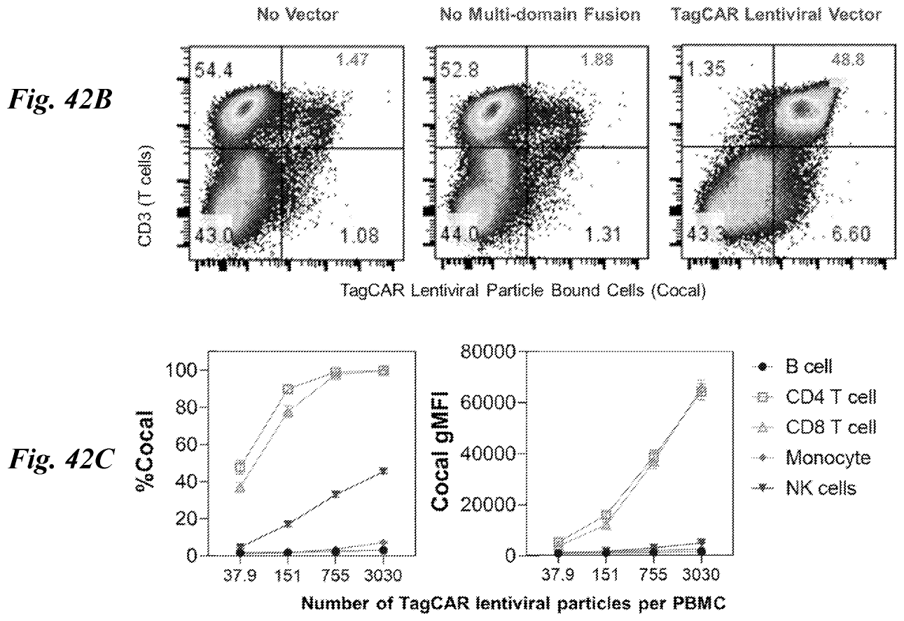

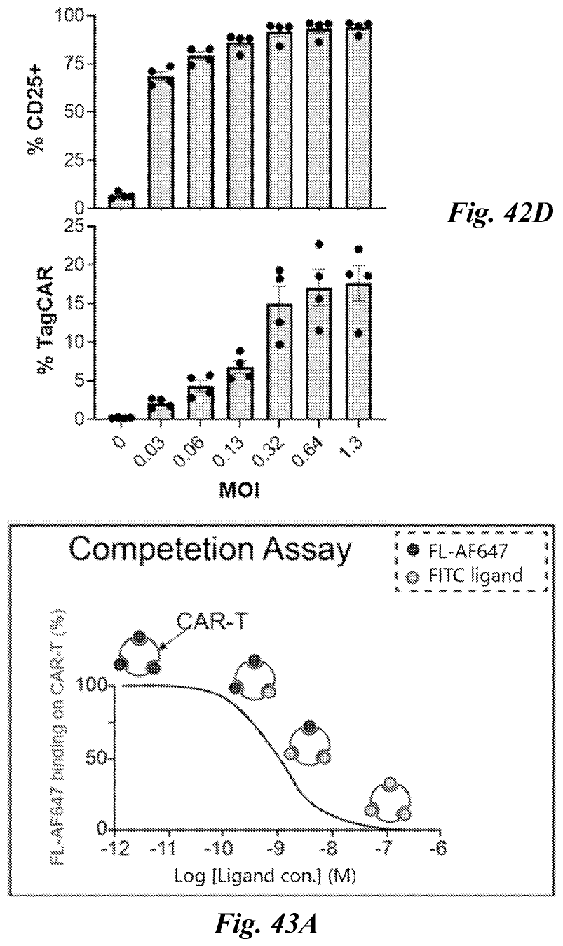

Selective generation of universal anti-fluorescein CAR (TagCAR) T cells using a lentiviral vector. An exemplary TagCAR lentiviral vector (FIG. 42A) was used to facilitate transduction and payload expression of TagCAR in T cells. The TagCAR lentiviral particles were pseudotyped with cocal glycoprotein and were surface-engineered to express a multidomain fusion protein (MDF) composed of an anti-CD3 single chain variable fragment (scFv) sandwiched between two T cell costimulatory ligands. The vector also contained the TagCAR payload as a polynucleotide encoding the TagCAR with the following components in N-terminal to C-terminal order: an scFv (e.g., anti-FITC E2), a hinge (spacer), a transmembrane domain, and an endodomain with a costimulatory signaling domain and a CD3zeta signaling domain (Z).

To assess selective T cell binding, activation, and transduction by TagCAR lentiviral particles, peripheral blood mononuclear cells (PBMCs) were transduced with either TagCAR lentiviral particles containing the TagCAR payload or, as negative controls, no TagCAR lentiviral vector or a TagCAR lentiviral vector that did not contain the MDF. Representative flow plots one-hour post-incubation of PBMCs with an anti-cocal antibody used to detect the lentiviral vector binding are shown in FIG. 42B. In addition, the percentage of circulating immune cell subsets depending on the amount of delivered TagCAR lentiviral particles is shown in FIG. 42C. Furthermore, T cell activation and transduction was measured, respectively, as a percentage of CD25+ 3 days post-transduction and TagCAR+ 7 days post-transfection (FIG. 42D). Together, these data suggest TagCAR lentiviral particles selectively generate TagCAR T cells following treatment of PBMCs.

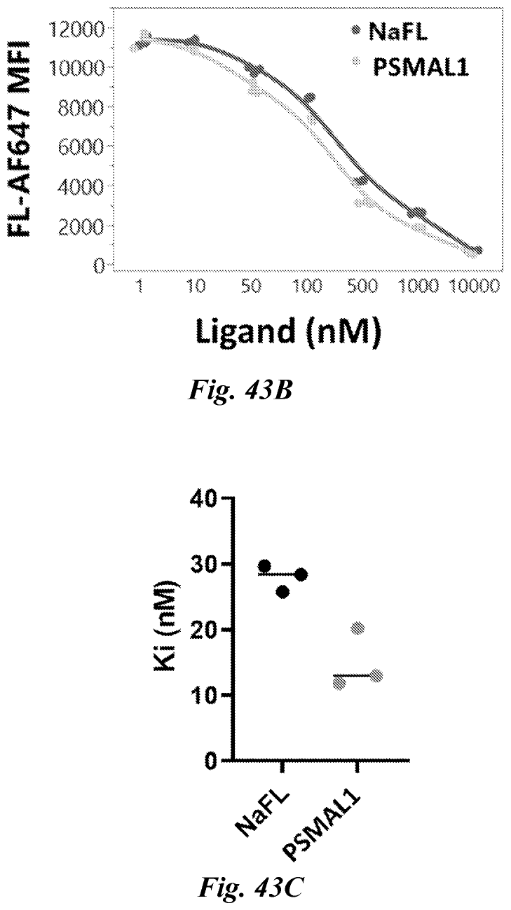

PSMAL1-PEG₆-FITC strongly binds to TagCAR T cells generated using TagCAR lentiviral particles. To demonstrate that TagCAR T cells generated using TagCAR lentiviral particles can strongly bind to PSMAL1-PEG₆-FITC, a competitive assay graphically depicted in FIG. 43A was used to define the interaction between the TagCAR and PSMAL1-PEG₆-FITC. Due to fluorescein fluorescence being quenched when bound by TagCAR, TagCAR T cells were instead incubated with saturating levels of FL-AF647, which was then competed off by increasing concentrations of PSMAL1-PEG₆-FITC. Results from the competitive assay are shown in FIGS. 43B-43C. As compared to the antigen-only control sodium fluorescein (NaFL), lower concentrations of PSMAL1-PEG₆-FITC were necessary to compete with FL-AF647, as detected by the mean fluorescent intensity (MFI) of FL-AF647. Additionally, the mean calculated inhibitor constant (Ki), or concentration at half max inhibition, from triplicates composed of TagCAR T cells derived from different donors was calculated for PSMAL1-PEG₆-FITC and NaFL using the equation: Ki=IC50/(1+[S]/Km), where [S]=[FL-AF647]=100 nM and the Km of FL-AF647 is 9.9 nM. PSMAL1-PEG₆-FITC had a lower Ki at 9.9 nM as compared to NaFL, suggesting PSMAL1-PEG₆-FITC strongly bound to TagCAR T cells generated using TagCAR lentiviral particles.

### Example 43

### Efficacy of TagCAR T Cells Generated Using TagCAR Lentiviral Particles Mediated by PSMAL1-PEG₆-FITC on MDA-PSMA Tumors In Vitro

This study was carried out to assess if the combination of PSMAL1-PEG₆-FITC administered with TagCAR T cells generated as described in Example 42 was effective in treating PSMA-positive tumor cells in vitro.

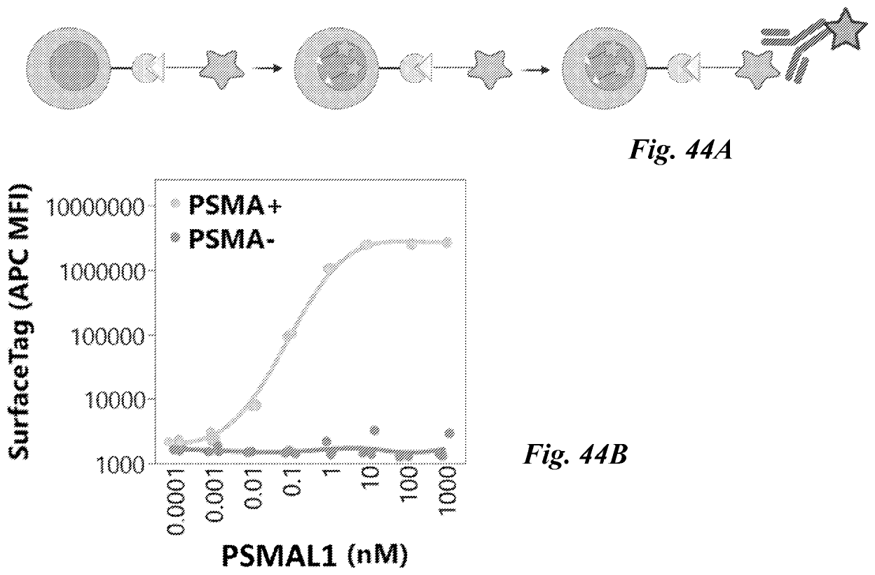

PSMAL1-PEG₆-FITC strongly localizes on the surface of PSMA-positive tumor cells. To characterize the on-cell binding affinity of PSMAL1-PEG₆-FITC to PSMA-expressing tumor cells, different concentrations of PSMAL1-PEG₆-FITC were incubated with MDA-MB-321 tumor cells that either over-expressed PSMA (PSMA⁺) or were wildtype (PSMA−). Levels of surface fluorescein antigen on MDA-MB-321 tumor cells were detected using an anti-fluorescein antibody (FIG. 44A). As shown in FIG. 44B, compared to the PSMA− control, PSMAL1-PEG₆-FITC was detected on the surface of PSMA⁺ tumor cells at increasing concentrations of ligand. Its affinity, or Kd, was calculated to be 1.292 nM, and its total antigen amount, or Bmax, on PSMA⁺ tumor cells was 2.6×10⁶ MFI. These data suggest that PSMAL1-PEG₆-FITC specifically localized on the surface of PSMA-expressing tumors.

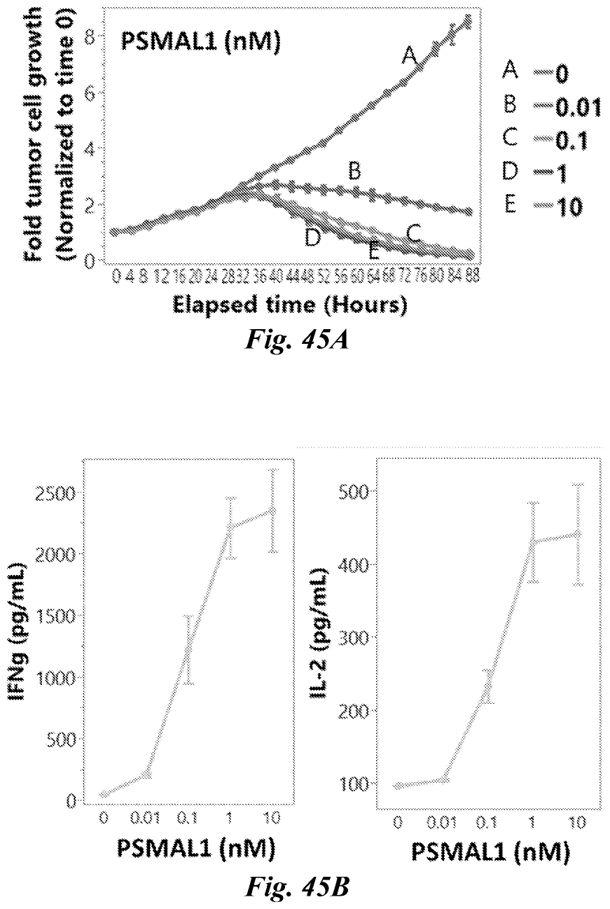

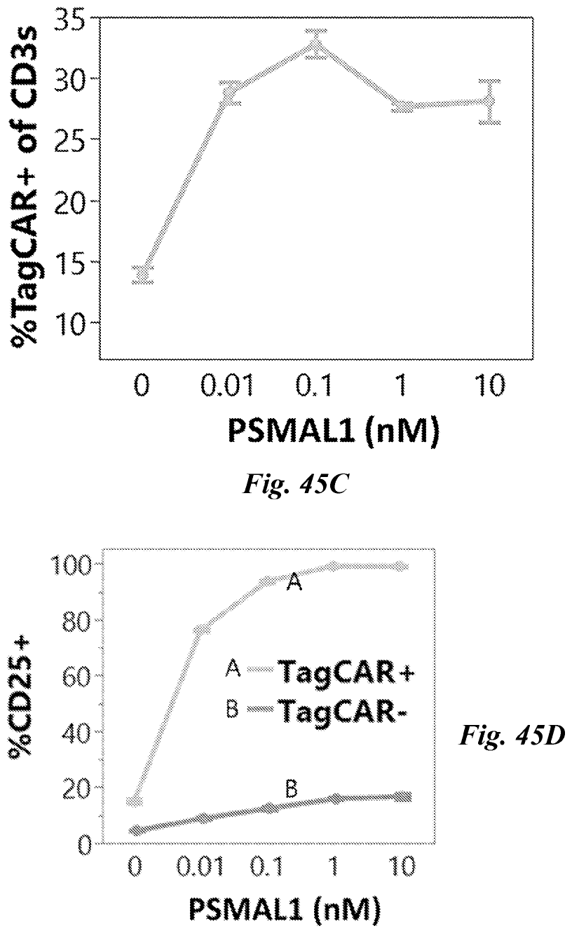

PSMAL1-PEG₆-FITC elicits dose-dependent TagCAR T cell function in vitro. The efficacy of TagCAR T cells generated as described in Example 42 mediated by PSMAL1-PEG₆-FITC on PSMA-positive tumor cells was investigated in vitro. The normalized fold tumor cell growth of PSMA-overexpressing (PSMA⁺) MDA-MB-231 tumor cells was measured every four hours during an 88-hour coculture of PSMA⁺ MD-MB-231 tumor cells and different concentrations of PSMAL1-PEG₆-FITC in the presence of TagCAR T cells generated as described in Example 42. As shown in FIG. 45A, tumor size decreased in the presence of PSMAL1-PEG₆-FITC, as compared to a negative control without the presence of PSMAL1-PEG₆-FITC.

In addition, cytokine levels were measured in culture supernatants 24 hours following the addition of TagCAR T cells generated as described in Example 42 and different concentrations of PSMAL1-PEG₆-FITC to PSMA MD-MB-231 tumor cells. As shown in FIG. 45B, levels of both interferon gamma (IFNg) and interleukin 2 (IL-2) increased with increasing doses of PSMAL1-PEG₆-FITC.

The presence of TagCAR+ cells (FIG. 45C) and presence of CD25+(FIG. 45D) in CD3+ T cells were also measured after 88 hours of coculture with PSMA⁺ MDA-MB-231 tumor cells with different concentrations of PSMAL1-PEG₆-FITC. CD3+ T cells with TagCAR had higher percentages of CD25+ as compared to CD3+ T cells without TagCAR.

Together, these data suggest TagCAR T cells can mediate dose-dependent cytolytic activity and cytokine release to PSMA-positive tumor cells, as well as inhibit growth of PSMA-positive tumors.

### Example 44

### FAP-FITC Mediates Killing of hFAP+ Tumor Cells by Anti-FITC CAR-T Cells

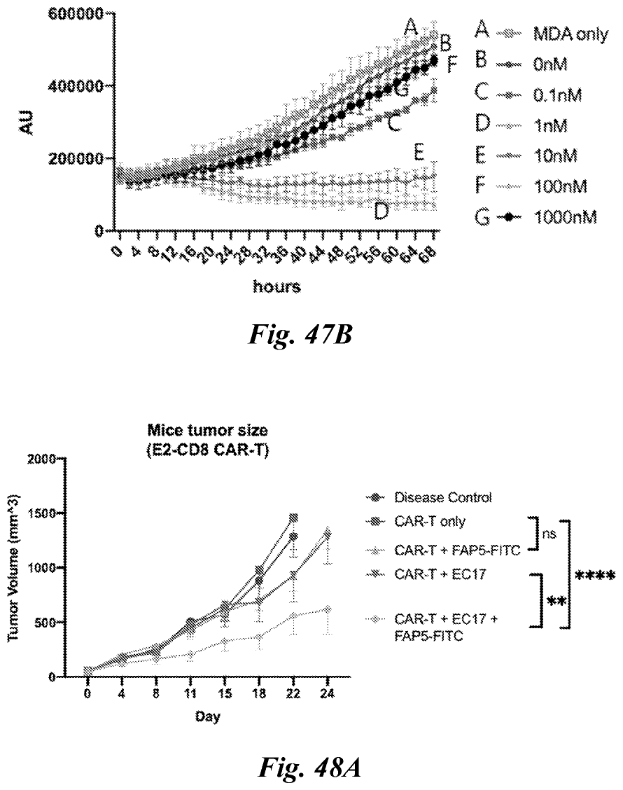

MDA-MB-231-hFAP-mCh cells were seeded on 96-well plates (~7,000 cells/well) overnight. 4M5.3 CAR-T cells were added to the target cells on day 18 at 1:1 effector:target cell ratio. FAP5-PEG₁₆-FITC was added to the target cells and CAR-T cells at different concentrations (0 nM, 0.1 nM, 1 nM, 10 nM, 100 nM, or 1,000 nM, all in triplicate) and incubated for 24 hours. Live cells were either determined by mCherry-positive cells by flow cytometry or mCherry-positive surface area measured by Incucyte, taking pictures every two hours. Percent killing was determined by (1−(live cells)/(live cells in tumor cell only well))*100%. The results are shown in FIGS. 47A and 47B.

As shown in FIGS. 47A-47B, FAP5-FITC mediated good killing of human FAP+ cells. The killing was maximal at 1 nM-10 nM of FAP5-FITC.

The above experiment was repeated with hFAP-over-expressing human fibrosarcoma cells. Similar results were obtained.

### Example 45

### FAP5-FITC Mediates FAP+ Tumor Elimination Via 4M5.3 or E2 CAR-T Cells without Toxicity

MDA-MB-231-hFAP cells were subcutaneously injected into NSG mice and allowed to grow to 150 mm³. Then 4M5.3 or E2 CAR-T cells (8 million) were injected intravenously, and FAP5-PEG₈-FITC was injected three times per week at 400 nmol/kg. The following five groups of mice (n=6) were used:
Disease control group (tumor cells only),
4M5.3 CAR-T cells only group (4M5.3 CAR-T cells and PBS),
4M5.3 CAR-T cells and FAP5-PEG₈-FITC group (dosing three times/week),
E2 CAR-T cells only group (E2 CAR-T cells and PBS), and
E2 CAR-T cells and FAP5-PEG₈-FITC group (dosing three times/week).

E2 CAR-T cells target and attack cancer cells expressing the estrogen receptor alpha (ERα), also known as E2, and are designed to recognize and bind cancer cells that overexpress ERα, which is often found in hormone receptor-positive breast cancer and other hormone-dependent cancers.

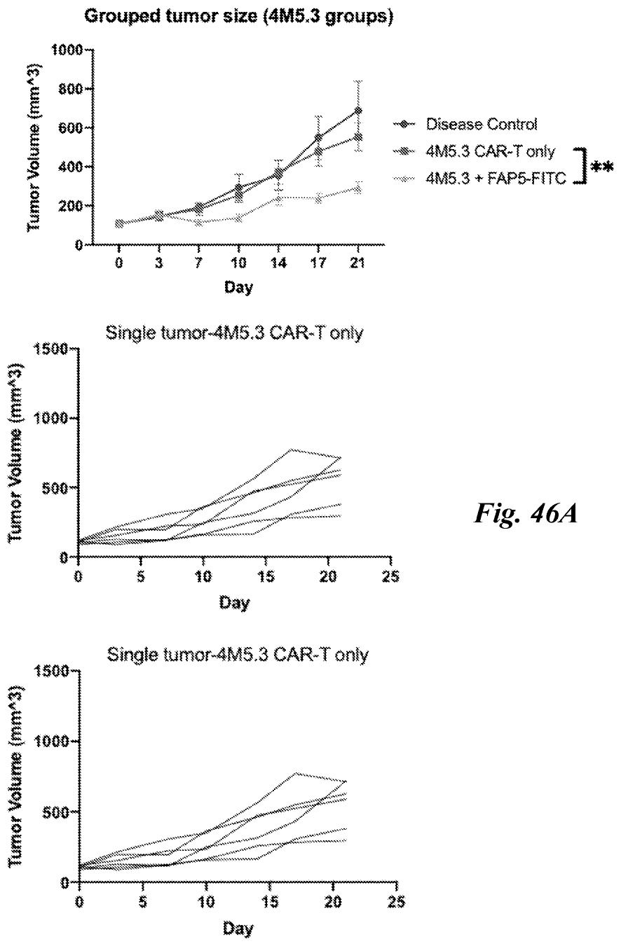

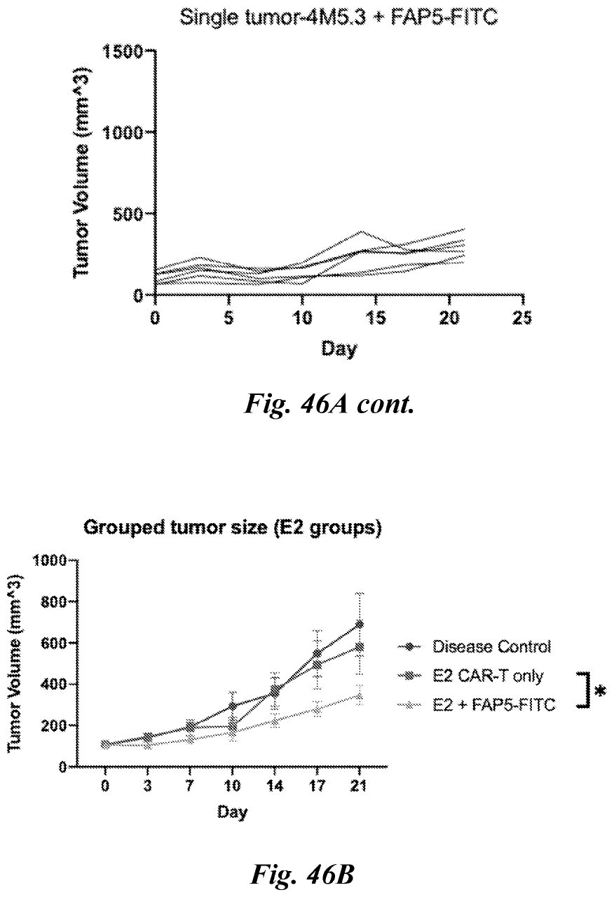

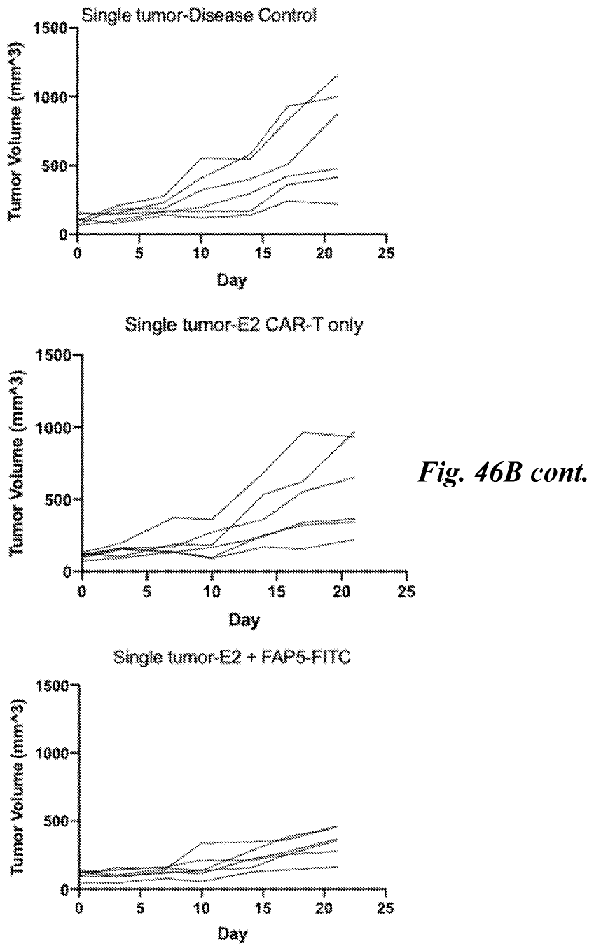

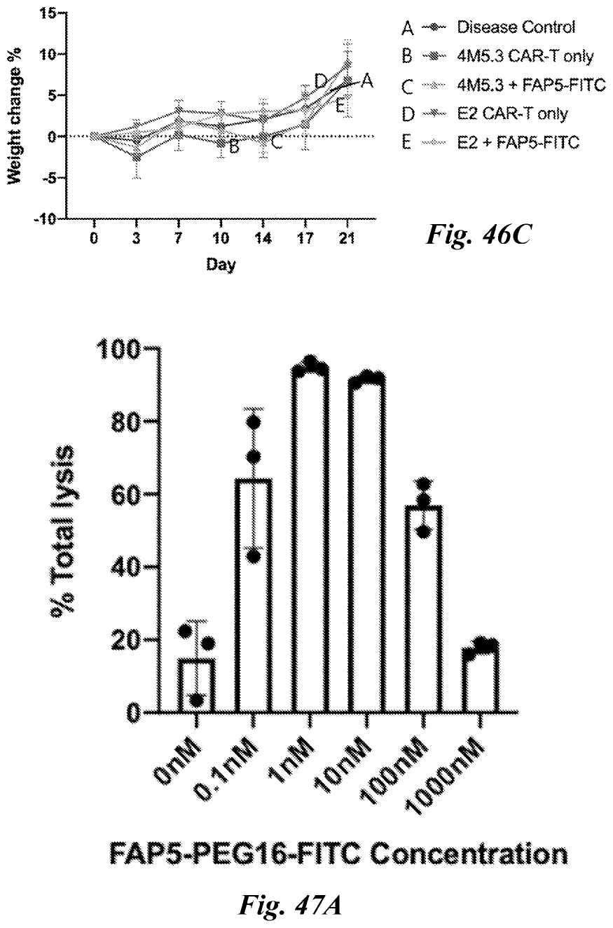

The results are shown in FIGS. 46A-46C. As shown in FIG. 46A, FAP5-PEG₈-FITC was able to mediate FAP+ tumor elimination via 4M5.3 CAR-T cells. As shown in FIG. 46B, FAP5-PEG₈-FITC was able to mediate FAP+ tumor elimination via E2 CAR-T cells. As shown in FIG. 46C, treatment with FAP5-PEG₈-FITC did not mediate any toxicity in mice with either 4M5.3 CAR-T cells or E2 CAR-T cells.

### Example 46

### FAP-FITC Treatment Enhanced CAR-T Cell Activation and Proliferation and Reduced Tumor Cell Proliferation by Eliminating FAP+ CAFs in the TME

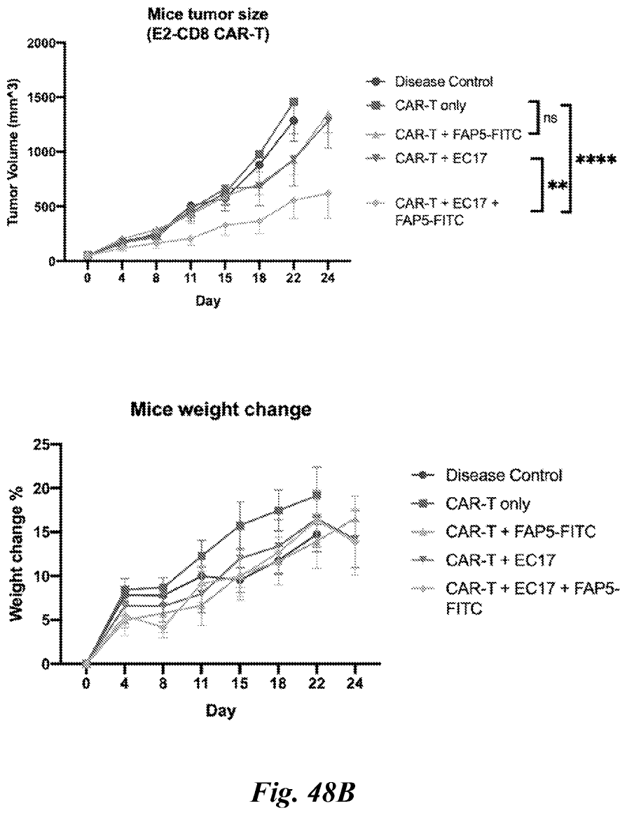

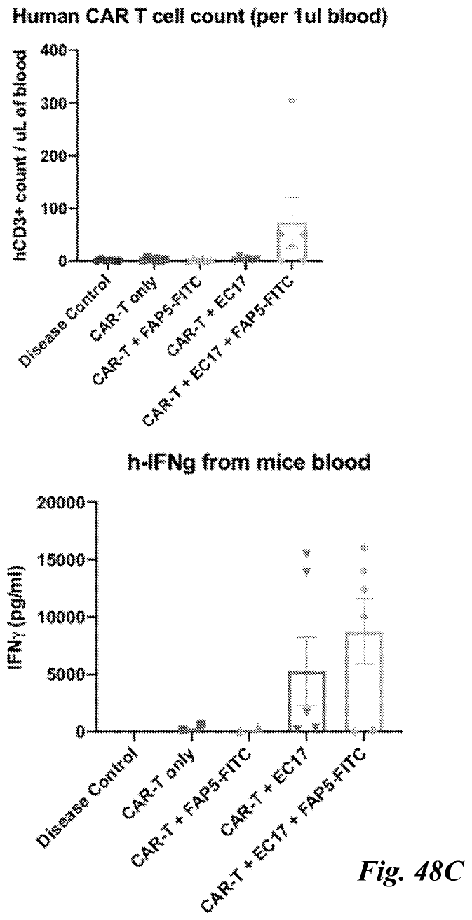

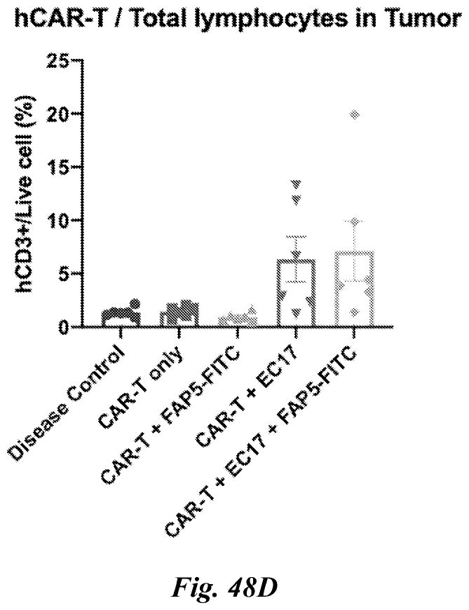

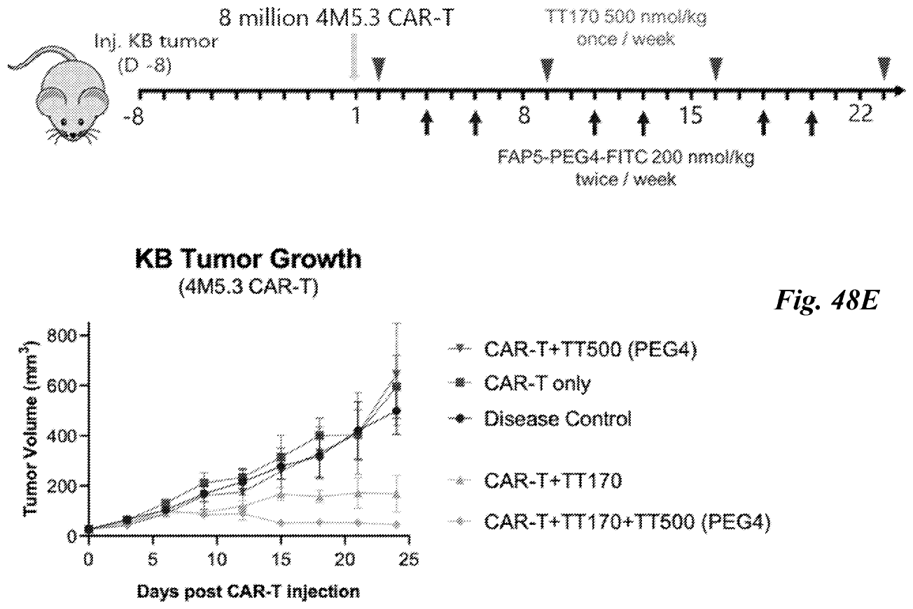

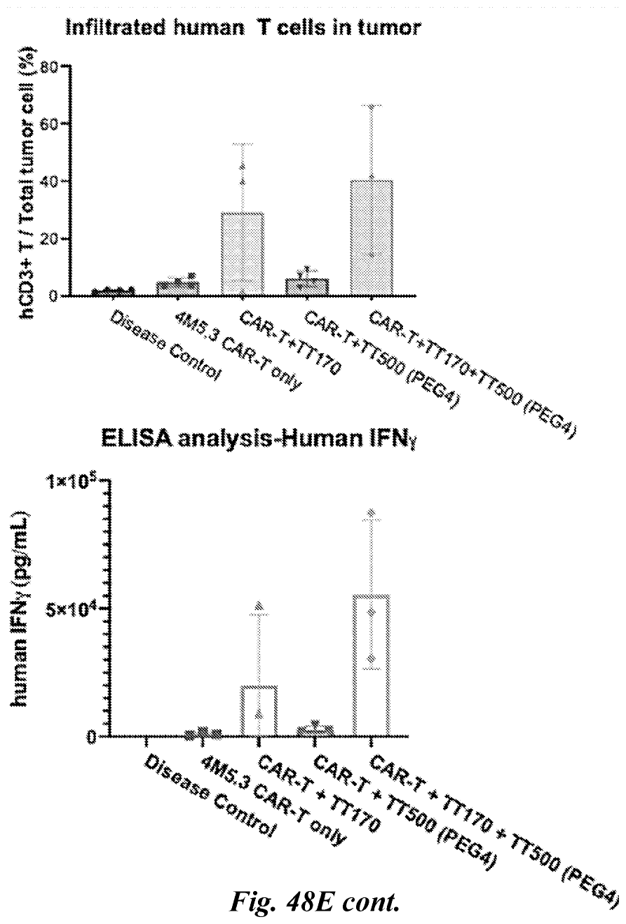

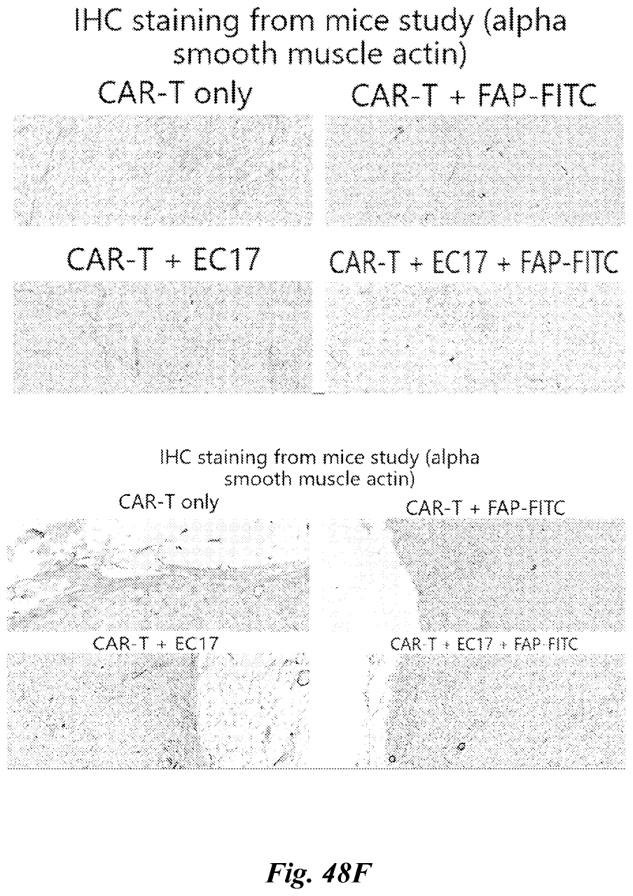

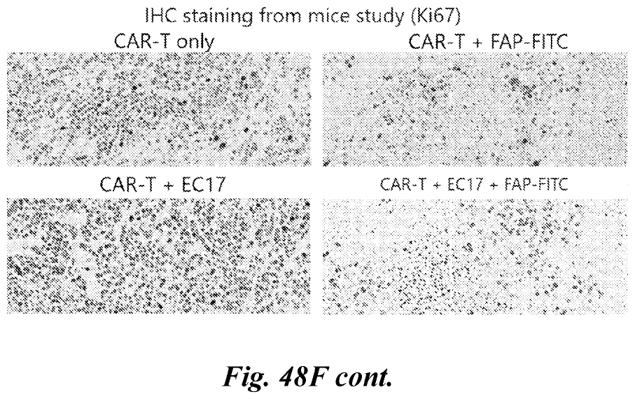

Mice blood samples were harvested at the endpoint of the in vivo study via cardiac puncture. Mice blood samples were then centrifuged at ~1,000 g for 10 minutes, after which serum was obtained for the detection of hIFNγ via ELISA assay (FIG. 48C). The blood samples were then incubated in red blood cell (RBC) lysis buffer according to manufacturer's protocol, washed, stained for zombie violet (i.e., a live/dead staining), and anti-human CD3 antibody (i.e., to detect human CAR-T cells) (FIG. 48C). The combination therapy of FAP-FITC+EC17 suppressed KB tumor growth without noticeable toxicity (FIGS. 48A-48B).

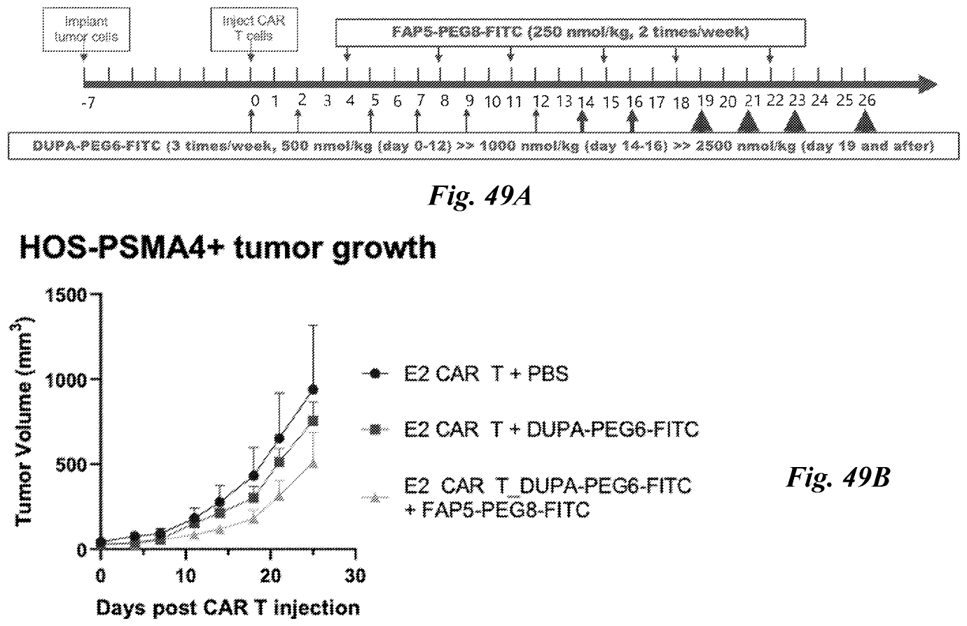

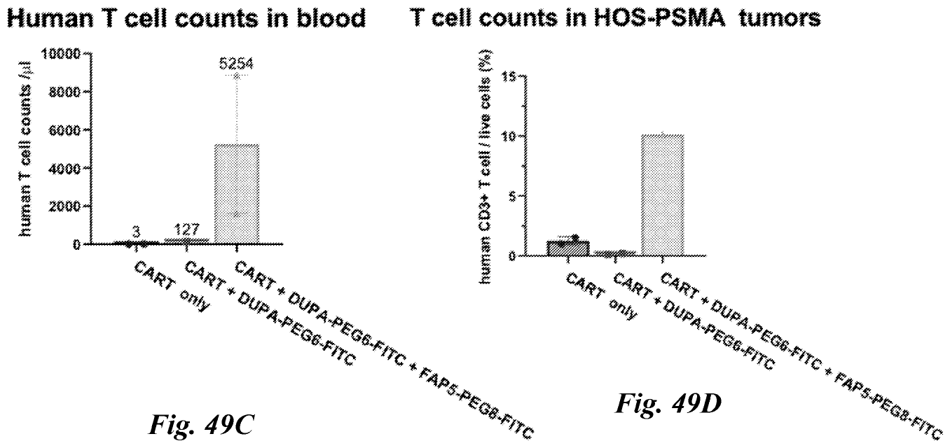

Mice tumors were harvested at the end point of the study after euthanasia of the mice. A small piece from each tumor sample was cut and fixed in 10% formalin for IHC slide preparation, and the rest of the tumor was digested using the human tumor dissociation kit from Miltenyi Biotec according to the manufacturer's protocol (Milteny Biotec, Bergisch Galdbach, Germany). For IHC slides, the CAFs were stained using anti-mouse alpha smooth muscle actin (a marker for CAFs), and the cancer cells were stained with anti-mouse Ki67 as a proliferation marker of cancer cells. Tumor cells from tumor dissociation were then stained for anti-human CD3 antibody for detection of human CAR-T cells. The results are shown in FIGS. 48A-49D.

As shown in FIGS. 48C-48D, mice treated with combination therapy had more CAR-T cell proliferation and higher levels of hIFNγ in their blood. CAR-T infiltration in KB tumors was similar between the EC17 treatment group and the EC17+FAP5-FITC treatment group; these results could be due to late-stage harvest of the tumor and most CAR-T cells no longer functioning or no longer viable. IHC staining with alpha smooth muscle actin showed a decrease in CAFs at the edges and in the middles of the tumors after treatment with FAP-FITC and a decrease in tumor proliferation rate (FIG. 48F). The decrease in tumor proliferation rate was due to CAF-killing, which eliminates the release of multiple growth factors from the CAFs to stimulate tumor cell growth. As shown in FIG. 48E, combination therapy with a 4M5.3 anti-FITC CAR-T cells showed similar results.

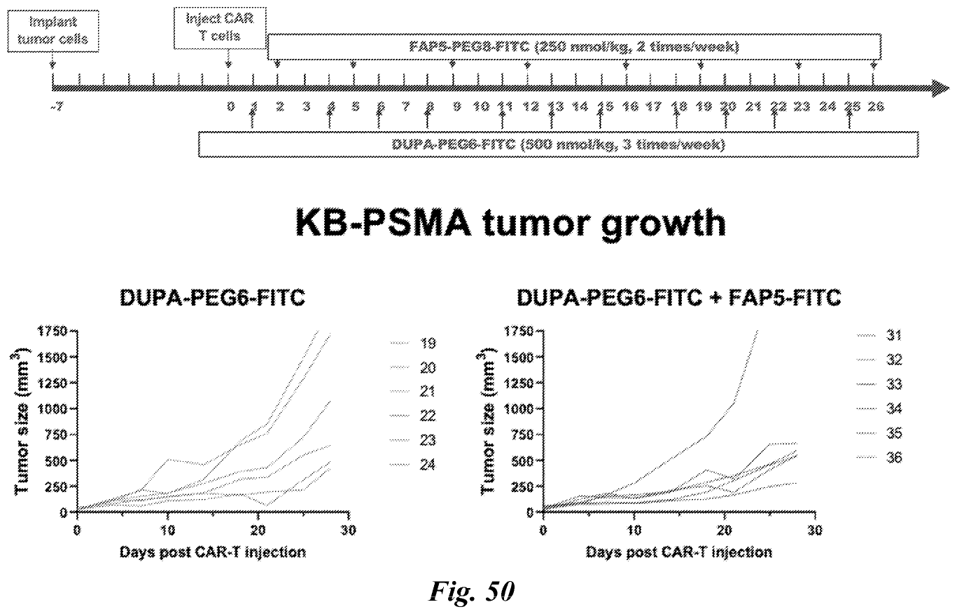

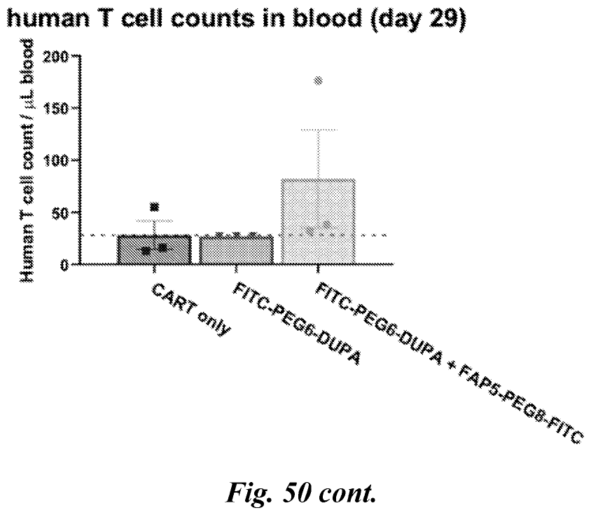

As shown in FIGS. 49A-49D, combination therapy with FAP5-PEG₈-FITC and DUPA-PEG₆-FITC also enhanced tumor elimination. Mice treated with combination therapy showed higher CAR-T cell proliferation in blood and in the tumor. Similar results were obtained in a different PSMA⁺ cold tumor model (KB-PSMA) (see FIG. 50).

### Example 47

### In Vivo Efficacy of Aza-PEG₆-FITC and orthoCAL-PEG₆-FITC in Combination with EC17 (Folate-FITC)

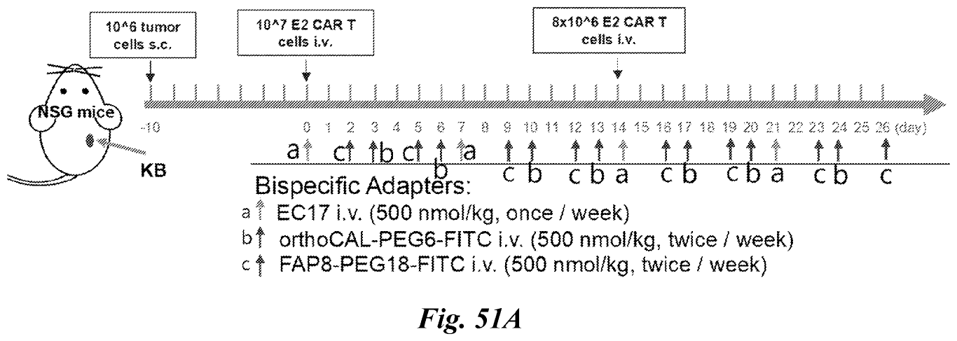

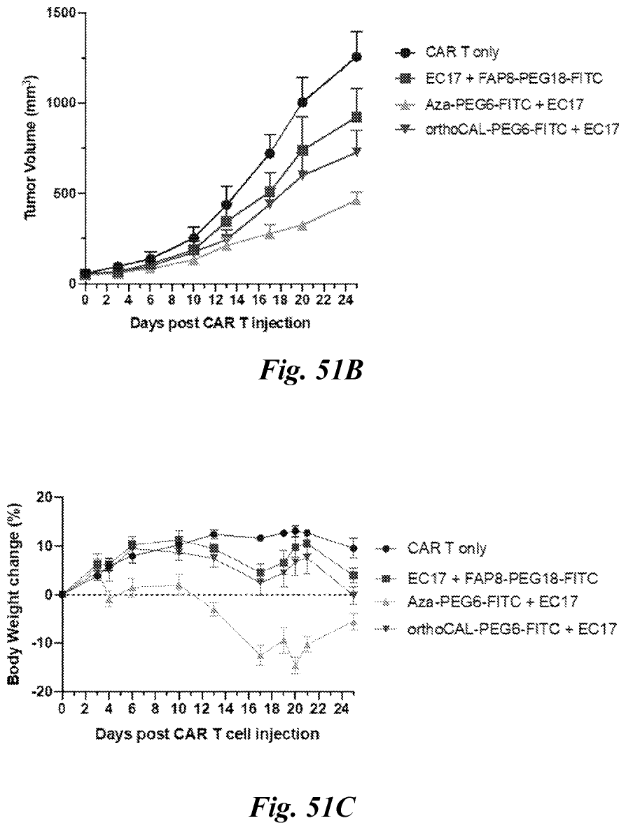

KB cells (1 million) were implanted into each NSG mouse by subcutaneous injection. When the tumor volumes reached about 50 mm³, the treatment groups were injected with 10 million anti-FITC CAR T-cells and indicated bispecific adapters as shown in FIG. 51A. The tumor volume and body weight were monitored regularly. Tumor volume was calculated using the formular: (length*width²)/2. The results are shown in FIGS. 51A-51C.

FIG. 51B is a graph of days post-CAR-T cell injection vs. tumor volume (mm³), which shows tumor growth curves of different treatment groups. Aza-PEG₆-FITC in combination with EC17 significantly inhibited the growth of KB tumors. OrthoCAL-PEG₆-FITC in combination with EC17 also showed slightly better inhibition of the growth of KB tumors. FIG. 51C is a graph of days post-CAR-T cell injection vs. body weight change (%), which shows body weight changes of mice in different treatment groups. Aza-PEG₆-FITC and EC17 induced body weight loss in mice. The body weight loss could be due to cytokine release from expanded CAR-T cells. The toxicity can be minimized by optimizing the dosing of the adapter.

### Example 48

### In Vivo Efficacy of Aza-PEG₆-FITC in Combination with FAP8-PEG₁₈-FITC

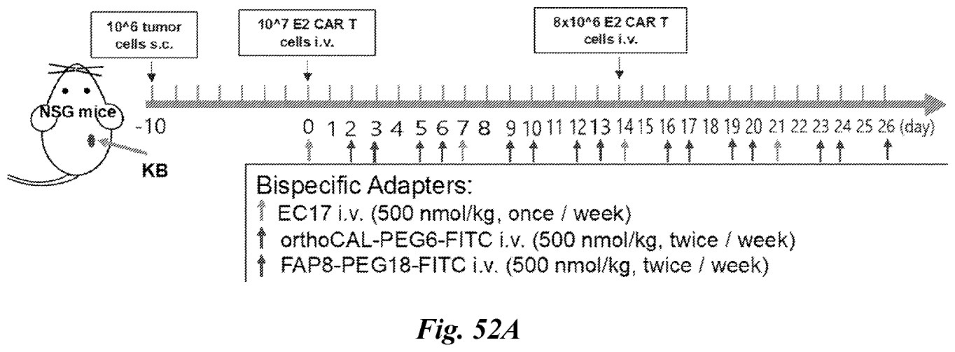

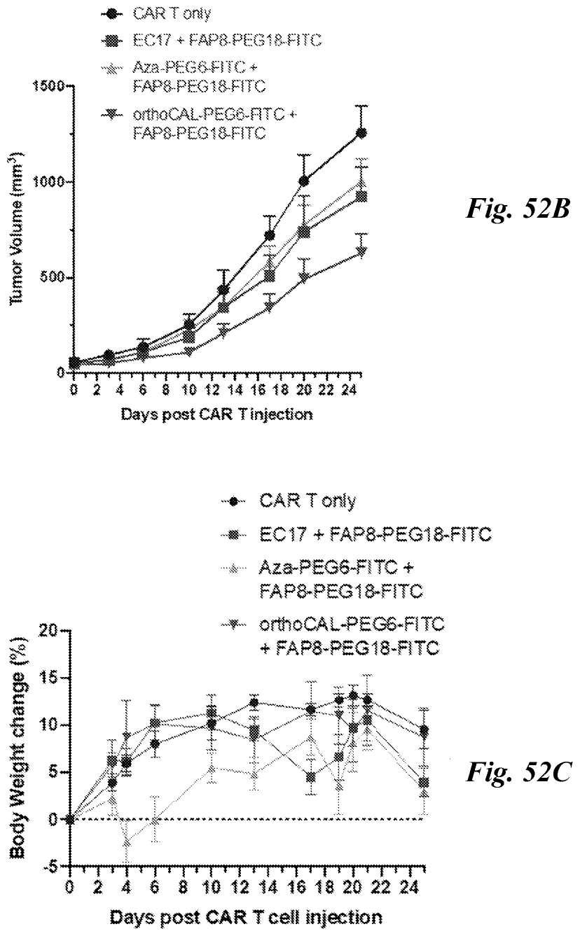

KB cells (1 million) were implanted into each NSG mouse by subcutaneous injection. When the tumor volumes reached about 50 mm³, the treatment groups were injected with 10 million anti-FITC CAR T-cells and indicated bispecific adapters as shown in FIG. 52A. The tumor volume and body weight were monitored regularly. Tumor volume was calculated using the formular: (length*width²)/2. The results are shown in FIGS. 52B-52C.

FIG. 52B is a graph of days post-CAR-T cell injection vs. tumor volume (mm³), which shows tumor growth curves of different treatment groups. Aza-PEG₆-FITC in combination with FAP8-PEG₁₈-FITC slightly inhibited the growth of KB tumors. The efficacy was similar to the combination of EC17 and FAP8-PEG₁₈-FITC. OrthoCAL-PEG₆-FITC in combination with FAP8-PEG₁₈-FITC showed better efficacy on inhibiting the growth of KB tumors. FIG. 52C is a graph of days post-CAR-T cell injection vs. body weight change (%), which shows body weight changes of mice in different treatment groups. All the combinations did not induce significant body weight loss in the treated mice.

### Example 49

### In Vivo Efficacy of Aza-PEG₆-FITC in Combination with EC17 (Folate-FITC) and FAP8-PEG₁₈-FITC

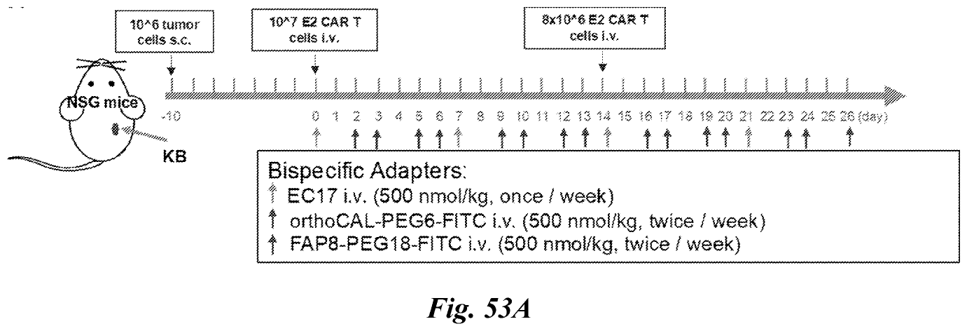

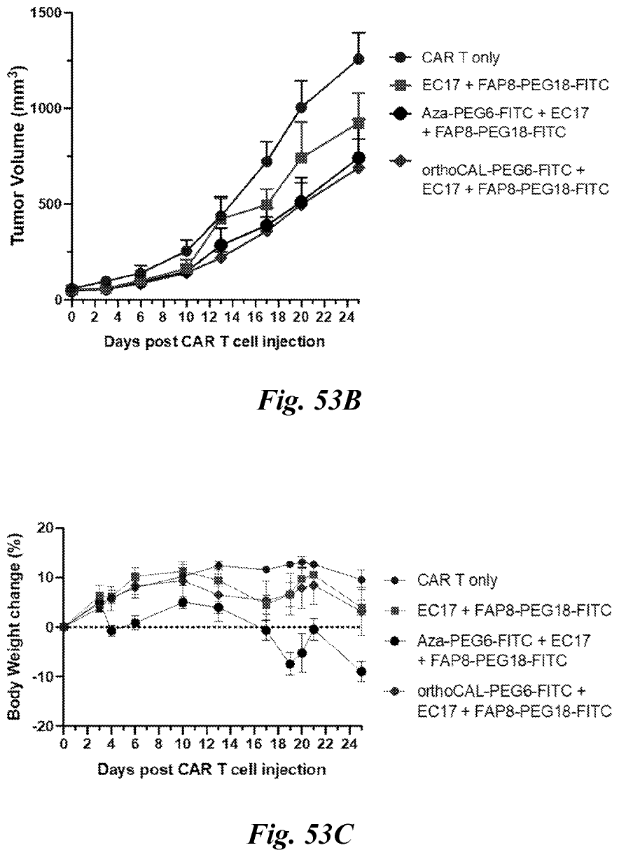

KB cells (1 million) were implanted into each NSG mouse by subcutaneous injection. When the tumor volumes reached about 50 mm³, the treatment groups were injected with 10 million anti-FITC CAR T-cells and indicated bispecific adapters as shown in FIG. 53A. The tumor volume and body weight were monitored regularly. Tumor volume was calculated using the formular: (length*width²)/2. The results are shown in FIGS. 53B-53C.

FIG. 53B is a graph of days post-CAR-T cell injection vs. tumor volume (mm³), which shows growth curves of different treatment groups. Aza-PEG₆-FITC in combination with FAP8-PEG₁₈-FITC and EC17 has similar efficacy as orthoCAL-PEG₆-FITC in combination with FAP8-PEG₁₈-FITC and EC7. They both showed slightly better efficacy than the combination of EC17 and FAP8-PEG₁₈-FITC on inhibiting the growth of KB tumors. FIG. 53C is a graph of days post-CAR-T cell injection vs. body weight change (%), which shows body weight changes of mice in different treatment groups. Mice injected with the combination of Aza-PEG₆-FITC, EC17 and FAP8-PEG₁₈-FITC showed body weight loss in the treatment. The body weight loss could be due to cytokine release from expanded CAR-T cells. The toxicity can be minimized by optimizing the dosing of the adapter.

### Example 50

### IHC of KB Tumor and MDA-MB231 Tumor

To characterize and compare infiltrating FAP+ CAFs in cold KB tumors and hot MDA-MB-231 tumors, KB tumors or MDA-MB-231 tumors were implanted on NOD scid gamma (NSG) mice, and the mice were treated with the universal anti-FITC CAR-T and EC17 (folate-fluorescein). Both tumors were then harvested from the mice, fixed with 10% formalin overnight, and rinsed with 70% ethanol for tumor fixation. Then, the tumor cells were sent for IHC staining for either anti-human CD3 antibody to detect human CAR-T cells or anti-mouse FAP antibody to detect mouse FAP+ CAFs.

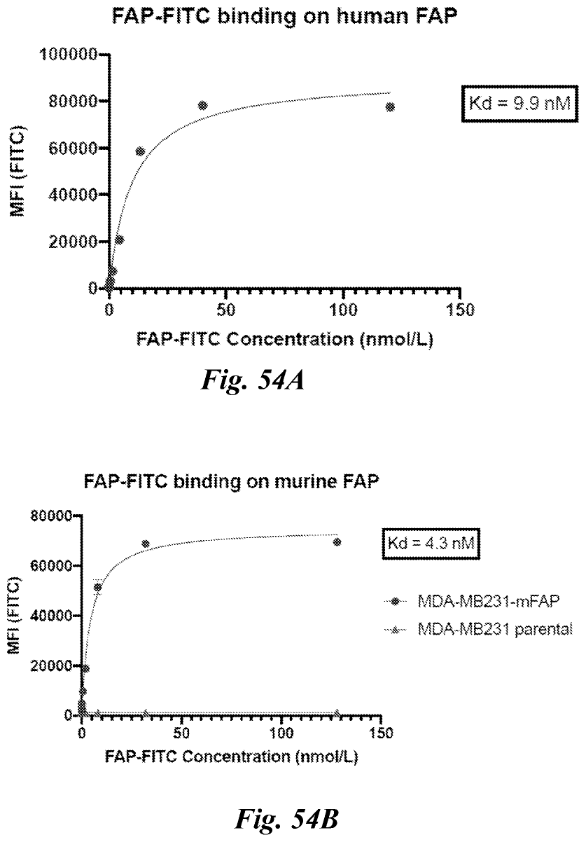

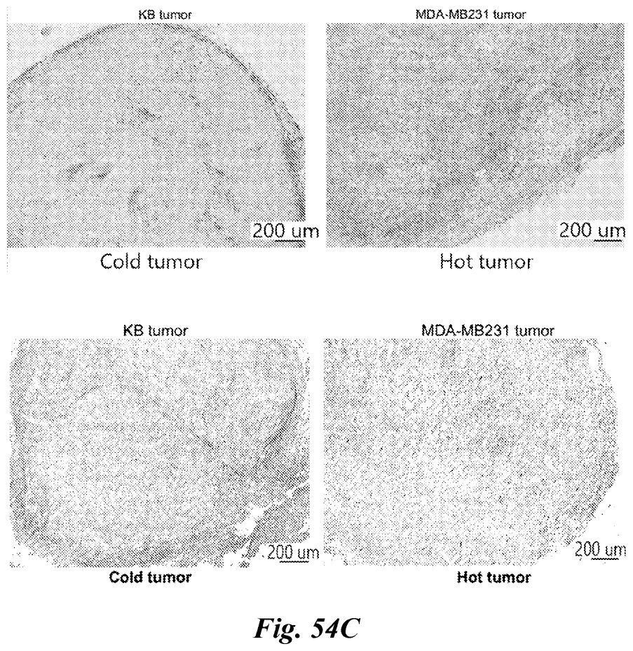

As shown in FIG. 54C, results from the IHC staining demonstrated a lack of CAR-T cell infiltration in the immunologically cold KB tumor but not in the hot MDA-MB-231 tumor. Elevated infiltration of FAP+ CAFs was found in cold KB tumor, forming a physical barrier around the tumor, but the infiltrating CAFs were significantly less on the hot MDA-MB-231 tumor. It was hypothesized that these infiltrating fibroblasts are one of the reasons for an immunologically cold solid tumor.

### Example 51

### FAP8-FITC Binding Affinity

To determine the binding affinity of FAP8-FITC on human FAP (hFAP) and murine FAP (mFAP), MDA-MB231-FAP cells (~0.1 million cells) were suspended and incubated with increasing concentrations of FAP8-FITC at room temperature for one hour. Cells were washed twice and subjected to flow cytometry for Bmax.

As shown in FIGS. 54A-54B, the FAP8-FITC binds hFAP and mFAP with high affinity and specificity. A decrease in Kd was observed with increasing linker length.

### Example 52

### FAP8-FITC Efficacy in Killing FAP+ Cells In Vitro

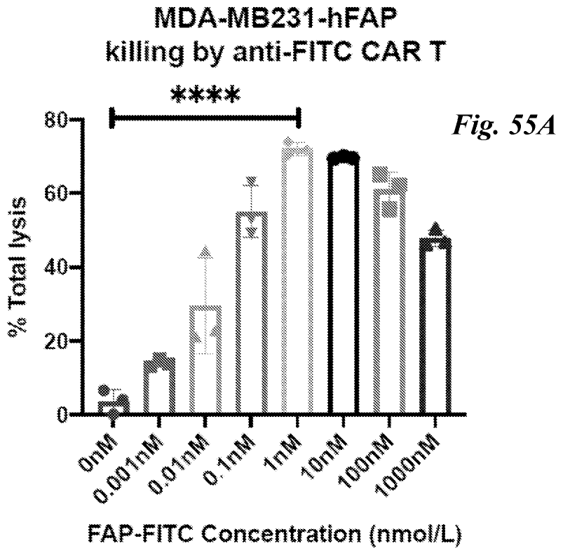

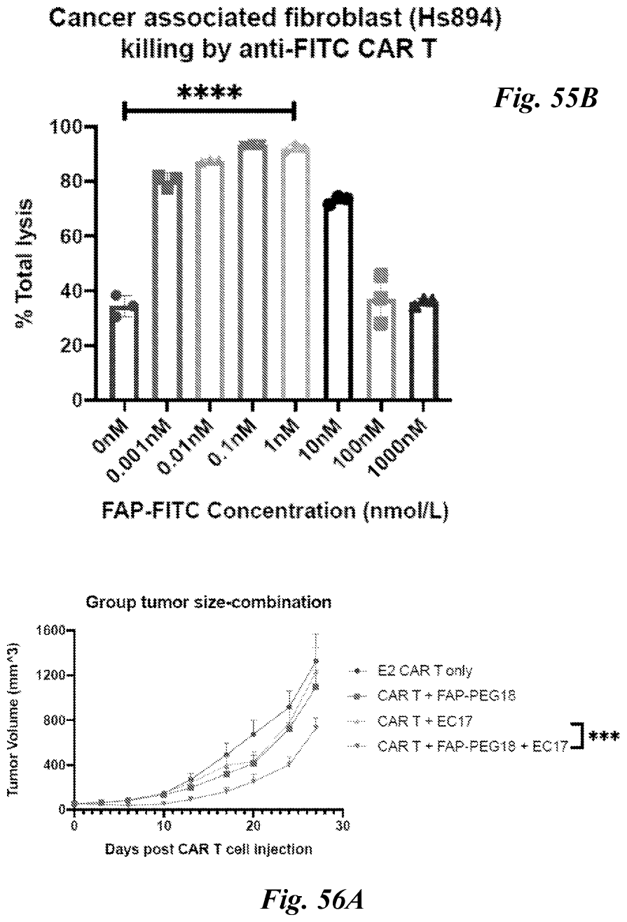

To determine the efficacy of FAP8-FITC in killing FAP+ cells in vitro, MDA-hFAP cells (~7,000 cells/well) and Hs894 CAFs (~4,000 cells/well) were separately seeded on 96-well plates overnight. FAP8-FITC was added to the target cells at various concentrations (0 nM, 0.001 nM, 0.01 nM, 0.1 nM, 1M nM, nM, 100 nM, and 1,000 nM: n=3). E2 CAR T was added to the target cells at a 1:3 E:T ratio for MDA-hFAP and a 2:1 E:T ratio for CAFs and co-incubated for 48 hours. Live cells were measured by CellTrace+ cells via flow cytometry. The FAP-FITC mediated killing of FAP-expressing cells. See FIGS. 55A-55B.

### Example 53

### Effect of FAP8-FITC and EC17 on KB Tumor

To compare FAP8-FITC with different PEG lengths in vivo, KB (tumor FAP⁻, FR⁺; CAF FAP⁺) tumors were implanted on NSG mice and allowed to grow to approximately 50 mm³. Mice were injected intravenously with 10×10⁶ E2 CAR T (VPN 404) with FAP8-FITC and/or EC17. Tumors were harvested with the disease control reached 1500 mm³ or when tumors were eliminated in the treatment group. The mice groups were as follows:
Group 1 (n=5) (FAP-FITC adaptor only): FAP8-FITC only (no CAR T)
Group 2 (n=6) (CAR T only): E2 CAR T+PBS
Group 3 (n=6) (FAP-FITC only): E2 CAR T+FAP-FITC
Group 4 (n=6) (EC 17 only): E2 CAR T+EC17
Group 5 (n=6) (FAP-FITC+EC17): E2 CAR T+FAP8-FITC+EC17

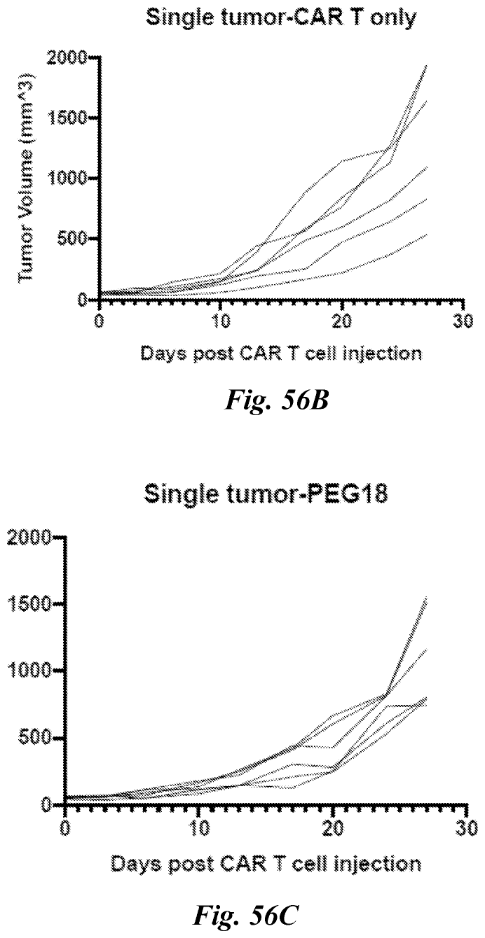

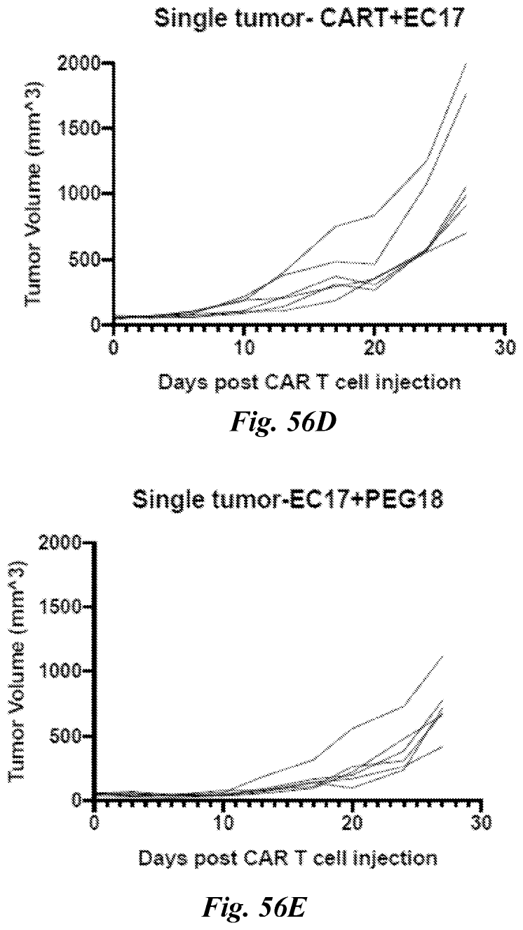

The results show that FAP8-FTC mediates a combination therapeutic effect in KB tumors. See FIGS. 56A-56E.

### Example 54

### Combination Therapy Yields Better CAR T Proliferation and Activation at Study Mid-Point

Blood was harvested from mice tail veins at study midpoint. Part of the blood was incubated with RBC lysis buffer for 30 minutes on ice and washed three times. The rest of the blood was centrifuged at 1,000 g for 10 minutes to collect serum for human IFNγ ELISA analysis. Cells were then stained with Zombie violet and Fc blocker on ice for 30 minutes and washed twice. Cells were then stained with anti-hCD3 for 30 minutes, washed three times, and subjected to flow cytometry.

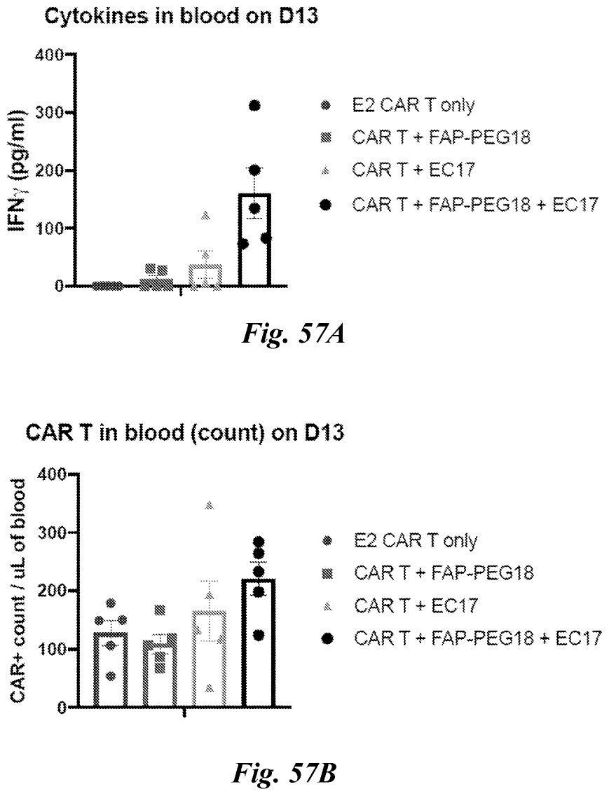

FAP8-FITC with PEG₁₅/PEG₁₈ showed similar cytokine release and CAR T count at study mid-point. See FIGS. 57A-57B.

### Example 55

### Combination Therapy Yields Better CAR T Proliferation and Activation at Study Endpoint

Blood was harvested from mice tail veins at study midpoint. Part of the blood was incubated with RBC lysis buffer for 30 minutes on ice and washed three times. The rest of the blood was centrifuged at 1,000 g for 10 minutes to collect serum for human IFNγ ELISA analysis. Cells were then stained with Zombie violet and Fc blocker on ice for 30 minutes and washed twice. Cells were then stained with anti-hCD3 for 30 minutes, washed three times, and subjected to flow cytometry.

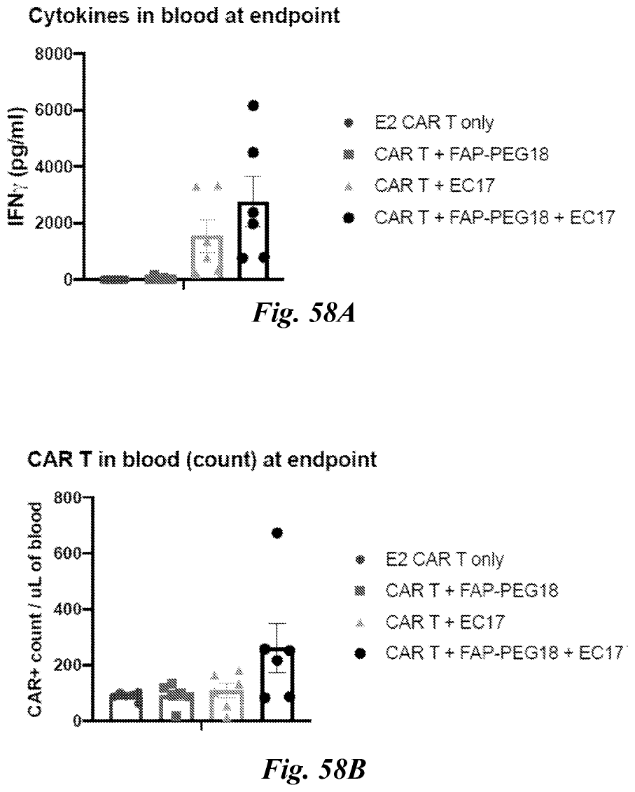

FAP8-PEG₁₈-FITC showed better cytokine release and CAR T count at study endpoint. See FIGS. 58A-58B.

### Example 56

### Combination Therapy Yields Better CAR T Infiltration in KB Tumor at Study Endpoint

Tumors were harvested from mice at endpoint and digested with human tumor dissociation kit (Miltenyi) with 50% Enzyme R to enhance lymphocyte recovery. Cells were then stained with Zombie violet and Fc blocker on ice for 30 minutes and washed twice. Cells were then stained with anti-E2 CAR antibody on ice for 30 minutes, washed three times, and subjected to flow cytometry.

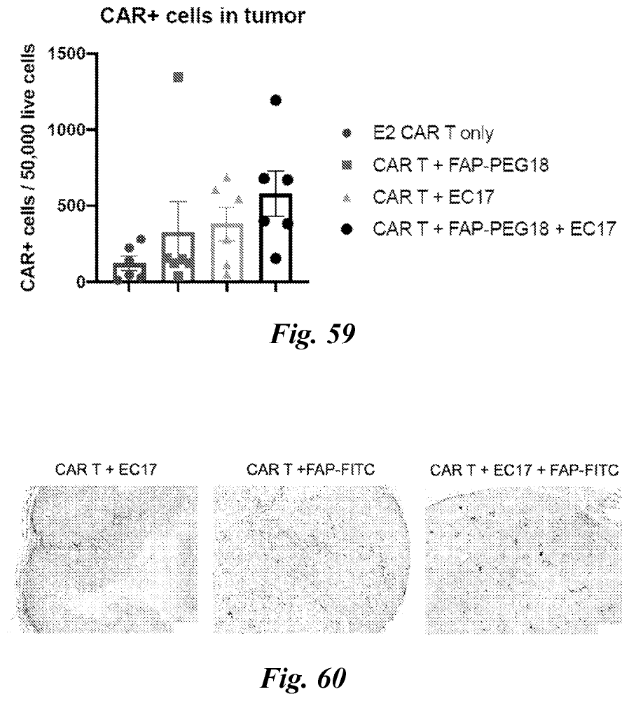

More CAR T cell infiltration was observed with the combination therapy of FAP8-FITC and EC17. See FIG. 59.

### Example 57

### IHC Staining

Tumor samples from the mice study (CAR T+EC17; CAR T+FAP-FITC; and CAR T+EC17+FAP-FITC) were fixed with 10% formalin, paraffined, and sectioned for IHC staining of cancer proliferation marker Ki67. A decrease of cancer proliferation was observed after FAP-FITC treatment, possibly due to elimination of CAFs that were known to secrete various growth factors (FIG. 60).

Staining of human T cell marker CD3 indicated an increase of T cell infiltration after FAP-FITC treatment, possibly due to elimination of CAFs that were known to form a physical barrier to block immune cell function (FIG. 60).

### ENUMERATED EMBODIMENTS OF THE CLAIMED INVENTION

Clause 1. A bispecific adapter comprising the following structure:

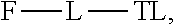

or a pharmaceutically acceptable salt or hydrate thereof, wherein:
F comprises a fluorescein, fluorescein isothiocyanate (FITC), or N-hydroxysuccinimide (NHS)-fluorescein,
L comprises a linker, and
TL comprises a targeting ligand comprising a radical of a fibroblast activation protein (FAP) ligand or a radical of a prostate-specific membrane antigen (PSMA) ligand.

Clause 2. The bispecific adapter of clause 1, wherein the targeting ligand comprises a radical of a FAP ligand comprising a structure of:

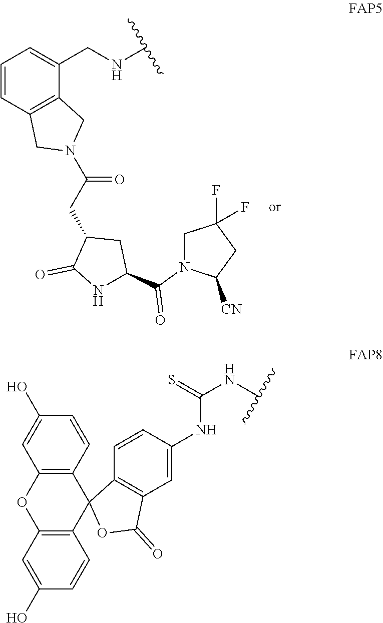

wherein

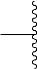

is the point of attachment to the linker.

Clause 3. The bispecific adaptor of clause 2, wherein the targeting ligand comprises a radical of a FAP5 ligand comprising a structure represented by formula I-B:

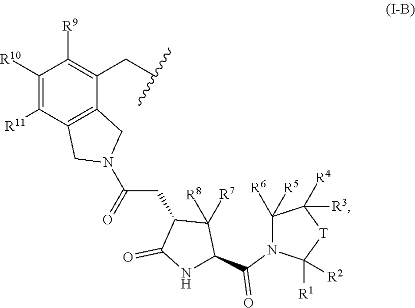

wherein:

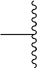

is the point of attachment to the linker;
T is substituted or unsubstituted methylene (—CH₂—), substituted or unsubstituted amino (—NH—), —O—, or —S—;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F and 5-tetrazolyl;
R³ and R⁴ are each independently selected from the group consisting of —H, —OH, F, Cl, Br, I, —C₁₋₆alkyl, —O—C₁₋₆alkyl, and —S—C₁₋₆ alkyl;
R⁵, R⁶, R⁷, and R⁸ are each independently selected from group consisting of H, alkyl and halo; and
R⁹, R¹⁰, and R¹¹ are each independently selected from group consisting of H, —C₁₋₆alkyl, —O—C₁₋₆alkyl, —S—C₁₋₆ alkyl, F, Cl, Br and I.

Clause 4. The bispecific adapter of clause 2, wherein the targeting ligand comprises a radical of a FAP5 ligand comprising a structure represented by the formula I-C:

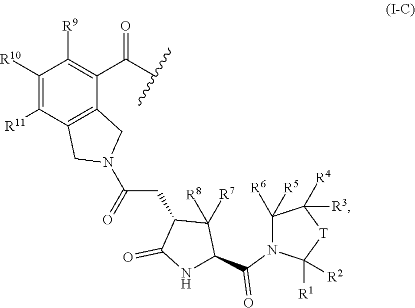

wherein:

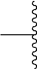

is the point of attachment to the linker;
T is substituted or unsubstituted methylene (—CH₂—), substituted or unsubstituted amino (—NH—), —O—, or —S—;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, and 5-tetrazolyl;
R³ and R⁴ are each independently selected from the group consisting of —H, —OH, F, Cl, Br, I, —C₁₋₆alkyl, —O—C₁₋₆alkyl, and —S—C₁₋₆alkyl;
R⁵, R⁶, R⁷, and R⁸ are each independently selected from group consisting of H, alkyl and halo; and
R⁹, R¹⁰, and R¹¹ are each independently selected from group consisting of H, —C₁₋₆alkyl, —O—C₁₋₆alkyl, —S—C₁₋₆ alkyl, F, Cl, Br and I.

Clause 5. The bispecific adapter of clause 2, wherein the targeting ligand comprises a radical of a FAP8 ligand comprising a structure:

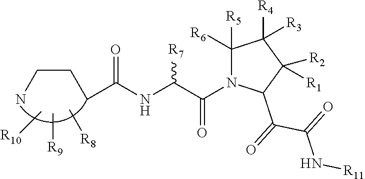

wherein:

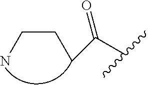 [SMILES: *C(=O)CCCN]

represents a functionalized 5- to 10-membered N-containing aromatic or non-aromatic mono- or bi-cyclic heterocycle, which optionally further comprises 1-3 heteroatoms selected from O, N, and S;
R₁ and R₂ are independently selected from the group consisting of —H, -D, —OH, —F, —Cl, —Br, —I, —C₁₋₆ alkyl, —O—C₁₋₆ alkyl, and —S—C₁₋₆ alkyl;
R₃ and R₄ are independently selected from the group consisting of —H, —OH, —F, —Cl, —Br, —I, —C₁₋₆ alkyl, —O—C₁₋₆ alkyl, and —S—C₁₋₆ alkyl;
R₅ and R₆ are independently selected from group consisting of —H, —OH, —F, —Cl, —Br, —I, —C₁₋₆ alkyl, —O—C₁₋₆ alkyl, and —S—C₁₋₆ alkyl;
R₇ is selected from the group consisting of —H, -D, OH, CH₂═, —CH₃, CH₃CH₂—, (CH₃)₂CH—(CH₃)₃C—, —CH₂Ph, and substituted —CH₂Ph;
R₈-R₁₀ are independently selected from group consisting of —H, —OH, —F, —Cl, —Br, —I, —NO₂, —SO₃H, —SO₂NH₂, —NH₂, —N₃, —NH═NH, —C₁₋₆ alkyl, —O—C₁₋₆ alkyl, and —S—C₁₋₆ alkyl; and
R₁₁ is selected from the group consisting of —H, -D, C₁-C₁₀ alkyl, C₃-C₁₀ cycloalkyl, adamantyl,

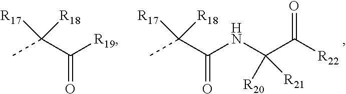

substituted or unsubstituted aryl, substituted or unsubstituted C₇-C₂₀ alkyl aryl, wherein the aryl is:

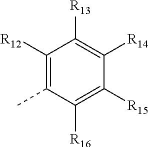

wherein:
R₁₂ and R₁₆ are independently selected from the group consisting of —H, -D, halogen, C₁-C₃ alkyl, C₁-C₃ alkoxy, —CF₃, and —C(═O)—OR₂₃, wherein R²³ is selected from the group consisting of H, D, halogen, C₁-C₄ alkyl, and C₁-C₃ alkoxy;
R₁₃, R₁₄ and R₁₅ are independently selected from the group consisting of —H, -D, halogen, —OMe, C₁-C₃ alkyl, C₁-C₃ alkoxy, —CF₃, and —C(═O)—OR₂₃, wherein R²³ is selected from the group consisting of —H, -D, halogen, C₁-C₄ alkyl, and C₁-C₃ alkoxy;
R₁₇, R₁₈, R₂₀, and R₂₁ are independently selected from —H and —CH₃; and
R₁₉ and R²² are independently selected from the group consisting of phenyl, dimethoxyphenyl, and aryl.

Clause 6. The bispecific adapter of clause 1, wherein the targeting ligand comprises a radical of a PSMA ligand and is ((S)-5-amino-1-carboxypentyl)carbamoyl)-L-glutamic acid (PSMAL1) or 2-[3-(1,3-dicarboxypropyl)ureido]pentanedioic acid (DUPA).

Clause 7. The bispecific adapter of any one of clauses 1-6 wherein the linker comprises or consists essentially of polyethylene glycol (PEG) or a PEG derivative such as, optionally: PEG₃ to PEG₁₆ and, optionally, PEG₄ to PEG₁₅ or PEG₃ to PEG₁₂; PEG₁₂, PEG₁₅, PEG₁₆, or PEG₁₅; PEG₄ to PEG₁₆; PEG₁₆; PEG₃ to PEG₁₅; PEG₁₅; PEG₃ to PEG₁₂; PEG₆; PEG₃ to PEG₈; or PEG₆.

Clause 8. The bispecific adaptor of any one of clauses 1-6 for use with an anti-fluorescein chimeric antigen receptor (CAR)-T cell in the treatment of cancer.

Clause 9. The bispecific adaptor of any one of clauses 1-5, for use with an anti-fluorescein CAR-T cell in the treatment of FAP-expressing cancer, wherein optionally the linker comprises or consists essentially of PEG₃ to PEG₁₅ and, optionally, PEG₁₅.

Clause 10. The bispecific adaptor of clause 1 or clause 6 for use with an anti-fluorescein CAR-T cell in the treatment of PSMA-expressing cancer, wherein optionally the linker comprises or consists essentially of PEG₃ to PEG₁₂ and, optionally, PEG₆ or PEG₃ to PEG₈ and, optionally, PEG₆.

Clause 11. A pharmaceutical composition for the treatment of cancer comprising the bispecific adapter of any one of clauses 1-10 and a pharmaceutically acceptable carrier or excipient.

Clause 12. A combination of bispecific adaptors for use with anti-fluorescein chimeric antigen-receptor (CAR)-T cells in the treatment of cancer, which combination comprises:
(i) a first bispecific adaptor comprising the bispecific adapter of claim 1 or a pharmaceutically acceptable salt or hydrate thereof, wherein the targeting ligand of the first bispecific adapter comprises a radical of a FAP ligand having a formula of:

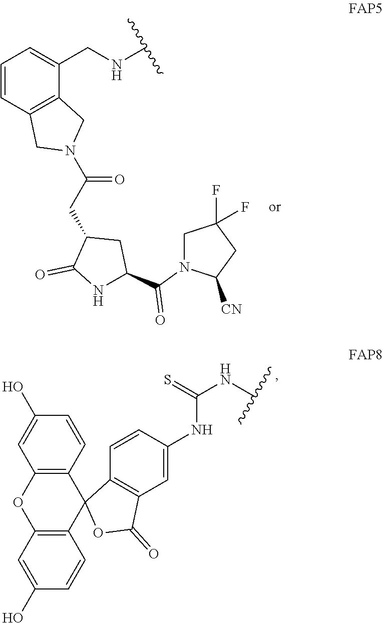

wherein

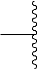

is the point of attachment to the linker; and
(ii) a second bispecific adapter comprising the following structure:

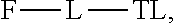

or a pharmaceutically acceptable salt or hydrate thereof, wherein:
   F comprises a fluorescein, FITC, or NHS-fluorescein,
   L comprises a linker, and
   TL comprises a targeting ligand comprising a radical of a folate receptor (FR) ligand or a prostate-specific membrane antigen (PSMA) ligand.

Clause 13. The combination of clause 12, wherein the radical of the FAP ligand of the first bispecific adaptor has a structure represented by the formula I-B:

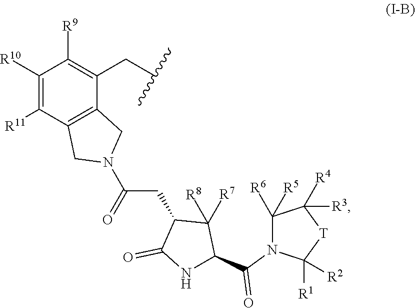

T is substituted or unsubstituted methylene (—CH₂—) substituted or unsubstituted amino (—NH—),
—O—, or —S—;
R¹ and R² are each independently selected from the group consisting of —H, —CN,
   —CHO, —B(OH)₂, —C(O)alkyl, —C(O)aryl-, —C═C—C(O)aryl, —C═C—S(O)₂aryl, —CO₂H, —SO₃H,
   —SO₂NH₂, —PO₃H₂, —SO₂F and 5-tetrazolyl;
R³ and R⁴ are each independently selected from the group consisting of —H, —OH, F, Cl, Br, I,
—C₁₋₆alkyl, —O—C₁₋₆alkyl, and —S—C₁₋₆alkyl;
R⁵, R⁶, R⁷, and R⁸ are each independently selected from group consisting of H, alkyl and halo; and
R⁹, R¹⁰, and R¹¹ are each independently selected from group consisting of H, —C₁₋₆alkyl, —O—C₁₋₆alkyl, —S—C₁₋₆ alkyl. F, Cl. Br and I; or
   a structure represented by the formula I-C:

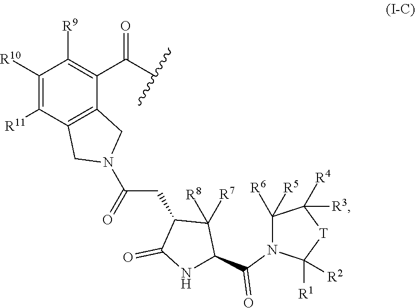

wherein:

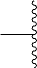

is the point of attachment to the linker;
T is substituted or unsubstituted methylene (—CH₂—), substituted or unsubstituted amino (—NH—), —O—, or —S—;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, and 5-tetrazolyl;
R³ and R⁴ are each independently selected from the group consisting of —H, —OH, F, Cl, Br, I, —C₁₋₆alkyl, —O—C₁₋₆alkyl, and —S—C₁₋₆alkyl;
R⁵, R⁶, R⁷, and R⁸ are each independently selected from group consisting of H, alkyl and halo; and
R⁹, R¹⁰, and R¹¹ are each independently selected from group consisting of H, —C₁₋₆ alkyl, —O—C₁₋₆alkyl, —S—C₁₋₆ alkyl, F, Cl, Br and I; or
   a structure represented comprising the following formula:

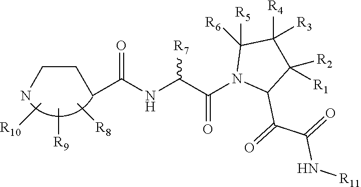

wherein:

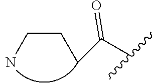 [SMILES: *C(=O)CCCN]

represents a functionalized 5- to 10-membered N-containing aromatic or non-aromatic mono- or bi-cyclic heterocycle, which optionally further comprises 1-3 heteroatoms selected from O, N, and S;
R₁ and R₂ are independently selected from the group consisting of —H, -D, —OH, —F, —Cl, —Br, —I, —C₁₋₆ alkyl, —O—C₁₋₆ alkyl, and —S—C₁₋₆ alkyl;
R₃ and R₄ are independently selected from the group consisting of —H, —OH, —F, —Cl, —Br, —I, —C₁₋₆ alkyl, —O—C₁₋₆ alkyl, and —S—C₁₋₆ alkyl;
R₅ and R₆ are independently selected from group consisting of —H, —OH, —F, —Cl, —Br, —I, —C₁₋₆ alkyl, —O—C₁₋₆ alkyl, and —S—C₁₋₆ alkyl;
R₇ is selected from the group consisting of —H, -D, OH, CH₂═, —CH₃, CH₃CH₂—, (CH₃)₂CH—, (CH₃)₃C—, —CH₂Ph, and substituted —CH₂Ph;
R₈-R₁₀ are independently selected from group consisting of —H, —OH, —F, —Cl, —Br,
—I, —NO₂, —SO₃H, —SO₂NH₂, —NH₂, —N₃, —NH═NH, —C₁₋₆ alkyl, —O—C₁₋₆ alkyl, and —S—C₁₋₆ alkyl; and
R₁₁ is selected from the group consisting of —H, -D, C₁-C₁₀ alkyl, C₃-C₁₀ cycloalkyl, adamantyl,

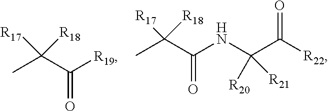

substituted or unsubstituted aryl, substituted or unsubstituted C₇-C₂₀ alkyl aryl, wherein the aryl is:

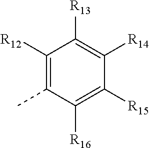

wherein:
R₁₂ and R₁₆ are independently selected from the group consisting of —H, -D, halogen, C₁-C₃ alkyl, C₁-C₃ alkoxy, —CF₃, and —C(═O)—OR₂₃, wherein R₂₃ is selected from the group consisting of H, D, halogen, C₁-C₄ alkyl, and C₁-C₃ alkoxy;
R₁₃, R₁₄ and R₁₅ are independently selected from the group consisting of —H, -D, halogen, —OMe. C₁-C₃ alkyl, C—C₃ alkoxy, —CF₃, and —C(═O)—OR₂₃, wherein R²³ is selected from the group consisting of —H, -D, halogen, C₁-C₄ alkyl, and C₁-C₃ alkoxy;
R₁₇, R₁₈, R₂₀, and R₂₁ are independently selected from —H and —CH₃; and
R₁₉ and R₂₂ are independently selected from the group consisting of phenyl, dimethoxyphenyl, and aryl.

Clause 14. The combination of clause 12, wherein the targeting ligand of the second bispecific adapter, or pharmaceutically acceptable salt or hydrate thereof, comprises a radical of a PSMA ligand.

Clause 15. The combination of clause 12, wherein the linker of the first bispecific adaptor comprises or consists essentially of PEG.

Clause 16. The combination of clause 14, wherein the targeting ligand is or comprises PSMAL1 or DUPA.

Clause 17. The combination of clause 12, wherein the targeting ligand of the second bispecific adaptor is a radical of a folate or a functional fragment or analog thereof.

Clause 18. The combination of clause 17, wherein the folate is folate, dihydrofolate tetrahydrofolate, 5, 10-methylene tetrahydrofolate (5,10-MTHF), 5-methyltetrahydrofolate (5-MTHF), or raltitrexed.

Clause 19. The combination of clause 16, wherein the PSMA ligand is DUPA and the linker comprises or consists essentially of PEG or a PEG derivative such as, optionally: PEG₃ to PEG₁₂ and, optionally, PEG₆; PEG₃ to PEG₁₆ and, optionally, PEG₄ to PEG₁₅ or PEG₃ to PEG₁₂; PEG₁₂, PEG₁₅, PEG₁₆, or PEG₁₈; PEG₄ to PEG₁₆ and, optionally, PEG₁₆; PEG₃ to PEG₁₅ and, optionally. PEG₁₅; PEG₃ to PEG₈ and, optionally, PEG₆.

Clause 20. The combination of any one of clauses 12-19 for use with an anti-fluorescein CAR-T cell in the treatment of cancer.

Clause 21. The combination of any one of clauses 12-19 for use with an anti-fluorescein CAR-T cell in the treatment of FAP-expressing cancer.

Clause 22. The combination of any one of clauses 12-16, and 19 for use with an anti-fluorescein CAR-T cell in the treatment of PSMA-expressing cancer.

Clause 23. The combination of any one of clauses 12-15, 17, and 18 for use with an anti-fluorescein CAR-T cell in the treatment of folate-expressing cancer.

Clause 24. The combination of any one of clauses 12-19, wherein the first and second bispecific adapters are formulated in separate pharmaceutical compositions.

Clause 25. A bispecific adapter for use with an anti-fluorescein CAR-T cell in the treatment of a FAP-expressing cancer, which adapter has or comprises one of the following structures:

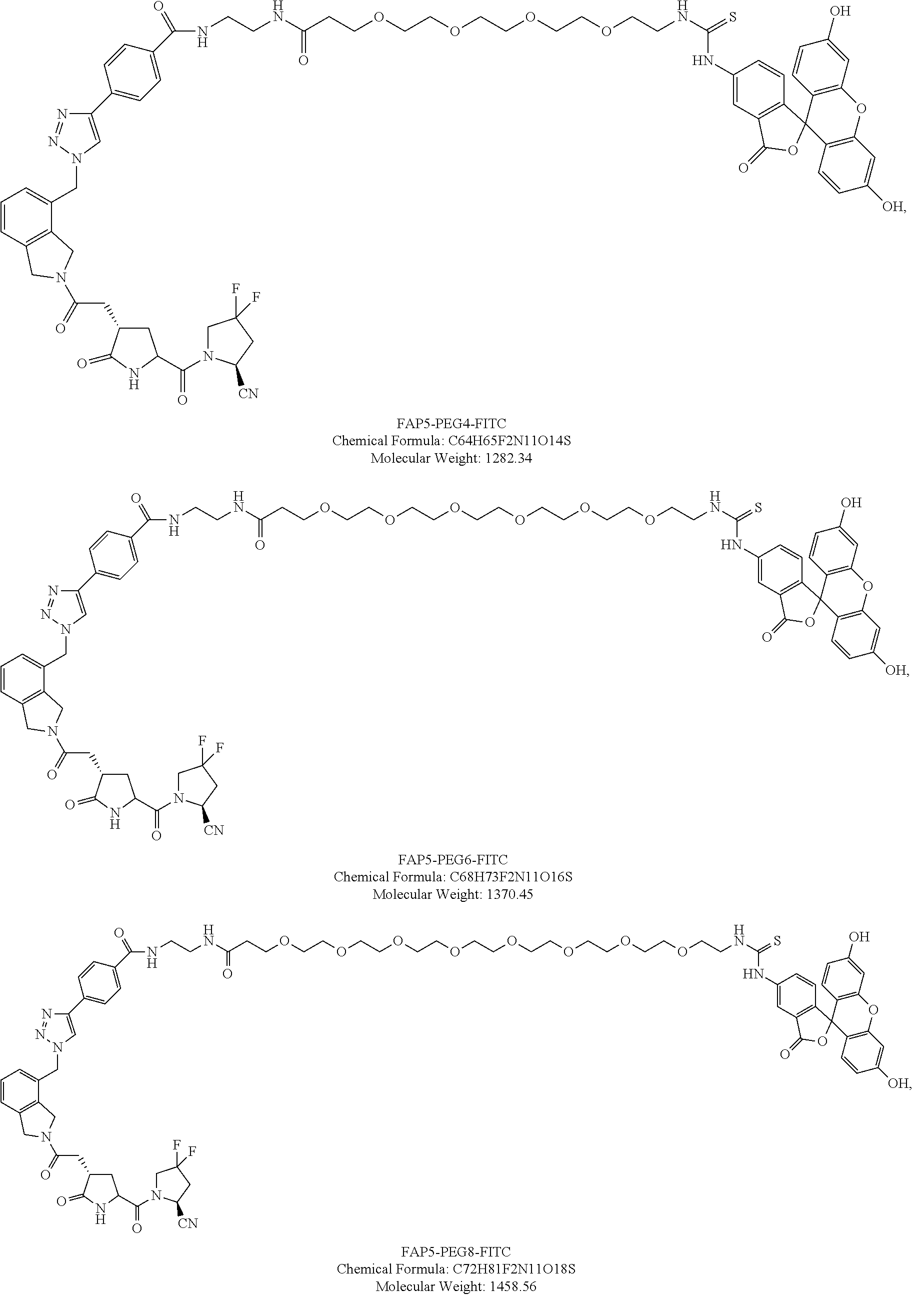

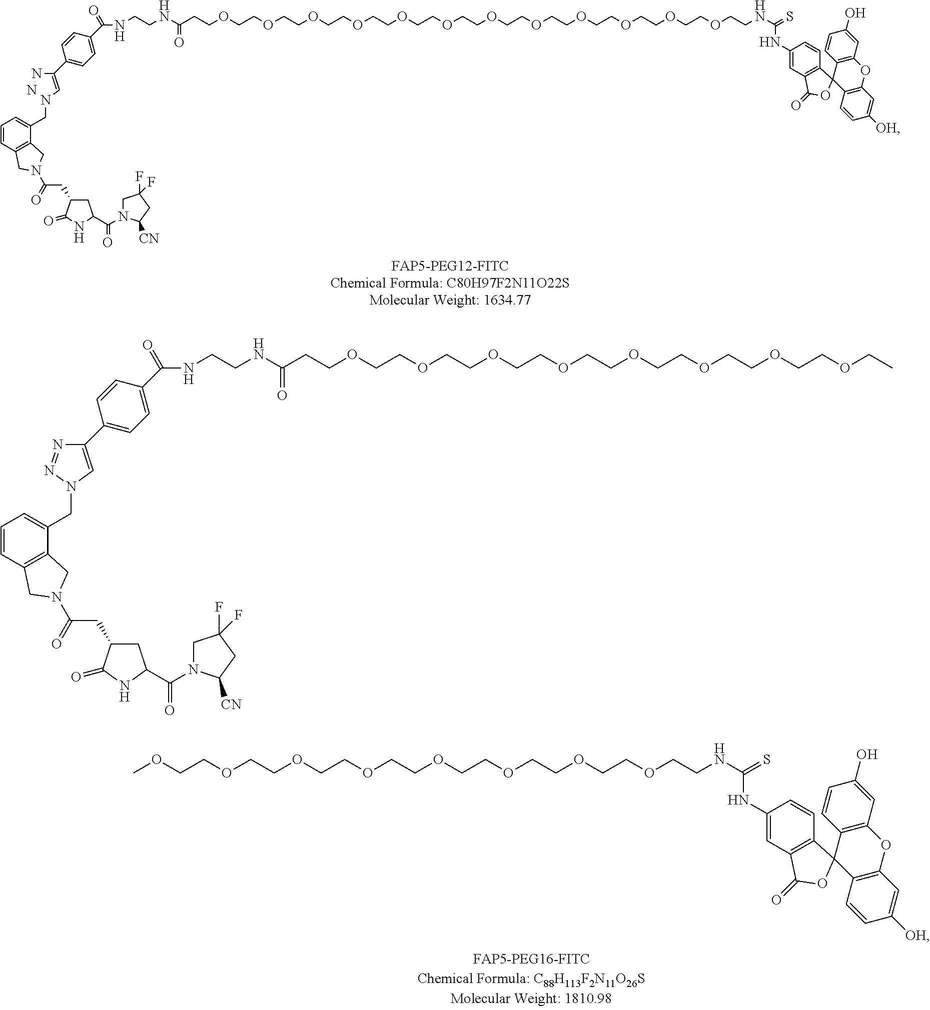

or comprising a pharmaceutically acceptable salt or hydrate of any of the foregoing structures.

Clause 26. A bispecific adapter for use with an anti-fluorescein CAR-T cell in the treatment of a FAP-expressing cancer, which adapter has or comprises one of the following structures:

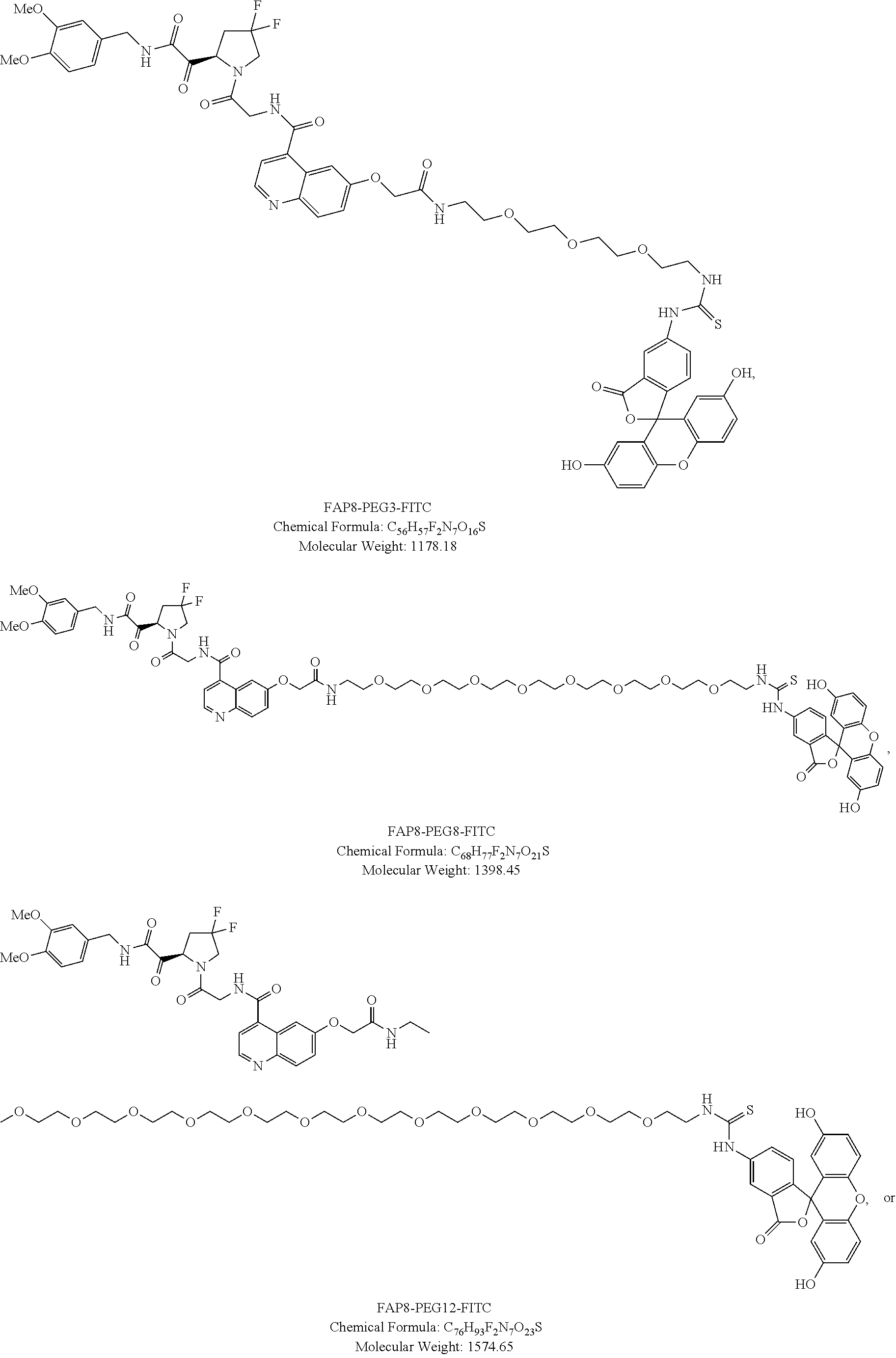 [SMILES: COc1ccc(CNC(=O)C(=O)[C@H]2CC(F)(F)CN2C(=O)CNC(=O)c2ccnc3ccc(OCC(=O)NCCOCCOCCOCCNC(=S)Nc4ccc5c(c4)C(=O)OC54c5cc(O)ccc5Oc5ccc(O)cc54)cc23)cc1OC ; COc1ccc(CNC(=O)C(=O)[C@H]2CC(F)(F)CN2C(=O)CNC(=O)c2ccnc3ccc(OCC(=O)NCCOCCOCCOCCOCCOCCOCCOCCOCCNC(=S)Nc4ccc5c(c4)C(=O)OC54c5cc(O)ccc5Oc5ccc(O)cc54)cc23)cc1OC ; CCNC(=O)COc1ccc2nccc(C(=O)NCC(=O)N3CC(F)(F)C[C@@H]3C(=O)C(=O)NCc3ccc(OC)c(OC)c3)c2c1 ; COCCOCCOCCOCCOCCOCCOCCOCCOCCOCCOCCOCCNC(=S)Nc1ccc2c(c1)C(=O)OC21c2cc(O)ccc2Oc2ccc(O)cc21 ; "or"]

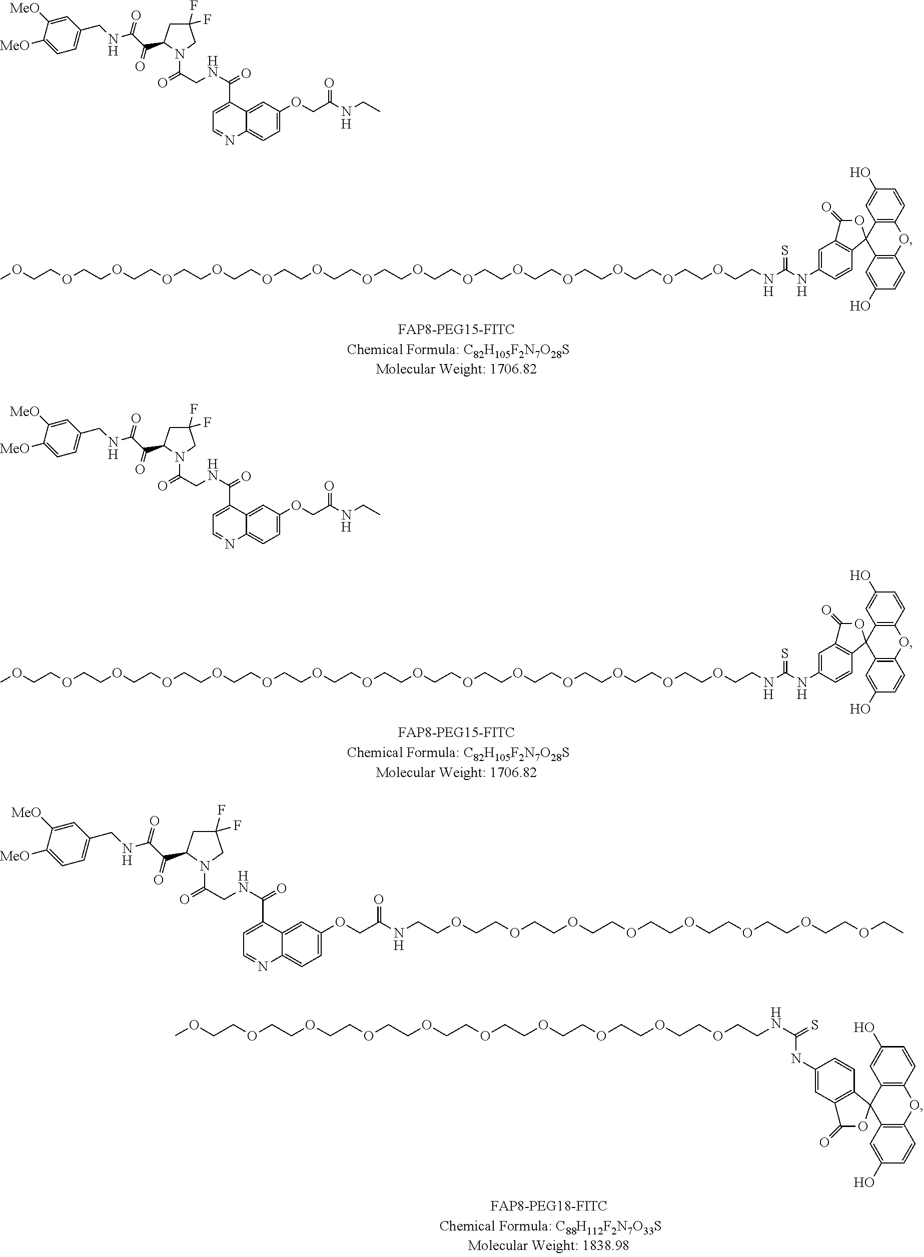

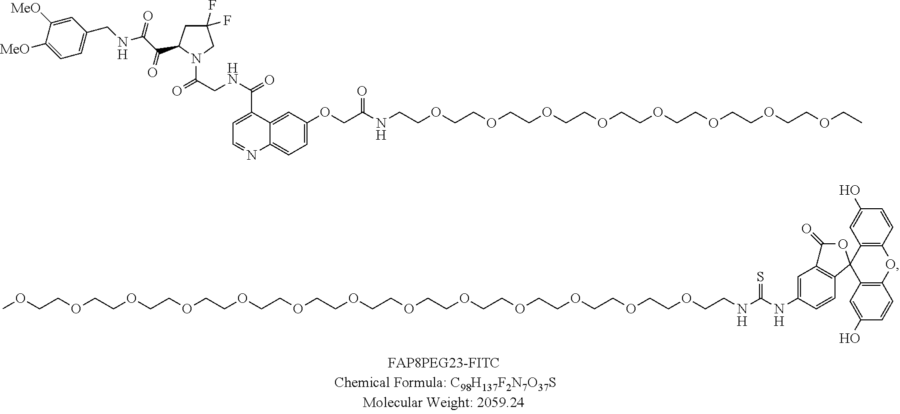

or comprising a pharmaceutically acceptable salt or hydrate of any of the foregoing structures.

Clause 27. A bispecific adapter for use with an anti-fluorescein CAR-T cell in the treatment of a PSMA cancer, which adapter has or comprises one of the following structures:

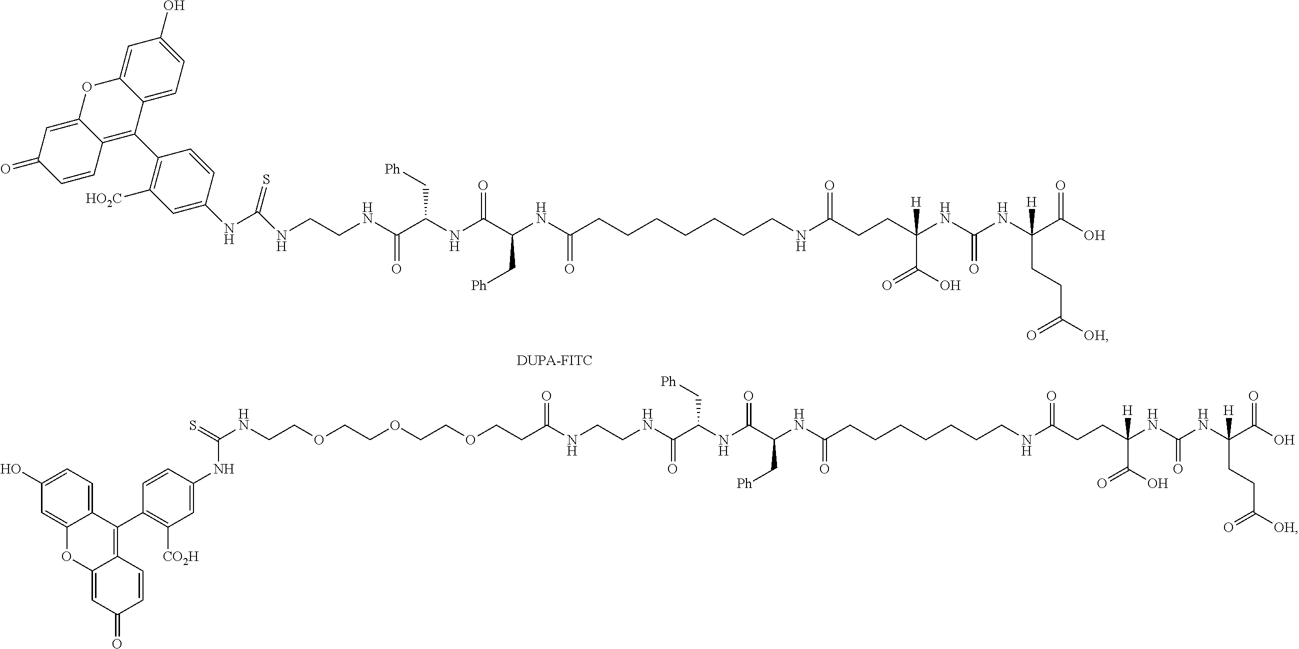 [SMILES: O=C(O)CC[C@H](NC(=O)N[C@@H](CCC(=O)NCCCCCCCC(=O)N[C@@H](Cc1ccccc1)C(=O)N[C@@H](Cc1ccccc1)C(=O)NCCNC(=S)Nc1ccc(-c2c3ccc(=O)cc-3oc3cc(O)ccc23)c(C(=O)O)c1)C(=O)O)C(=O)O ; O=C(O)CC[C@H](NC(=O)N[C@@H](CCC(=O)NCCCCCCCC(=O)N[C@@H](Cc1ccccc1)C(=O)N[C@@H](Cc1ccccc1)C(=O)NCCNC(=O)CCOCCOCCOCCNC(=S)Nc1ccc(-c2c3ccc(=O)cc-3oc3cc(O)ccc23)c(C(=O)O)c1)C(=O)O)C(=O)O]

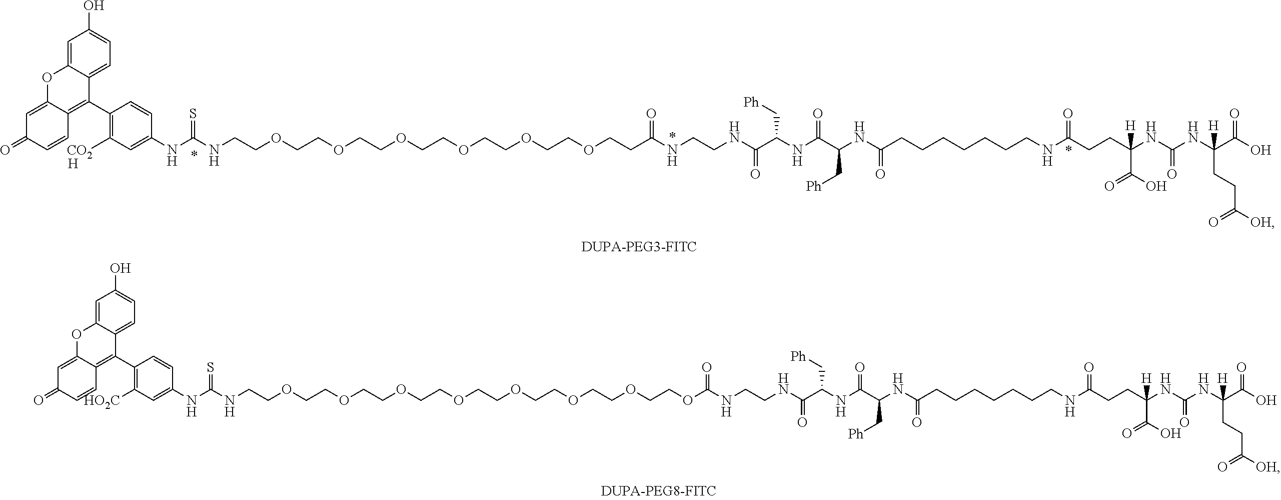

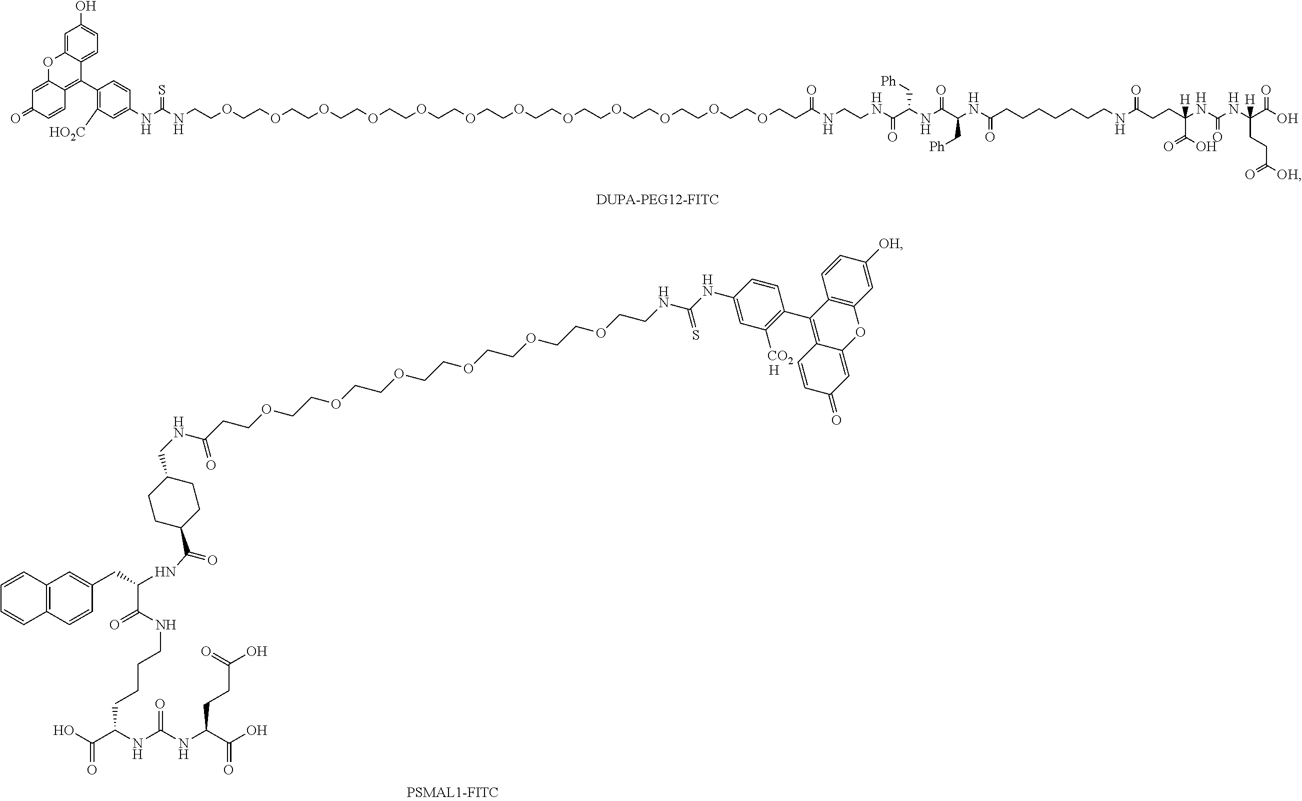

or comprising a pharmaceutically acceptable salt or hydrate of any of the foregoing.

Clause 28. A bispecific adapter for use with an anti-fluorescein CAR-T cell in the treatment of PSMA-expressing cancer, which adapter has the structure:

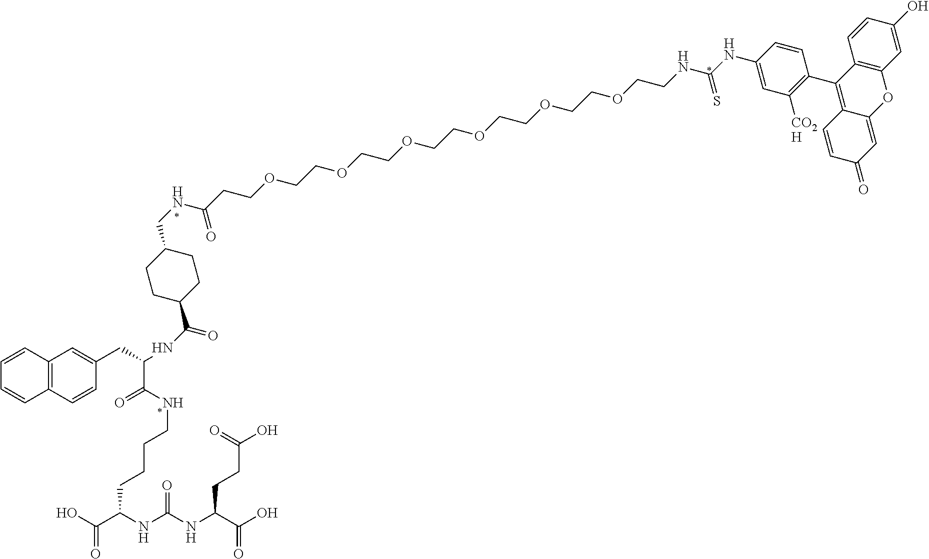 [SMILES: O=C(O)CC[C@H](NC(=O)N[C@@H](CCCCNC(=O)[C@H](Cc1ccc2ccccc2c1)NC(=O)[C@H]1CC[C@H](CNC(=O)CCOCCOCCOCCOCCOCCOCCNC(=S)Nc2ccc(-c3c4ccc(=O)cc-4oc4cc(O)ccc34)c(C(=O)O)c2)CC1)C(=O)O)C(=O)O]

is a pharmaceutically acceptable salt or hydrate thereof.

Clause 29. A bispecific adapter for use with an anti-fluorescein CAR-T cell in the treatment of PSMA-expressing cancer, which adapter has the structure:

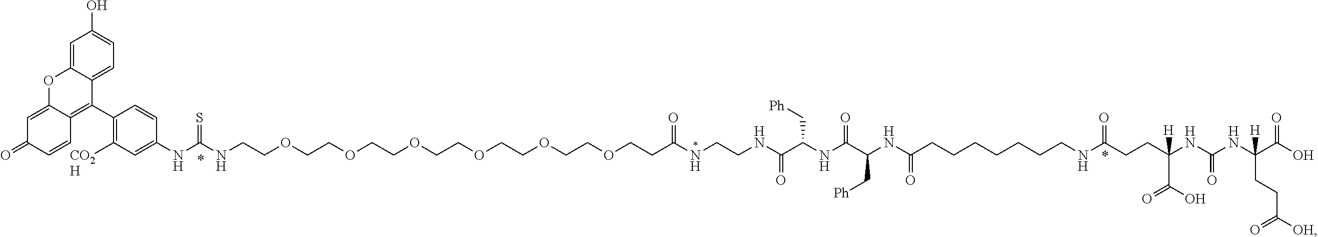

or is a pharmaceutically acceptable salt or hydrate thereof.

Clause 30. A kit comprising: (i) at least one dosage unit of a bispecific adapter of any one of clauses 1-10 or 25-29, a pharmaceutical composition comprising a bispecific adapter of any one of clauses 1-10 or 25-29 and a pharmaceutically acceptable carrier or excipient, or a combination of any one of clauses 12-24; and (ii) at least one dosage unit of an anti-fluorescein CAR-T cells or a pharmaceutical composition comprising anti-fluorescein CAR-T cells and a pharmaceutically acceptable carrier or excipient; wherein (i) and (ii) are optionally in separate containers.

Clause 31. A method of treating cancer in a subject comprising administering to the subject cancer-treatment effective amounts of: (i) anti-fluorescein CAR-T cells or a pharmaceutical composition comprising anti-fluorescein CAR-T cells and a pharmaceutically acceptable carrier or excipient; and (ii) a bispecific adapter of any one of clauses 1-10 and 25-29, a pharmaceutical composition of clause 11, or a combination of any one of clauses 12-24; whereupon the subject is treated for cancer.

Clause 32. The method of clause 31, wherein the CAR comprises: a recognition region comprising a single chain fragment variable (scFv) region of an anti-fluorescein antibody; a co-stimulation domain and the co-stimulation domain is CD28, CD137 (4-1BB), CD134 (OX40), or CD278 (ICOS); and/or an activation signaling domain that is a T cell CD3ζ chain or an Fc receptor γ.

Clause 33. The method of clause 31, wherein the fluorescein of the bispecific adapter binds the anti-fluorescein CAR-T cell with affinity upon exposure thereto, and the targeting ligand of the bispecific adapter links the bound anti-fluorescein CAR-T cell to a targeted cancer cell or CAF upon the targeted ligand of the bispecific adapter binding a receptor on such targeted cancer cell or CAF with affinity.

Clause 34. The method of clause 33, wherein the receptor on the targeted cancer cell or CAF is an overexpressed FAP, an over-expressed PSMA, and/or a FR.

Clause 35. The method of clause 31, wherein (i) and (ii) are administered simultaneously or sequentially, in either order, by the same or different routes.

Clause 36. The method of clause 31, wherein (ii) comprises the combination of any one of clauses 25-29 and the first and second bispecific adapters are administered to the subject simultaneously by the same or different routes.

Clause 37. The method of clause 31, wherein (ii) comprises the combination of any one of clauses 25-29 and the first and second bispecific adapters are administered to the subject sequentially, in either order, by the same or different routes.

Clause 38. The method of any one of clauses 31-37, wherein (i) and (ii) are each administered intravenously.

Clause 39. The method of clause 31, wherein the cancer is a FAP-expressing cancer and at least one bispecific adapter of (ii) comprises a radical of a FAP ligand.

Clause 40. The method of clause 31, wherein the cancer is a PSMA-expressing cancer and at least one bispecific adapter of (ii) comprises a radical of a PSMA ligand.

Clause 41. The method of clause 31, wherein the cancer is a folate receptor-expressing cancer and (ii) comprises a combination of any one of clauses 12-15, 17, and 18.

Clause 42. A method of treating FAP-expressing cancer in a subject, which method comprises administering to the subject cancer-treatment effective amounts of (i) anti-fluorescein CAR-T cells or a pharmaceutical composition comprising anti-fluorescein CAR-T cells and a pharmaceutically acceptable carrier or excipient; and (ii) the bispecific adapter of any one of clauses 1-5 or a pharmaceutical composition comprising the same and a pharmaceutically acceptable carrier or excipient, whereupon the subject is treated for cancer.

Clause 43. The method of clause 42, wherein the CAR has a recognition region and the recognition region is a scFv region of an anti-fluorescein antibody.

Clause 44. The method of clause 42 or 43, wherein the CAR comprises: a co-stimulation domain and the co-stimulation domain is CD28, CD137 (4-1BB), CD134 (OX40), or CD278 (ICOS); and/or an activation signaling domain and the activation signaling domain is a T cell CD3 chain or an Fc receptor γ.

Clause 45. A method of treating cancer in a subject comprising administering to the subject cancer-treatment effective amounts of: (i) anti-fluorescein CAR-T cells or a pharmaceutical composition comprising anti-fluorescein CAR-T cells and a pharmaceutically acceptable carrier or excipient; and (ii) a combination of any one of clauses 12-24; whereupon the subject is treated for cancer.

Clause 46. The method of clause 45, wherein the CAR comprises: a recognition region comprising a scFv region of an anti-fluorescein antibody; a co-stimulation domain and the co-stimulation domain is CD28, CD137 (4-1BB), CD134 (OX40), or CD278 (ICOS); and/or an activation signaling domain that is a T cell CD3 chain or an Fc receptor γ.

Clause 47. The method of clause 45, wherein (i) and (ii) are administered simultaneously or sequentially, in either order, by the same or different routes.

Clause 48. The method of clause 45, wherein the first and second bispecific adapters of the combination are administered to the subject simultaneously by the same or different routes.

Clause 49. The method of clause 45, wherein the first and second bispecific adapters of the combination are administered to the subject sequentially, in either order, by the same or different routes.

Clause 50. The method of any one of clauses 45-49, wherein (i) and (ii) are each administered intravenously.

Clause 51. The method of any one of clauses 31-49 further comprising imaging the cancer in the subject.

Clause 52. The method of clause 51, wherein imaging the cancer comprises imaging by optical imaging, positron emission tomography (PET), or single photon emission computed tomography (SPECT).

Clause 53. The method of any one of clauses 31-49, wherein the cancer is ovarian cancer, endometrial cancer, breast cancer, glioma such as, optionally, stage 3-4 glioma, or clear cell renal cell carcinoma such as, optionally, stage 3-4 clear cell renal cell carcinoma.

Clause 54. A method for enhancing CAR-T cell activation comprising: providing a bispecific adapter of any one of clauses 1-10 and 25-29, a pharmaceutical composition of clause 11, or a combination of any one of clauses 12-24; and exposing anti-fluorescein CAR-T cells or a pharmaceutical composition comprising anti-fluorescein CAR-T cells and a pharmaceutically acceptable carrier or excipient to the bispecific adaptor, pharmaceutical composition or combination; wherein the CAR-T cell experiences enhanced activation following exposure as compared to a CAR-T cell not exposed to the bispecific adapter.

Clause 55. The method of clause 54, wherein the anti-fluorescein CAR-T cells are in systemic circulation in a subject when exposed to the bispecific adaptor.

1. A bispecific adapter comprising the following structure: 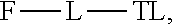
or a pharmaceutically acceptable salt or hydrate thereof, wherein:
F comprises a fluorescein, fluorescein isothiocyanate (FITC), or N-hydroxysuccinimide (NHS)-fluorescein,
L comprises a linker, and
TL comprises a targeting ligand comprising a radical of a fibroblast activation protein (FAP) ligand or a radical of a prostate-specific membrane antigen (PSMA) ligand.

2. The bispecific adapter of claim 1, wherein the targeting ligand comprises a radical of a FAP ligand comprising a structure of: 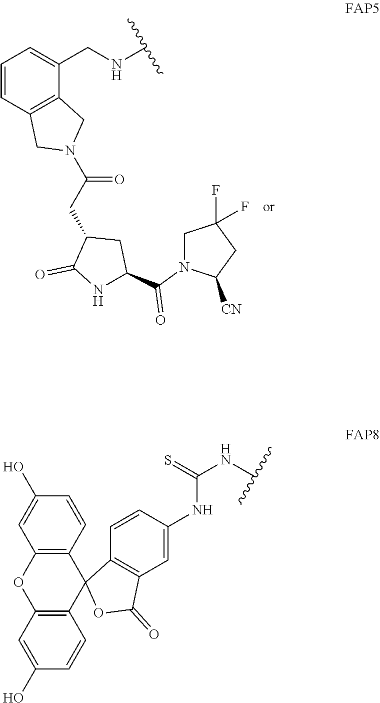
wherein 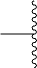
is the point of attachment to the linker.

3. The bispecific adaptor of claim 2, wherein the targeting ligand comprises a radical of a FAP5 ligand comprising a structure represented by formula I-B: 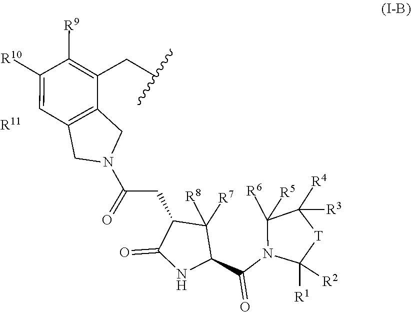
wherein: 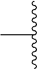
is the point of attachment to the linker;
T is substituted or unsubstituted methylene (—CH₂—), substituted or unsubstituted amino (—NH—), —O—, or —S—;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F and 5-tetrazolyl;
R³ and R⁴ are each independently selected from the group consisting of —H, —OH, F, Cl, Br, I, —C₁₋₆alkyl, —O—C₁₋₆alkyl, and —S—C₁₋₆alkyl;
R⁵, R⁶, R⁷, and R⁸ are each independently selected from group consisting of H, alkyl and halo; and
R⁹, R¹⁰, and R¹¹ are each independently selected from group consisting of H, —C₁₋₆alkyl, —O—C₁₋₆alkyl, —S—C₁₋₆ alkyl, F, Cl, Br and I.

4. The bispecific adapter of claim 2, wherein the targeting ligand comprises a radical of a FAP5 ligand comprising a structure represented by the formula I-C: 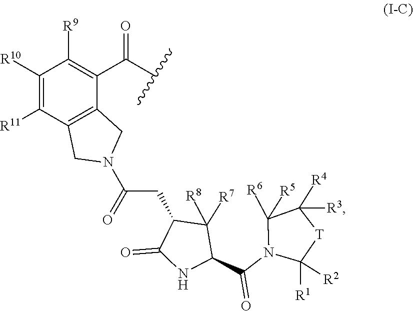
wherein: 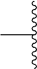
is the point of attachment to the linker;
T is substituted or unsubstituted methylene (—CH₂—), substituted or unsubstituted amino (—NH—), —O—, or —S—;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, and 5-tetrazolyl;
R³ and R⁴ are each independently selected from the group consisting of —H, —OH, F, Cl, Br, I, —C₁₋₆alkyl, —O—C₁₋₆alkyl, and —S—C₁₋₆alkyl;
R⁵, R⁶, R⁷, and R⁸ are each independently selected from group consisting of H, alkyl and halo; and
R⁹, R¹⁰, and R¹¹ are each independently selected from group consisting of H, —C₁₋₆alkyl, —O—C₁₋₆alkyl, —S—C₁₋₆ alkyl, F, Cl, Br and I.

5. The bispecific adapter of claim 2, wherein the targeting ligand comprises a radical of a FAP8 ligand comprising a structure: 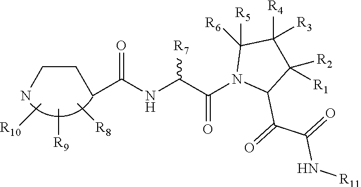
wherein: 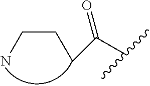 [SMILES: *C(=O)CCCN]
represents a functionalized 5- to 10-membered N-containing aromatic or non-aromatic mono- or bi-cyclic heterocycle, which optionally further comprises 1-3 heteroatoms selected from O, N, and S;
R₁ and R₂ are independently selected from the group consisting of —H, -D, —OH, —F, —Cl, —Br, —I, —C₁₋₆ alkyl, —O—C₁₋₆ alkyl, and —S—C₁₋₆ alkyl;
R₃ and R₄ are independently selected from the group consisting of —H, —OH, —F, —Cl, —Br, —I, —C₁₋₆ alkyl, —O—C₁₋₆ alkyl, and —S—C₁₋₆ alkyl;
R₅ and R₆ are independently selected from group consisting of —H, —OH, —F, —Cl, —Br, —I, —C₁₋₆ alkyl, —O—C₁₋₆ alkyl, and —S—C₁₋₆ alkyl;
R₇ is selected from the group consisting of —H, -D, OH, CH₂═, —CH₃, CH₃CH₂—, (CH₃)₂CH—, (CH₃)₃C—, —CH₂Ph, and substituted —CH₂Ph;
R₈-R₁₀ are independently selected from group consisting of —H, —OH, —F, —Cl, —Br, —I, —NO₂, —SO₃H, —SO₂NH₂, —NH₂, —N₃, —NH═NH, —C₁₋₆ alkyl, —O—C₁₋₆ alkyl, and —S—C₁₋₆ alkyl; and
R₁₁ is selected from the group consisting of —H, -D, C₁-C₁₀ alkyl, C₃-C₁₀ cycloalkyl, adamantyl, 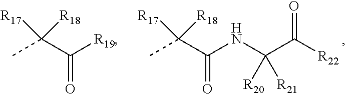
substituted or unsubstituted aryl, substituted or unsubstituted C₇-C₂₀ alkyl aryl, wherein the aryl is: 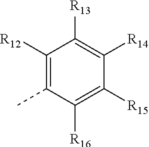
wherein:
R₁₂ and R₁₆ are independently selected from the group consisting of —H, -D, halogen, C₁-C₃ alkyl, C₁-C₃ alkoxy, —CF₃, and —C(═O)—OR₂₃, wherein R₂₃ is selected from the group consisting of H, D, halogen, C₁-C₄ alkyl, and C₁-C₃ alkoxy;
R₁₃, R₁₄ and R₁₅ are independently selected from the group consisting of —H, -D, halogen, —OMe, C₁-C₃ alkyl, C₁-C₃ alkoxy, —CF₃, and —C(═O)—OR₂₃, wherein R₂₃ is selected from the group consisting of —H, -D, halogen, C₁-C₄ alkyl, and C₁-C₃ alkoxy;
R₁₇, R₁₈, R₂₀, and R₂₁ are independently selected from —H and —CH₃; and
R₁₉ and R₂₂ are independently selected from the group consisting of phenyl, dimethoxyphenyl, and aryl.

6. The bispecific adapter of claim 1, wherein the targeting ligand comprises a radical of a PSMA ligand and is ((S)-5-amino-1-carboxypentyl)carbamoyl)-L-glutamic acid (PSMAL1) or 2-[3-(1,3-dicarboxypropyl)ureido]pentanedioic acid (DUPA).

7. The bispecific adapter of any one of claims 1-6 wherein the linker comprises or consists essentially of polyethylene glycol (PEG) or a PEG derivative such as, optionally:
PEG₃ to PEG₁₆ and, optionally, PEG₄ to PEG₁₅ or PEG₃ to PEG₁₂;
PEG₁₂, PEG₁₅, PEG₁₆, or PEG₁₈;
PEG₄ to PEG₁₆;
PEG₁₆;
PEG₃ to PEG₁₅;
PEG₁₅;
PEG₃ to PEG₁₂;
PEG₆;
PEG₃ to PEG₈; or
PEG₆.

8. The bispecific adaptor of any one of claims 1-6 for use with an anti-fluorescein chimeric antigen receptor (CAR)-T cell in the treatment of cancer.

9. The bispecific adaptor of any one of claims 1-5, for use with an anti-fluorescein CAR-T cell in the treatment of FAP-expressing cancer, wherein optionally the linker comprises or consists essentially of PEG₃ to PEG₁₅ and, optionally, PEG₁₅.

10. The bispecific adaptor of claim 1 or claim 6 for use with an anti-fluorescein CAR-T cell in the treatment of PSMA-expressing cancer, wherein optionally the linker comprises or consists essentially of PEG₃ to PEG₁₂ and, optionally, PEG₆ or PEG₃ to PEG₈ and, optionally, PEG₆.

11. A pharmaceutical composition for the treatment of cancer comprising the bispecific adapter of any one of claims 1-10 and a pharmaceutically acceptable carrier or excipient.

12. A combination of bispecific adaptors for use with anti-fluorescein chimeric antigen-receptor (CAR)-T cells in the treatment of cancer, which combination comprises:
(i) a first bispecific adaptor comprising the bispecific adapter of claim 1 or a pharmaceutically acceptable salt or hydrate thereof, wherein the targeting ligand of the first bispecific adapter comprises a radical of a FAP ligand having a formula of: 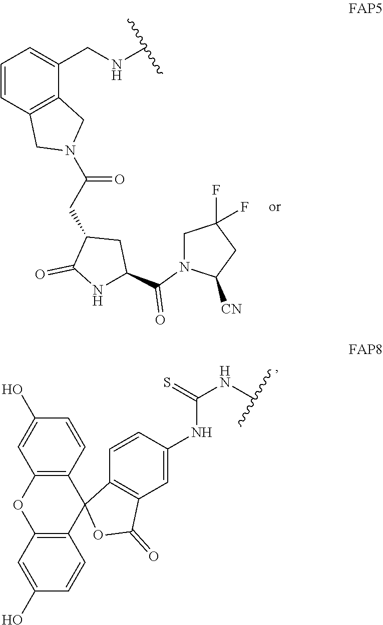
wherein 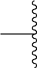
is the point of attachment to the linker; and
(ii) a second bispecific adapter comprising the following structure: 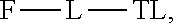
or a pharmaceutically acceptable salt or hydrate thereof, wherein:
F comprises a fluorescein, FITC, or NHS-fluorescein,
L comprises a linker, and
TL comprises a targeting ligand comprising a radical of a folate receptor (FR) ligand or a prostate-specific membrane antigen (PSMA) ligand.

13. The combination of claim 12, wherein the radical of the FAP ligand of the first bispecific adaptor has a structure represented by the formula I-B: 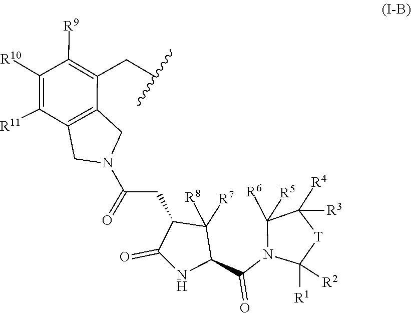
T is substituted or unsubstituted methylene (—CH₂—), substituted or unsubstituted amino (—NH—),
—O—, or —S—;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H,
—SO₂NH₂, —PO₃H₂, —SO₂F and 5-tetrazolyl;
R³ and R⁴ are each independently selected from the group consisting of —H, —OH, F, Cl, Br, I,
—C₁₋₆alkyl, —O—C₁₋₆alkyl, and —S—C₁₋₆alkyl;
R⁵, R⁶, R⁷, and R⁸ are each independently selected from group consisting of H, alkyl and halo; and
R⁹, R¹⁰, and R¹¹ are each independently selected from group consisting of H, —C₁₋₆alkyl, —O—C₁₋₆alkyl, —S—C₁₋₆ alkyl, F, Cl, Br and I; or
a structure represented by the formula I-C: 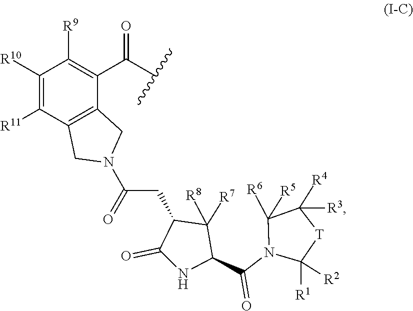
wherein: 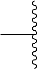
is the point of attachment to the linker;
T is substituted or unsubstituted methylene (—CH₂—), substituted or unsubstituted amino (—NH—), —O—, or —S—;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, and 5-tetrazolyl;
R³ and R⁴ are each independently selected from the group consisting of —H, —OH, F, Cl, Br, I, —C₁₋₆alkyl, —O—C₁₋₆alkyl, and —S—C₁₋₆alkyl;
R⁵, R⁶, R⁷, and R⁸ are each independently selected from group consisting of H, alkyl and halo; and
R⁹, R¹⁰, and R¹¹ are each independently selected from group consisting of H, —C₁₋₆ alkyl, —O—C₁₋₆alkyl, —S—C₁₋₆ alkyl, F, Cl, Br and I; or
a structure represented comprising the following formula: 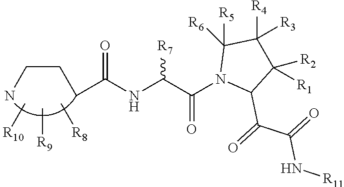
wherein: 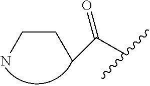 [SMILES: *C(=O)CCCN]
represents a functionalized 5- to 10-membered N-containing aromatic or non-aromatic mono- or bi-cyclic heterocycle, which optionally further comprises 1-3 heteroatoms selected from O, N, and S;
R₁ and R₂ are independently selected from the group consisting of —H, -D, —OH, —F, —Cl, —Br, —I, —C₁₋₆ alkyl, —O—C₁₋₆ alkyl, and —S—C₁₋₆ alkyl;
R₃ and R₄ are independently selected from the group consisting of —H, —OH, —F, —Cl, —Br, —I, —C₁₋₆ alkyl, —O—C₁₋₆ alkyl, and —S—C₁₋₆ alkyl;
R₅ and R₆ are independently selected from group consisting of —H, —OH, —F, —Cl, —Br, —I, —C₁₋₆ alkyl, —O—C₁₋₆ alkyl, and —S—C₁₋₆ alkyl;
R₇ is selected from the group consisting of —H, -D, OH, CH₂═, —CH₃, CH₃CH₂—, (CH₃)₂CH—, (CH₃)₃C—, —CH₂Ph, and substituted —CH₂Ph;
R₈-R₁₀ are independently selected from group consisting of —H, —OH, —F, —Cl, —Br, —I, —NO₂, —SO₃H, —SO₂NH₂, —NH₂, —N₃, —NH═NH, —C₁₋₆ alkyl, —O—C₁₋₆ alkyl, and —S—C₁₋₆ alkyl; and
R₁₁ is selected from the group consisting of —H, -D, C₁-C₁₀ alkyl, C₃-C₁₀ cycloalkyl, adamantyl, 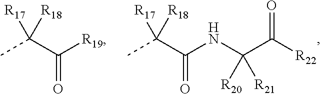
substituted or unsubstituted aryl, substituted or unsubstituted C₇-C₂₀ alkyl aryl, wherein the aryl is: 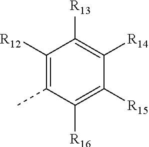
wherein:
R₁₂ and R₁₆ are independently selected from the group consisting of —H, -D, halogen, C₁-C₃ alkyl, C₁-C₃ alkoxy, —CF₃, and —C(═O)—OR₂₃, wherein R₂₃ is selected from the group consisting of H, D, halogen, C₁-C₄ alkyl, and C₁-C₃ alkoxy;
R₁₃, R₁₄ and R₁₅ are independently selected from the group consisting of —H, -D, halogen, —OMe, C₁-C₃ alkyl, C₁-C₃ alkoxy, —CF₃, and —C(═O)—OR₂₃, wherein R₂₃ is selected from the group consisting of —H, -D, halogen, C₁-C₄ alkyl, and C₁-C₃ alkoxy;
R₁₇, R₁₈, R₂₀, and R₂₁ are independently selected from —H and —CH₃; and
R₁₉ and R₂₂ are independently selected from the group consisting of phenyl, dimethoxyphenyl, and aryl.

14. The combination of claim 12, wherein the targeting ligand of the second bispecific adapter, or pharmaceutically acceptable salt or hydrate thereof, comprises a radical of a PSMA ligand.

15. The combination of claim 12, wherein the linker of the first bispecific adaptor comprises or consists essentially of polyethylene glycol (PEG).

16. The combination of claim 14, wherein the targeting ligand is or comprises (((S)-5-amino-1-carboxypentyl)carbamoyl)-L-glutamic acid (PSMAL1) or 2-[3-(1,3-dicarboxypropyl)ureido]pentanedioic acid (DUPA).

17. The combination of claim 12, wherein the targeting ligand of the second bispecific adaptor is a radical of a folate or a functional fragment or analog thereof.

18. The combination of claim 17, wherein the folate is folate, dihydrofolate tetrahydrofolate, 5, 10-methylene tetrahydrofolate (5,10-MTHF), 5-methyltetrahydrofolate (5-MTHF), or raltitrexed.

19. The combination of claim 16, wherein the PSMA ligand is DUPA and the linker comprises or consists essentially of PEG or a PEG derivative such as, optionally:
PEG₃ to PEG₁₂ and, optionally, PEG₆;
PEG₃ to PEG₁₆ and, optionally, PEG₄ to PEG₁₅ or PEG₃ to PEG₁₂;
PEG₁₂, PEG₁₅, PEG₁₆, or PEG₁₈;
PEG₄ to PEG₁₆ and, optionally, PEG₁₆;
PEG₃ to PEG₁₅ and, optionally, PEG₁₅;
PEG₃ to PEG₈ and, optionally, PEG₆.

20. The combination of any one of claims 12-19 for use with an anti-fluorescein chimeric antigen receptor (CAR)-T cell in the treatment of cancer.

21. The combination of any one of claims 12-19 for use with an anti-fluorescein CAR-T cell in the treatment of FAP-expressing cancer.

22. The combination of any one of claims 12-16, and 19 for use with an anti-fluorescein CAR-T cell in the treatment of PSMA-expressing cancer.

23. The combination of any one of claims 12-15, 17, and 18 for use with an anti-fluorescein CAR-T cell in the treatment of folate-expressing cancer.

24. The combination of any one of claims 12-19, wherein the first and second bispecific adapters are formulated in separate pharmaceutical compositions.

25. A bispecific adapter for use with an anti-fluorescein chimeric antigen receptor (CAR)-T cell in the treatment of a fibroblast activation protein (FAP)-expressing cancer, which adapter has or comprises one of the following structures: 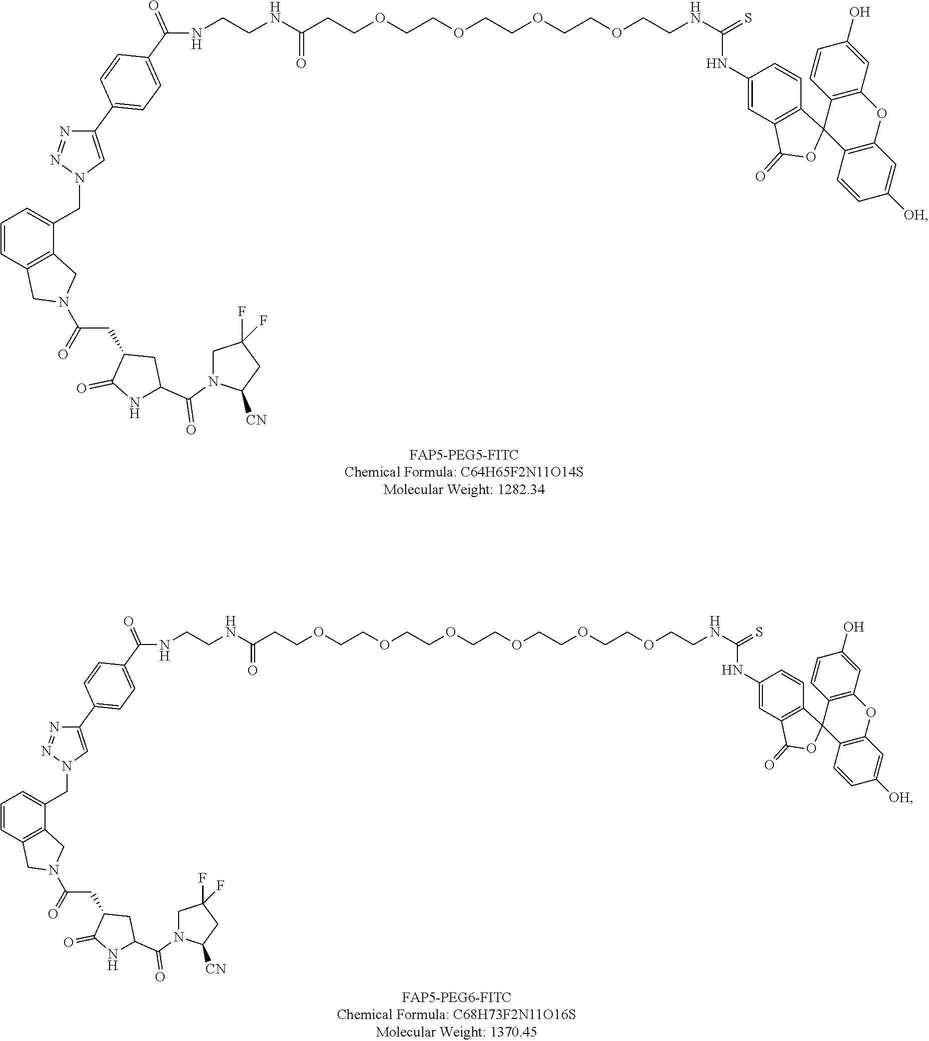 [SMILES: N#C[C@@H]1CC(F)(F)CN1C(=O)C1C[C@@H](CC(=O)N2Cc3cccc(Cn4cc(-c5ccc(C(=O)NCCNC(=O)CCOCCOCCOCCOCCNC(=S)Nc6ccc7c(c6)C(=O)OC76c7ccc(O)cc7Oc7cc(O)ccc76)cc5)nn4)c3C2)C(=O)N1 ; N#C[C@@H]1CC(F)(F)CN1C(=O)C1C[C@@H](CC(=O)N2Cc3cccc(Cn4cc(-c5ccc(C(=O)NCCNC(=O)CCOCCOCCOCCOCCOCCOCCNC(=S)Nc6ccc7c(c6)C(=O)OC76c7ccc(O)cc7Oc7cc(O)ccc76)cc5)nn4)c3C2)C(=O)N1] 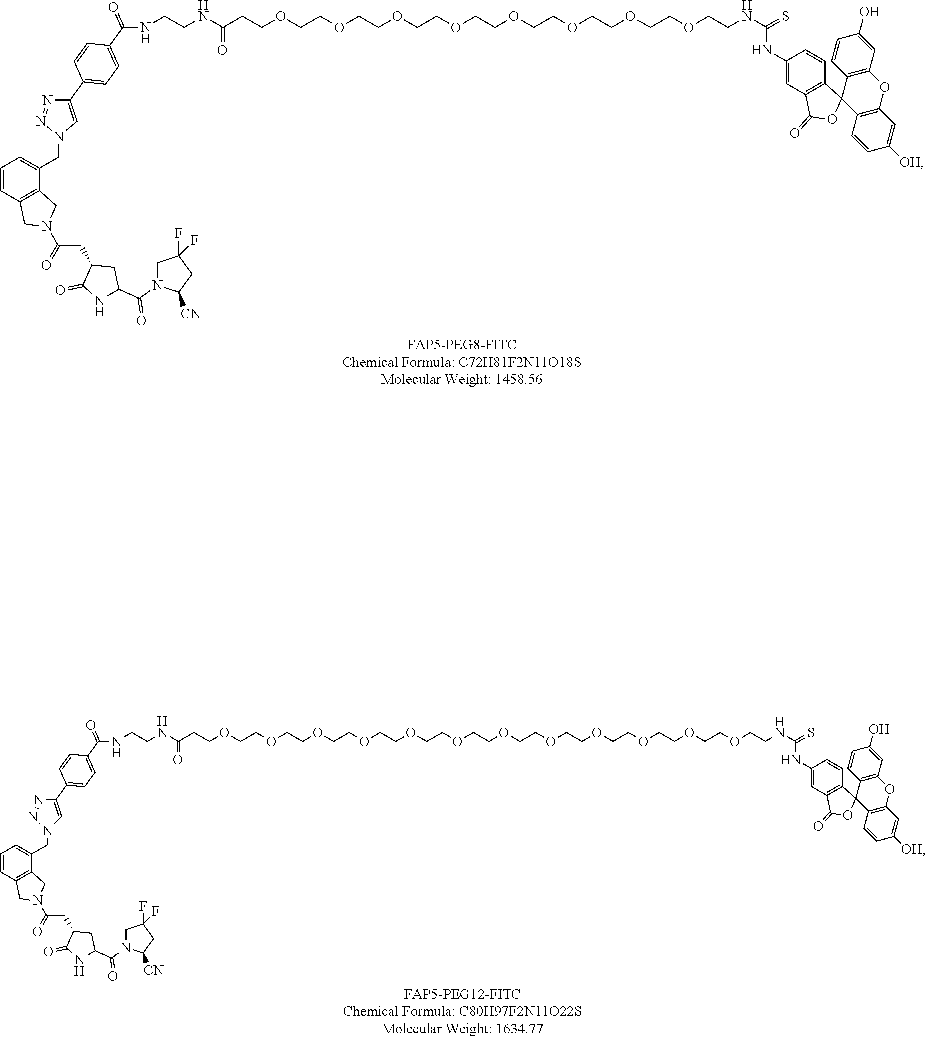 [SMILES: N#C[C@@H]1CC(F)(F)CN1C(=O)C1C[C@@H](CC(=O)N2Cc3cccc(Cn4cc(-c5ccc(C(=O)NCCNC(=O)CCOCCOCCOCCOCCOCCOCCOCCOCCNC(=S)Nc6ccc7c(c6)C(=O)OC76c7ccc(O)cc7Oc7cc(O)ccc76)cc5)nn4)c3C2)C(=O)N1 ; N#C[C@@H]1CC(F)(F)CN1C(=O)C1C[C@@H](CC(=O)N2Cc3cccc(Cn4cc(-c5ccc(C(=O)NCCNC(=O)CCOCCOCCOCCOCCOCCOCCOCCOCCOCCOCCOCCOCCNC(=S)Nc6ccc7c(c6)C(=O)OC76c7ccc(O)cc7Oc7cc(O)ccc76)cc5)nn4)c3C2)C(=O)N1] 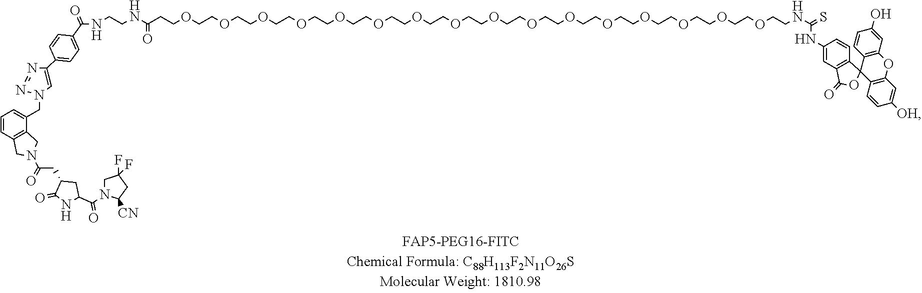 [SMILES: N#C[C@@H]1CC(F)(F)CN1C(=O)C1C[C@@H](CC(=O)N2Cc3cccc(Cn4cc(-c5ccc(C(=O)NCCNC(=O)CCOCCOCCOCCOCCOCCOCCOCCOCCOCCOCCOCCOCCOCCOCCOCCOCCNC(=S)Nc6ccc7c(c6)C(=O)OC76c7ccc(O)cc7Oc7cc(O)ccc76)cc5)nn4)c3C2)C(=O)N1]
or comprising a pharmaceutically acceptable salt or hydrate ofany of the foregoing structures.

26. A bispecific adapter for use with an anti-fluorescein chimeric antigen receptor (CAR)-T cell in the treatment of a fibroblast activation protein (FAP)-expressing cancer, which adapter has or comprises one of the following structures: 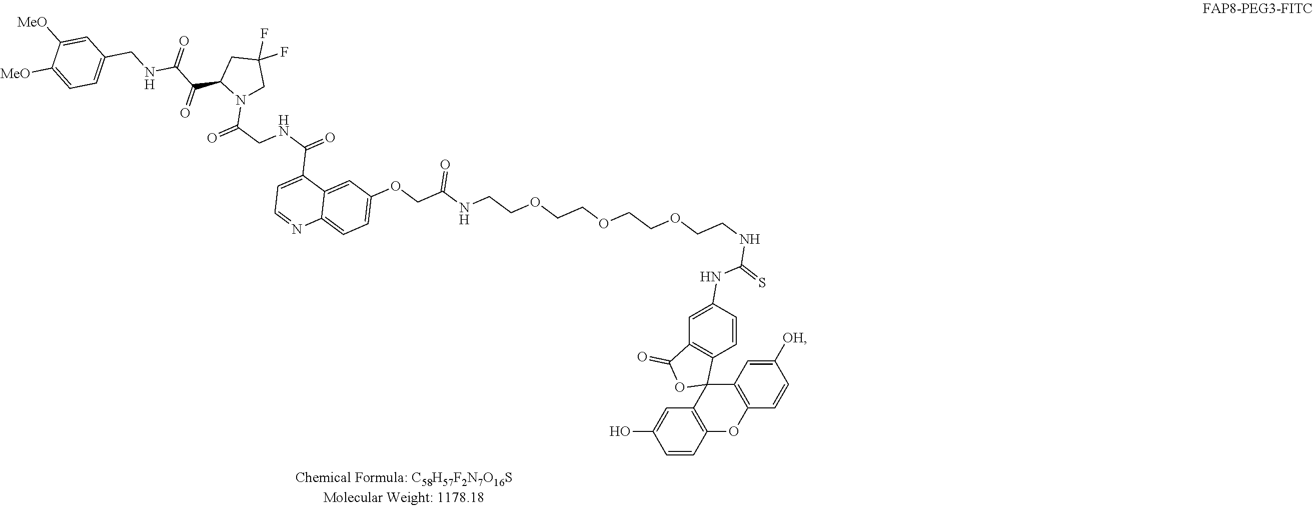 [SMILES: COc1ccc(CNC(=O)C(=O)[C@H]2CC(F)(F)CN2C(=O)CNC(=O)c2ccnc3ccc(OCC(=O)NCCOCCOCCOCCNC(=S)Nc4ccc5c(c4)C(=O)OC54c5cc(O)ccc5Oc5ccc(O)cc54)cc23)cc1OC] 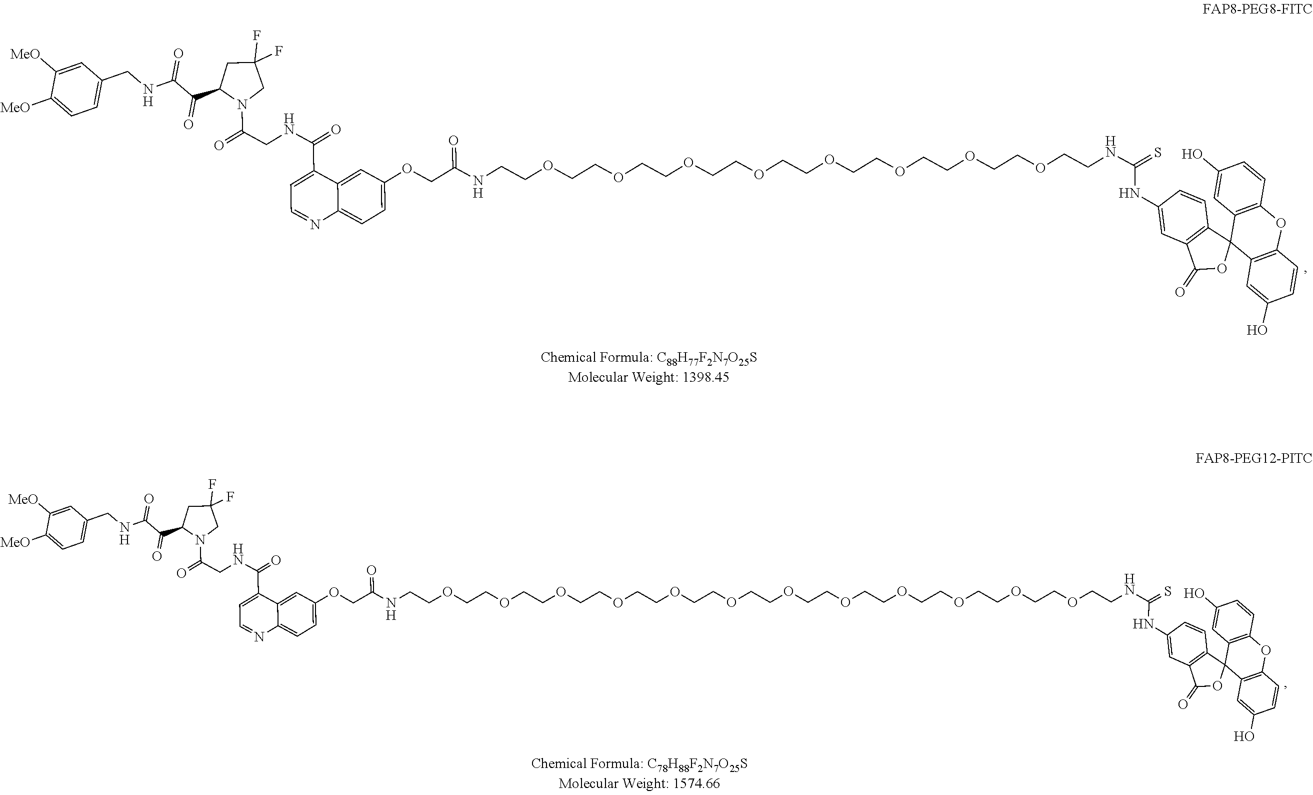 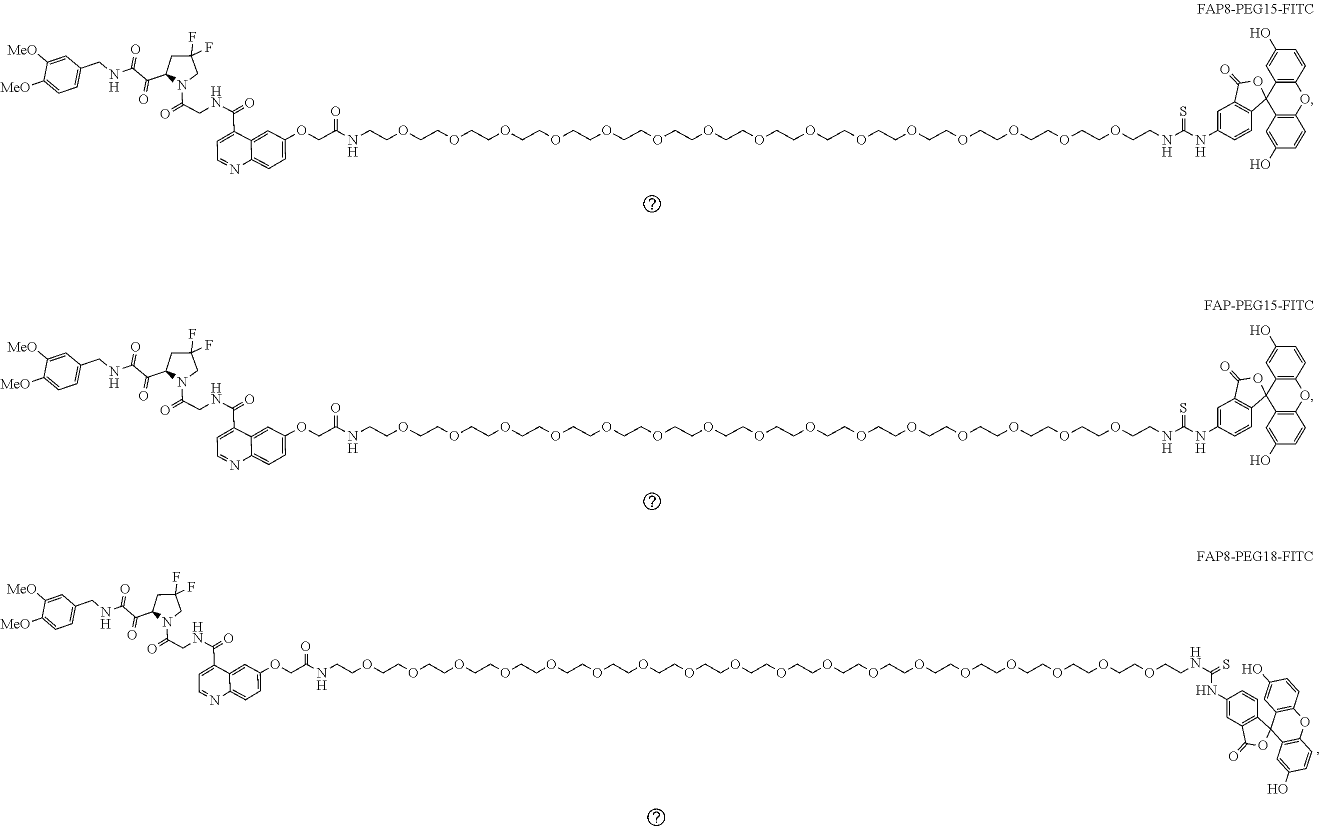 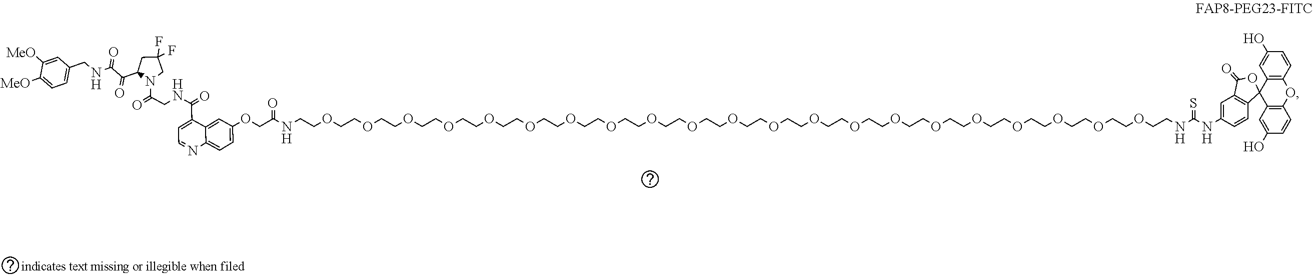 [SMILES: COc1ccc(CNC(=O)C(=O)[C@H]2CC(F)(F)CN2C(=O)CNC(=O)c2ccnc3ccc(OCC(=O)NCCOCCOCCOCCOCCOCCOCCOCCOCCOCCOCCOCCOCCOCCOCCOCCOCCOCCOCCOCCOCCOCCNC(=S)Nc4ccc5c(c4)C(=O)OC54c5cc(O)ccc5Oc5ccc(O)cc54)cc23)cc1OC]
or comprising a pharmaceutically acceptable salt or hydrate of any of the foregoing structures.

27. A bispecific adapter for use with an anti-fluorescein chimeric antigen receptor (CAR)-T cell in the treatment of a prostate-specific membrane antigen (PSMA) cancer, which adapter has or comprises one of the following structures: 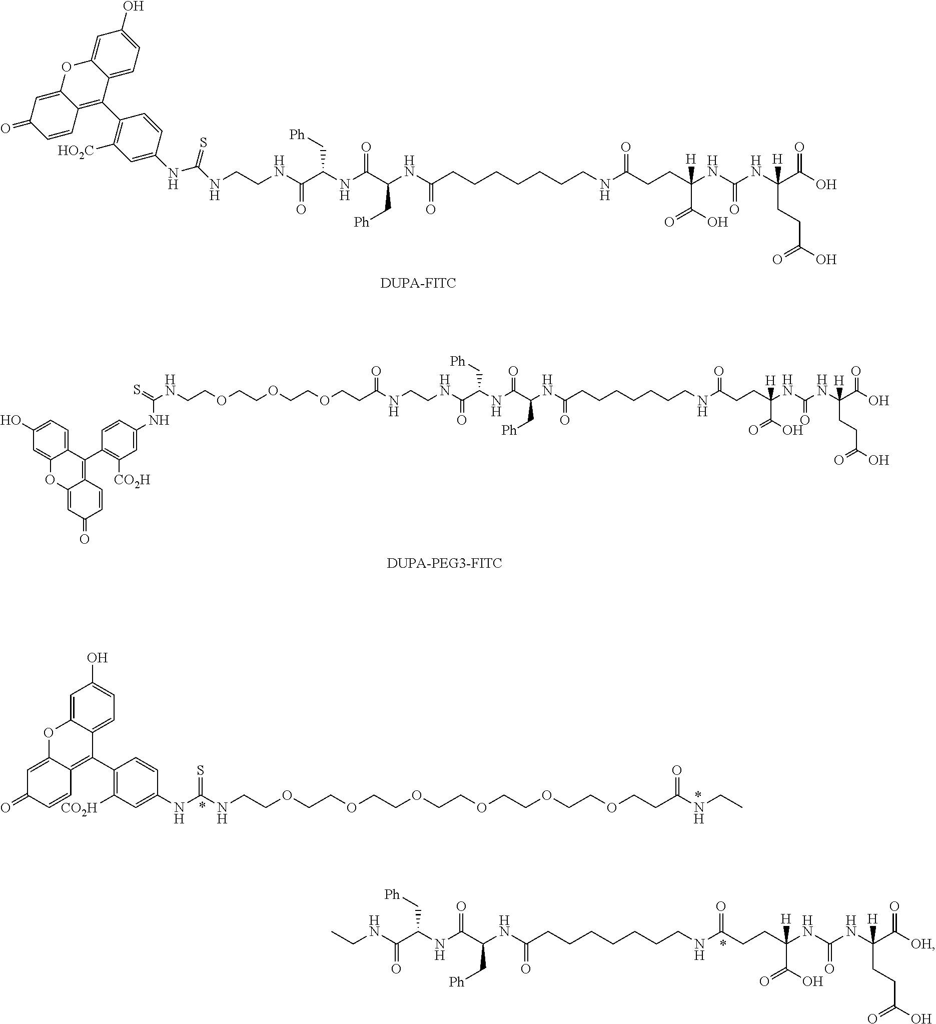 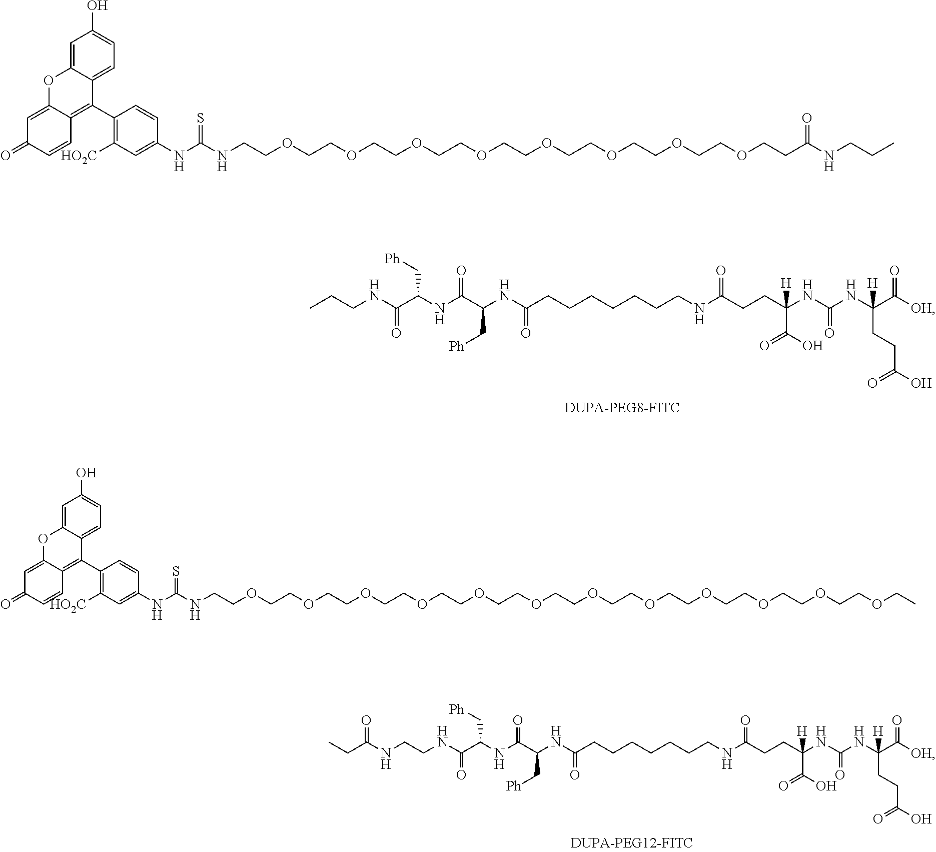 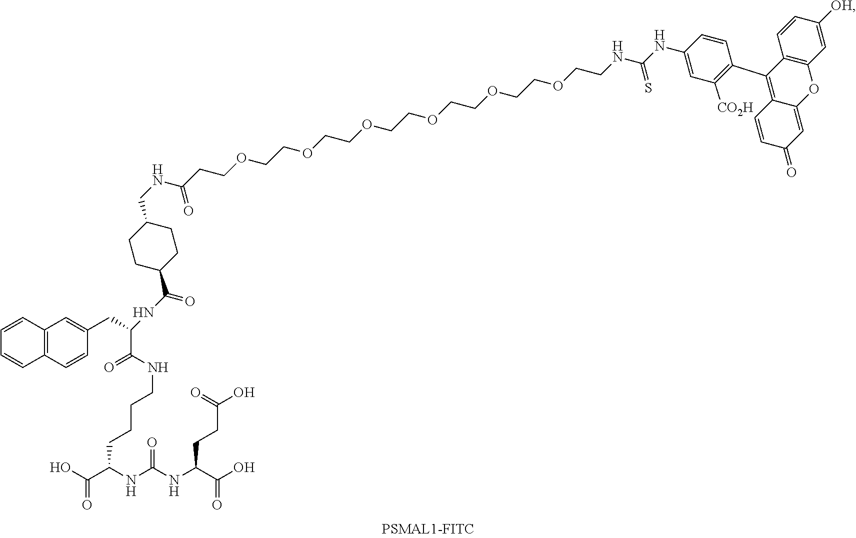 [SMILES: O=C(O)CC[C@H](NC(=O)N[C@@H](CCCCNC(=O)[C@H](Cc1ccc2ccccc2c1)NC(=O)[C@H]1CC[C@H](CNC(=O)CCOCCOCCOCCOCCOCCOCCNC(=S)Nc2ccc(-c3c4ccc(=O)cc-4oc4cc(O)ccc34)c(C(=O)O)c2)CC1)C(=O)O)C(=O)O]
or comprising a pharmaceutically acceptable salt or hydrate of any of the foregoing.

28. A bispecific adapter for use with an anti-fluorescein chimeric antigen receptor (CAR)-T cell in the treatment of prostate-specific membrane antigen (PSMA)-expressing cancer, which adapter has the structure: 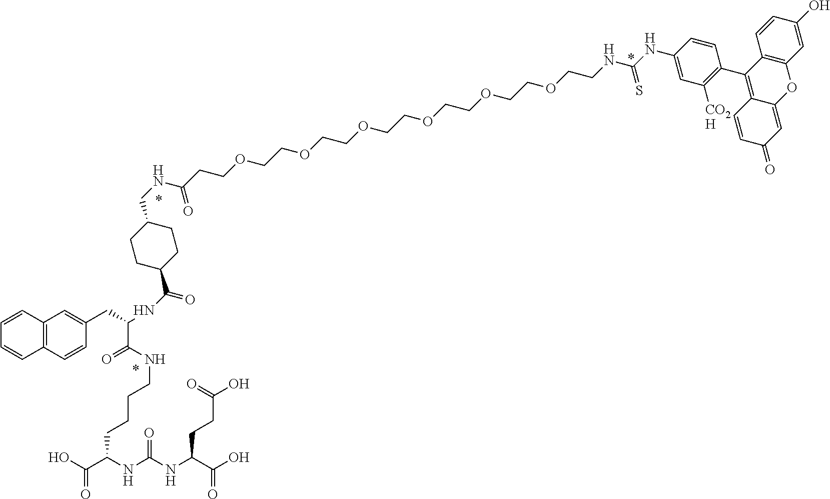 [SMILES: O=C(O)CC[C@H](NC(=O)N[C@@H](CCCCNC(=O)[C@H](Cc1ccc2ccccc2c1)NC(=O)[C@H]1CC[C@H](CNC(=O)CCOCCOCCOCCOCCOCCOCCNC(=S)Nc2ccc(-c3c4ccc(=O)cc-4oc4cc(O)ccc34)c(C(=O)O)c2)CC1)C(=O)O)C(=O)O]
a pharmaceutically acceptable salt or hydrate thereof.

29. A bispecific adapter for use with an anti-fluorescein chimeric antigen receptor (CAR)-T cell in the treatment of prostate-specific membrane antigen (PSMA)-expressing cancer, which adapter has the structure: 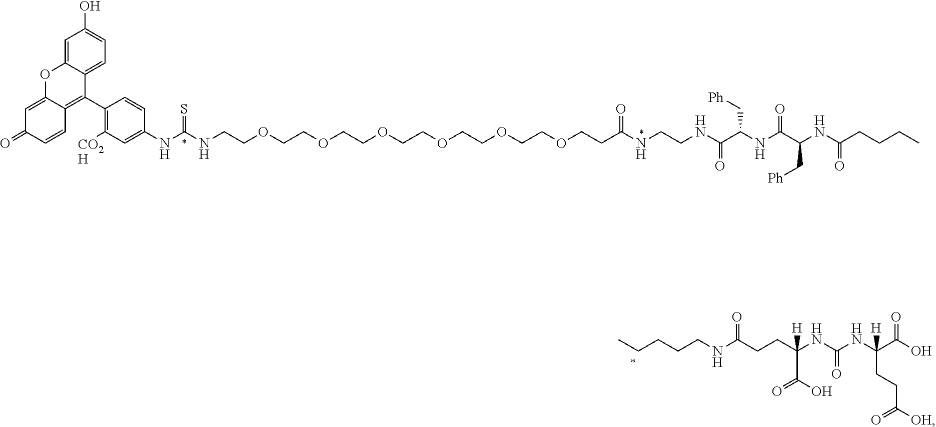
or is a pharmaceutically acceptable salt or hydrate thereof.

30. A kit comprising:
(i) at least one dosage unit of a bispecific adapter of any one of claims 1-10 or 25-29, a pharmaceutical composition comprising a bispecific adapter of any one of claims 1-10 or 25-29 and a pharmaceutically acceptable carrier or excipient, or a combination of any one of claims 12-24; and
(ii) at least one dosage unit of an anti-fluorescein chimeric antigen receptor (CAR)-T cells or a pharmaceutical composition comprising anti-fluorescein CAR-T cells and a pharmaceutically acceptable carrier or excipient;
wherein (i) and (ii) are optionally in separate containers.

31. A method of treating cancer in a subject comprising administering to the subject cancer-treatment effective amounts of:
(i) anti-fluorescein chimeric antigen receptor (CAR)-T cells or a pharmaceutical composition comprising anti-fluorescein CAR-T cells and a pharmaceutically acceptable carrier or excipient; and
(ii) a bispecific adapter of any one of claims 1-10 and 25-29, a pharmaceutical composition of claim 11, or a combination of any one of claims 12-24;
whereupon the subject is treated for cancer.

32. The method of claim 31, wherein the CAR comprises:
a recognition region comprising a single chain fragment variable (scFv) region of an anti-fluorescein antibody;
a co-stimulation domain and the co-stimulation domain is CD28, CD137 (4-1BB), CD134 (OX40), or CD278 (ICOS); and/or
an activation signaling domain that is a T cell CD3ζ chain or an Fc receptor 7.

33. The method of claim 31, wherein the fluorescein of the bispecific adapter binds the anti-fluorescein CAR-T cell with affinity upon exposure thereto, and the targeting ligand of the bispecific adapter links the bound anti-fluorescein CAR-T cell to a targeted cancer cell or cancer-associated fibroblast (CAF) upon the targeted ligand of the bispecific adapter binding a receptor on such targeted cancer cell or CAF with affinity.

34. The method of claim 33, wherein the receptor on the targeted cancer cell or CAF is an overexpressed fibroblast activation protein (FAP), an over-expressed prostate-specific membrane antigen (PSMA), and/or a folate receptor (FR).

35. The method of claim 31, wherein (i) and (ii) are administered simultaneously or sequentially, in either order, by the same or different routes.

36. The method of claim 31, wherein (ii) comprises the combination of any one of claims 25-29 and the first and second bispecific adapters are administered to the subject simultaneously by the same or different routes.

37. The method of claim 31, wherein (ii) comprises the combination of any one of claims 25-29 and the first and second bispecific adapters are administered to the subject sequentially, in either order, by the same or different routes.

38. The method of any one of claims 31-37, wherein (i) and (ii) are each administered intravenously.

39. The method of claim 31, wherein the cancer is a FAP-expressing cancer and at least one bispecific adapter of (ii) comprises a radical of a FAP ligand.

40. The method of claim 31, wherein the cancer is a prostate-specific membrane antigen (PSMA)-expressing cancer and at least one bispecific adapter of (ii) comprises a radical of a PSMA ligand.

41. The method of claim 31, wherein the cancer is a folate receptor-expressing cancer and (ii) comprises a combination of any one of claims 12-15, 17, and 18.

42. A method of treating fibroblast activation protein (FAP)-expressing cancer in a subject, which method comprises administering to the subject cancer-treatment effective amounts of
(i) anti-fluorescein chimeric antigen receptor (CAR)-T cells or a pharmaceutical composition comprising anti-fluorescein CAR-T cells and a pharmaceutically acceptable carrier or excipient; and
(ii) the bispecific adapter of any one of claims 1-5 or a pharmaceutical composition comprising the same and a pharmaceutically acceptable carrier or excipient, whereupon the subject is treated for cancer.

43. The method of claim 42, wherein the CAR has a recognition region and the recognition region is a single chain fragment variable (scFv) region of an anti-fluorescein antibody.

44. The method of claim 42 or 43, wherein the CAR comprises:
a co-stimulation domain and the co-stimulation domain is CD28, CD137 (4-1BB), CD134 (OX40), or CD278 (ICOS); and/or
an activation signaling domain and the activation signaling domain is a T cell CD3ζ chain or an Fc receptor 7.

45. A method of treating cancer in a subject comprising administering to the subject cancer-treatment effective amounts of:
(i) anti-fluorescein chimeric antigen receptor (CAR)-T cells or a pharmaceutical composition comprising anti-fluorescein CAR-T cells and a pharmaceutically acceptable carrier or excipient; and
(ii) a combination of any one of claims 12-24;
whereupon the subject is treated for cancer.

46. The method of claim 45, wherein the CAR comprises:
a recognition region comprising a single chain fragment variable (scFv) region of an anti-fluorescein antibody;
a co-stimulation domain and the co-stimulation domain is CD28, CD137 (4-1BB), CD134 (OX40), or CD278 (ICOS); and/or
an activation signaling domain that is a T cell CD3ζ chain or an Fc receptor 7.

47. The method of claim 45, wherein (i) and (ii) are administered simultaneously or sequentially, in either order, by the same or different routes.

48. The method of claim 45, wherein the first and second bispecific adapters of the combination are administered to the subject simultaneously by the same or different routes.

49. The method of claim 45, wherein the first and second bispecific adapters of the combination are administered to the subject sequentially, in either order, by the same or different routes.

50. The method of any one of claims 45-49, wherein (i) and (ii) are each administered intravenously.

51. The method of any one of claims 31-49 further comprising imaging the cancer in the subject.

52. The method of claim 51, wherein imaging the cancer comprises imaging by optical imaging, positron emission tomography (PET), or single photon emission computed tomography (SPECT).

53. The method of any one of claims 31-49, wherein the cancer is ovarian cancer, endometrial cancer, breast cancer, glioma such as, optionally, stage 3-4 glioma, or clear cell renal cell carcinoma such as, optionally, stage 3-4 clear cell renal cell carcinoma.

54. A method for enhancing chimeric antigen receptor (CAR)-T cell activation comprising:
providing a bispecific adapter of any one of claims 1-10 and 25-29, a pharmaceutical composition of claim 11, or a combination of any one of claims 12-24; and
exposing anti-fluorescein CAR-T cells or a pharmaceutical composition comprising anti-fluorescein CAR-T cells and a pharmaceutically acceptable carrier or excipient to the bispecific adaptor, pharmaceutical composition or combination;
wherein the CAR-T cell experiences enhanced activation following exposure as compared to a CAR-T cell not exposed to the bispecific adapter.

55. The method of claim 54, wherein the anti-fluorescein CAR-T cells are in systemic circulation in a subject when exposed to the bispecific adaptor.
